(19) United States
(12) Patent Application Publication
(10) Pub. No.: US 2012/0096591 A1
(43) Pub. Date: Apr. 19, 2012

(54) Identification and Validation of Novel Targets for Agrochemicals

(76) Inventors: Dirk Inze, Moorsel-Aalst (BE); Willem Broekaert, Dilbeek (BE)

(21) Appl. No.: 13/210,461

(22) Filed: Aug. 16, 2011

Related U.S. Application Data
(63) Continuation of application No. 10/510,871, filed on Jun. 2, 2005, now abandoned.

(30) Foreign Application Priority Data
Apr. 10, 2002 (EP) 02 447 062.7

(51) Int. Cl. A01H 1/06; A01H 5/00; C12N 15/12

(52) U.S. Cl. 800/288; 536/23.5; 800/298

(57) ABSTRACT

The invention relates to a method for identifying and validating plant targets for agrochemicals, comprising the steps of determining gene or protein expression profiles in function of the progression of an essential biological process in a plant subsequent downregulation of expression of said gene or protein in a plant cell. More particularly, the effects of downregulation of the candidate target gene were directly monitored on plants locally infected with a vector mediating viral induced gene suppression in that infected plant area. The invention also relates to isolated plant genes encoding proteins involved in plant growth and development. The invention also relates to plants tolerant to agrochemicals such as herbicides or pesticides.

The invention relates to isolated plant genes encoding proteins essential for plant growth and development and to methods for identifying and validating these genes/proteins as target genes/proteins for agrochemicals, such as herbicides. A target for an agrochemical is a gene or a protein where the agrochemical interferes with when applied to the target organism.

For the identification and validation of useful agrochemicals, the agrochemical industry traditionally relied on in vivo screening methods wherein chemical compounds were brought into direct contact with the living target organisms (e.g. plants for herbicide screening, insects for insecticide screening, etc.). However due to (i) the dramatic increase in the number of compounds that need to be screened to find a successful new agrochemical product, and (ii) the need to rely on very small quantities of compound such as are available in a combinatorial chemistry based compound libraries, and (iii) the need to identify compounds with a novel mode of action, the industry has developed a considerable interest in using more efficient and faster in vitro screening methods.

To render such in vitro screening methods more successful, it is essential to carefully select the tested target gene/proteins and/or the tested agrochemicals. It has been described that a more practical in vitro approach for finding new agrochemicals would involve identification of target genes/proteins against which the agrochemical compounds could possibly work. For this process identification of suitable target genes/proteins, the conventional methods make use of gene knock-outs of the target organism. Gene knock-out libraries are generally made as a random collection of thousands of gene knock-outs. In these methods it is investigated if the gene/protein is essential for the growth and/or viability of the organism, since the knockout of an essential gene (when present in a homozygous state) leads to a lethal or otherwise detrimental effect on the organism. The indication that said gene/protein is essential to the organisms makes it a suitable target for an agrochemical. These conventional methods are still cumbersome and time consuming because of the use of gene-knockouts. Other techniques that are useful to estimate the essential character of a gene or its corresponding protein are based on the downregulation of said gene or protein for example via anti-sense expression technology (WO0107601).

To render an in vitro screening for agrochemicals more successful, it is essential to carefully select the tested target gene/proteins. Therefore a more practical in vitro approach for finding new agrochemicals could be a multistep process involving the steps of (1) identification of target genes/proteins against which the agrochemical compounds could possibly work, (2) validation of the candidate target gene as being an essential gene/protein for the organism and (3) use of these target genes/proteins in an in vitro screening procedure in which the chemical compounds are tested.

It is the aim of the present invention to develop a process for the more efficient identification of candidate target genes/proteins for agrochemicals, combined with the more efficient validation of the target genes/proteins. It is a further aim of the invention to provide this process in order to design more efficiently the screening procedure with the agrochemical compound.

The method of the present invention is based on the direct use of genetic information for example generated by expression profiling of the candidate target genes/proteins, for the identification and the validation of the targets.

Therefore according to a first embodiment of the present invention, there is now provided a method for identifying and validating plant genes/proteins as targets for agrochemicals, said method comprising the steps of:
a. determining gene or protein expression profiles during a biological process of a plant or plant cell, said biological process being necessary for the viability or the growth of the plant or plant cell;
b. selecting genes or proteins having altered expression during said biological process,
c. cloning said selected gene or the nucleic acid encoding said protein in its full-length or partial form,
d. incorporating said nucleic acid in a vector designed for downregulation of expression of said nucleic acid or the sequence homologous to said nucleic acid in a plant or plant cell.

The aim of methods of the present invention is the identification of target gene(s)/protein(s) out of a broad range of candidate plant genes/proteins. The identification step is achieved by the techniques of expression profiling described in the following embodiments. Since the method of the present invention can be used for identification of genes/proteins or proteins, the term “target” as used herein can mean a gene as well as a gene product, namely a protein, polypeptide or peptide. With the expression “target for an agrochemical” is meant a protein as well as a gene or nucleic acid encoding such protein, and when such target is inhibited, stimulated or otherwise disrupted in its normal activity by an agrochemical compound, this would lead to a desired effect in a target organism. The invention aims at efficiently identifying targets for agrochemicals. Said agrochemicals can be herbicides or pesticides as well as growth stimulators or growth regulators.

Target identification means selecting candidate targets from a larger number of genes/proteins or proteins on the basis of certain properties that give such a molecule a higher probability of being a suitable target than other molecules which do not exhibit said properties.

A herbicide target is a protein or gene that when inhibited, stimulated or otherwise disrupted in its normal activity by a compound would kill the (weedy) target plant or have a strong negative effect on its growth, said compound would therefore be a candidate herbicide. An insecticide target is a protein or gene that when inhibited, stimulated or otherwise disrupted in its normal activity by a compound would kill the insect pest or have a strong negative effect on its growth, said compound would therefore be a candidate insecticide. A plant growth regulator (PGR) target is a protein or gene that when inhibited, stimulated or otherwise disrupted in its normal activity by a compound would promote or alter in a desirable way the growth of plant, said compound would therefore be a candidate PGR.

Nowadays a lot of genomic information, e.g. gene sequences, expression profiles, homologies and putative functionality, is available from genomic sequencing and expression studies in several target organisms. It is therefore of interest to develop a new method to identify and validate genes/proteins as candidate targets for agrochemicals, such methods being based on a direct use of such genomic information. This use of genomic information, e.g. the expression level of a gene, allows the selection of a limited set of appropriate candidate genes/proteins. Only this limited set of genes is then tested in the validation step, contributing to a higher efficiency and success rate of the screening procedure for agrochemicals. Furthermore, the genetic information, e.g. the functional data of the putative target gene/protein, is used as a basis to design more efficiently the in vitro screening procedure with the agrochemical compound(s) under investigation.

The present invention discloses methods that allow for the identification and validation of target genes/proteins for agrochemicals out of the broad range of possible genes/proteins and proteins. It therefore allows genes or proteins to be selected for the development of suitable in vitro screening methods for the screening of novel and efficient agrochemicals.

According to a first step of the methods of the present invention target genes or gene products are identified by using transcript profiling of the genomic content of a cell. By using this technique one immediately obtains genomic data (sequences and expression level) as well as a functional indication of the candidate target gene or gene product. Thus this method is useful for a first identification and selection of possible agrochemical target genes/proteins, since it provides as a bonus genomic and functional data on the candidate target. A good candidate target gene is a gene of which the expression varies significantly over the course of an essential biological process of the cell, since that is an indication that the gene/protein is involved in that biological process The present application describes for the first time that the determination of an expression profile of a gene during the progression of an essential biological process is used to identify possible agrochemical targets.

The expression profiling in the target identification steps of the method of the present invention is carried out in function of the progression of a process that is essential for plant growth and/or plant development and/or plant viability. In one preferred embodiment of the present invention, the essential process that is monitored in the target identification step is the process of cell division. Accordingly, in a particular embodiment of the invention, the method to identify target genes/proteins for agrochemicals is based on the transcript profiling of genes/proteins that are specifically involved in cell division. Therefore the invention provides a method as mentioned above, wherein said biological process cell division.

Other biological processes that may be monitored for the identification and validation of agrochemical targets are for instance processes that are essential for seed germination, leaf formation, etc.

The term expression profiling means determining the time and/or place when or where a gene or a protein is active. Particularly for a gene, this is achieved by monitoring the level of transcripts and therefore in the case of gene expression profiling the term transcript profiling or mRNA profiling is used.

Generally, the expression profiling in the methods of the present invention is carried out in function of the progression of a process that is essential for plant growth and/or development and/or plant viability. To achieve this, the process of interest is synchronized in a sufficient number of cells (for example in a cell culture) or organisms to allow collecting samples for expression profiling representing various stages of said process. Target identification then consists in selecting those genes or proteins that show significant changes in expression levels in function of the progression of the process of interest. It are those genes or proteins that are likely to be strongly involved or to be essential in said process.

The term “essential” means that if the gene or the gene product cannot function as normal in the cell or organism, this will have significant implication in the cell growth or cell development or other vital functions of the cell or organism.

According to the invention, the expression profiling can be studied at the level of m-RNA, using transcript profiling techniques, or alternatively at the level of protein, using proteomics-based approaches.

In one preferred embodiment of the invention, m-RNA profiling is used for identification of target genes/proteins and expression levels may be quantified via techniques that are well known to the man skilled in the art. For instance, mRNA-profiling can be performed using micro-array or macro-array technologies, this method however requires that the gene sequences are known (full length sequences or at least partial sequences) and are physically available for coating on the micro or macro array surface. Standard chips are being commercialized for Arabidopsis, and sufficient sequence information is now available for different plant species (including rice) to allow sufficient sequence data for this approach. Another approach for mRNA profiling is the use of AFLP-based transcript profiling as described in example 1. In this approach short sequence tags are monitored. In a next step these short sequence tags may be matched with full-length genes/proteins if required. Gene or protein selection thus be based on either full-length or partial sequences and it is well within the realm of the person skilled in the art to find a full length sequence based on the knowledge of a partial sequence.

Therefore, one aspect of the invention is the direct use of genetic information to select candidate targets for agrochemicals. As mentioned above this genetic information can be generated by a number of techniques. Accordingly, the present invention encompasses a method as mentioned above, wherein the expression profiles are determined by means of micro-array, macro array or c-DNA-AFLP.

According to another embodiment of the invention, proteomic based approaches may be used to identify candidate target proteins for agrochemicals.

It is now demonstrated that for the purposes of identifying a target gene for agrochemicals a synchronized culture of dividing plant cells is used to isolate samples and to monitor the expression of the transcripts of those cells during the progression of the cell division.

Therefore according to a particular embodiment, the invention also encompasses a method for the identification and validation of plant agrochemical targets, wherein said gene or protein expression profiling is based on nucleic acid or protein samples collected from a synchronized culture of dividing plant cells.

In one embodiment of the invention, the samples used for expression profiling are obtained from a synchronized culture of rice cells, tobacco cells, Arabidopsis cells or cells from any other plant species. The cell culture should be synchronized in order to obtain samples containing a sufficient amount of cells that are at the same stage of the biological process, so that the various samples taken for expression profiling are representative for the various stages of the essential biological process. In a particular embodiment of the present invention the samples are obtained from cells that are synchronized for cell division. In a preferred embodiment of the invention expression profiling is done on synchronized dividing cells. Certain cell lines are particularly suitable for synchronization of cell division, for instance synchronization of tobacco Bright Yellow-2 cell lines as described in example 1. Therefore most preferably, the synchronized cells are tobacco BY2 cells. By using synchronized tobacco BY2 cells and performing a cDNA-AFLP-based genome-wide expression analysis, the inventors built a large collection of plant cell cycle-modulated genes/proteins. Approximately 1340 periodically expressed genes/proteins were identified, including known cell cycle control genes as well as numerous novel genes. A number of plant-specific genes were found for the first time to be cell cycle modulated. Other transcript tags were derived from unknown plant genes showing homology to cell cycle-regulatory genes of other organisms. Many of the genes encode novel or uncharacterised proteins, indicating that several processes underlying cell division are still largely unknown. These sequences are presented herein as SEQ ID NO 1 to SEQ ID NO 785.

While, according to the invention, the basic criterion for identifying an agrochemical target gene or gene product consists in the differential expression levels of the gene or the protein observed during the progression of an essential biological progress, secondary selection criteria can be used and combined with this primary criterion.

One such secondary criterion may be to make a selection of genes or proteins that are found not to exhibit a high degree of homology with genes or proteins from other organisms (such as mammals) as this criterion is likely to reduce the probability that the agrochemical compounds active on the “plant-specific” target genes or gene products would also exhibit toxic effects against other organisms, for example mammals.

Another secondary selection criterion could exist in focusing on a particular phase of the essential biological process as mentioned above. For instance, when cell division modulated genes/proteins are under investigation as potential agrochemical target genes/proteins, one could preferably use those cell division modulated genes/proteins which exhibit high expression during the G1 phase, S phase, G2 phase or M phase or at the transition stages of these phases. In one embodiment of the present invention, the focus may be on the G2/M transition phase, since this phase in the plant cell cycle is considered to have more “plant specific” elements than other phases of the cell cycle and is therefore more likely to yield plant specific candidate target genes and proteins. Whereas the core cell cycle genes/proteins and the basic regulatory mechanisms controlling cell cycle progression are conserved among higher eukaryotes, basic developmental differences between plants and other organisms imply that plant-specific regulatory pathways exist that control cell division. Especially for events occurring at mitosis, plants are expected to have developed unique mechanisms regulating karyo- and cytokinesis. A typical plant cell is surrounded by a rigid wall and can as such not divide by constriction. Instead, a new cell wall between daughter nuclei is formed by a unique cytoskeletal structure called the phragmoplast, whose position is dictated by another cytoskeletal array called the preprophase band. Another major difference between plant and animal mitosis is found in the structure of the mitotic spindles: in animals, they are tightly centred at the centrosome, whereas in plants they have a diffuse appearance.

Therefore a suitable second criterion to combine with the first criterion may be to select genes/proteins that are involved in the mitosis step of the cell cycle and/or that are involved in the building of the cell wall during mitosis.

Likewise a secondary selection criterion to be combined with the first criterion may be the selection of genes or proteins from a dicotyledonous plant that do not exhibit a high degree of homology with genes or proteins from a monocotyledonous plant (or vice versa). This secondary criterion is especially relevant when identifying agrochemical target genes or proteins with the intention to selectively identify targets that would allow for subsequence screening of selective herbicides or plant growth regulators. For instance, this strategy is advantageous to find targets and agrochemicals for selective weed control, such as herbicides that kill dicotyledonous weeds in monocotyledonous crops or vice versa.

Therefore according to further embodiments, the present invention encompasses methods as mentioned above, wherein the target gene or protein meets any one or more of the above mentioned secondary selection criteria, such as being plant specific, being mitosis specific or being dicot specific etc.

The possibility for combination of criteria used for selecting target genes or proteins renders the method of the present invention more powerful than classical methods. According to a preferred embodiment the technique of the present invention allows identifying genes/proteins, to be used as agrochemical target genes/proteins, these genes being genes/proteins that are involved in cell division and control of cell cycle progression, and these genes being novel and these genes being plant specific. Therefore the method of the present invention is characterized in that it allows identifying new and unexpected agrochemical targets.

In the target gene identification step according to the present invention, genes or proteins are selected for which there is a high probability of being essential. It should be clear that the above-mentioned examples are given by way of illustration and are not meant to be limiting in any way.

Further, according to a second step in the method of the invention, the candidate agrochemical target gene or gene product is subsequently validated as being essential for the growth and/or development and/or viability of the organism. This is achieved by cloning the identified candidate target gene in a vector construct designed to downregulate said target gene in a plant or plant cell, followed by inoculating the plant with this construct and monitoring whether downregulation of the gene results in negative effects on plant growth and/or development and/or viability. A valid target gene is a target gene that causes significant effects on growth of plants or plant cells when downregulated. The present application describes for the first time the use of a particularly fast and efficient downregulation method to validate possible agrochemical targets.

Accordingly, the present invention encompasses a method as mentioned above for the identification and validation of plant targets for agrochemicals, wherein said downregulation involves a viral-induced gene silencing mechanism.

Thus, starting from a number of candidate target genes/proteins identified in the first step of the method of the invention, the target validation step aims at confirming and demonstrating the essential nature of the gene by demonstrating that severe down-regulation of the expression level of the gene has a significant effect on the organism.

In particular, when one is interested in developing a screening assay for herbicides, downregulation of the candidate target gene in a plant may result in a lethal effect, a severe inhibition of plant growth or any other (obviously) negative phenotypic effects. Alternatively, when one is interested in developing a screening assay for plant growth regulators, the effect of downregulating the target gene may be modulation or even stimulation of growth in general or modulation or even stimulation of a particular process associated with plant growth and/or development and/or architecture and/or physiology and/or biochemistry or any other phenotypic effect.

The man skilled in the art will be aware of various methods to achieve downregulation of a given gene or protein, such methods include essentially co-suppression based approaches or anti-sense based approaches as well as any other method resulting in gene silencing. Other examples of downregulation in a cell are well documented in the art and include, for example, RNAi techniques, the use of ribozymes etc. Gene silencing may also be achieved by insertion mutagenesis (for example, T-DNA insertion or transposon insertion) or by gene silencing strategies as described by, among others, Angell and Baulcombe, 1998 (WO 98/36083), Lowe et al., 1989 (WO 98/53083), Lederer et al., 1999 (WO 99/15682) or Wang et al., 1999 (WO 99/53050). Expression of an endogenous gene may also be reduced if the endogenous gene contains a mutation.

The effect of gene downregulation can be observed in stably transformed plants which can be obtained by means of various well known techniques, these techniques generally involving a plant transformation step and a plant regeneration step.

Genes/proteins which exhibit a severe negative effect when downregulated may however significantly reduce transformation and/or regeneration efficiency. Therefore, a relevant parameter indicative for the essential nature of the gene, may be a severe reduction in transformation efficiency when said particular gene is used in a down-regulation construct. In order to avoid the (negative) effect on transformation efficiency in the transformation and regeneration process, an inducible promoter system can be used. Induction of promoter activity can then be applied at a later stage (after transformation) in order to observe the effect of gene downregulation once the transformed plant or plantlet started to develop.

Further, another method for testing the effect of downregulation of a target gene, which can be used in the methods of the present invention, is based on a rapid transient transformation process and does not rely on the somewhat lengthy process of stable transformation. The use of this method for target validation in plants is part of this invention, regardless of whether target identification has been performed according to this invention.

Accordingly, in a preferred embodiment, the downregulation method is based on co-suppression and on rapid transient transfection of plant cells. The preferred method to validate genes/proteins as targets for agrochemicals is based on the cloning of the identified candidate target gene in a vector construct containing a viral replicase that is involved in the very efficient downregulation of the candidate target gene in the infected plant or plant cell via the mechanism of co-suppression. One advantage of this method for downregulation, is the fact that the infection of the host cells or the plant can be performed locally for example by inoculating the vector directly on the leaves. This allows a very fast evaluation of the effect of downregulating the candidate target since no complete transgenic plants have to be generated. Also this technique allows an easy way of monitoring the effect of the downregulated candidate target by simply looking at the changes of the infected place, for example monitoring the lethal effects on the infected leaf).

Therefore in a preferred embodiment, the downregulation method is based on co-suppression. In a more preferred embodiment of the invention this co-suppression technique is fast and easy to evaluate the effect of downregulation, so that it is suitable for dealing with high numbers of genes/proteins. This can be achieved by using viral induces gene silencing mechanisms (VIGS) and by infecting the plant directly and locally, for example on the leaves. Therefore, according to another embodiment, the present invention relates to the use of a viral-induced gene silencing system for validating plant targets for agrochemicals.

This method for severe downregulation via transient expression of the gene in the presence of certain viral elements is referred to as “virus-induced gene silencing mechanism” (VIGS) and is previously described in Ratcliff et al., Plant J., 25 237-245, 2001. Briefly, virus vectors carrying host-derived sequence inserts induce silencing of the corresponding genes/proteins in infected plants. This virus-induced gene silencing is a manifestation of an RNA-mediated defence mechanism that is related to post-transcriptional gene silencing in transgenic plants. Ratcliff et al., developed an infectious cDNA clone of Tobacco rattle virus (TRV) that has been modified to facilitate insertion of non-viral sequences and subsequent infection in plants. This vector mediates VIGS of endogenous genes/proteins in the absence of virus-induced symptoms. Unlike the other RNA virus vectors that have been used previously for VIGS, the TRV construct is able to target most RNA's in the growing points of the plant. A more detailed description of this downregulation mechanism is given in example 2.

According to particular embodiments of the present invention, the VIGS system is applied in Arabidopsis or in tobacco for the purposes of validation of a candidate agrochemical target gene.

According to a further preferred embodiment, there is provided a method for validation of a candidate agriochemical target gene, wherein the gene is downregulated in a plant via the use of infectious DNA of virus is Tobacco Rattle Virus and wherein said plant is tobacco.

The present invention relates to a combination of the above-mentioned identification and validation steps, which are especially selected so that they lead to an efficient selection of candidate target genes for agrochemicals. The outcome of the transcript profiling provides the necessary information and forms the basis for the second step, namely the validation of the target gene via incorporation of the gene sequence in the downregulation construct. The combination of these two techniques is especially useful for selecting suitable target genes/proteins for agrochemicals in a high throughput fashion. This technique thus overcomes the technical limitations of previously described techniques such as the knock-out libraries and the antisense strategies without genetic information of the genes. This new combination offers a time-saving strategy for identification of a candidate target gene and the more direct information output in the form of a real sequence, the immediate cloning of the gene in the downregulation construct and immediate application of the downregulating construct on the target organism.

The combination of these steps offers the unique opportunity to provide many high quality target genes/proteins for agrochemicals in a commercially and economically advantageous way. Furthermore, inherent to the techniques of the present invention is that the qualified target genes/proteins are accompanied with the necessary information to design a suitable in vitro screening assay with the agrochemical. This information consists of the expression characteristics of the genes/proteins and their function and importance in the essential biological process that was monitored during the transcript profiling.

In this way, the methods of the present invention overcome the practical and commercial limitations of the existing techniques.

Once this level of target validation is reached, the validated target can be selected for the development of an appropriate high-throughput in vitro screening method, wherein the agrochemical is tested. Therefore, the present invention also encompasses a method for screening candidate agrochemical compounds, comprising the use of any of the identification procedures and/or validation procedures as mentioned above. More particularly, the present invention encompasses a method for screening agrochemical compounds, comprising the use of any one or more of the sequences represented in SEQ ID NO 1 to 785.

Various methods can be used to develop suitable in vitro assays for screening the chemical compounds, depending on what is known about the biological activity of the target gene. For example, when the target is an enzyme, measurement of the enzymatic activity of the target could form the basis of the in vitro screening assay with the chemical compound.

Therefore, the methods of the present invention, the genes/proteins and the information generated by the combined identification and validation methods of the present invention, allow one to design and/or fine tune a screening for testing and/or developing agrochemicals (for example herbicides). For example if the expression pattern and the role of the target gene in the essential biological process is known, it is much easier to set up an in vitro screening assay to monitor the effect of a candidate herbicide on the target cells. Therefore it is expected that much more refined and/or efficient herbicides will be characterized using the methods of the present invention.

Also because of the knowledge of its function, one can further design the screened agrochemical compound to improve its activity for instance to improve its binding capacity to the target.

Therefore, the present invention encompasses a method for screening candidate agrochemical compounds comprising the use of any of the methods as mentioned above.

The invention may also be applied for the development of agrochemical (for example herbicide or pesticide) tolerant plants, plant tissues, plant seeds and plant cells.

Herbicides that exhibit greater potency can also have greater crop phytotoxicity. A solution to this problem is to develop crops that are resistant or tolerant to herbicides. Crop hybrids or varieties that are tolerant to the herbicides allow, for instance, for the use of herbicides that kill weeds without attendant risk of damaging the crop. Further it should be clear that when a plant is overexpressing the target of a particular herbicide, the tolerance of said plant against said herbicide will also be enhanced.

Therefore the present invention also relates to the use of the agrochemical (e.g. herbicide) target genes/proteins as identified by the method of the present invention for generating transgenic plants that are tolerant or resistant to an agrochemical (e.g. herbicide). Example of genes and gene sequences identified by the combined identification and validation methods of the present invention and which can be used as agrochemical target or that can be used to obtain herbicide tolerant plants comprise the sequences as represented in any of SEQ ID NOs 1 to 785.

These sequences are derived from tobacco, but the one skilled in the art can easily find via homology search in databases or homology search in a cDNA library the homologues genes of other plant species, for instance monocot sequences (e.g the corresponding rice or corn sequence), and use them for the same purposes as described herein. These homology searches can be done for example with a BLAST program (Altschul et al., Nucl. Acids Res., 25 3389-3402, 1997) on a sequence database such as the GenBank database. Homology studies as referred to above can be performed using sequences present in public and/or proprietary databases and using several bioinformatics algorithms, well known to the man skilled in the art. Methods for the alignment of sequences are well known in the art, such methods include GAP, BESTFIT, BLAST, FASTA and TFASTA. GAP uses the algorithm of Needleman and Wunsch (J. Mol. Biol. 48: 443-453, 1970) to find the alignment of two complete sequences that maximizes the number of matches and minimizes the number of gaps. The BLAST algorithm calculates percent sequence identity and performs a statistical analysis of the similarity between the two sequences. The software for performing BLAST analysis is publicly available through the National Centre for Biotechnology Information.

Further, some of the tobacco sequences identified by the method of the present invention might be partial but again, the full-length sequence can easily be found based on the partial sequence. For example “transcript building” can be done based on homology search on EST databases, cDNA's or gene predictions. These databases and programs are publicly available e.g. http://www.tigr.org/.

Therefore the present invention relates to the use of the nucleic acids as identified and disclosed herein and represented in SEQ ID NO 1 to 785, and also to the use of the full length genes regenerated from the partial sequences as well as to the use of the homologues sequences isolated from the same or from other plants.

In another embodiment, the present invention relates to a nucleic acid identified according to the method of the invention. Thus the invention encompasses an isolated nucleic acid identifiable by any of the methods as mentioned above.

In another embodiment, the invention relates to a nucleic acid identified according to the method of the invention, comprising the nucleic acid sequence chosen from the group of SEQ ID NO 1 to 785 or a full length sequence thereof, or a functional homologue thereof, or a functional fragment thereof, or an immunologically active fragment thereof. Thus the invention encompasses an isolated nucleic acid, comprising at least part of a nucleic acid sequence chosen from the group of SEQ ID NO 1 to 785 a homologue, functional fragment or derivative thereof.

With “a functional fragment” is meant any part of the sequence that is responsible for the biological function or for an aspect of the biological function of the nucleic acid sequence.

Further, the invention encompasses a method for the production of an agrochemical resistant plant, comprising the use of any one or more of SEQ ID NO 1 to 785 or a homologue, functional fragment or derivative thereof or one or more of the proteins encoded by SEQ ID NO 1 to 785 or a homologue, functional fragment or derivative thereof.

In one embodiment of the present invention the sequences, the full-length sequences and the homologues are used to develop herbicide tolerant plants.

Further the invention encompasses a plant tolerant to an agrochemical, in which the expression level of one or more of the nucleic acids corresponding the SEQ ID NO 1 to 785 or the homologue, functional fragment or derivative thereof, is modulated. Further the invention encompasses any part or more preferably any harvestable part of these plants.

Therefore the invention also relates to the use of these sequences, the full-length sequences and the homologues as targets for agrochemicals The invention encompasses the use of a nucleic acid as mentioned above or the protein encoded by said isolated nucleic acid as a target for an agrochemical compound, preferably, wherein the agrochemical compound is a herbicide.

Further, the invention relates to the use of these sequences to develop screening assays for the identification and/or development of agrochemicals. The invention encompasses a method for screening candidate agrochemical compounds comprising the use of any one or more of SEQ ID NO 1 to 785 or a homologue, functional fragment or derivative thereof or one or more of the proteins corresponding to SEQ ID NO 1 to 785 or a homologue, functional fragment or derivative thereof.

The present invention will be further illustrated by the following figures, wherein,

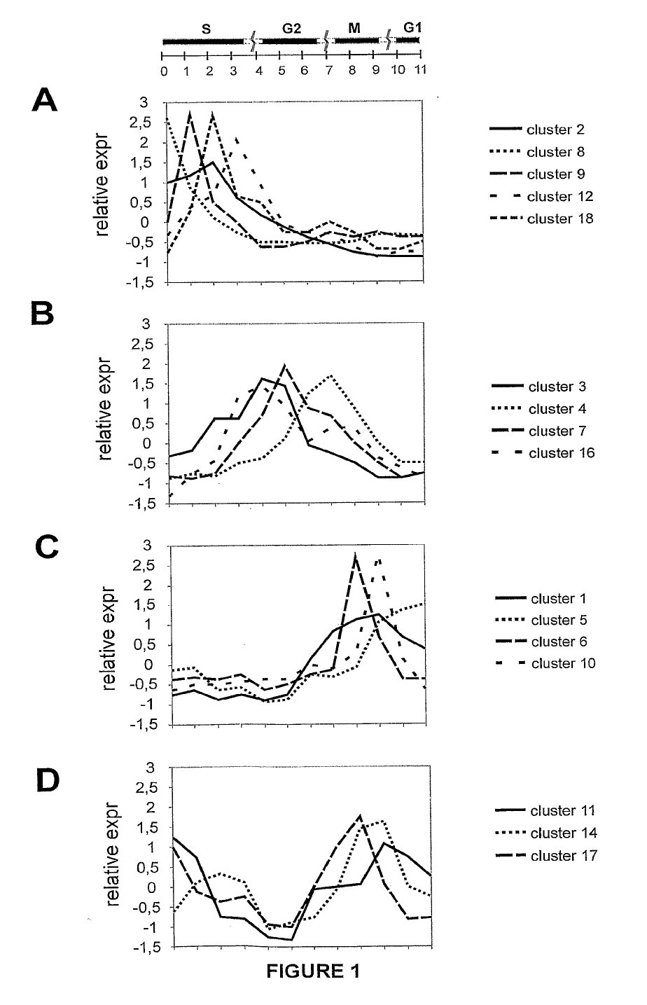

FIG. 1 shows the gene expression profiles obtained by quality-based clustering of all transcript tags monitored in a transcript profiling experiment as described in example 1. Shown are the trend lines of 16 clusters containing 97% of the genes and covering the entire time course as indicated on top. S-phase-specific gene clusters are grouped in A, gene clusters with peak expression between S- and M-phase are grouped in B, whereas group C contains the M- and G1-phase-specific clusters. D: Three small clusters of genes with peak expression during two cell cycle phases.

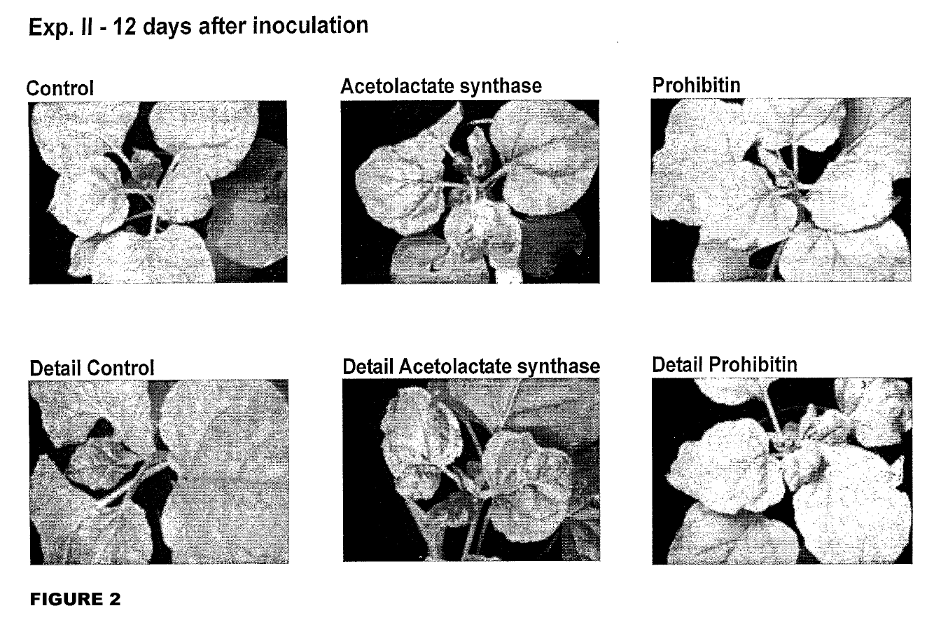

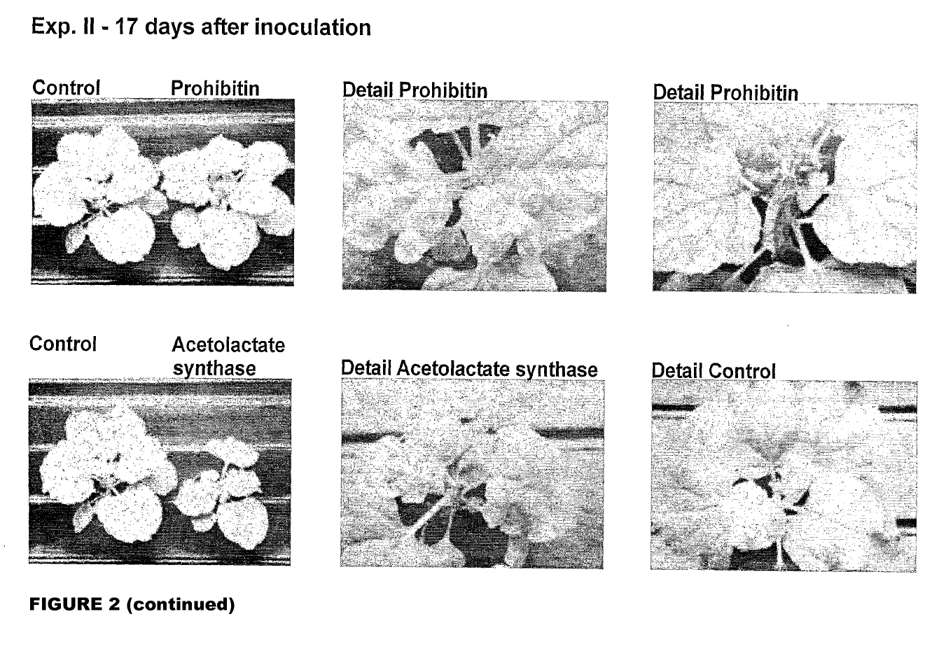

FIG. 2 shows the phenotypes of tobacco plants inoculated with a acetolactate synthase (SEQ ID NO 18) downregulation construct and phenotypes of tobacco plants inoculated with a prohibitin (SEQ ID NO 21) downregulation construct. The phenotypes were observed 12 days after inoculation (upper panel) or 17 days after inoculation (lower panel).

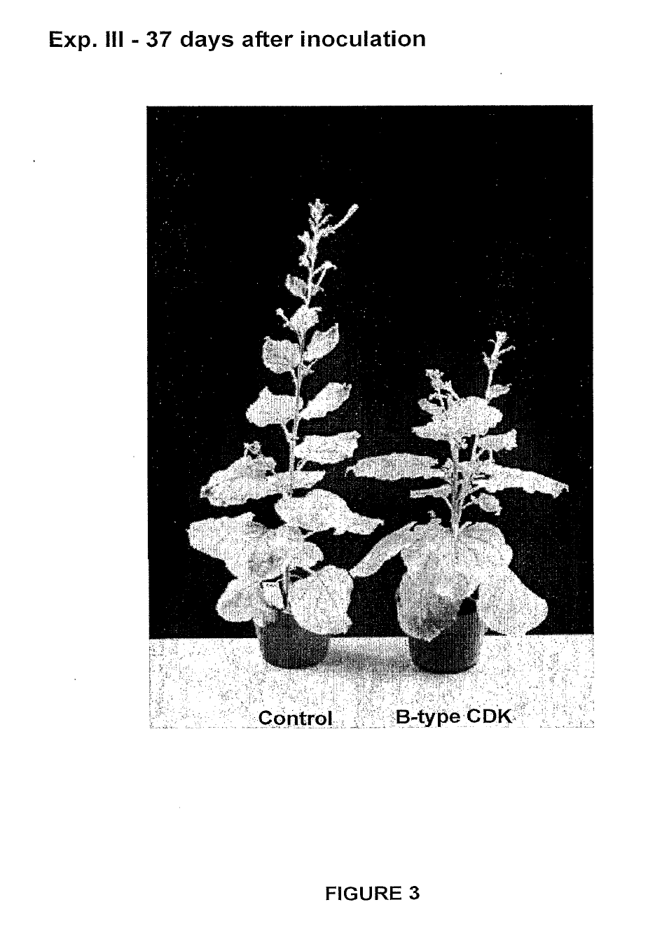

FIG. 3 shows the phenotype of tobacco plants inoculated with a B-type CDK (SEQ ID NO 11) donwregulation contruct. The observations were made 37 days after inoculation.

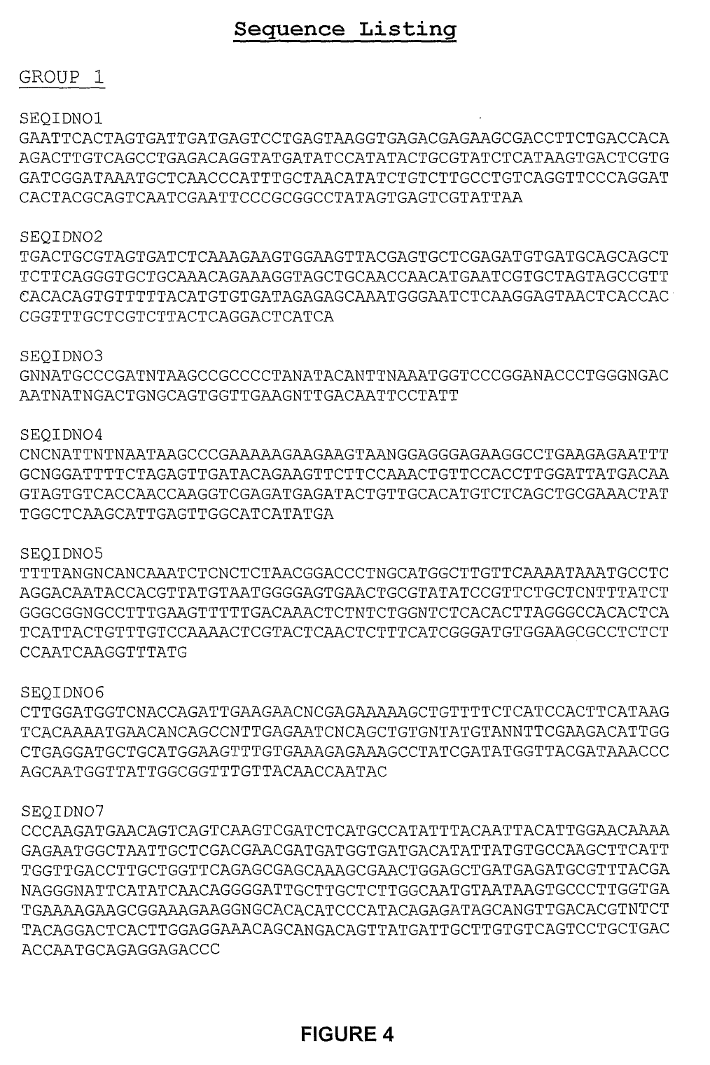

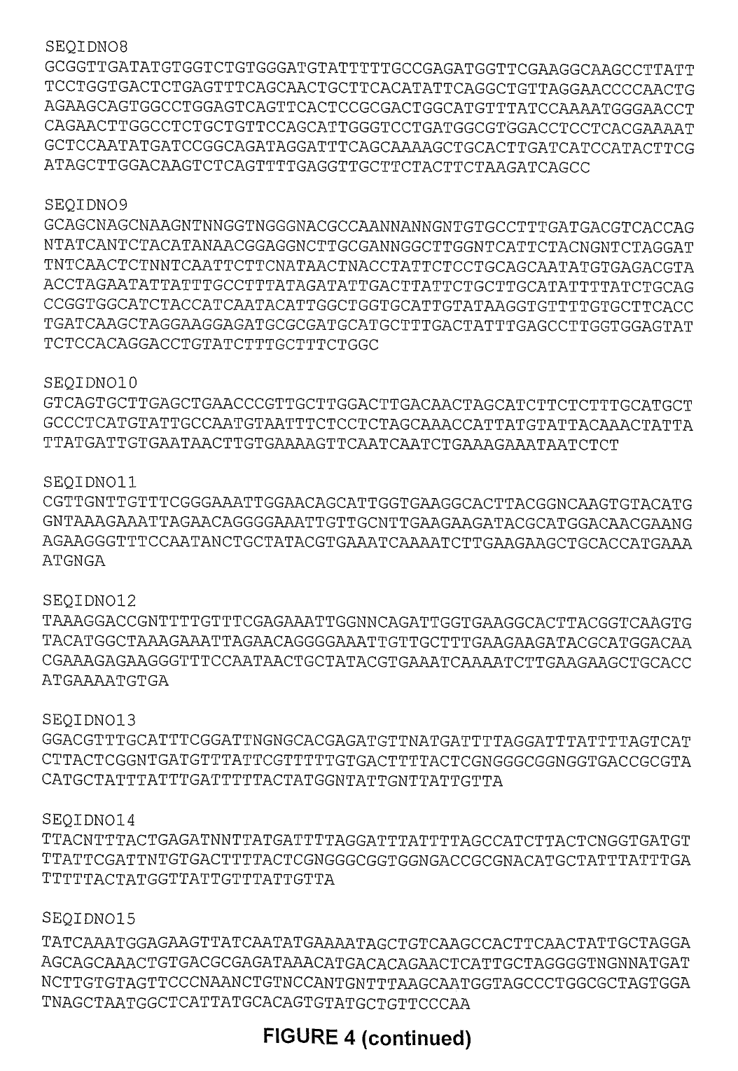

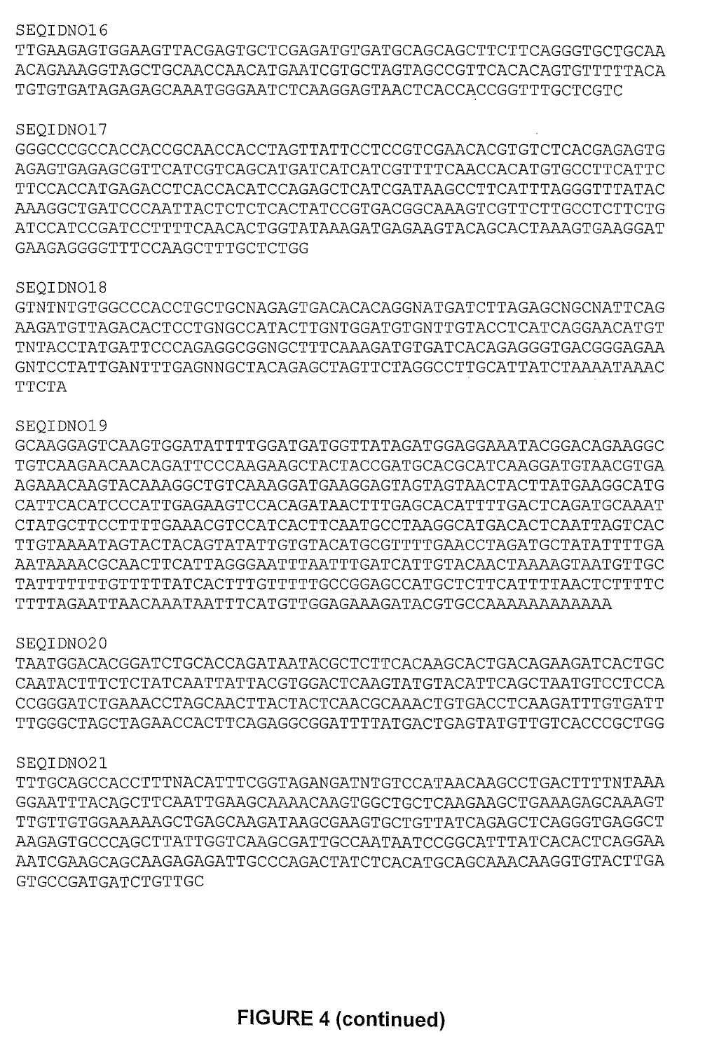

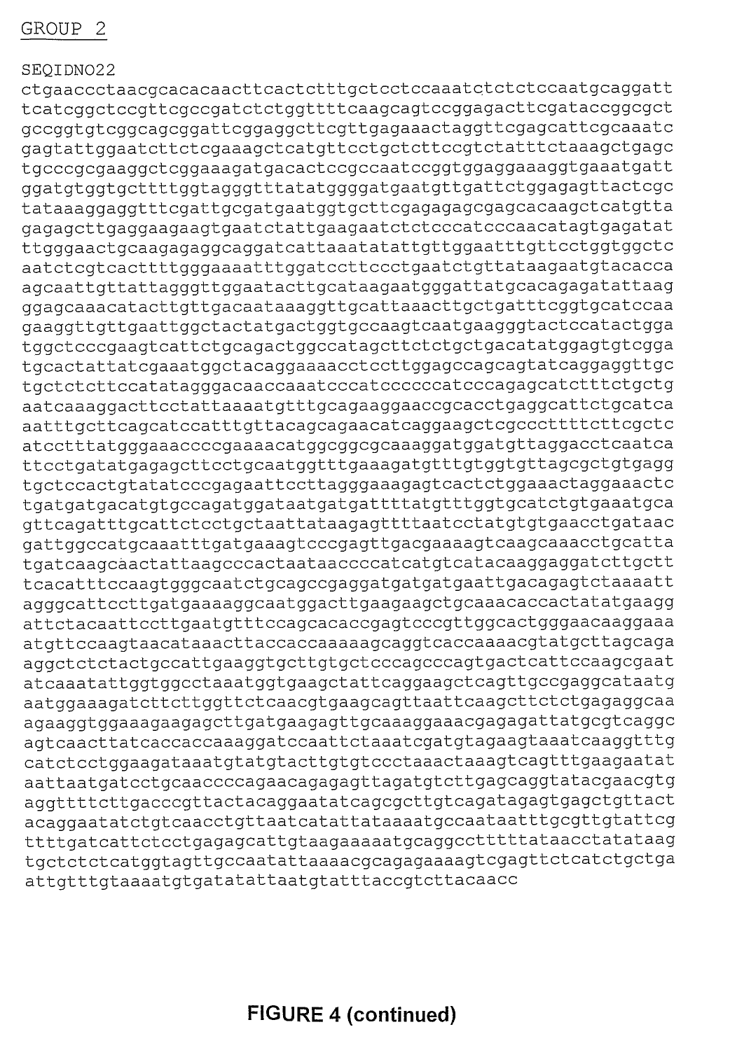

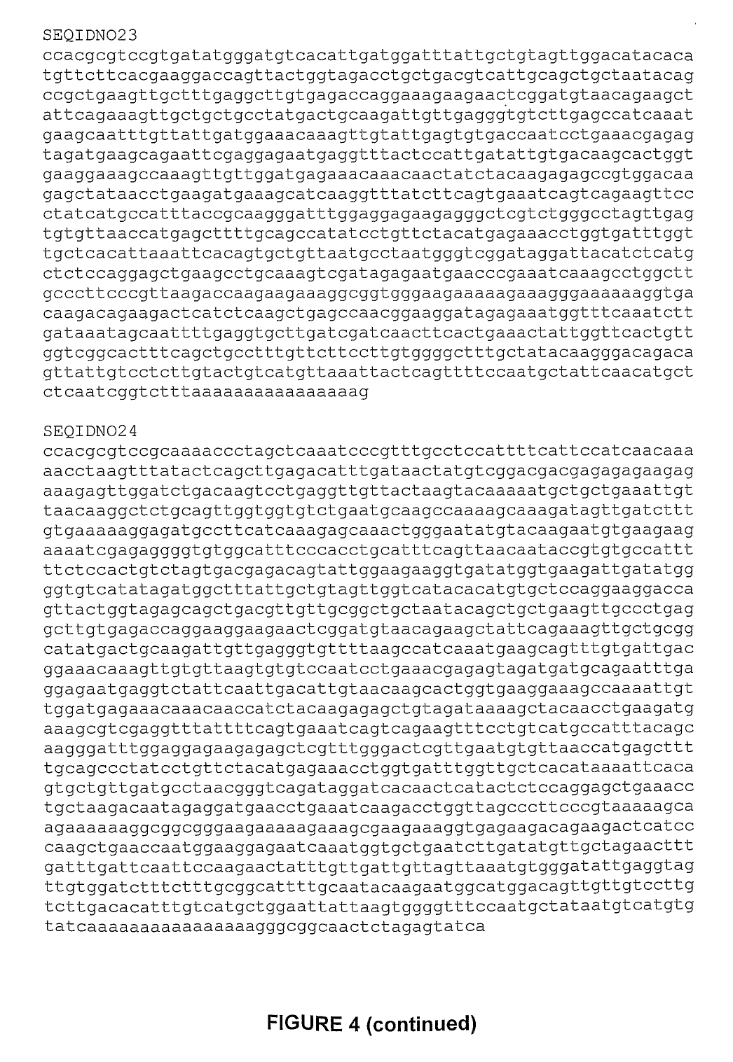

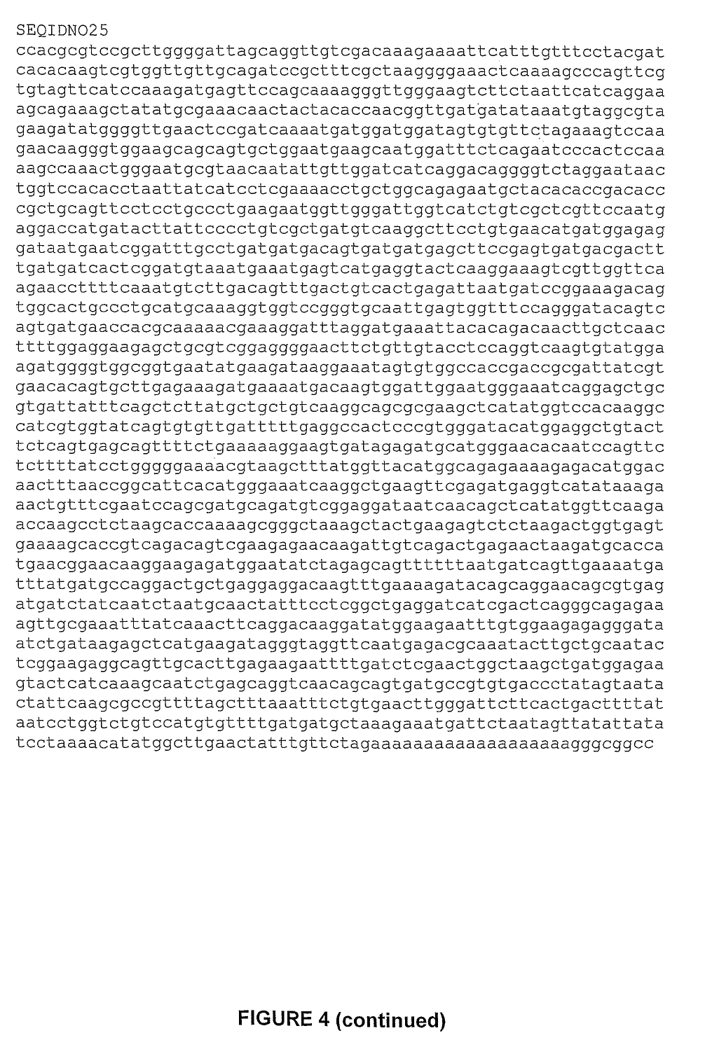

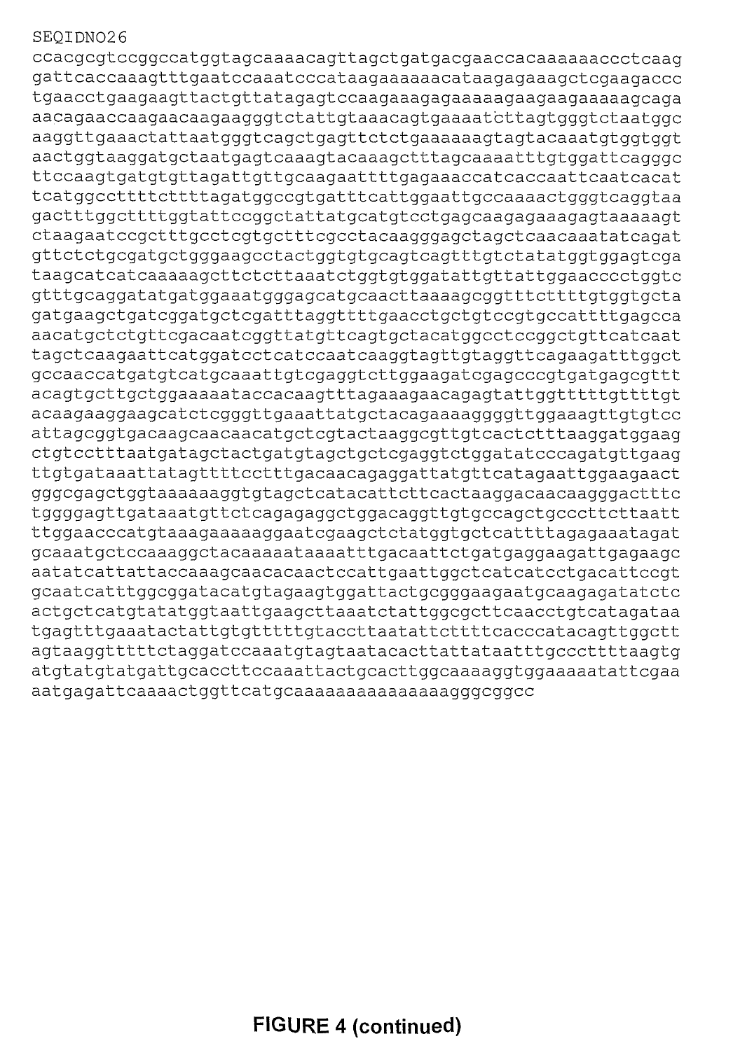

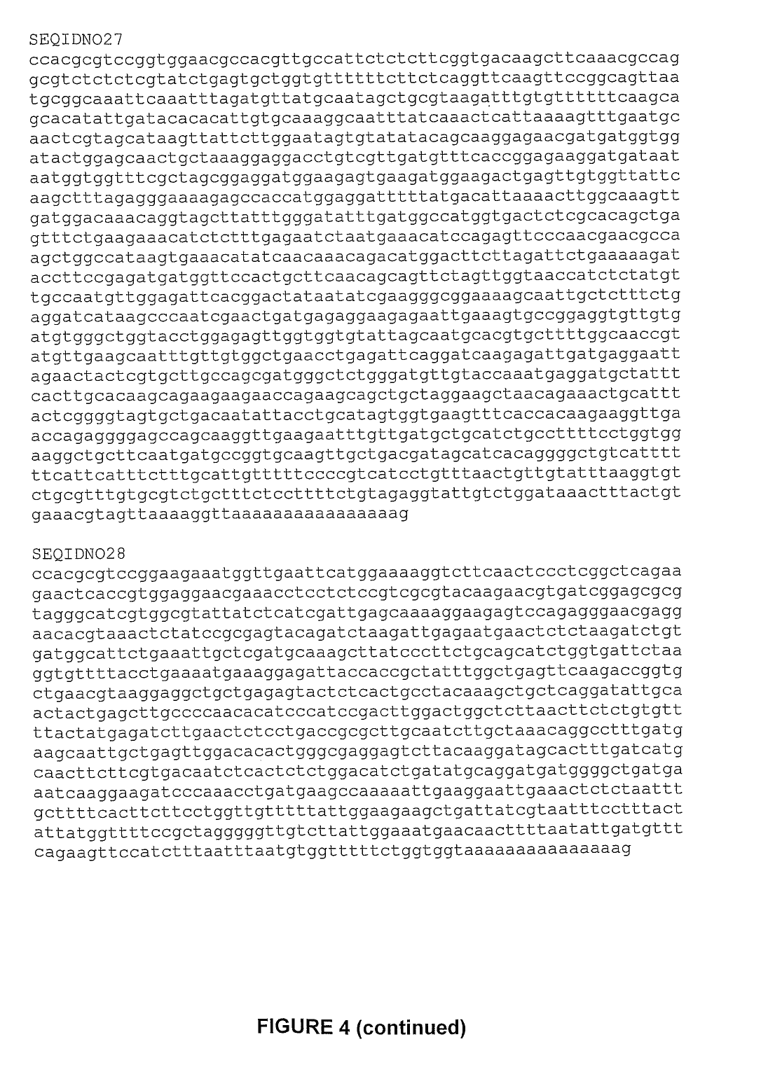

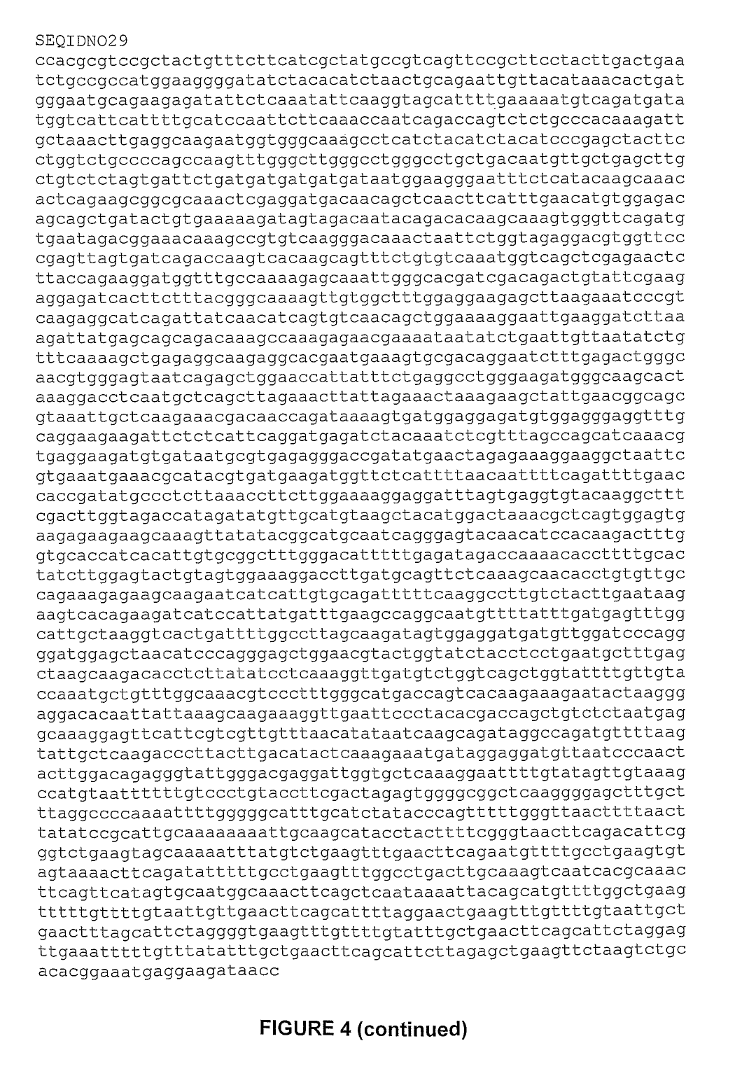

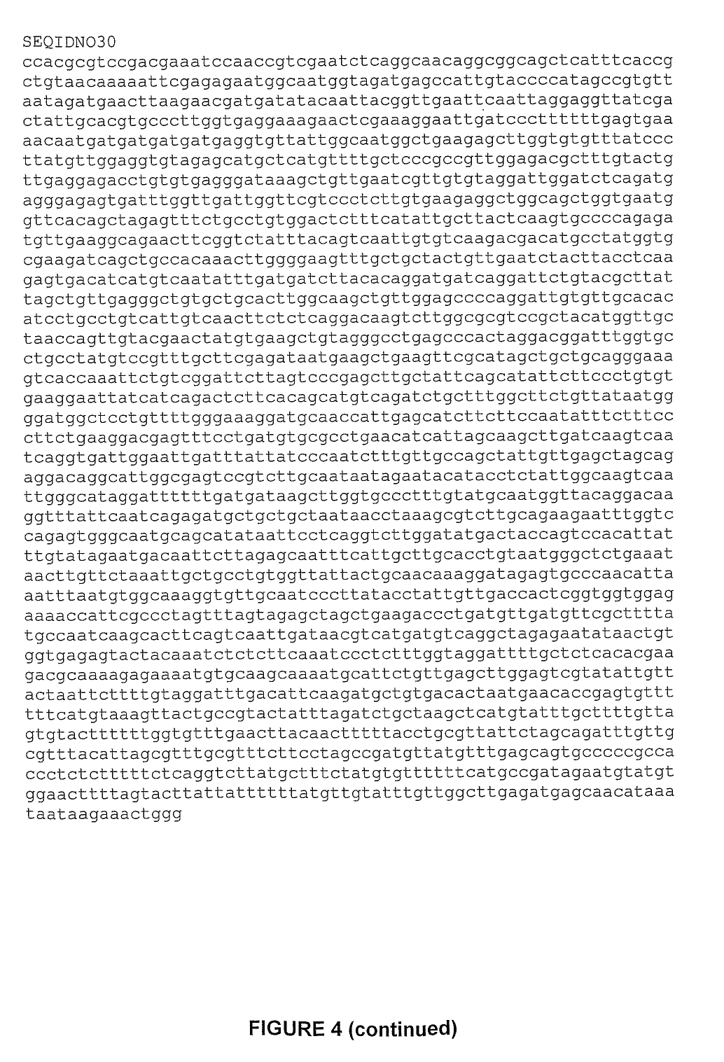

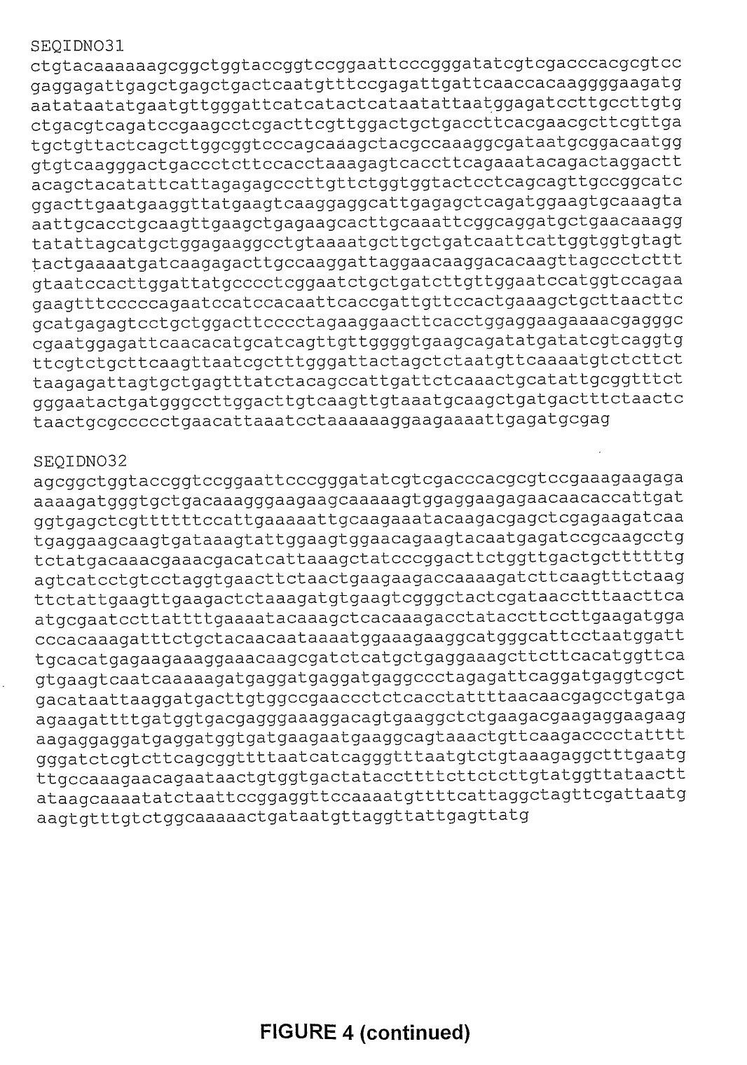

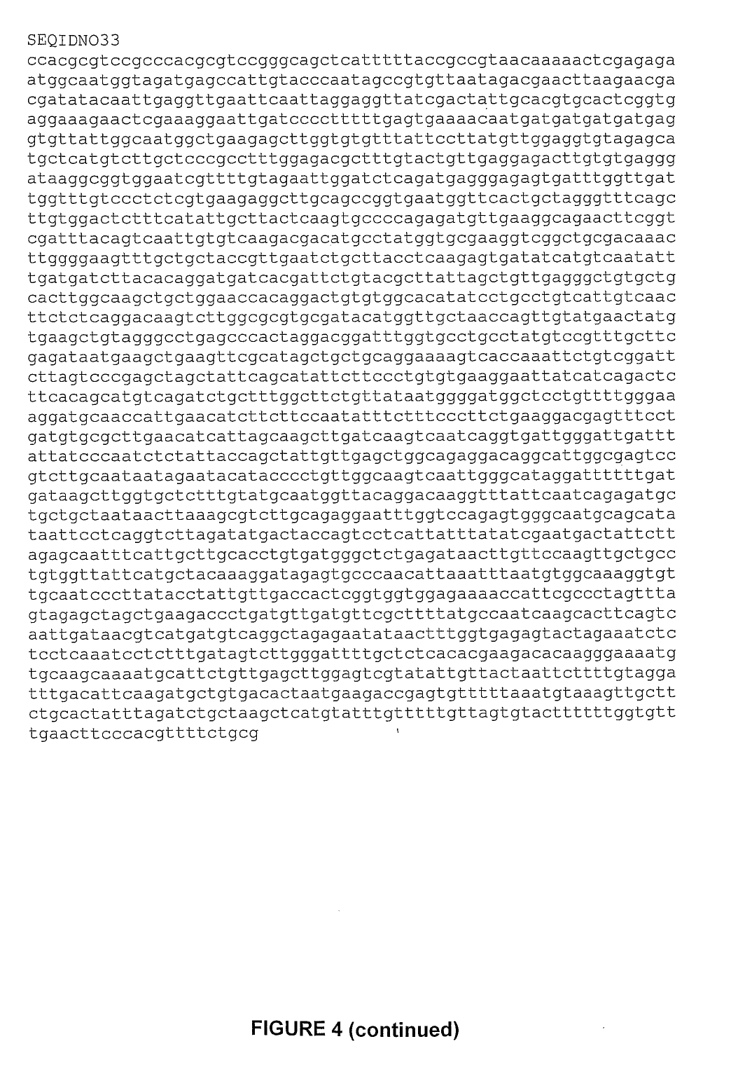

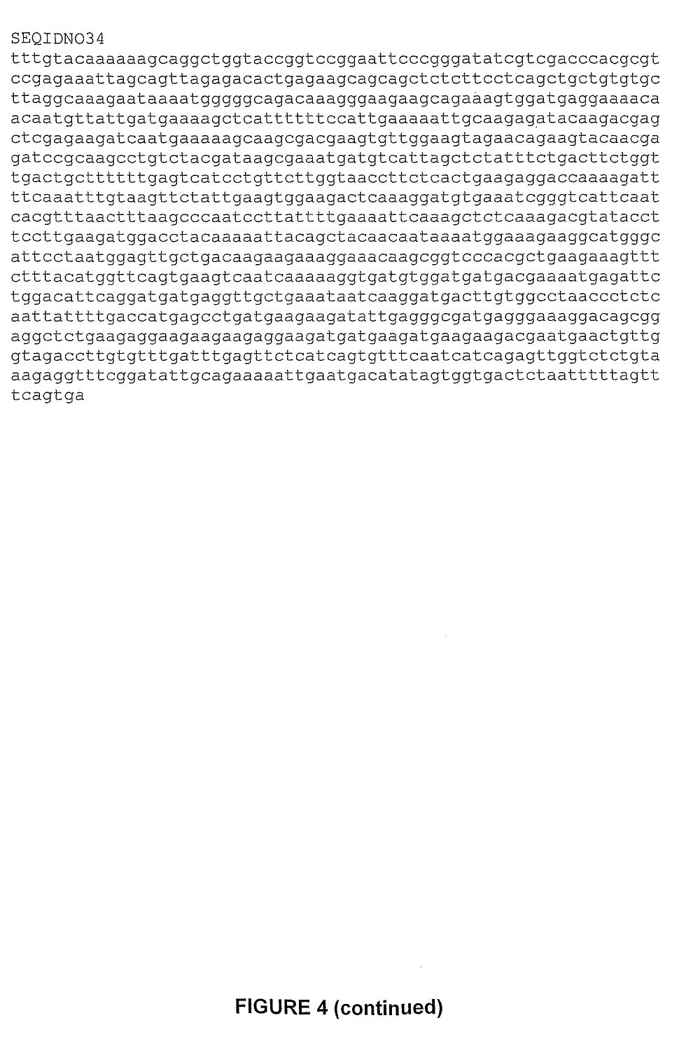

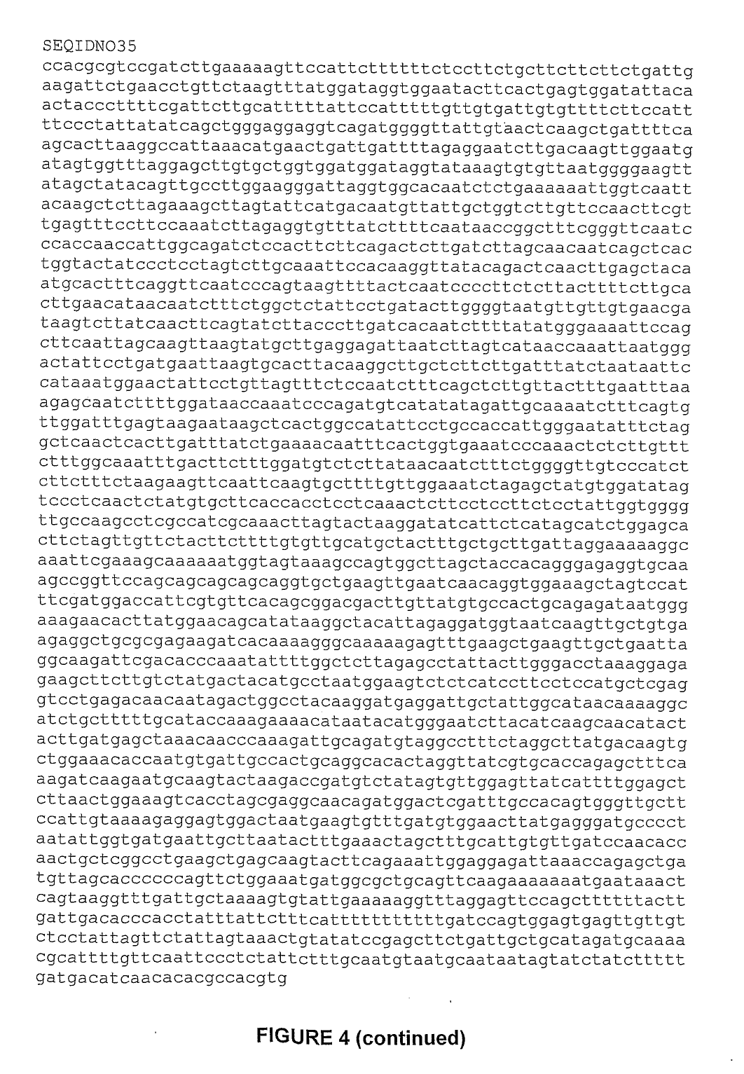

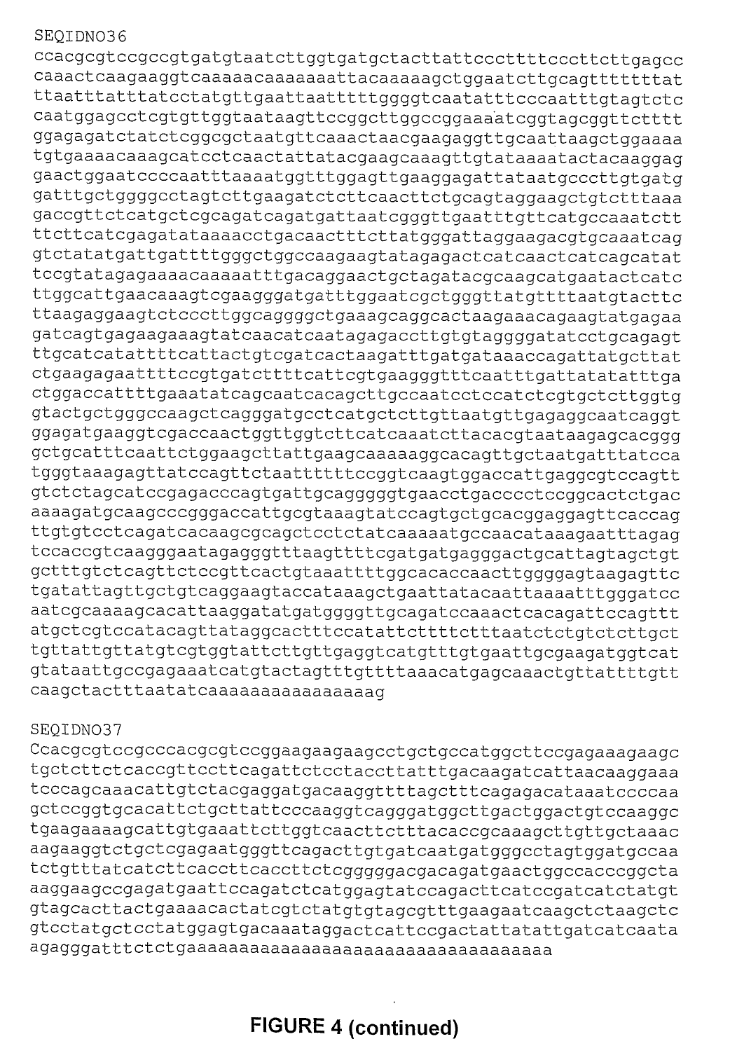

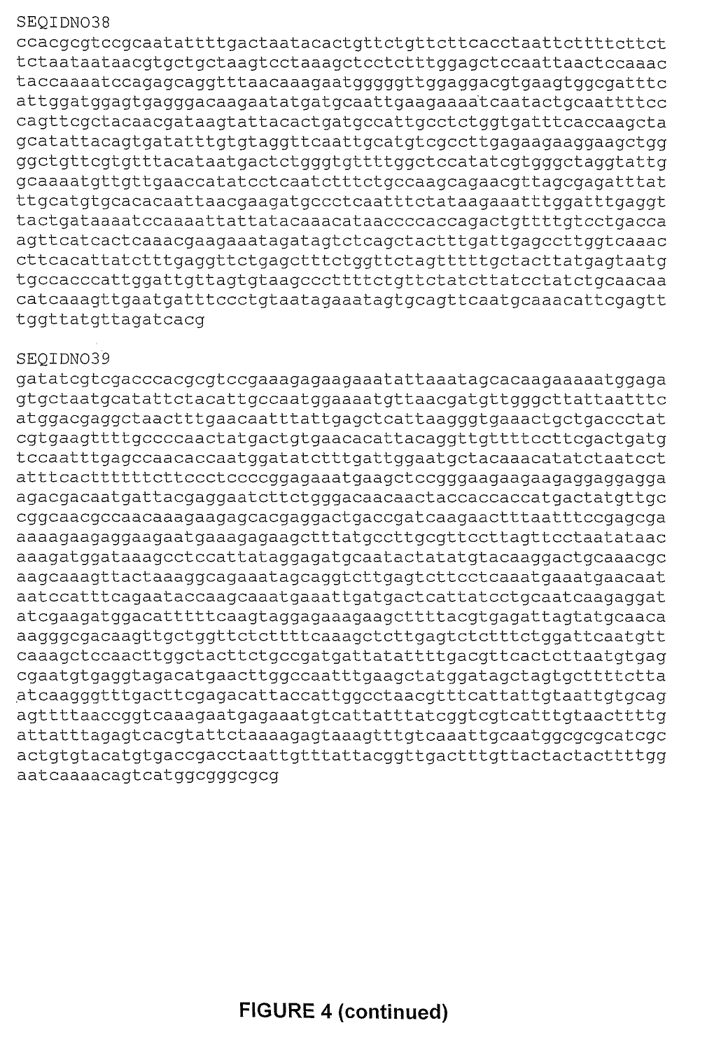

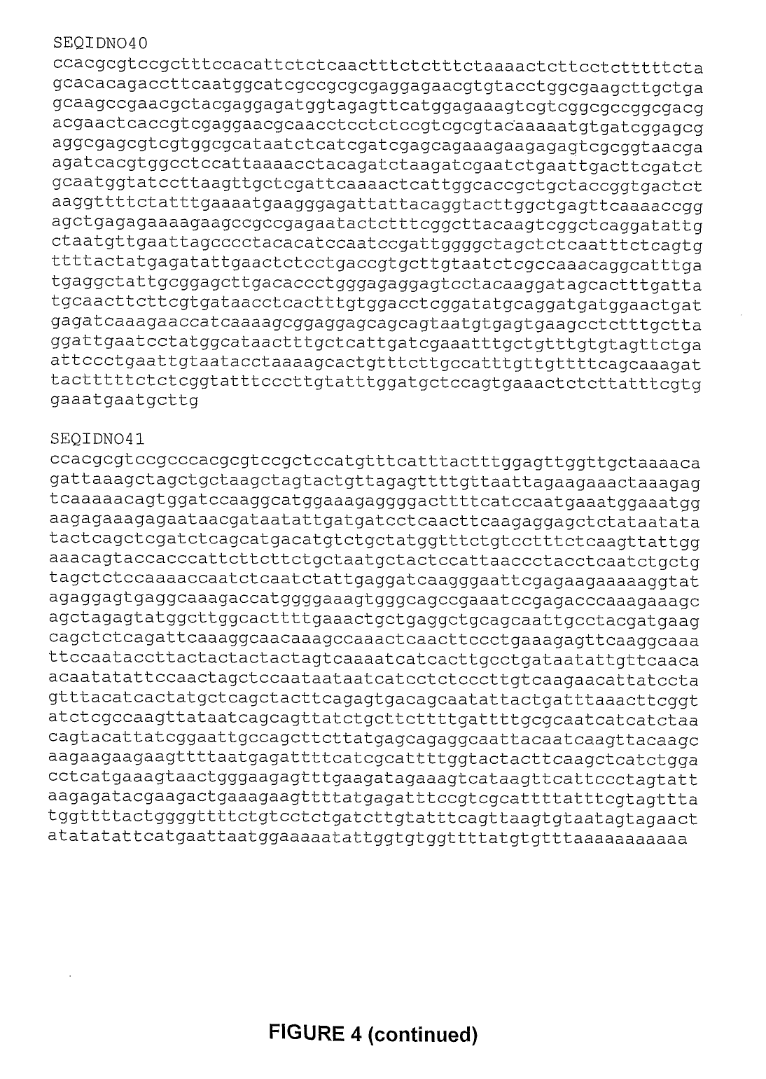

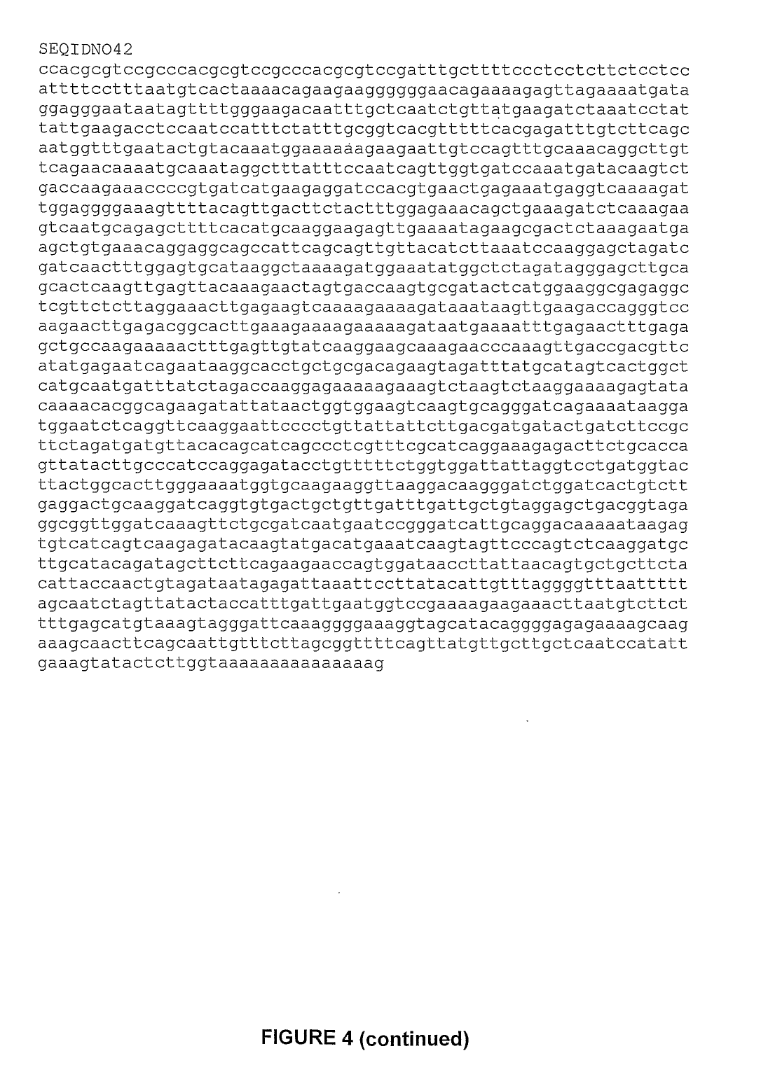

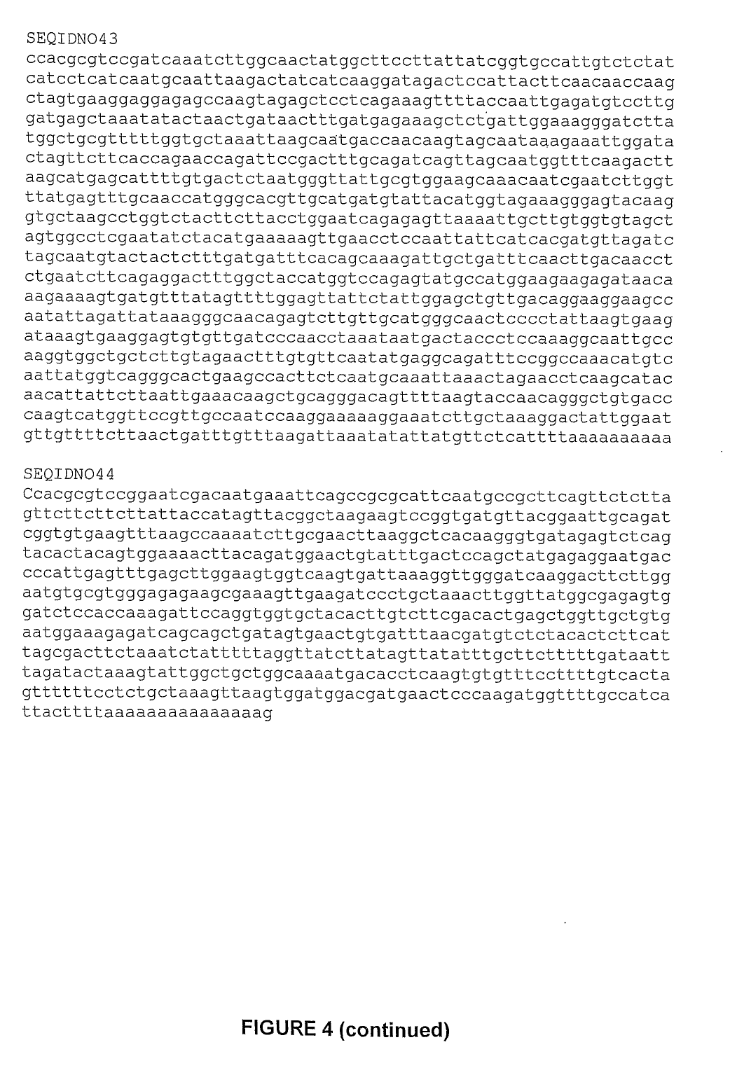

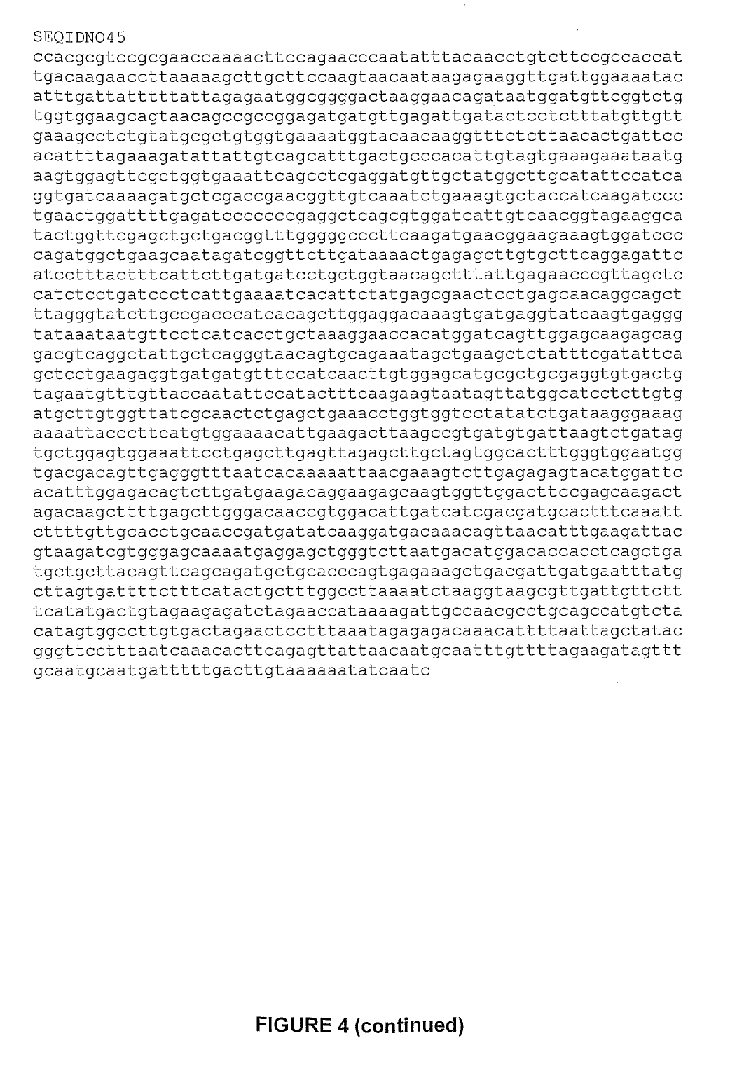

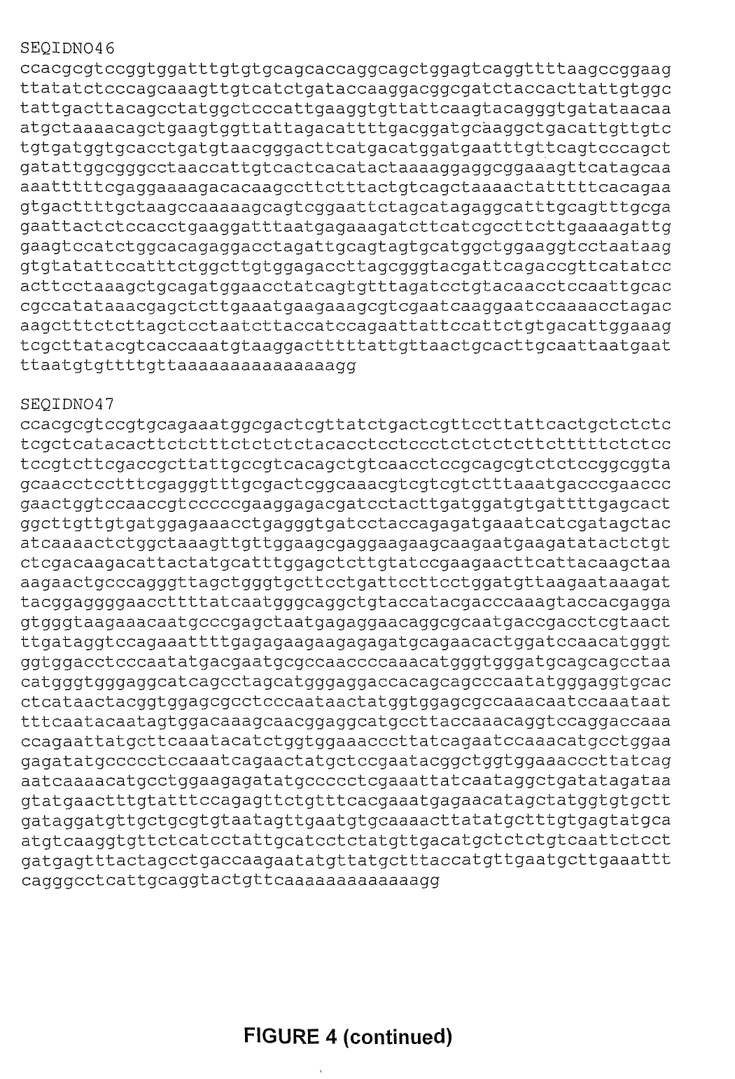

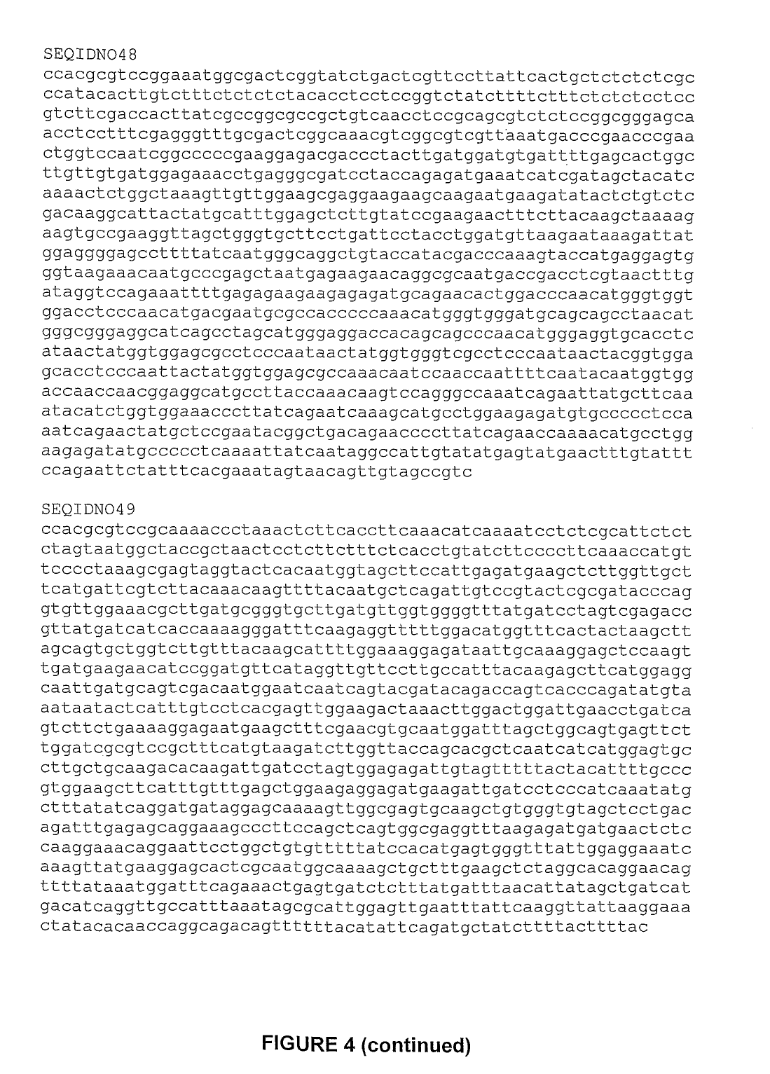

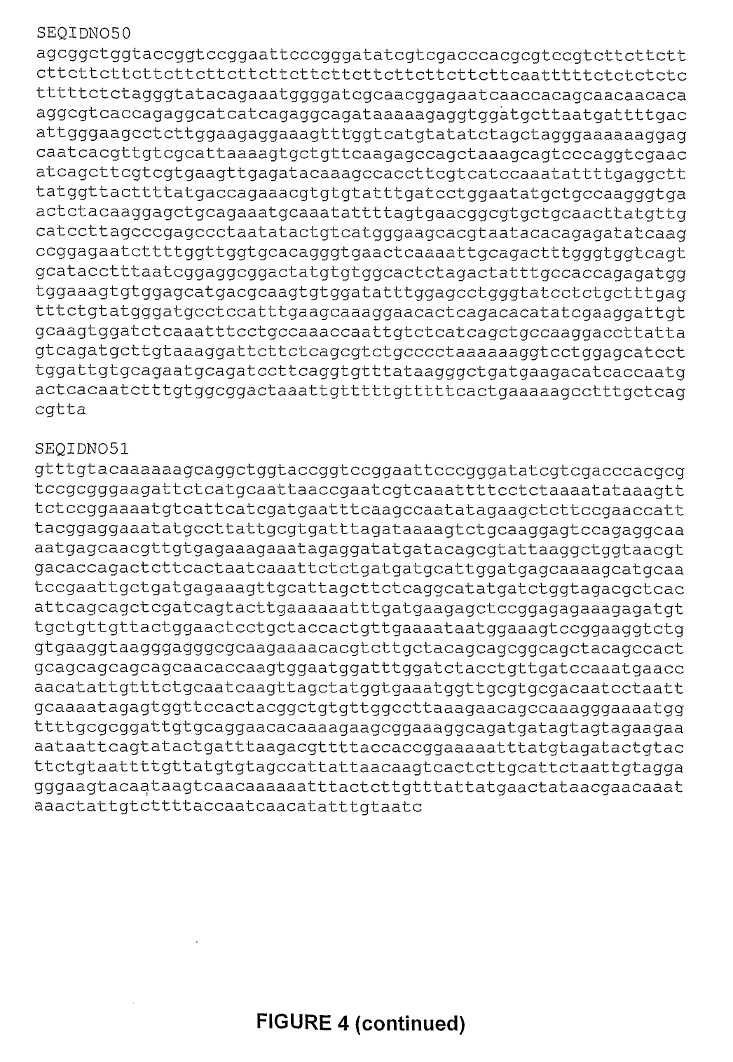

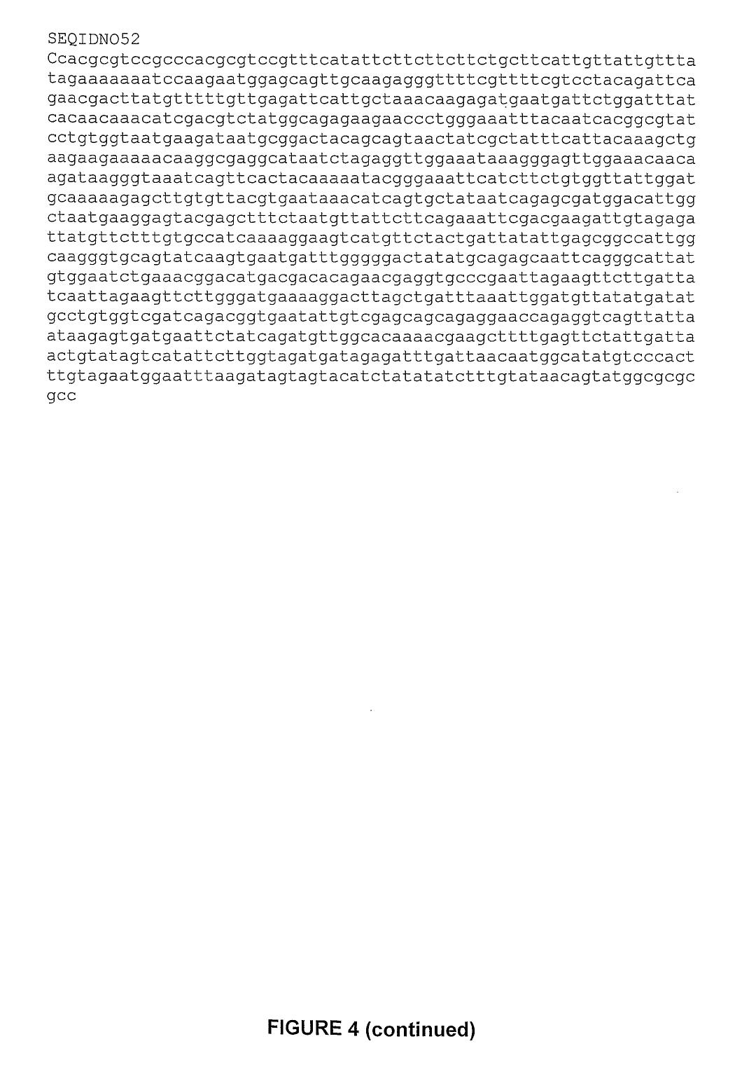

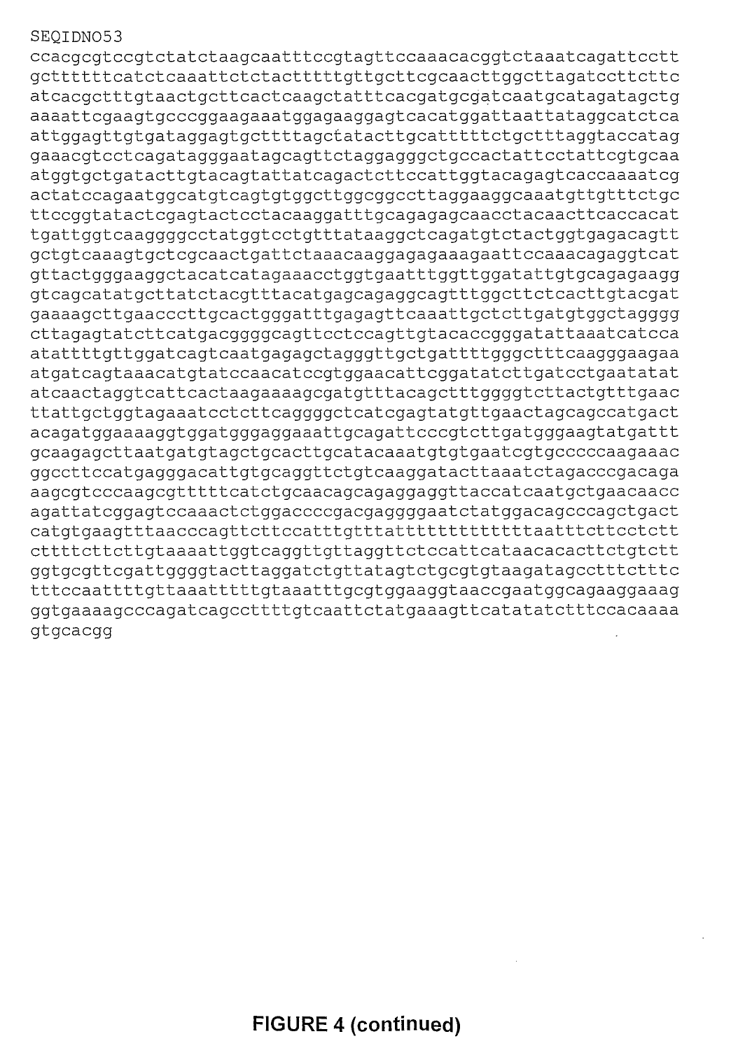

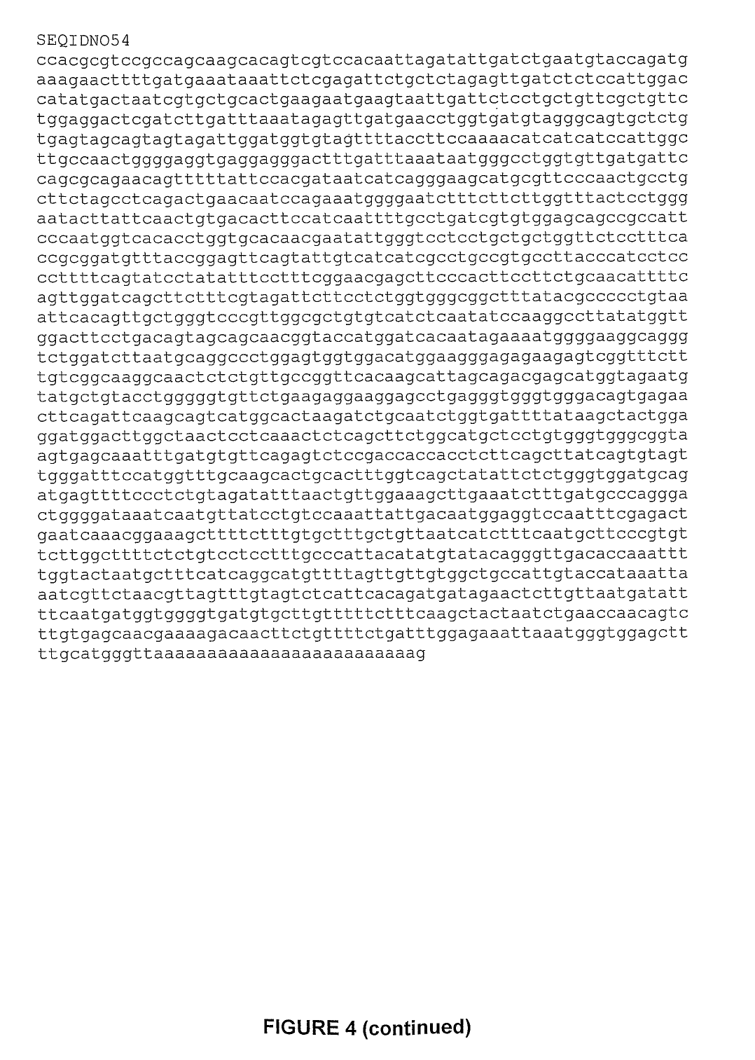

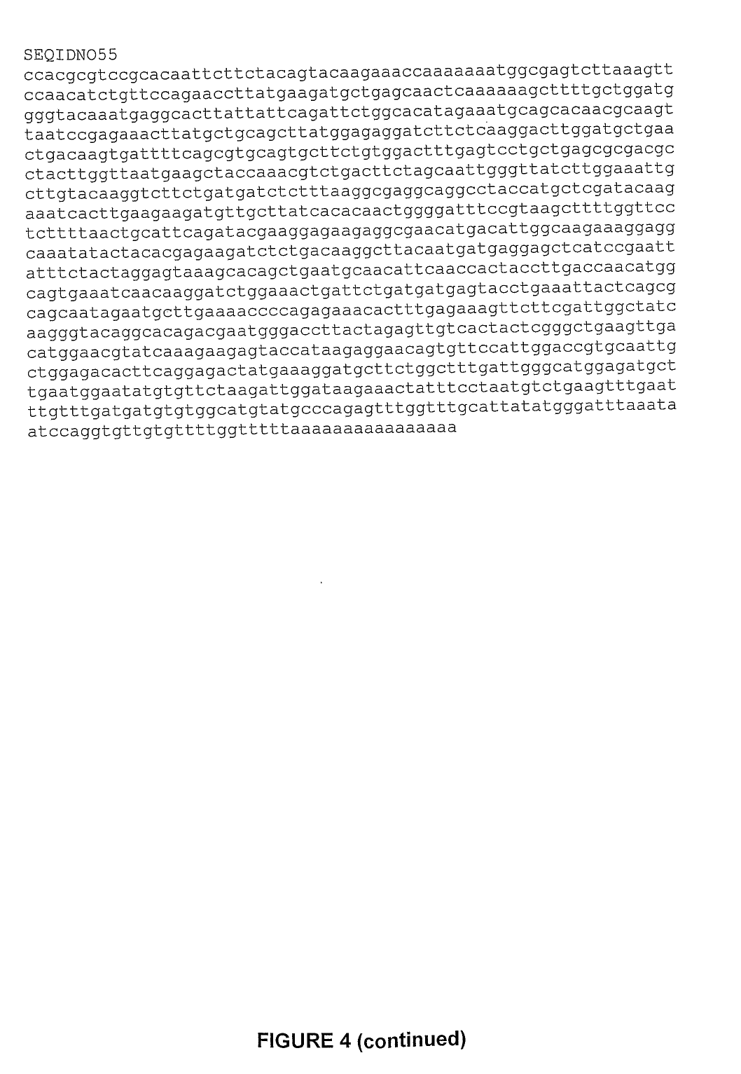

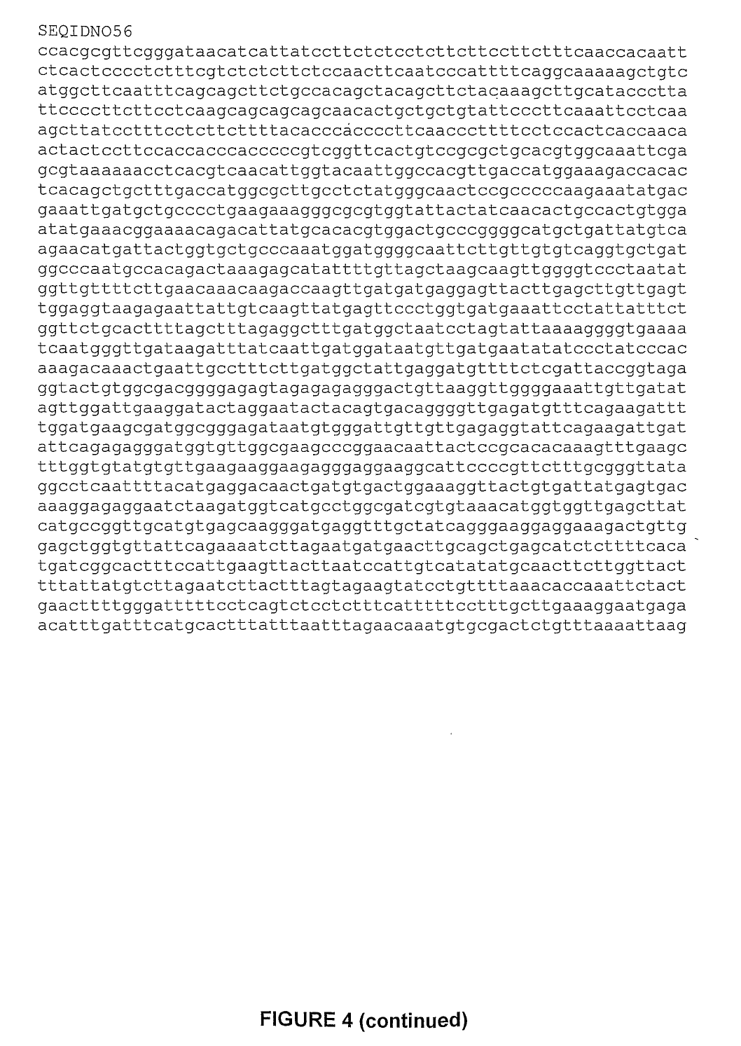

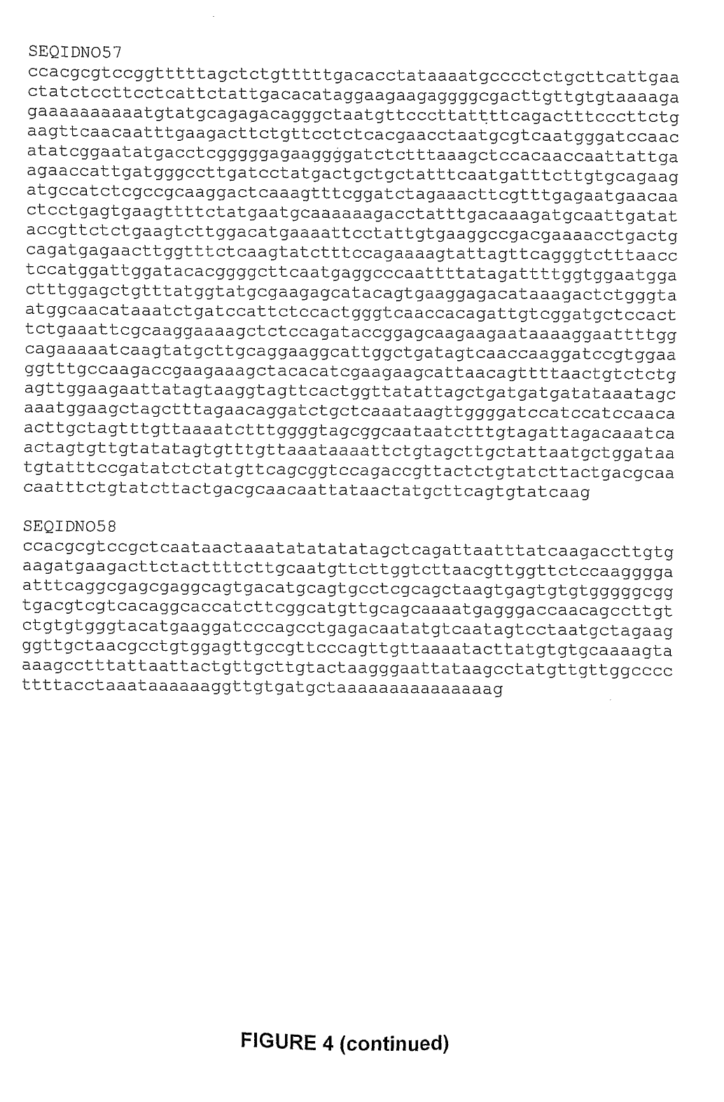

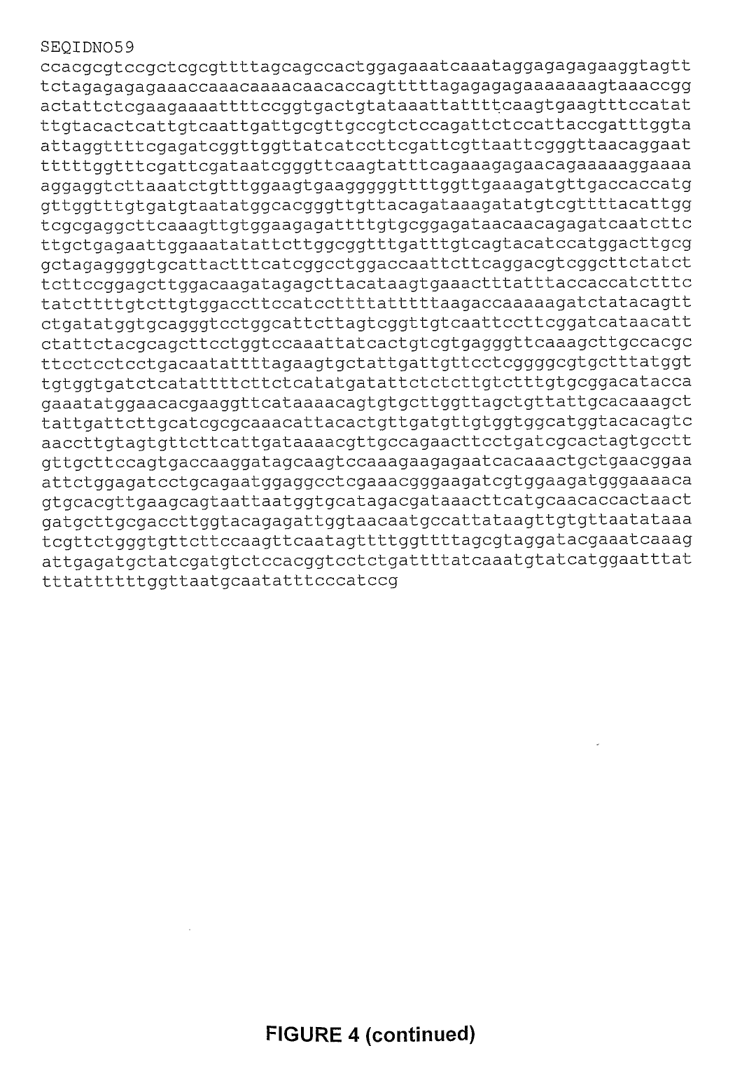

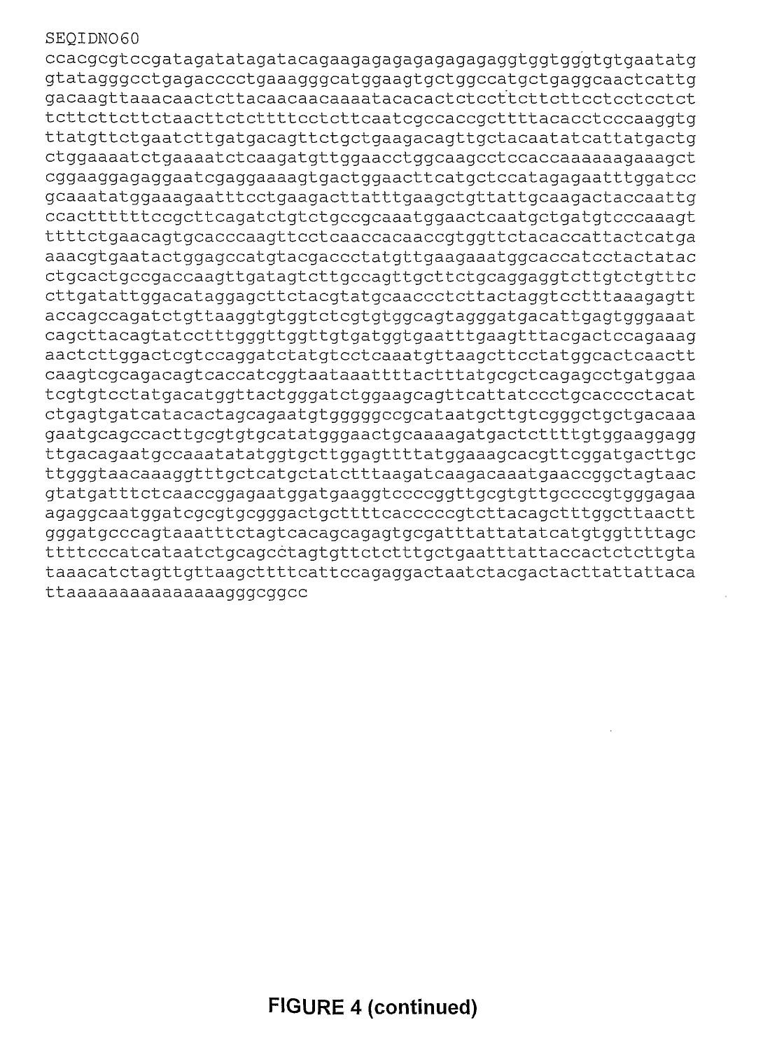

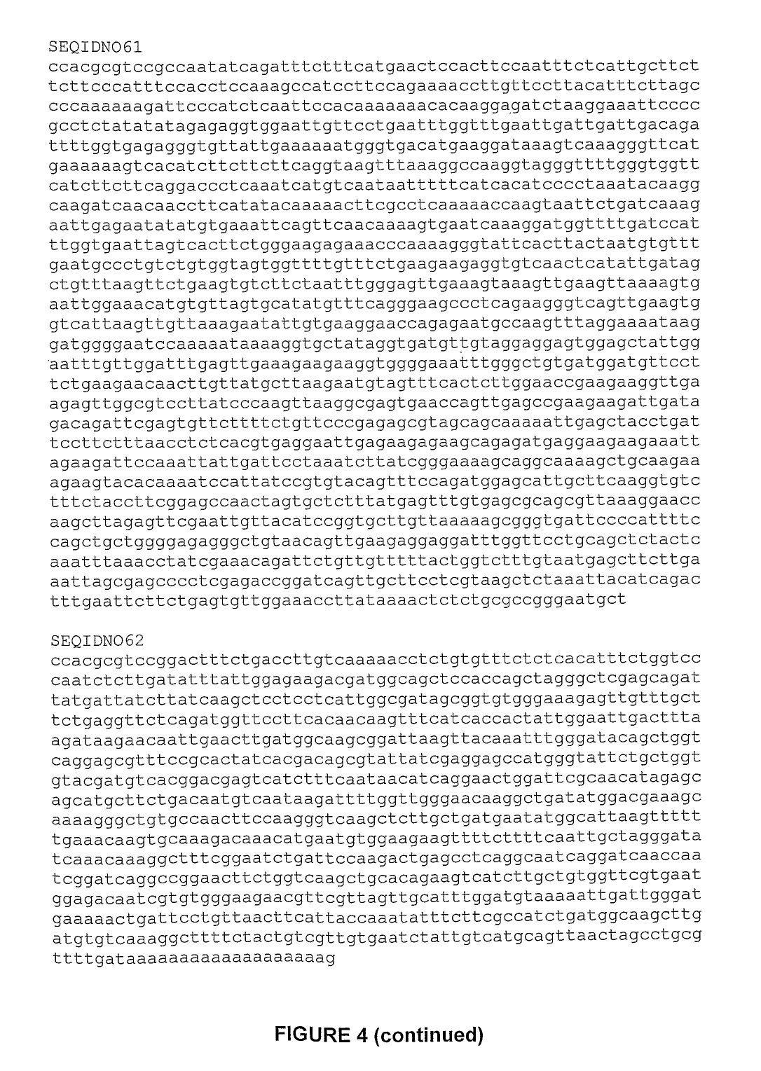

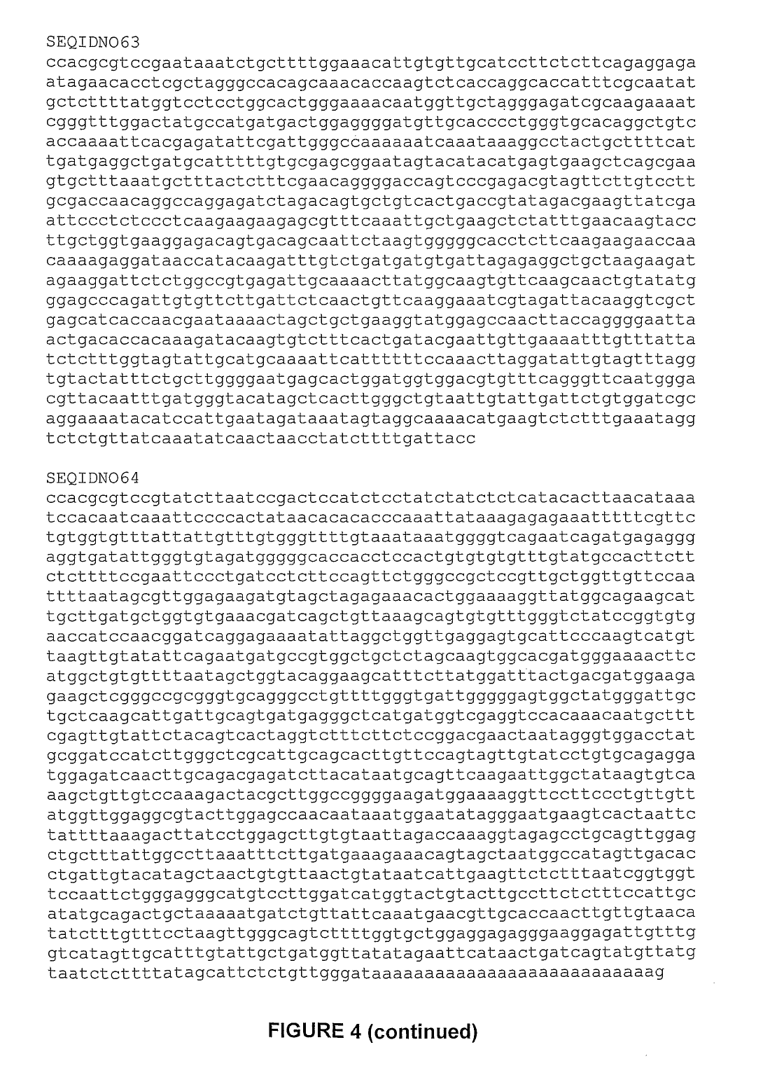

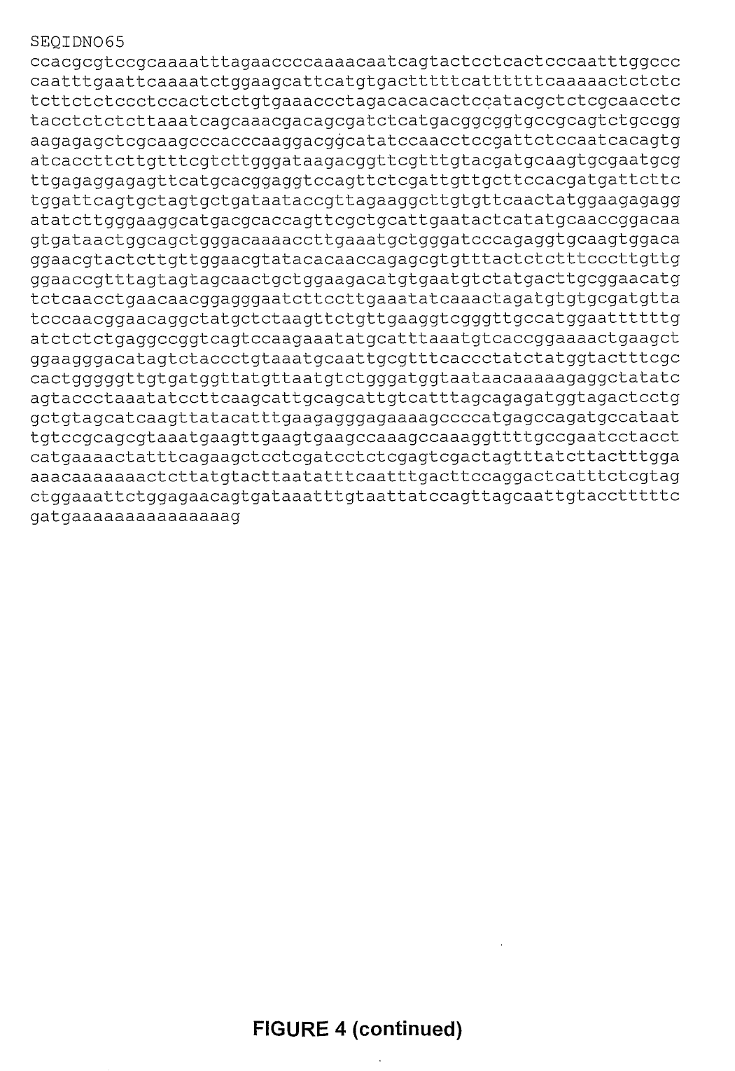

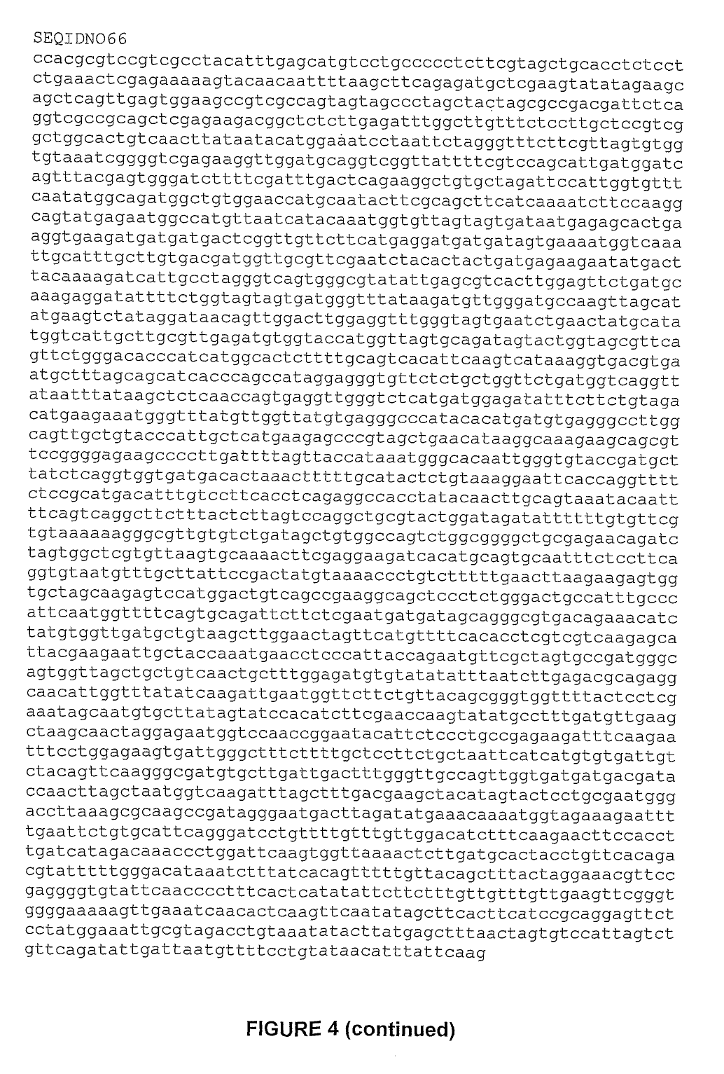

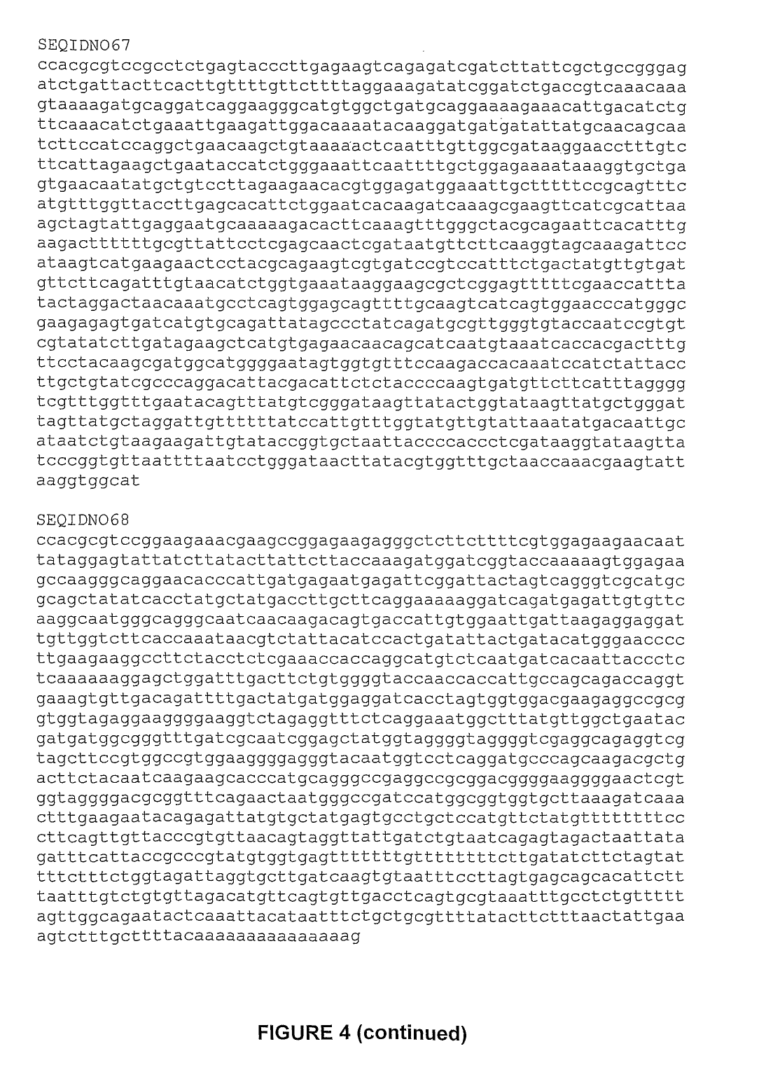

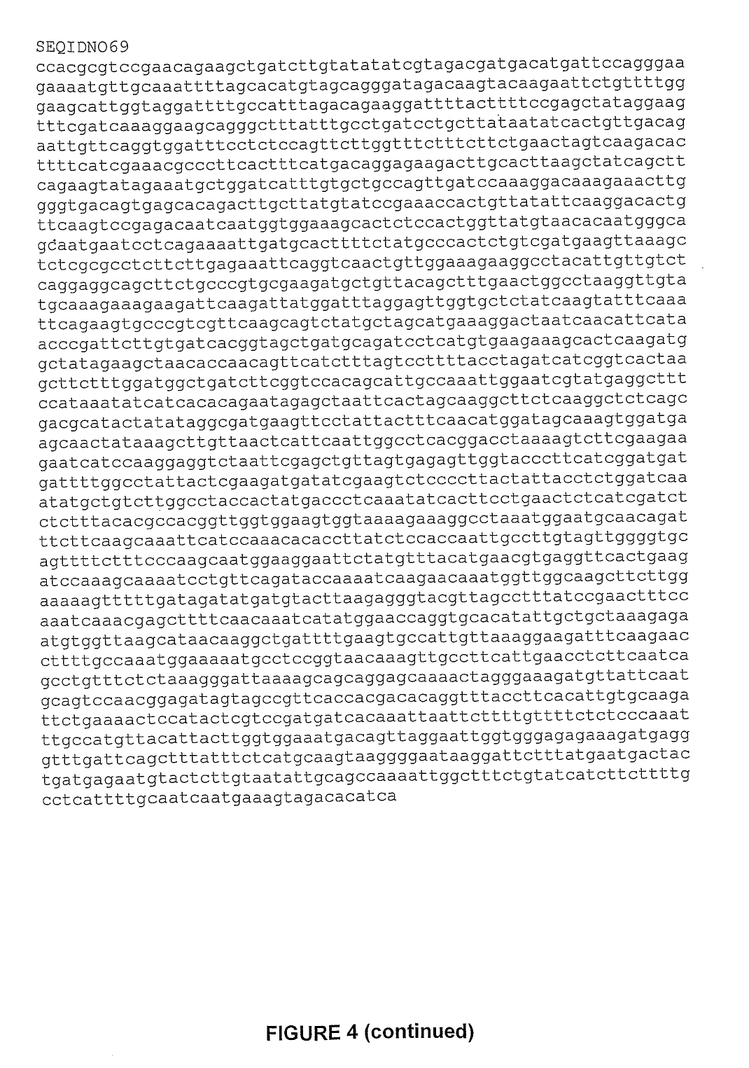

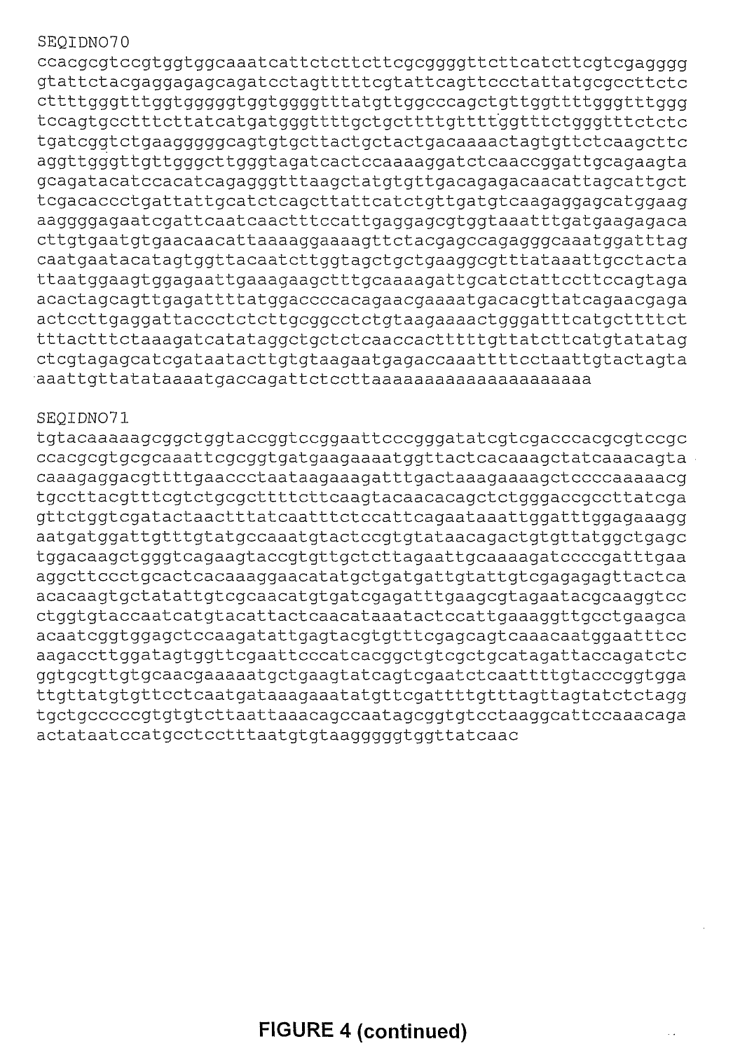

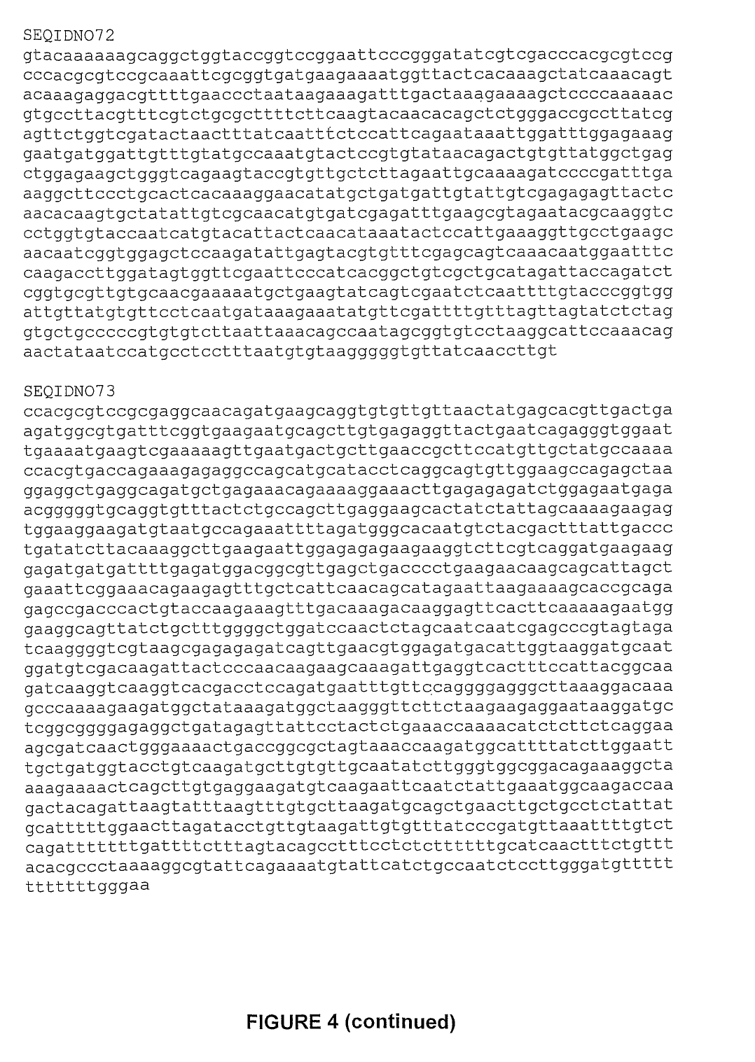

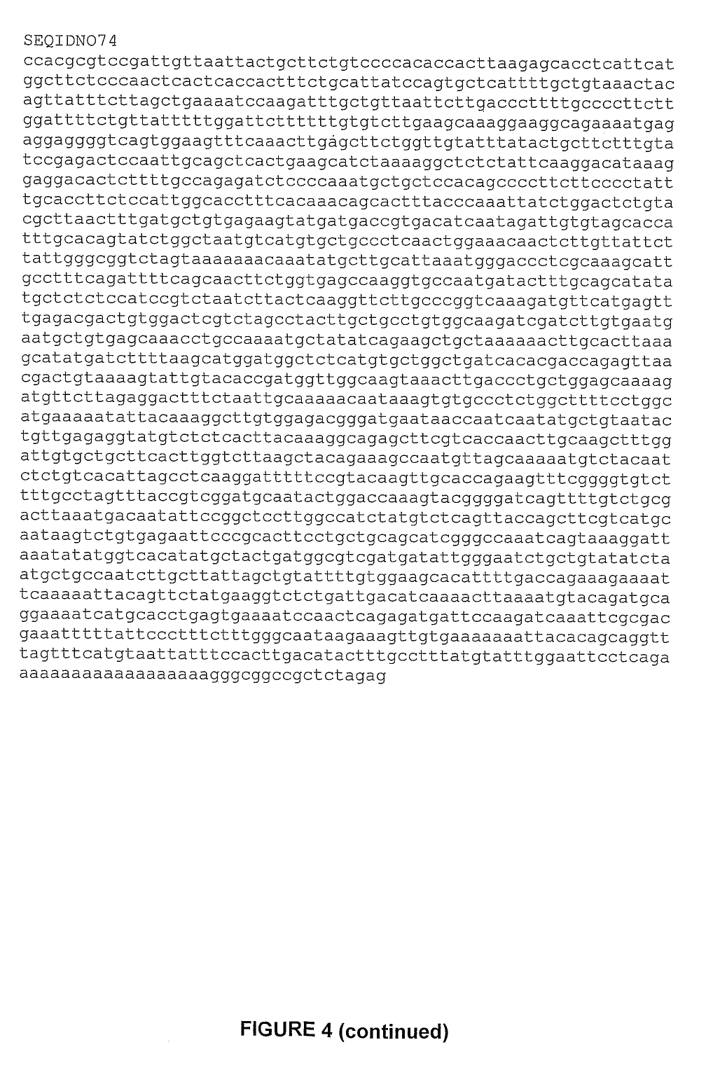

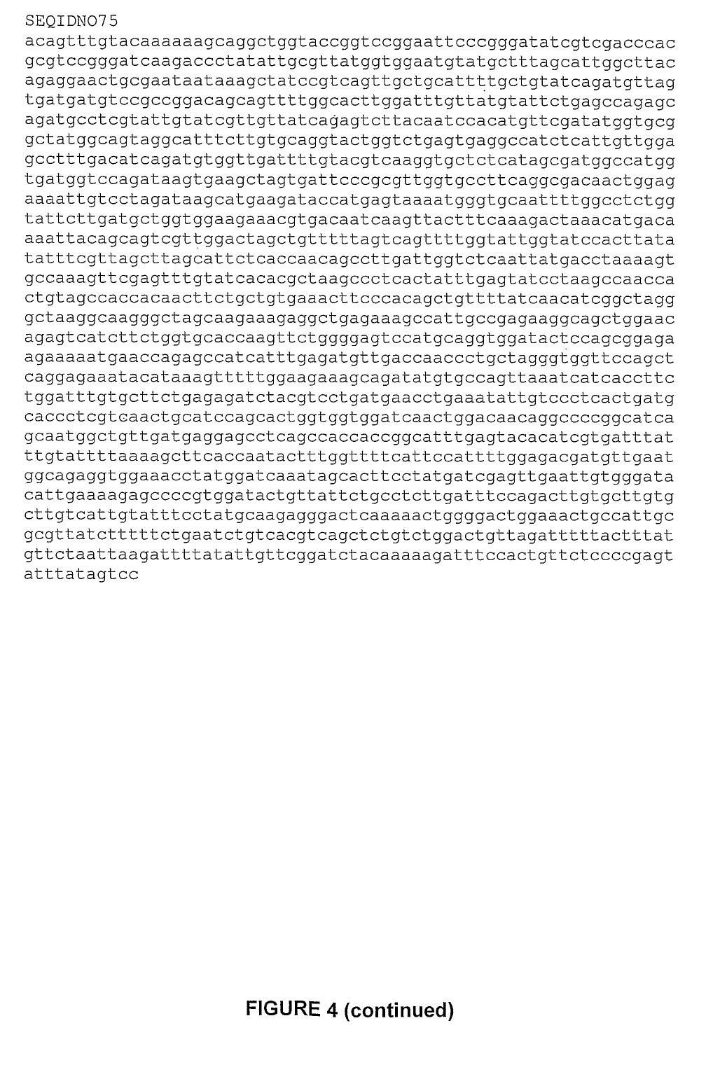

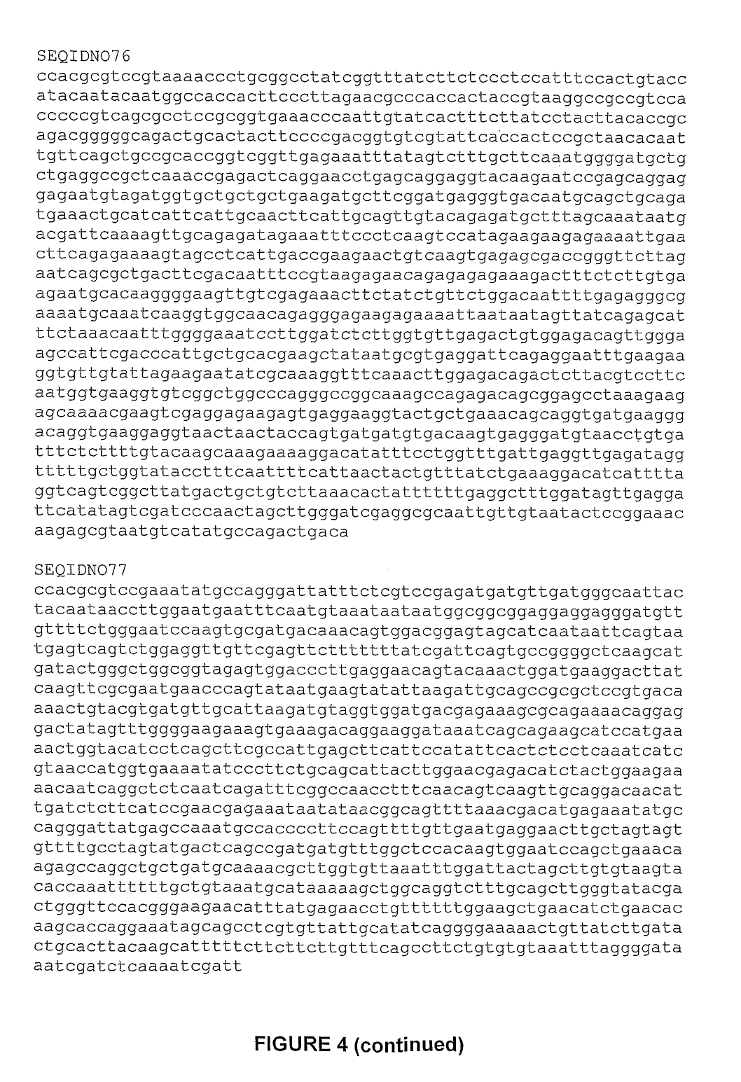

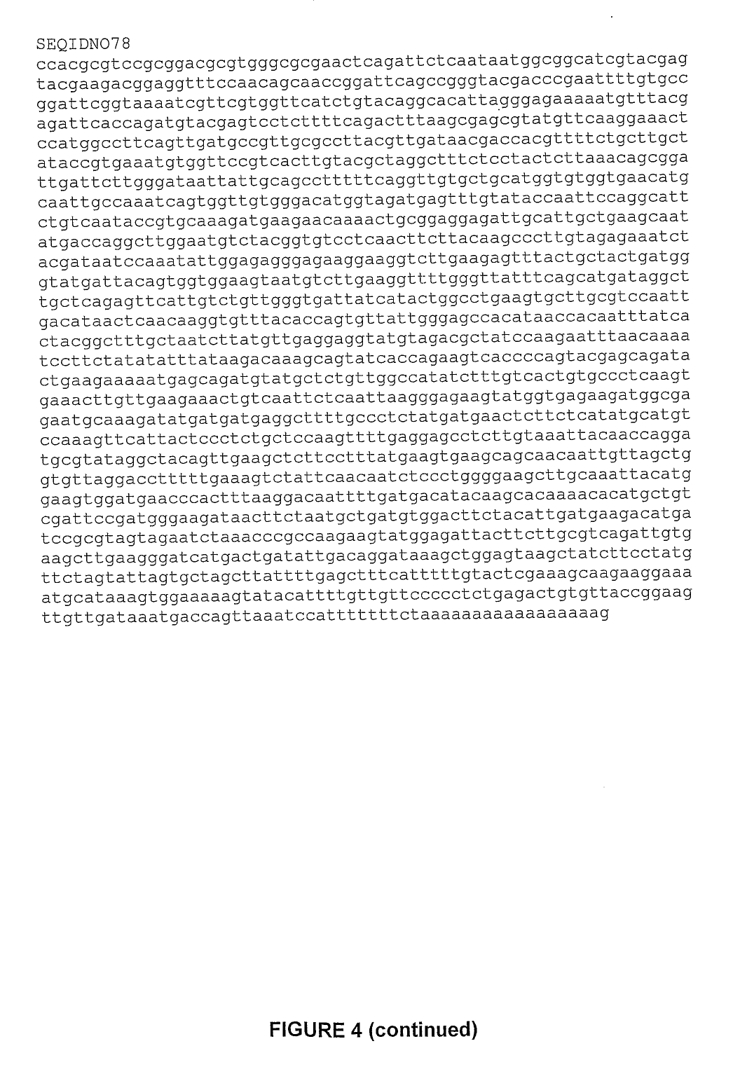

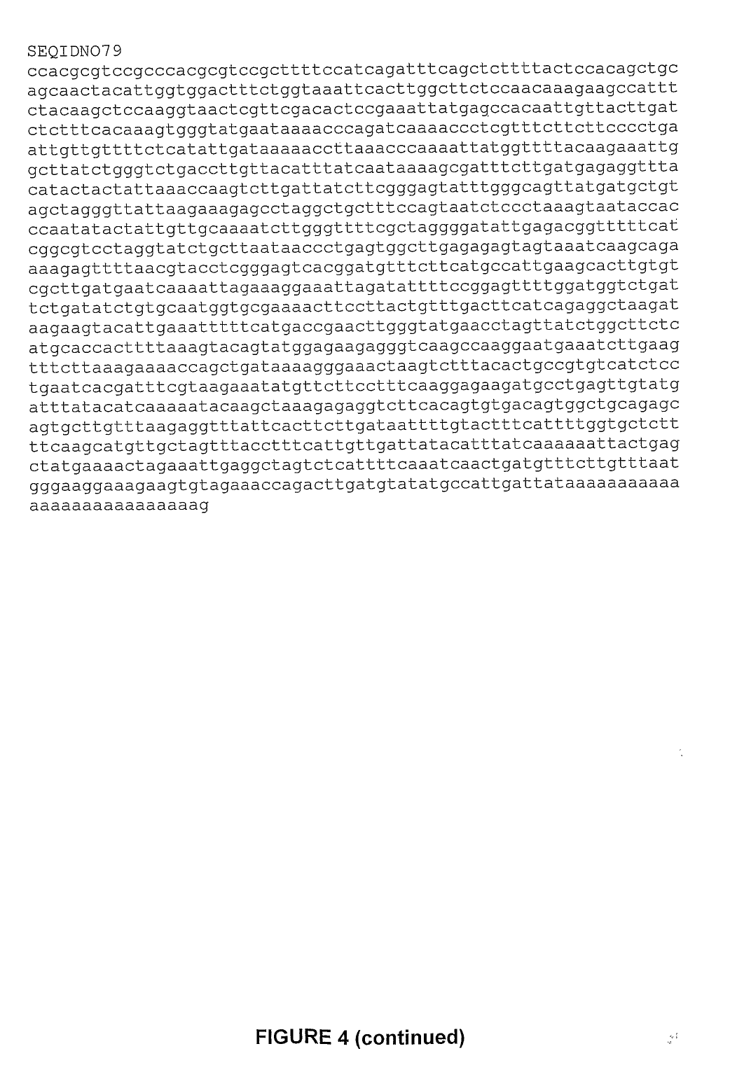

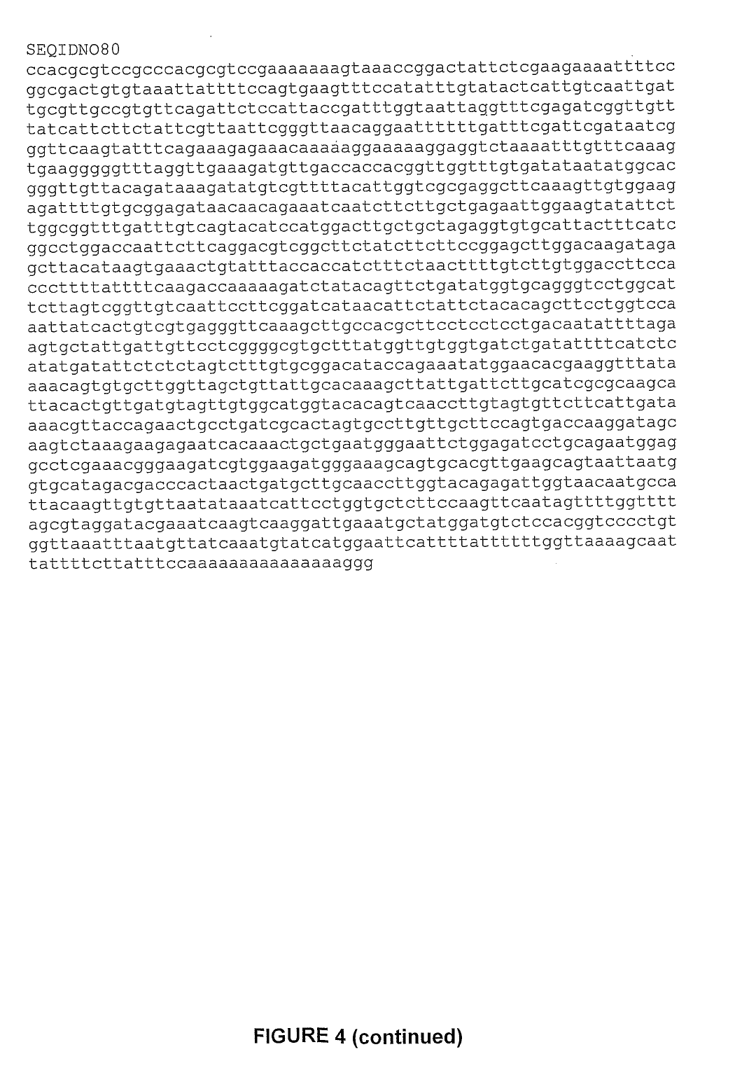

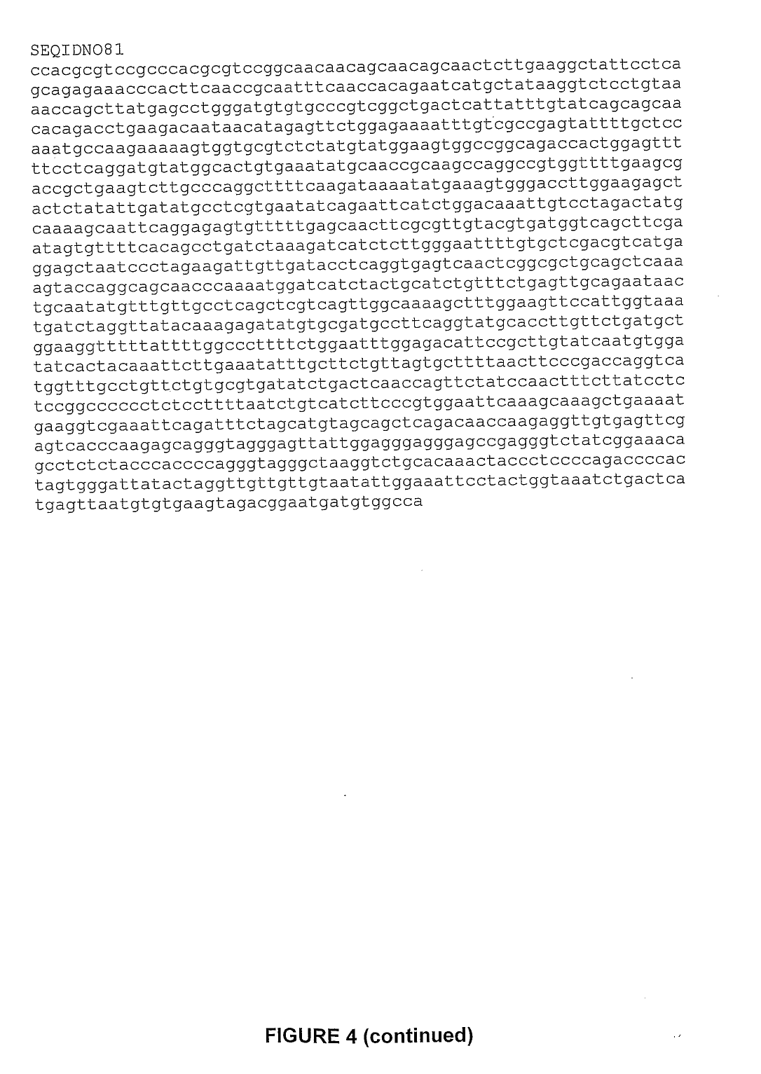

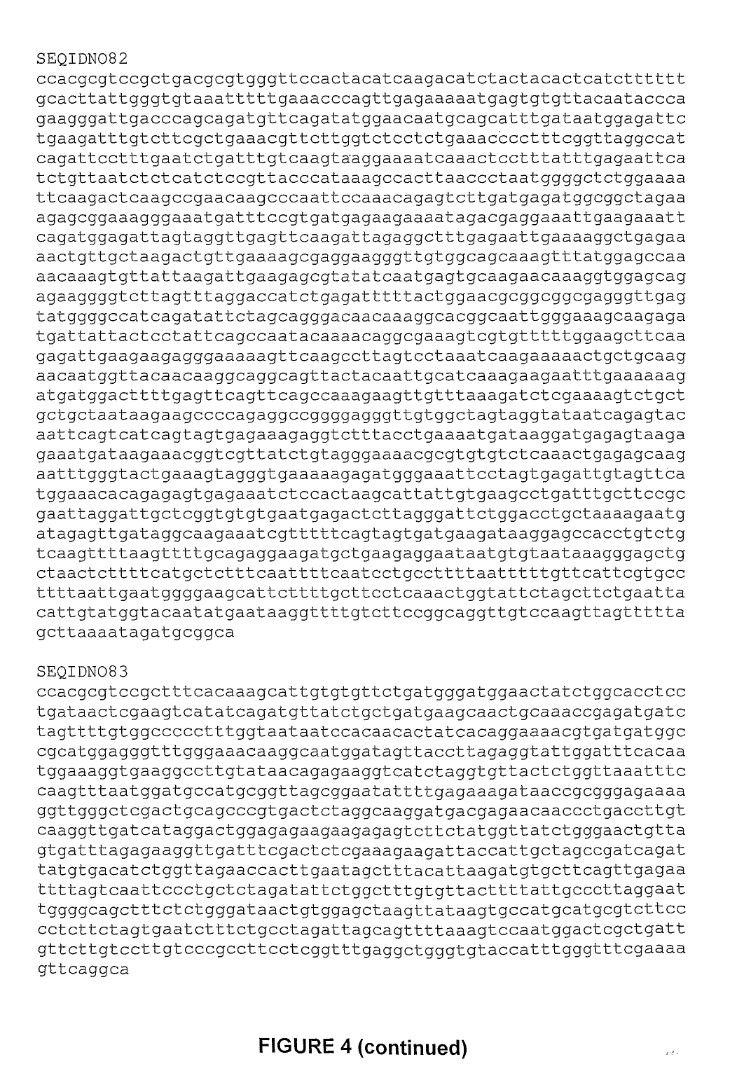

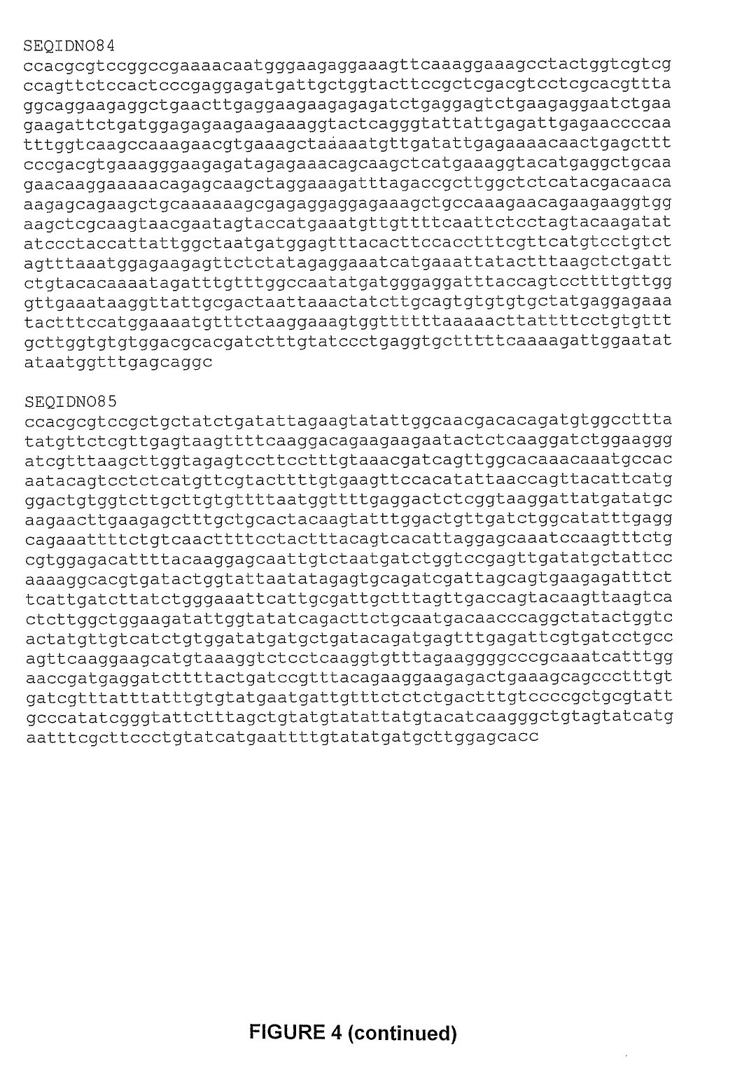

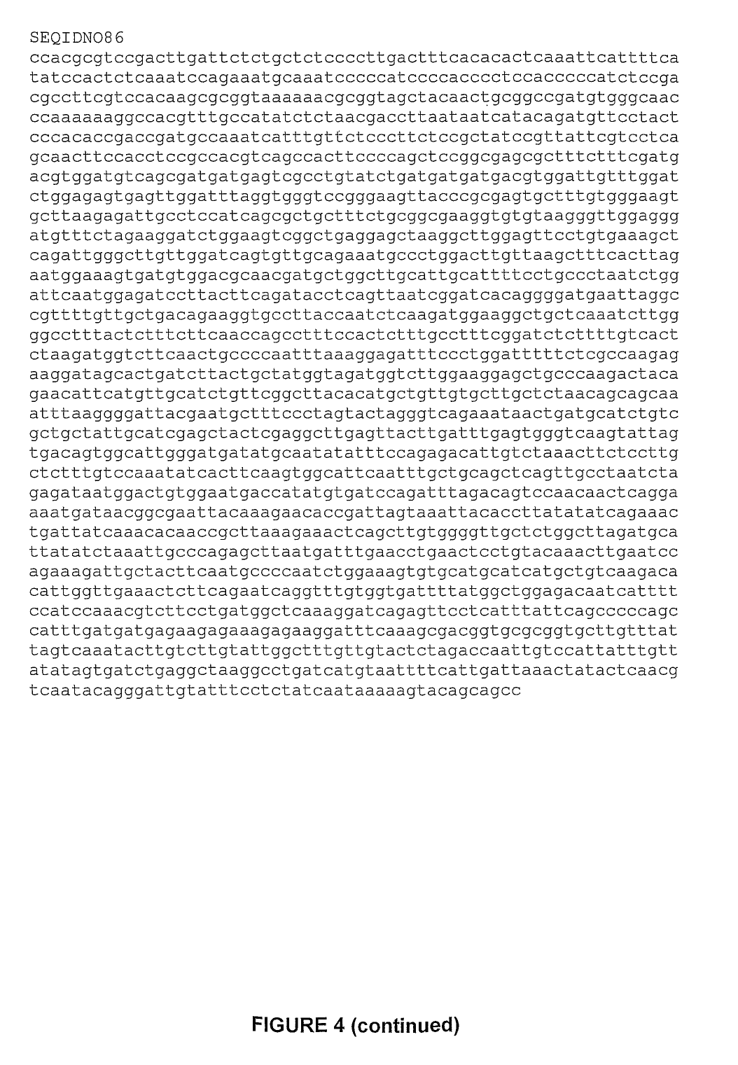

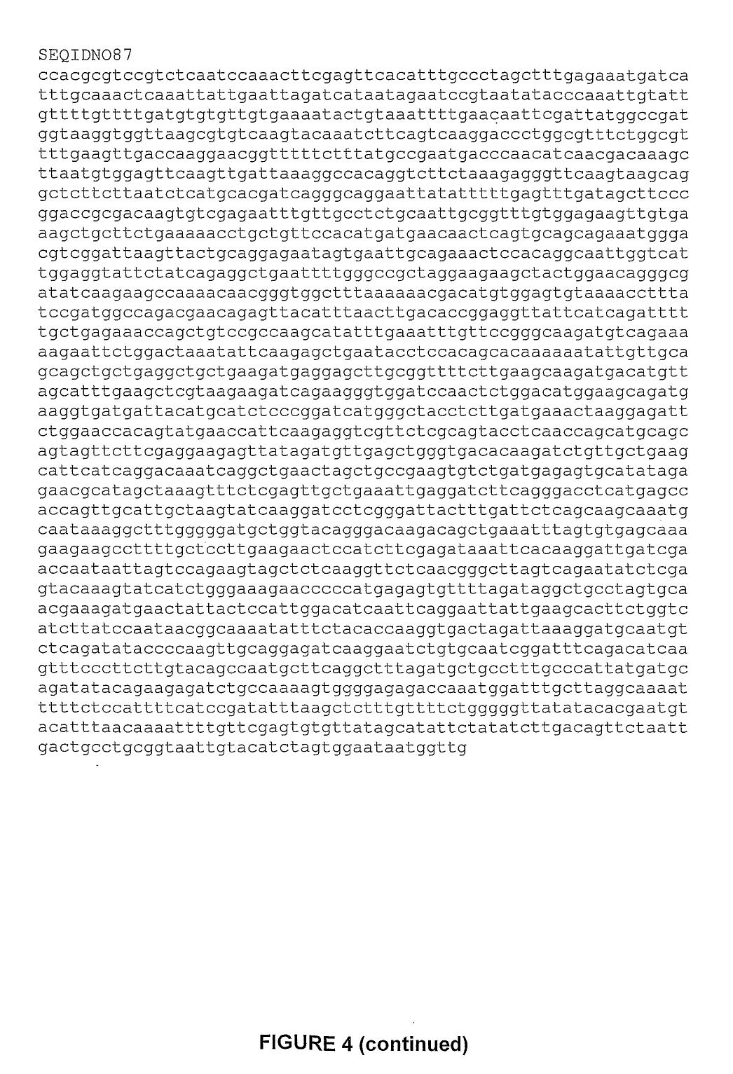

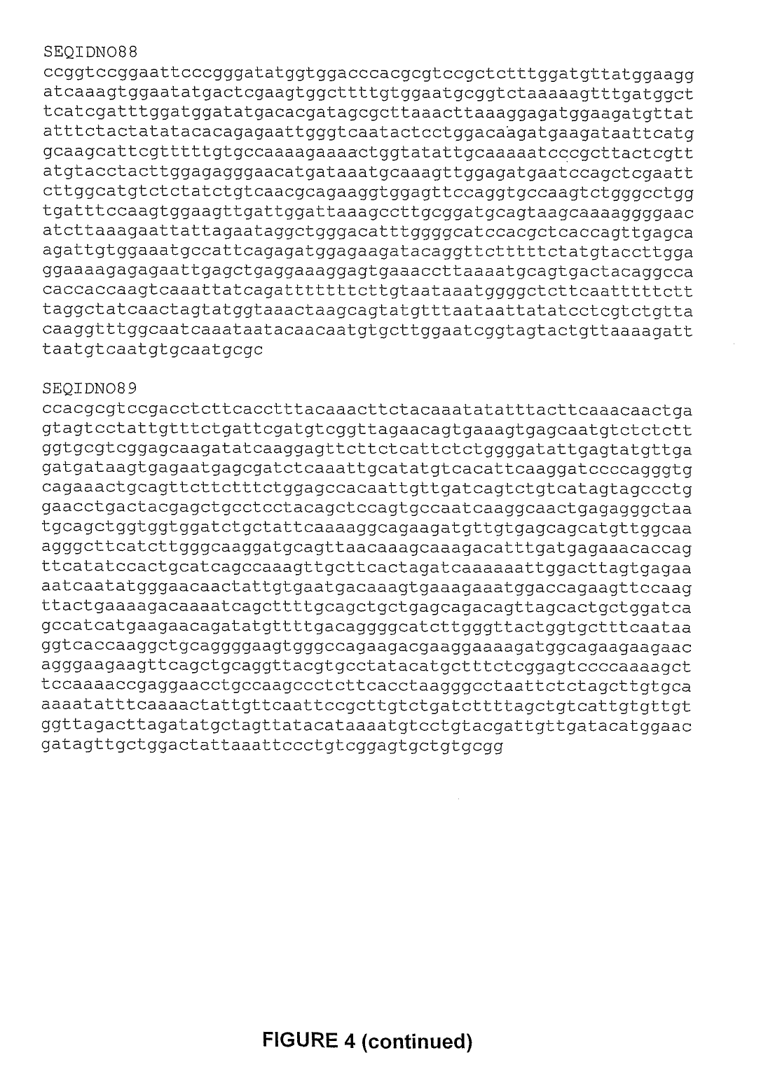

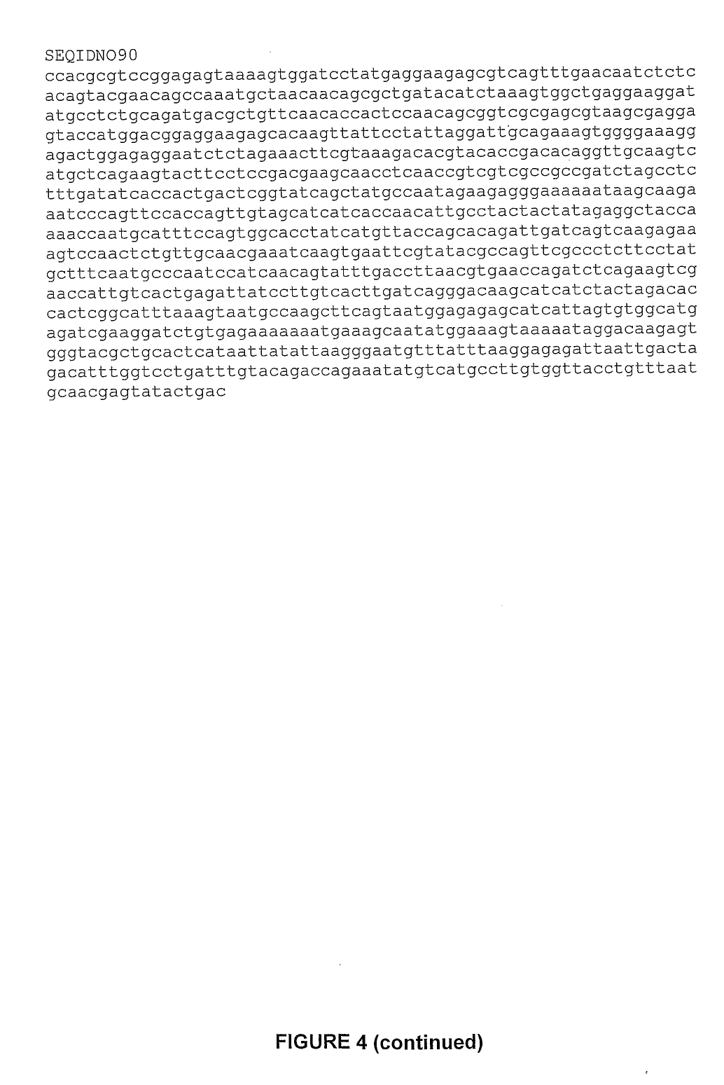

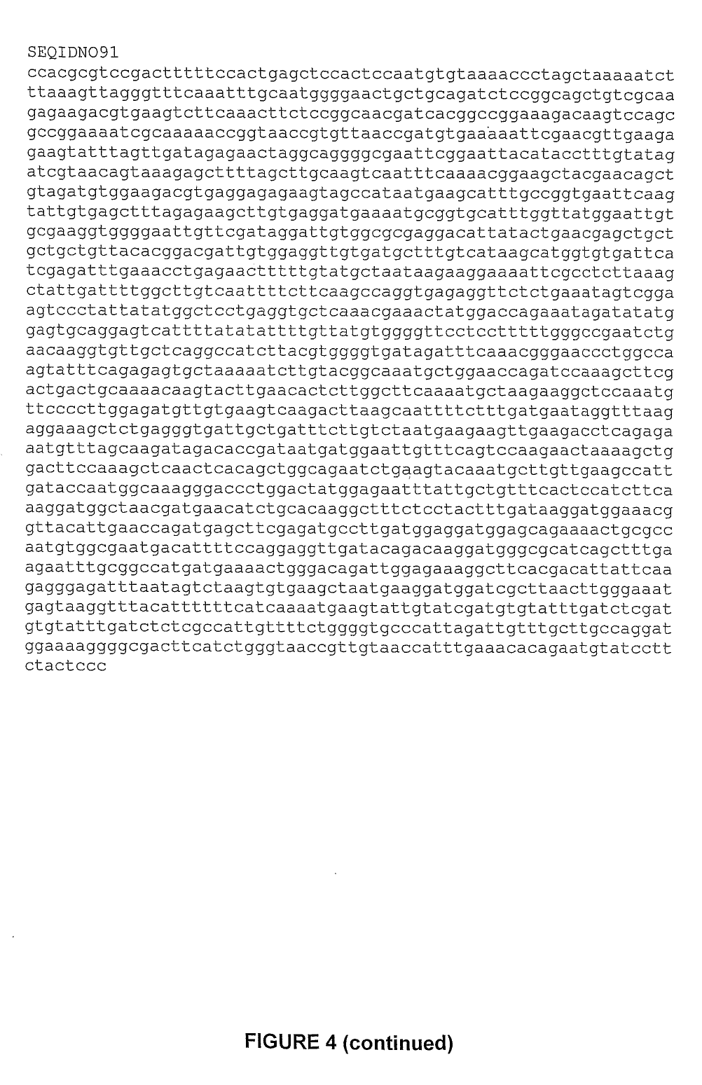

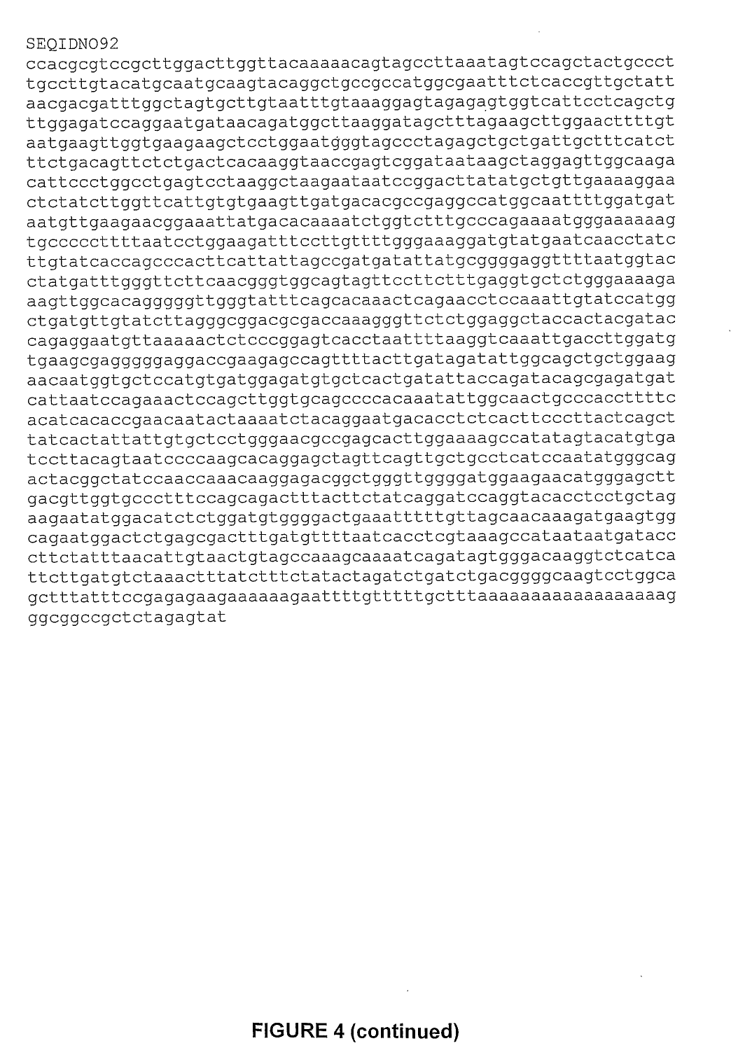

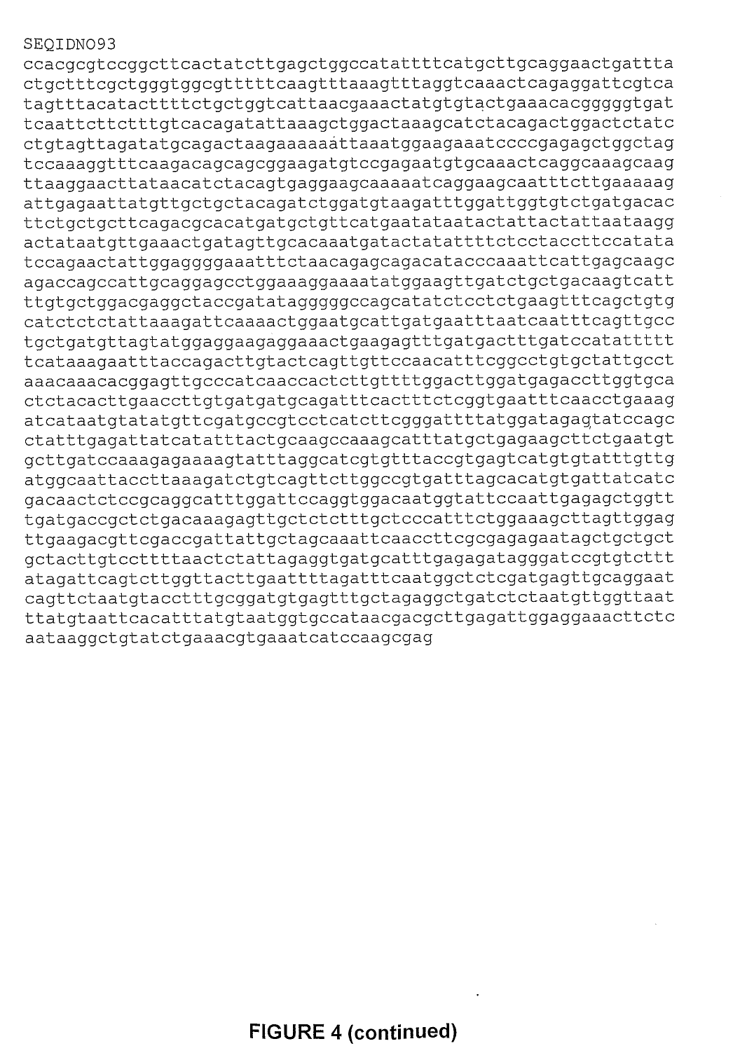

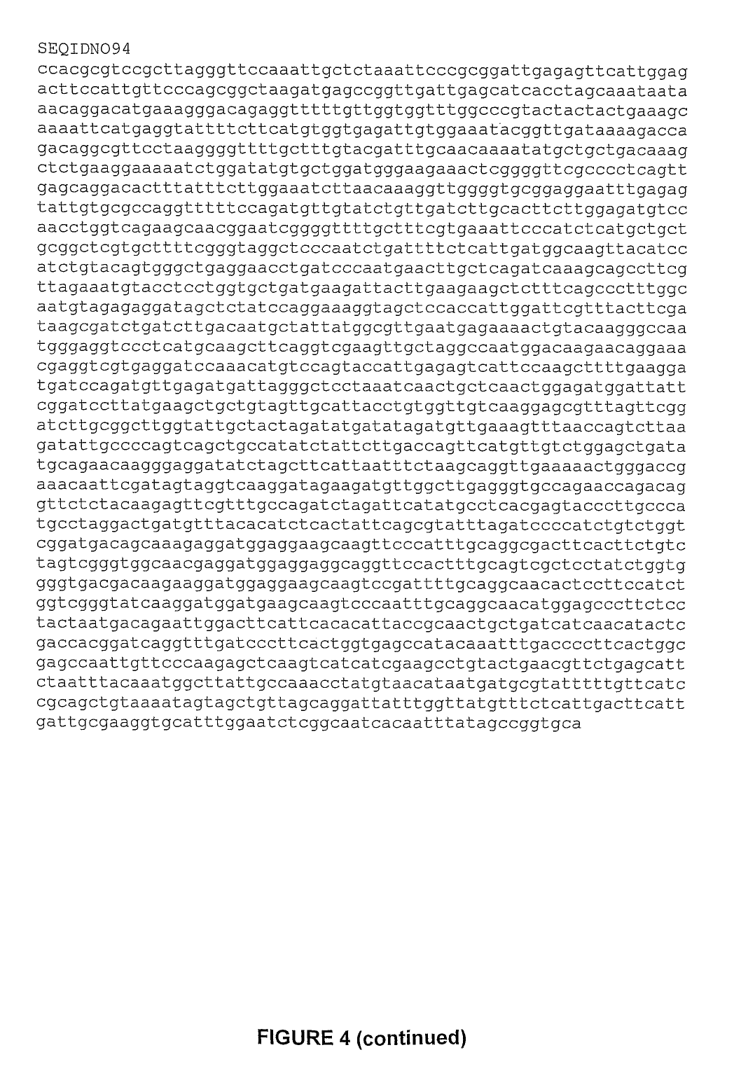

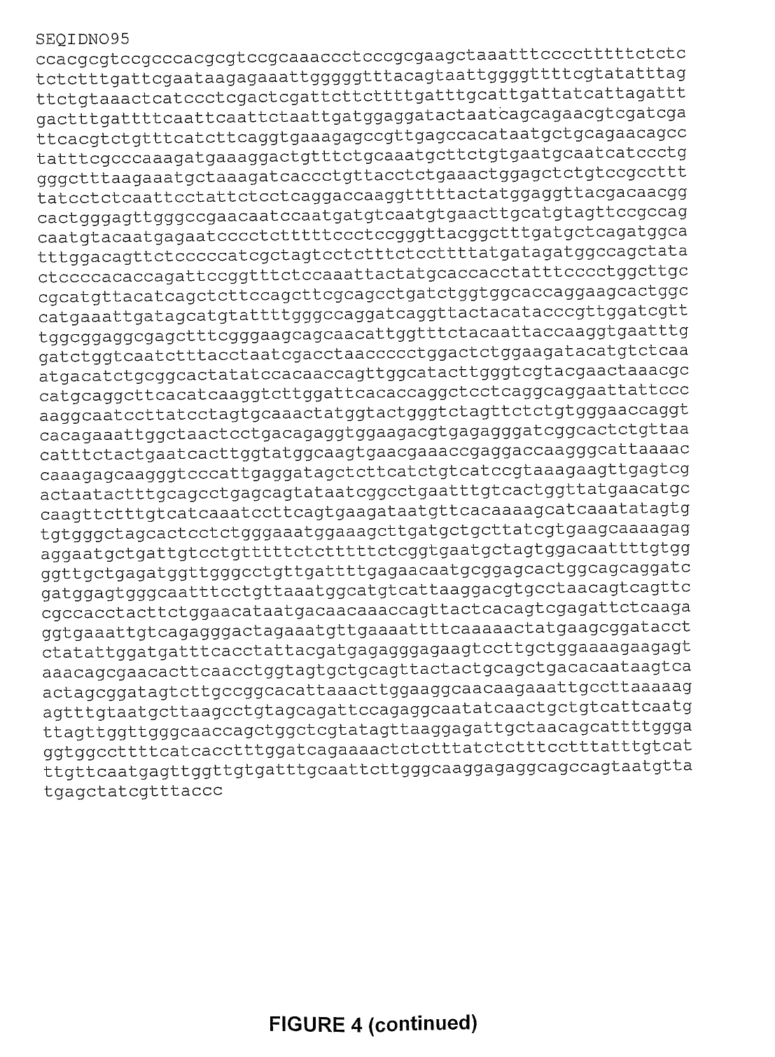

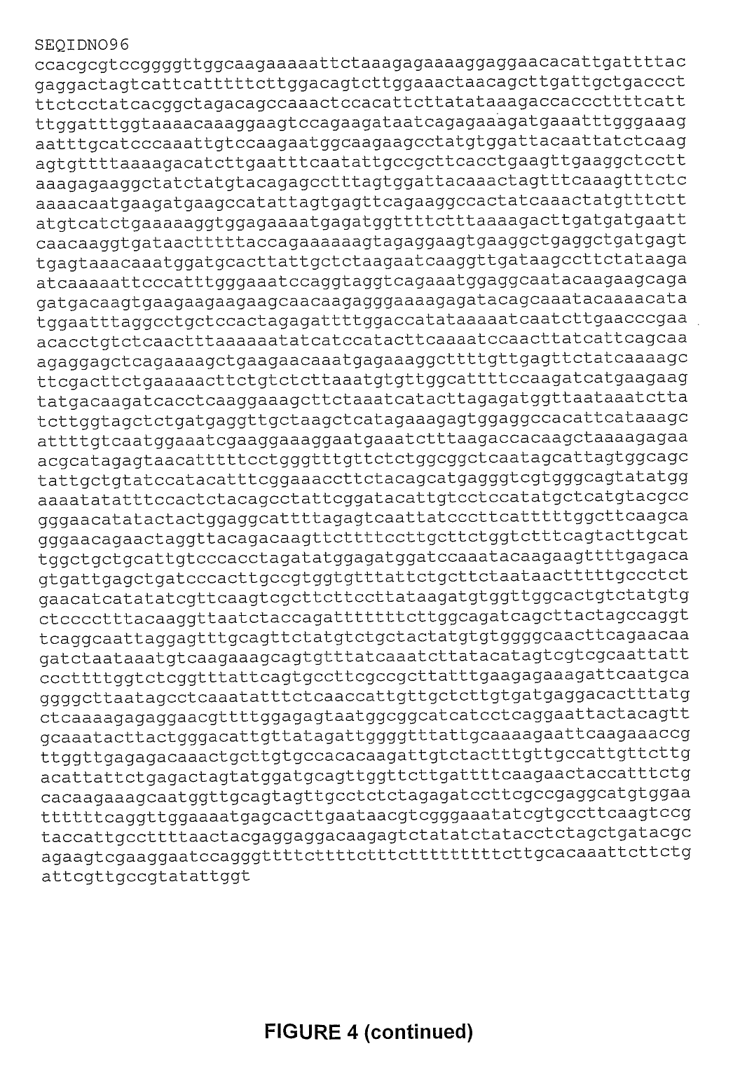

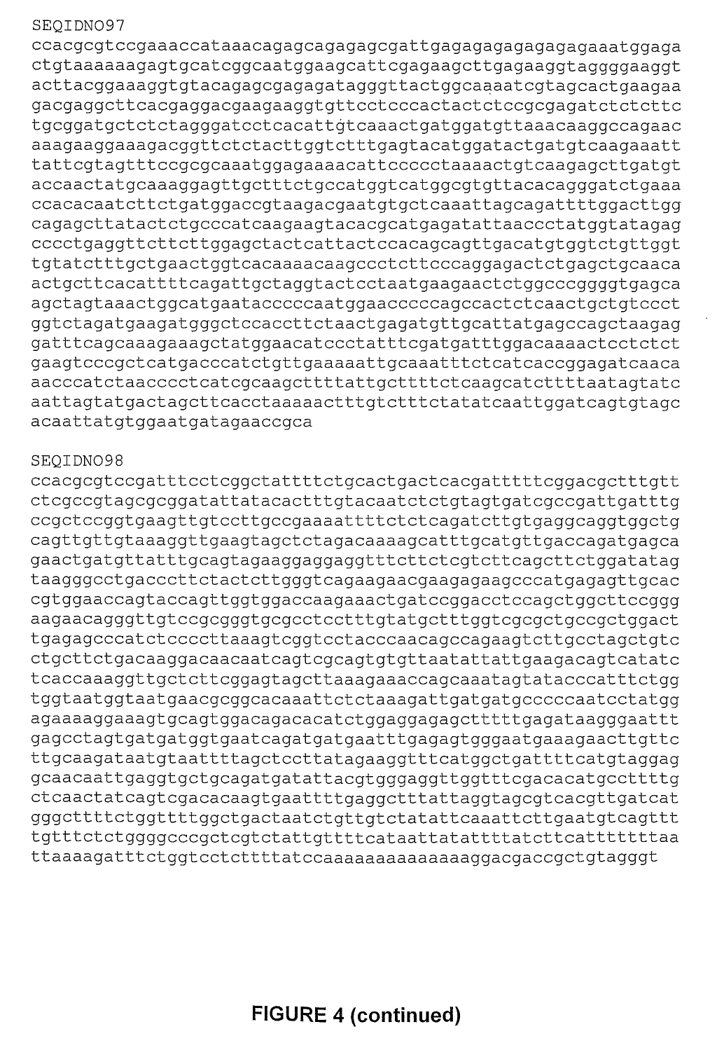

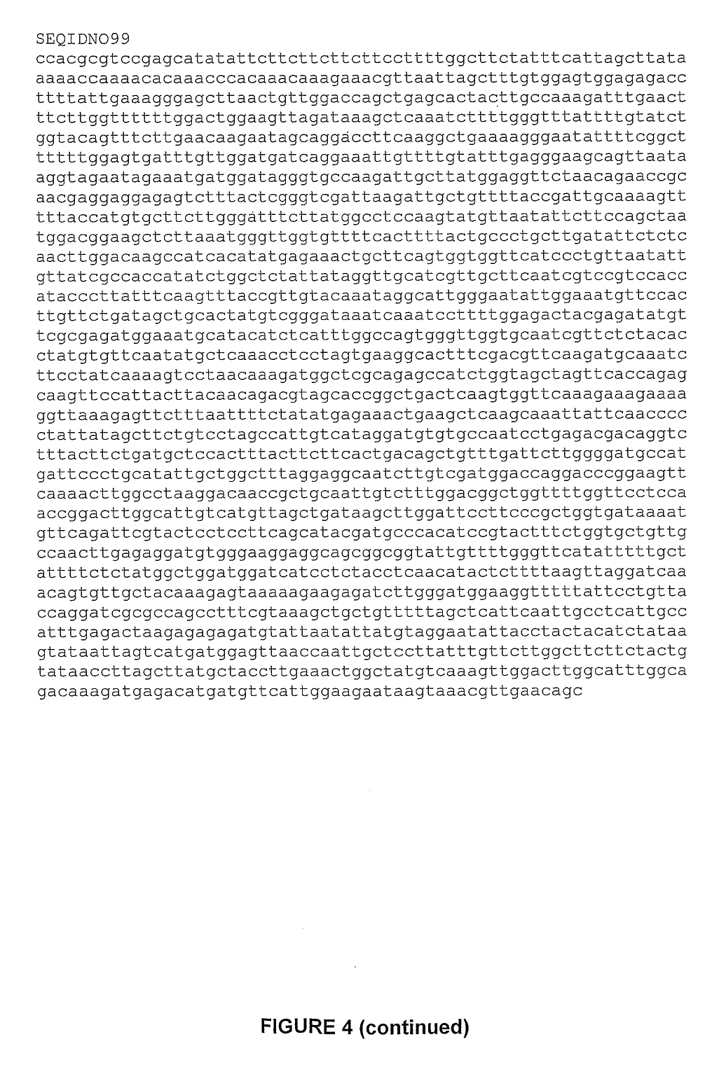

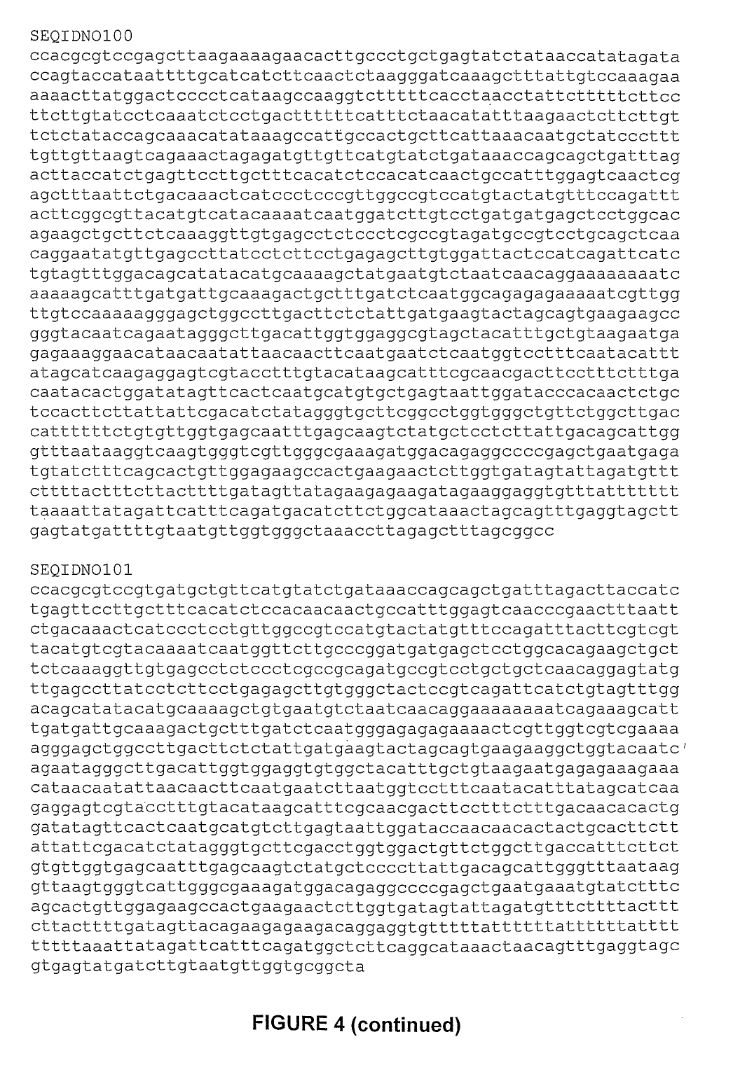

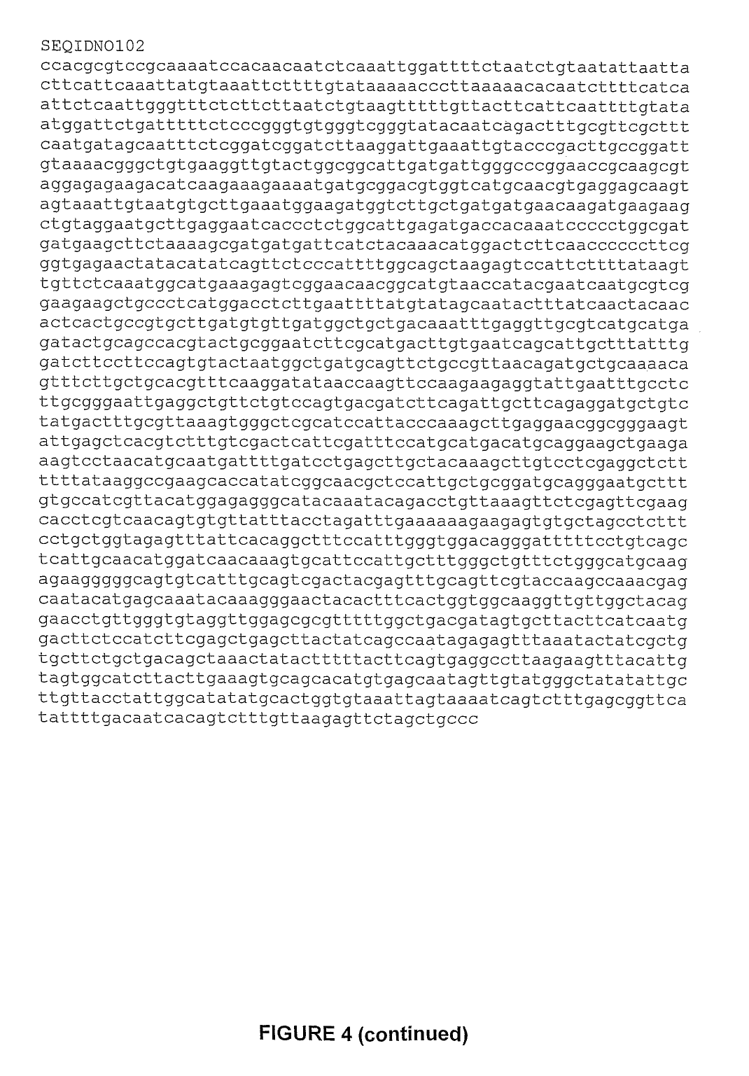

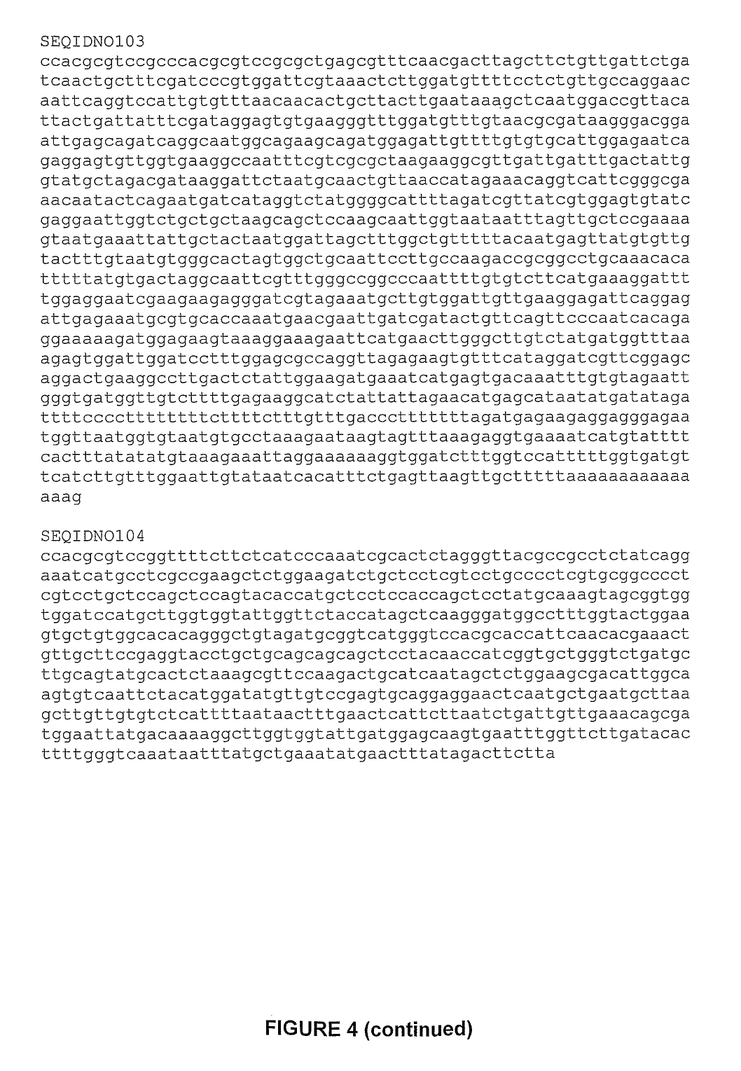

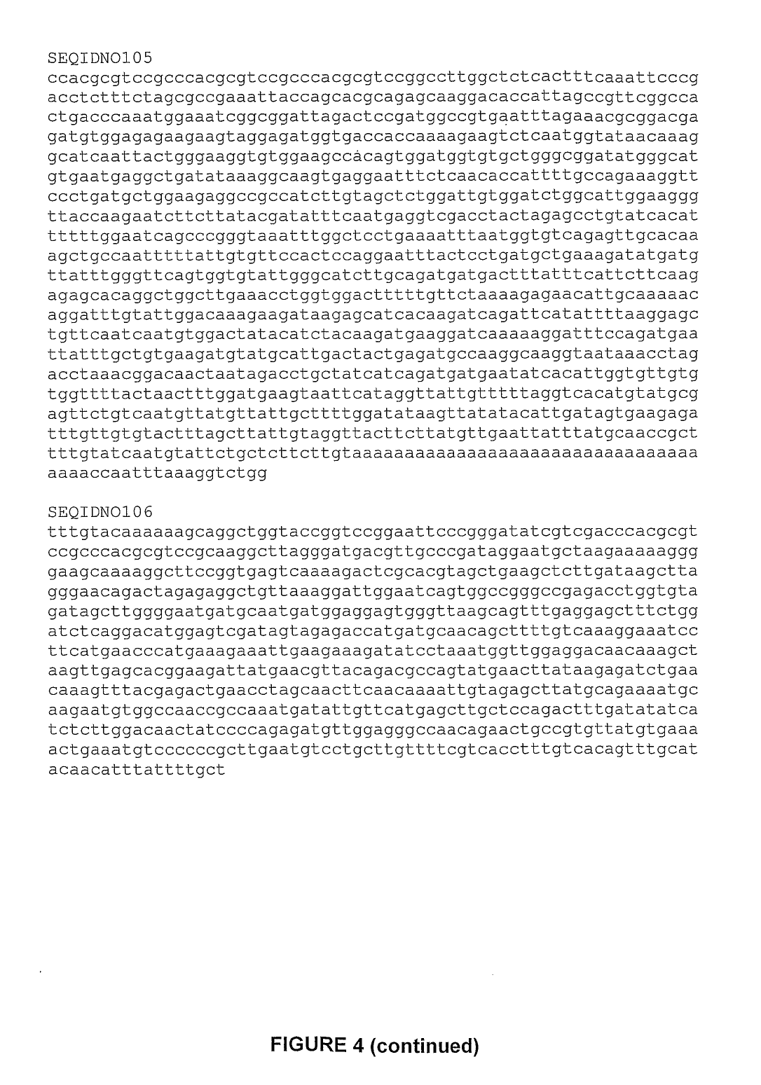

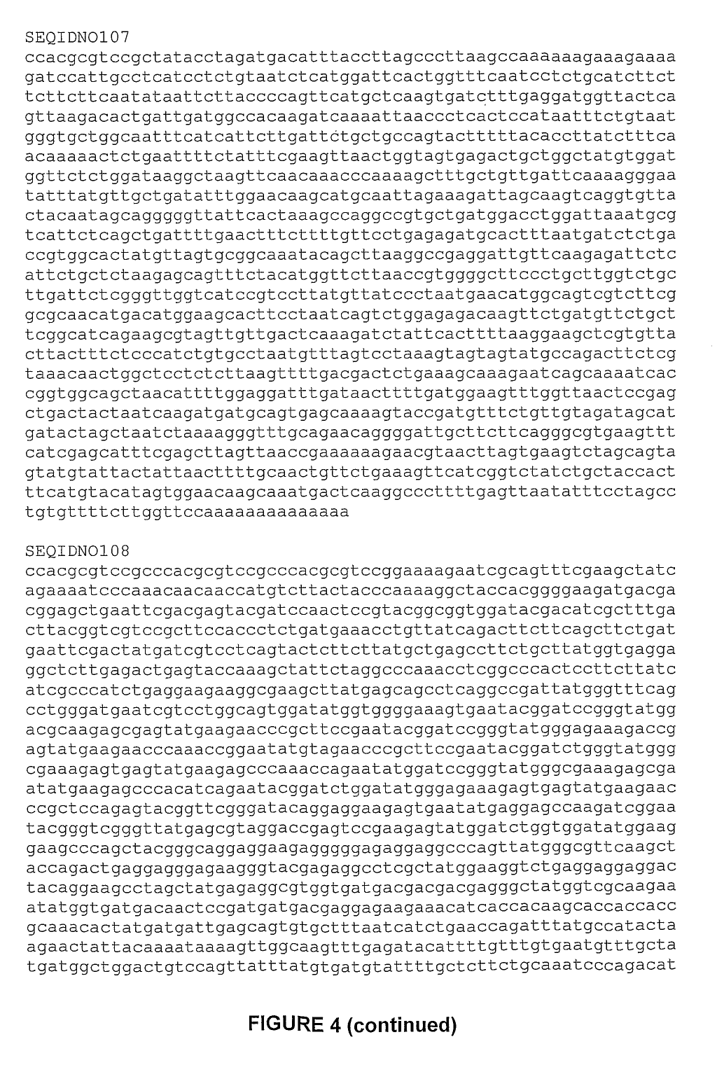

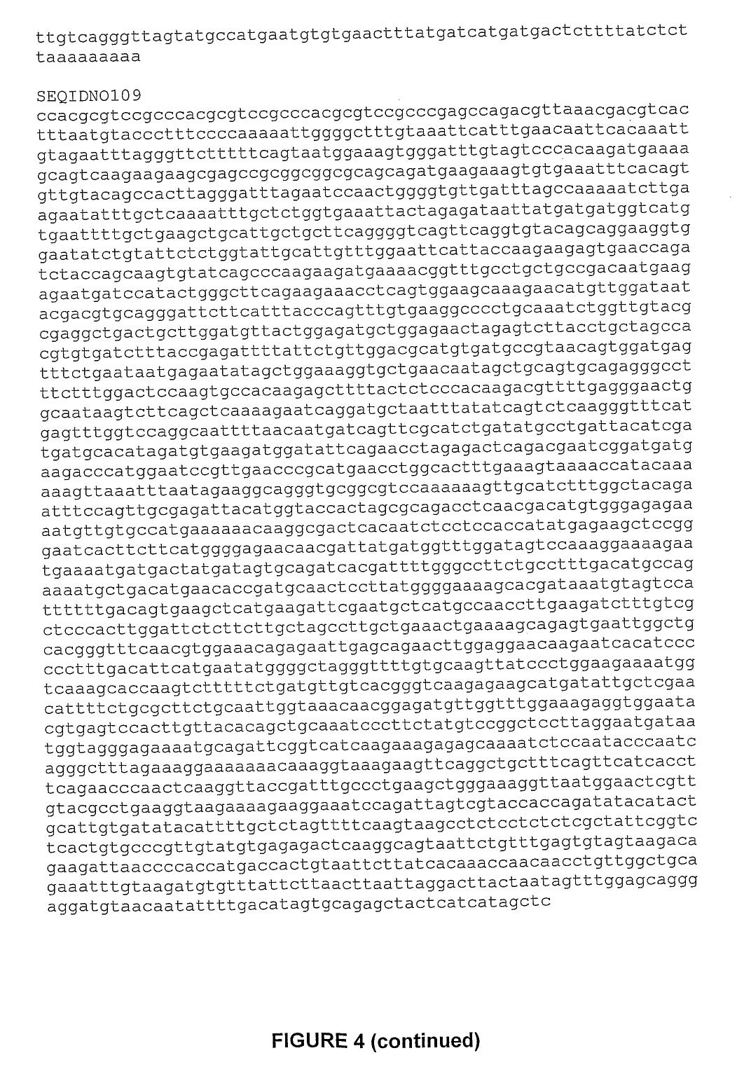

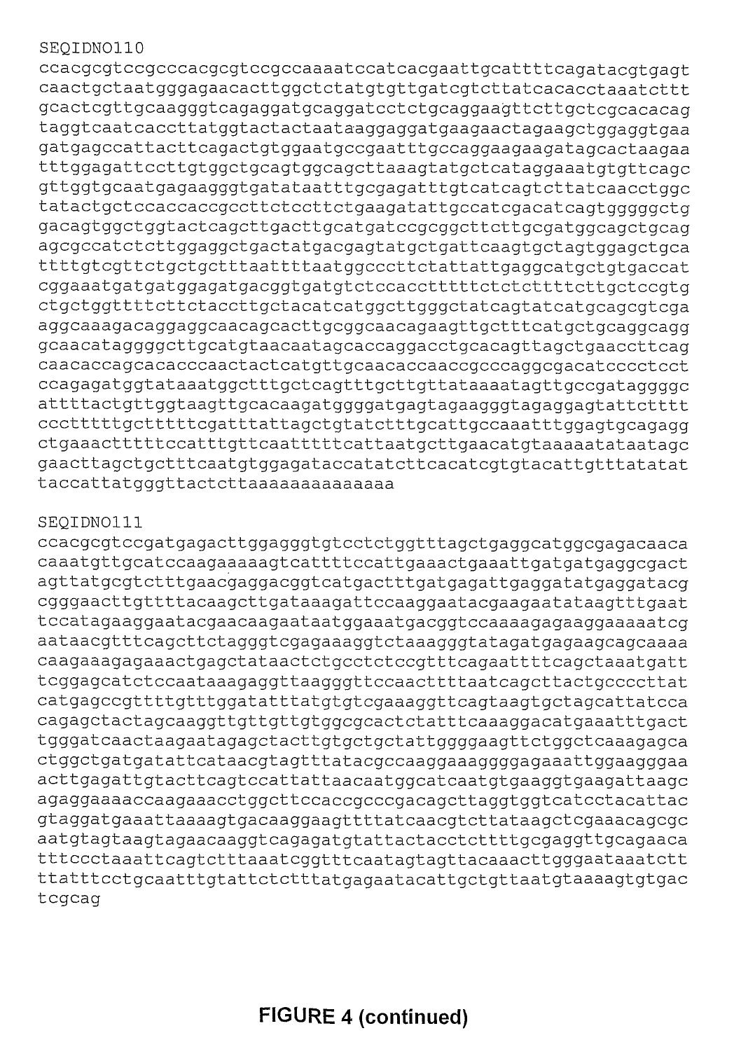

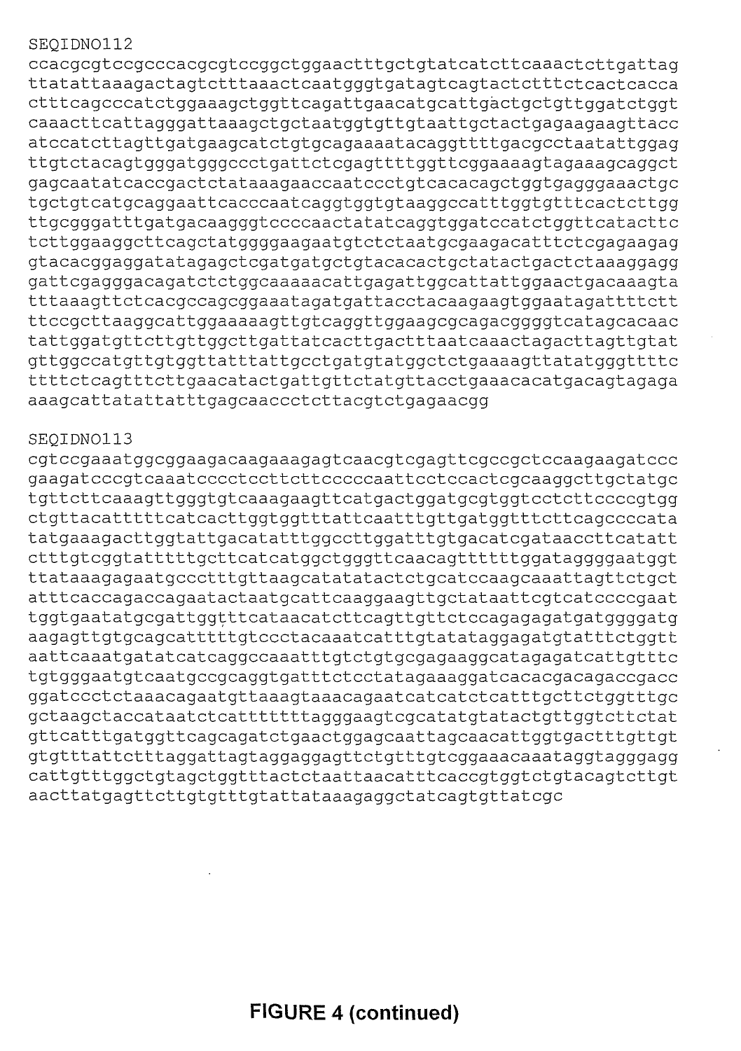

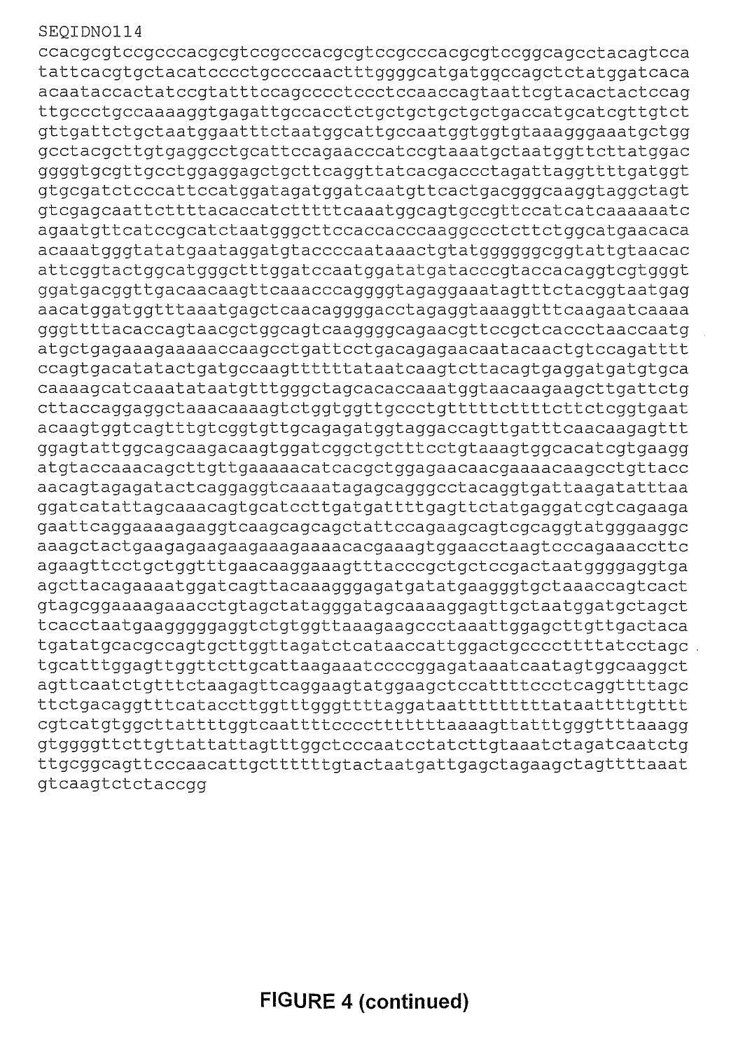

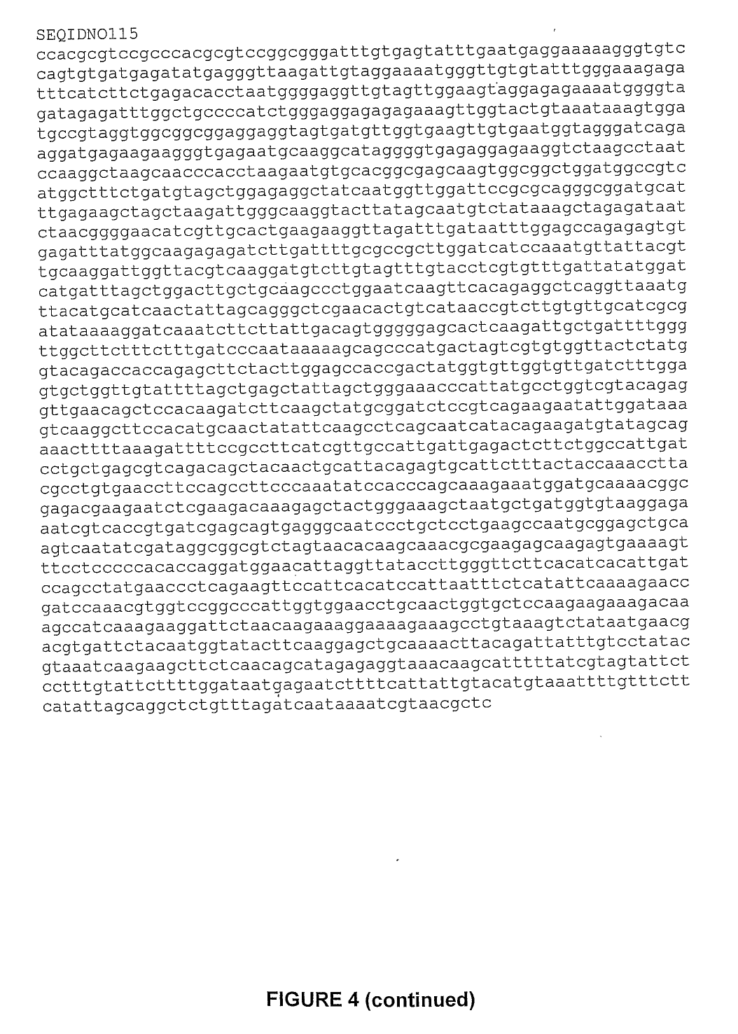

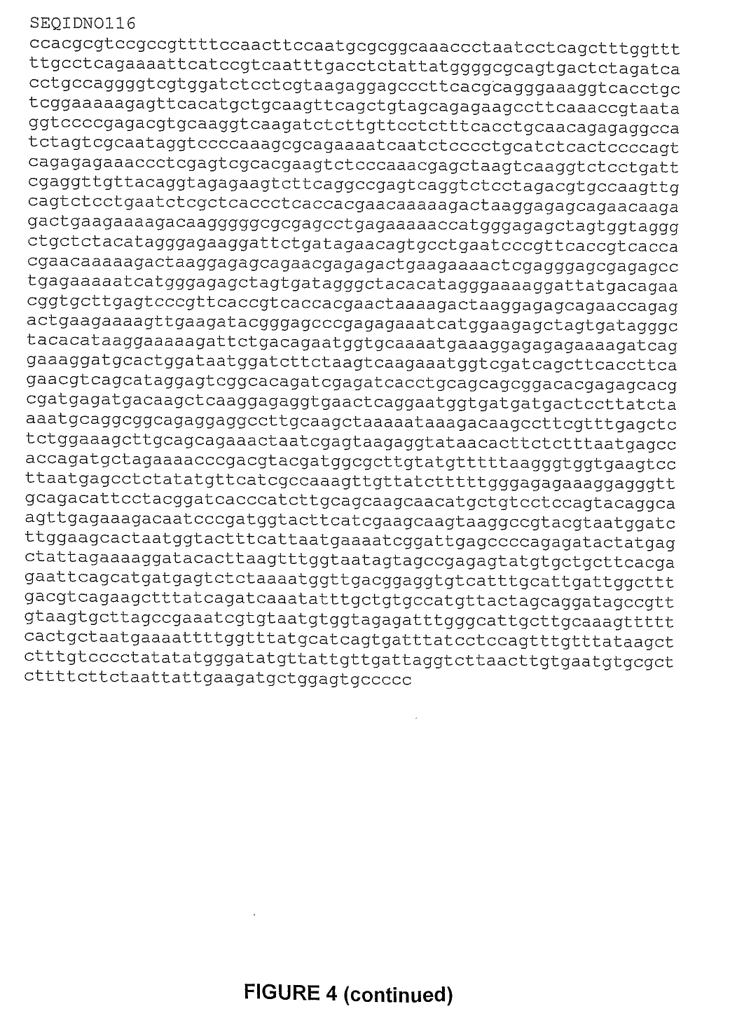

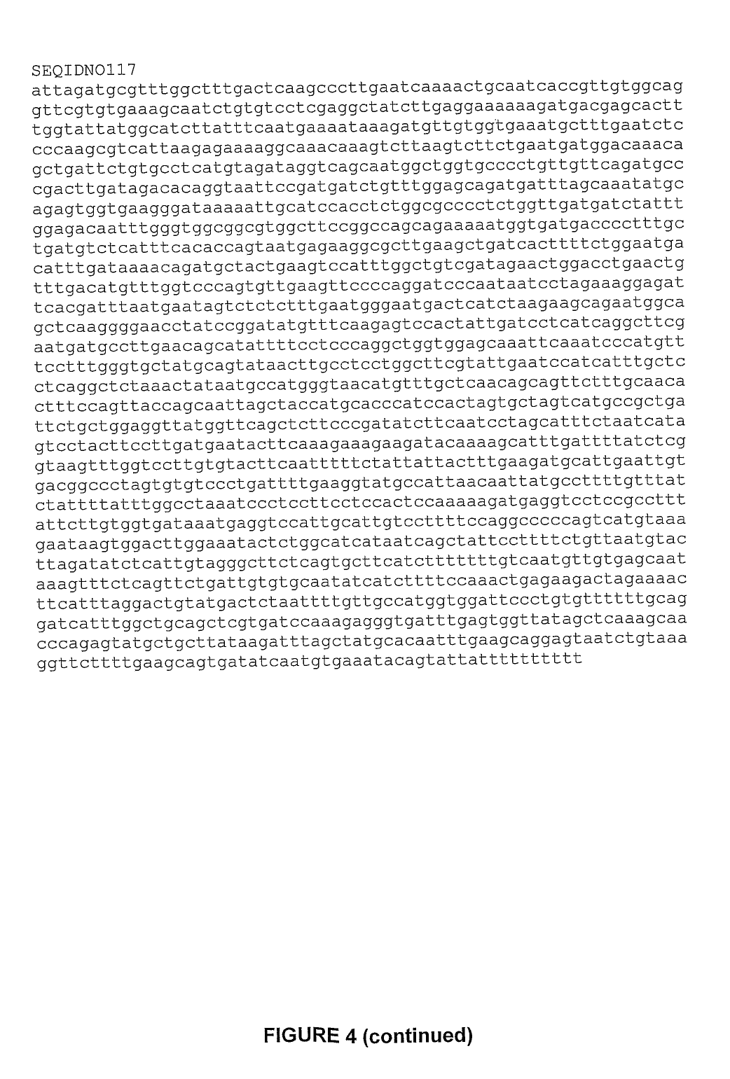

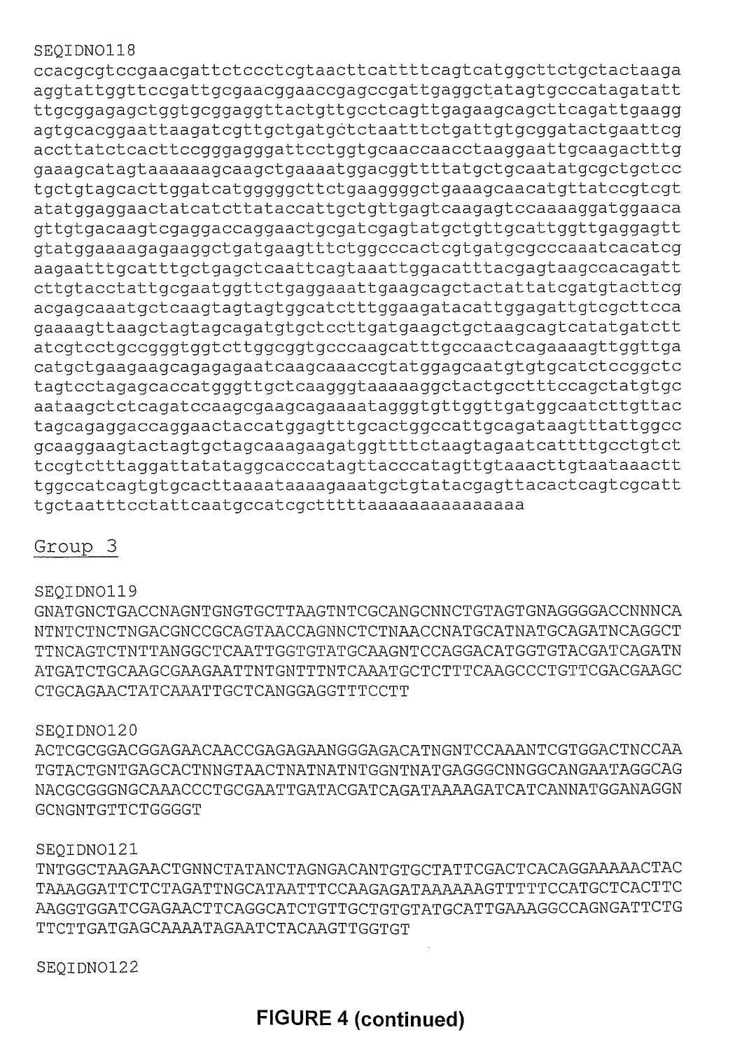

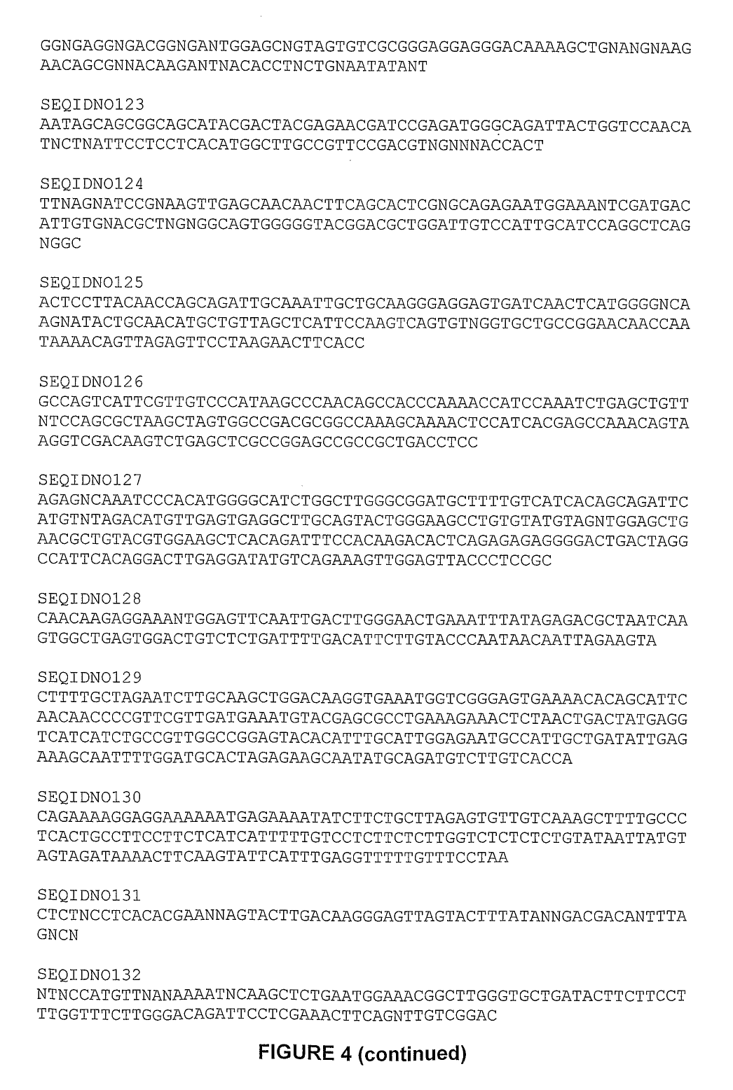

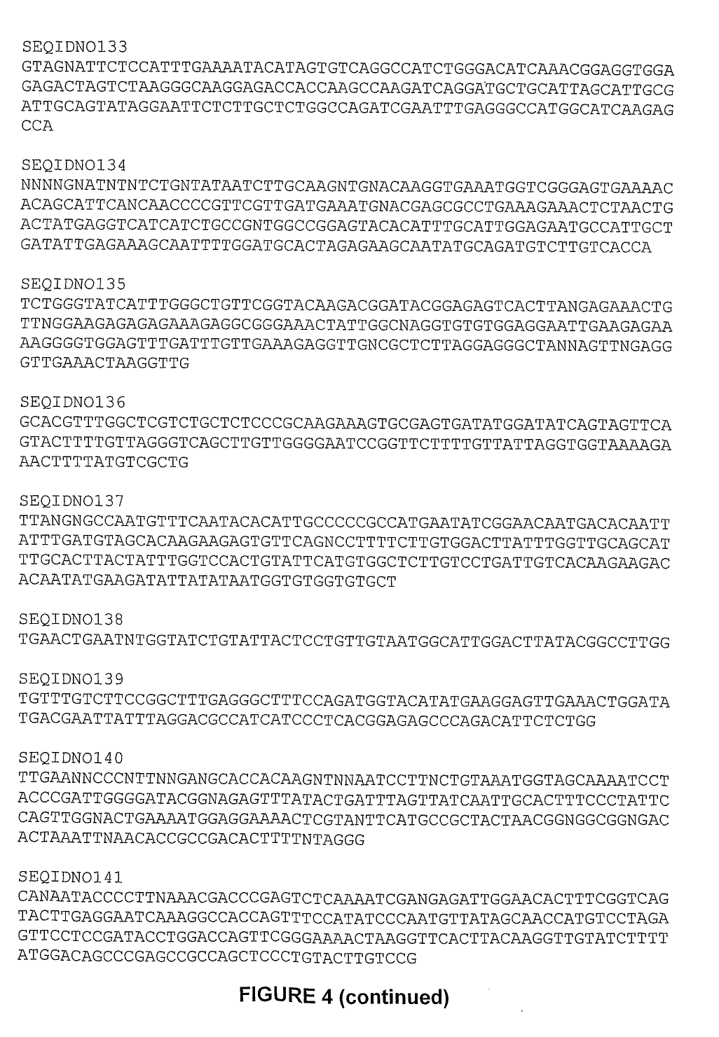

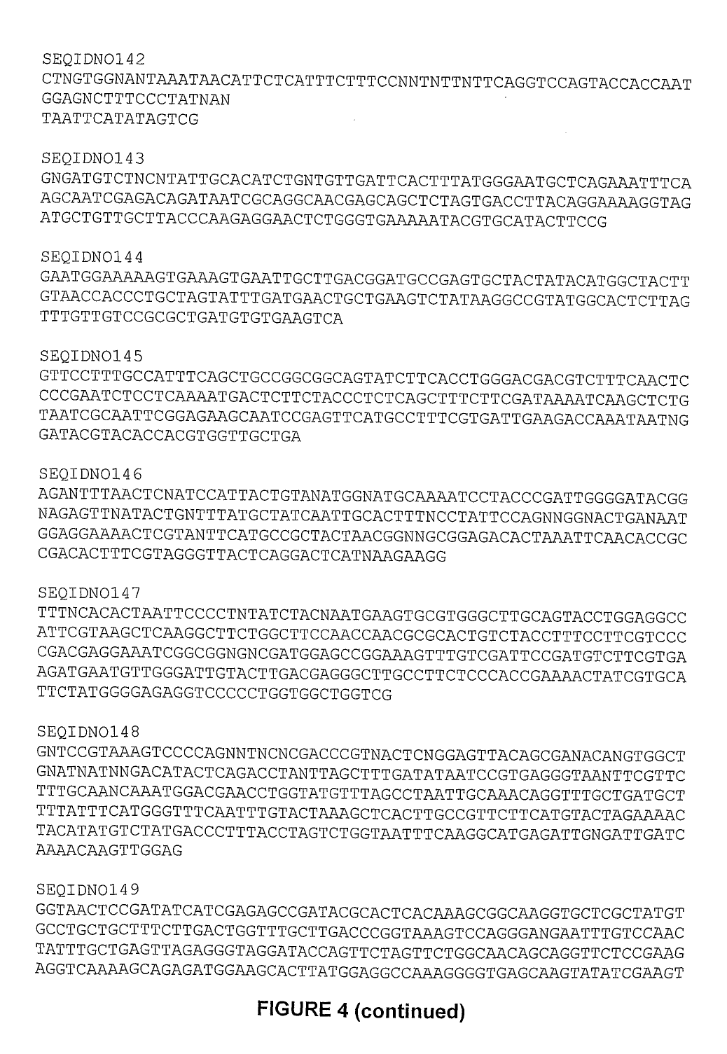

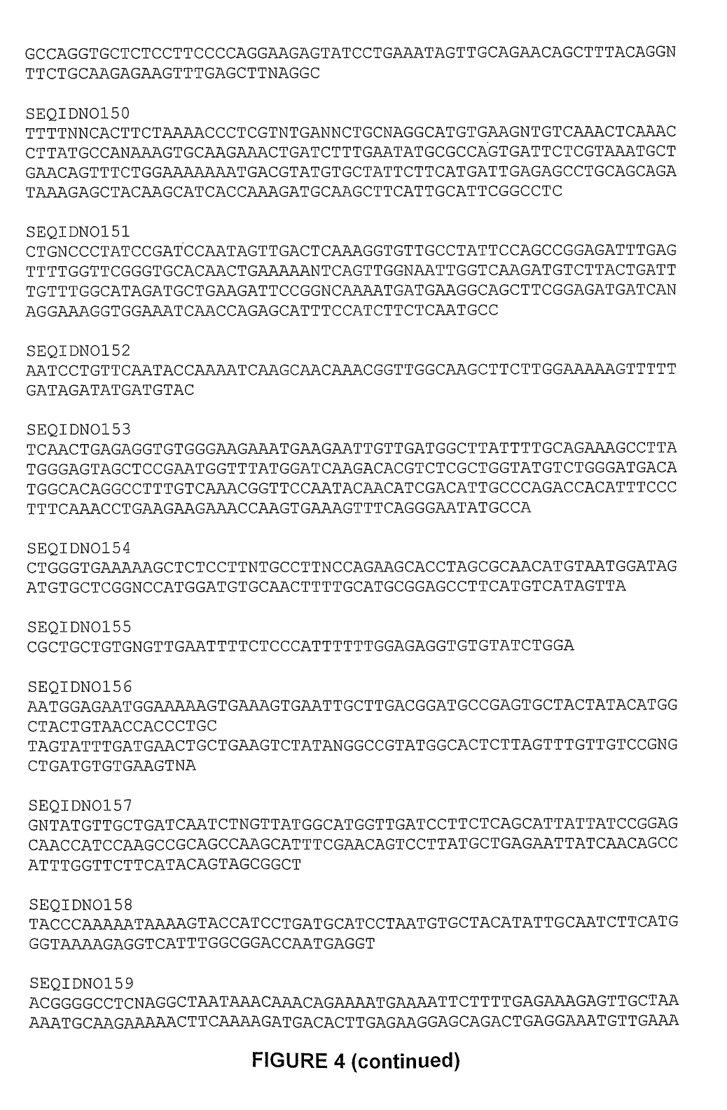

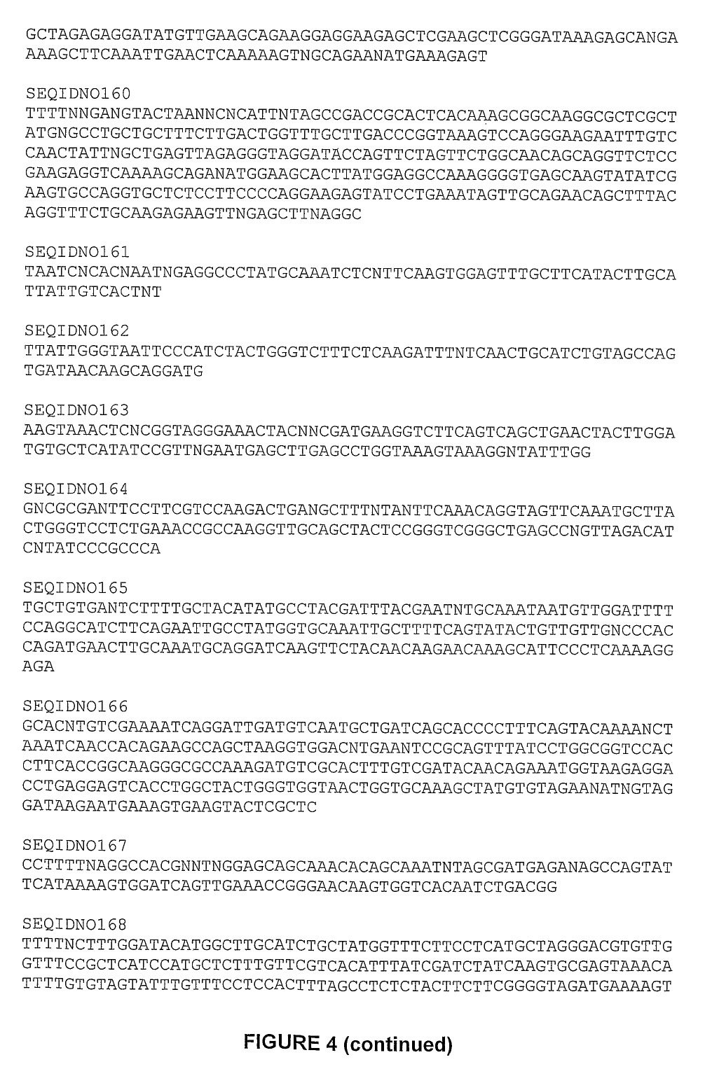

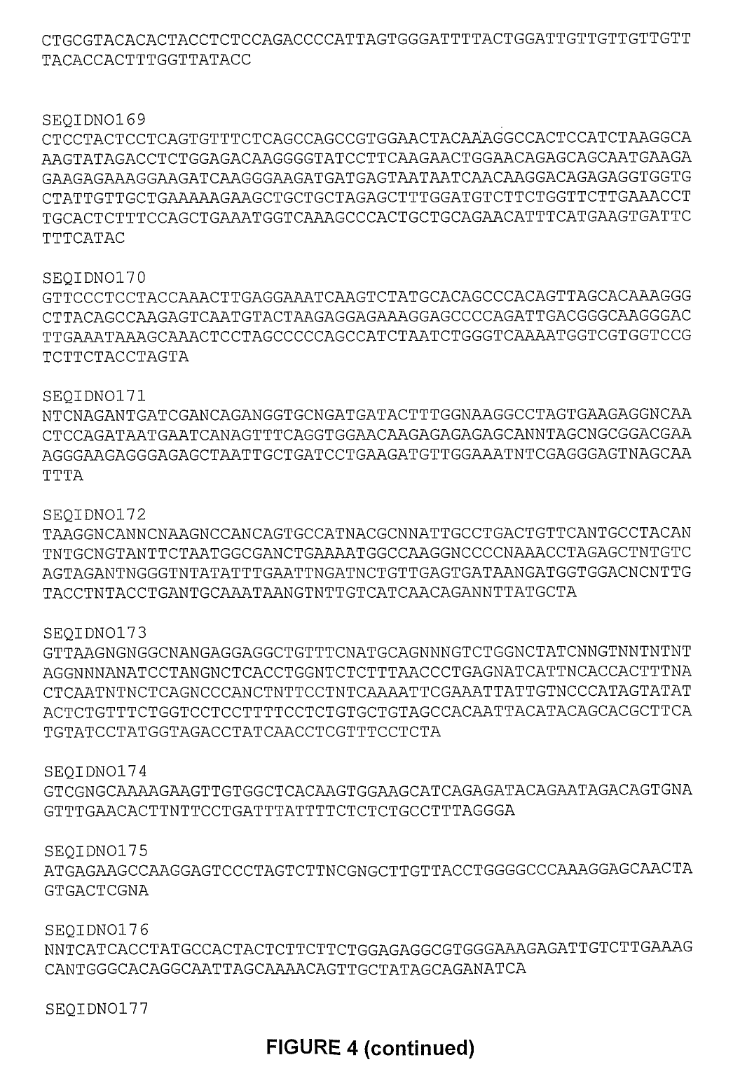

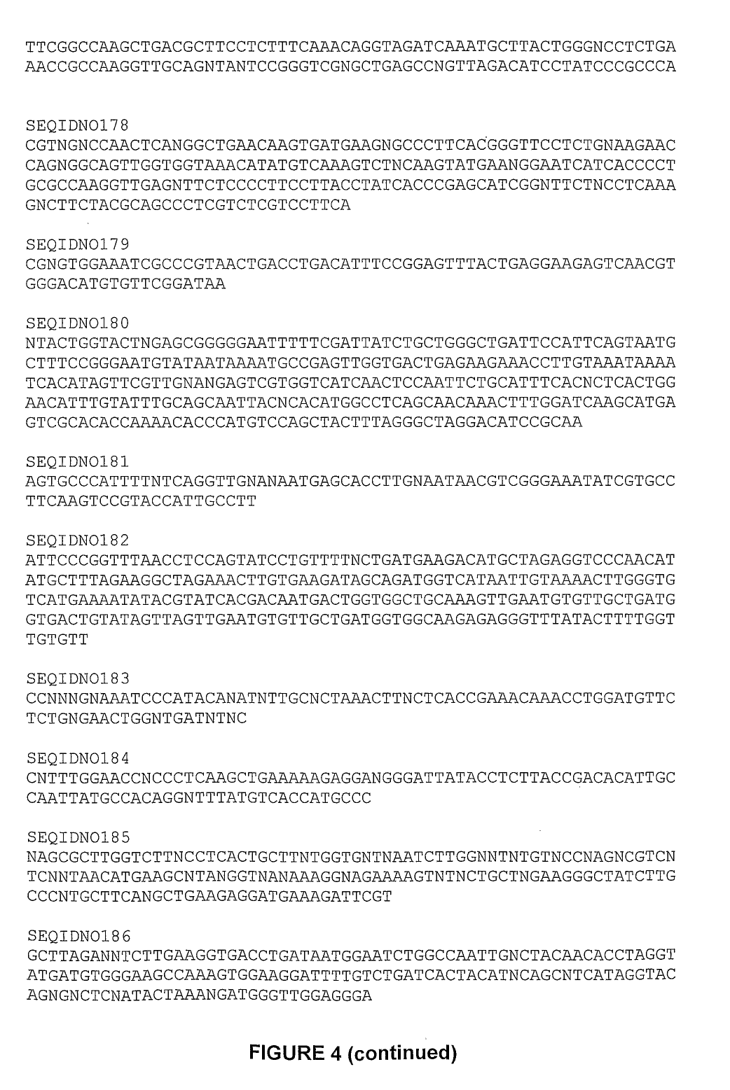

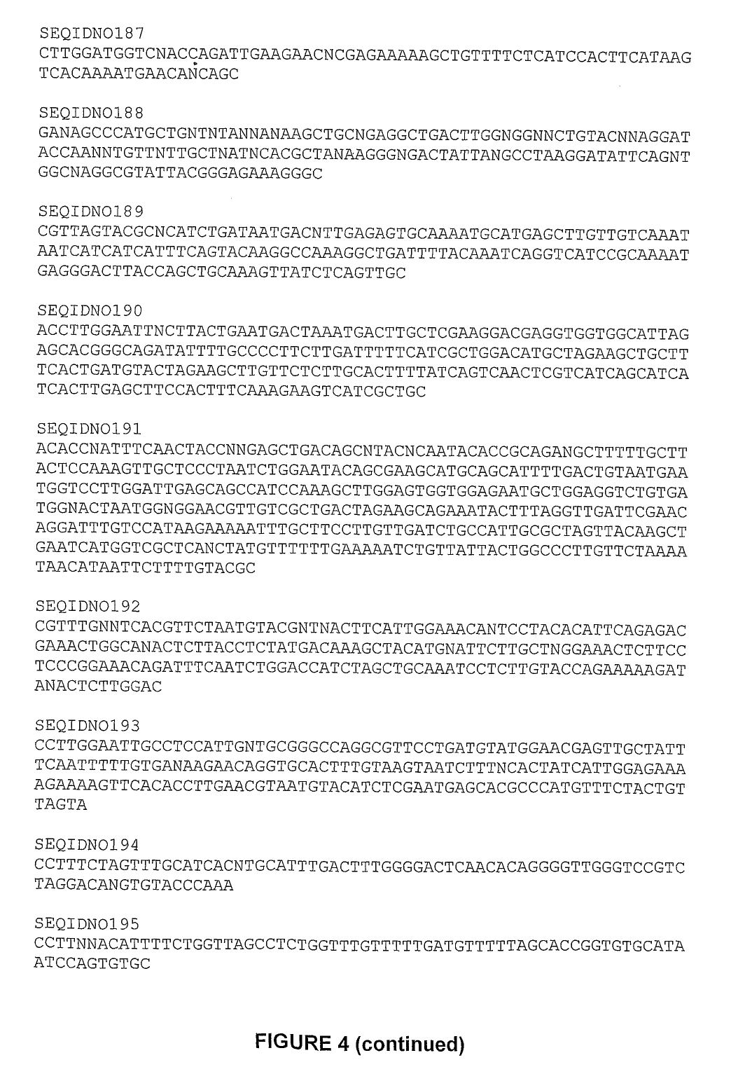

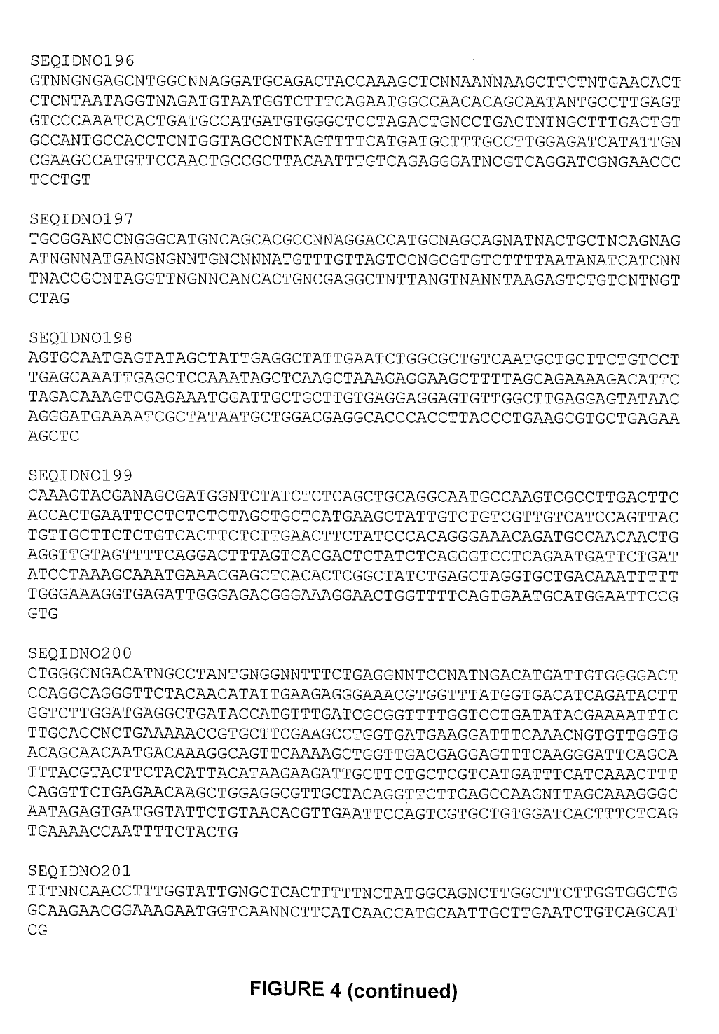

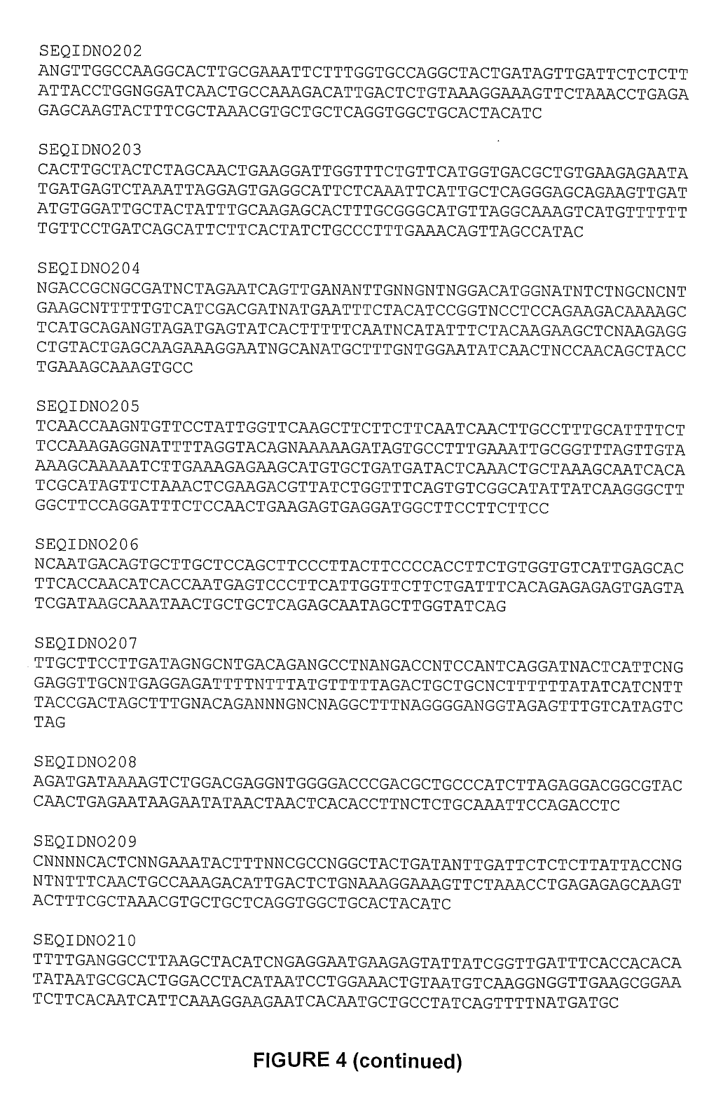

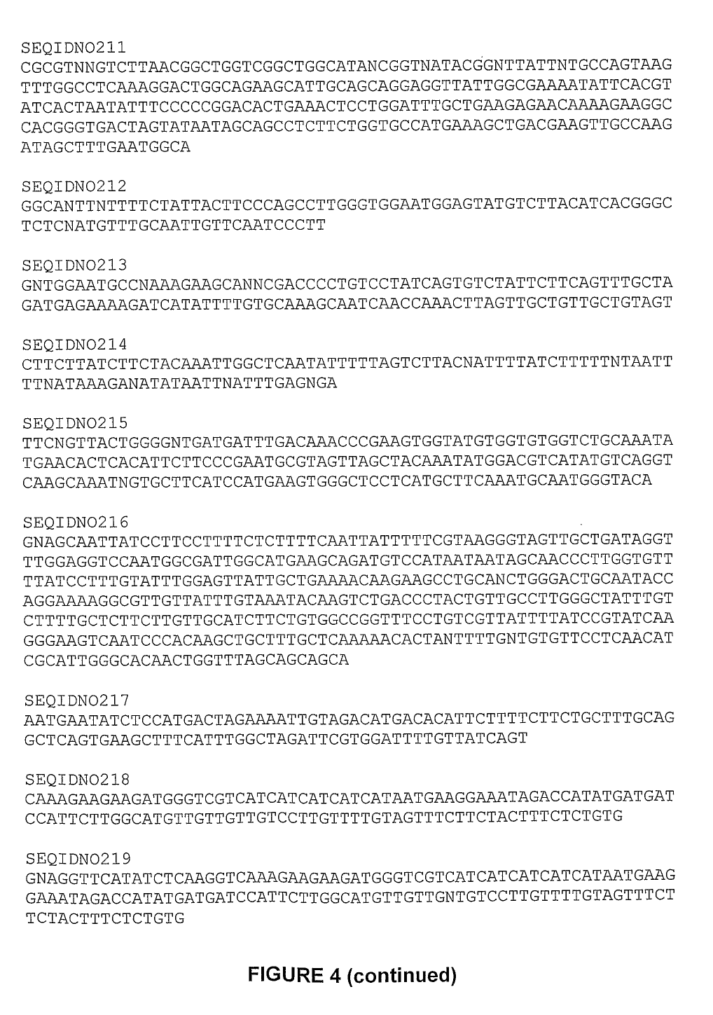

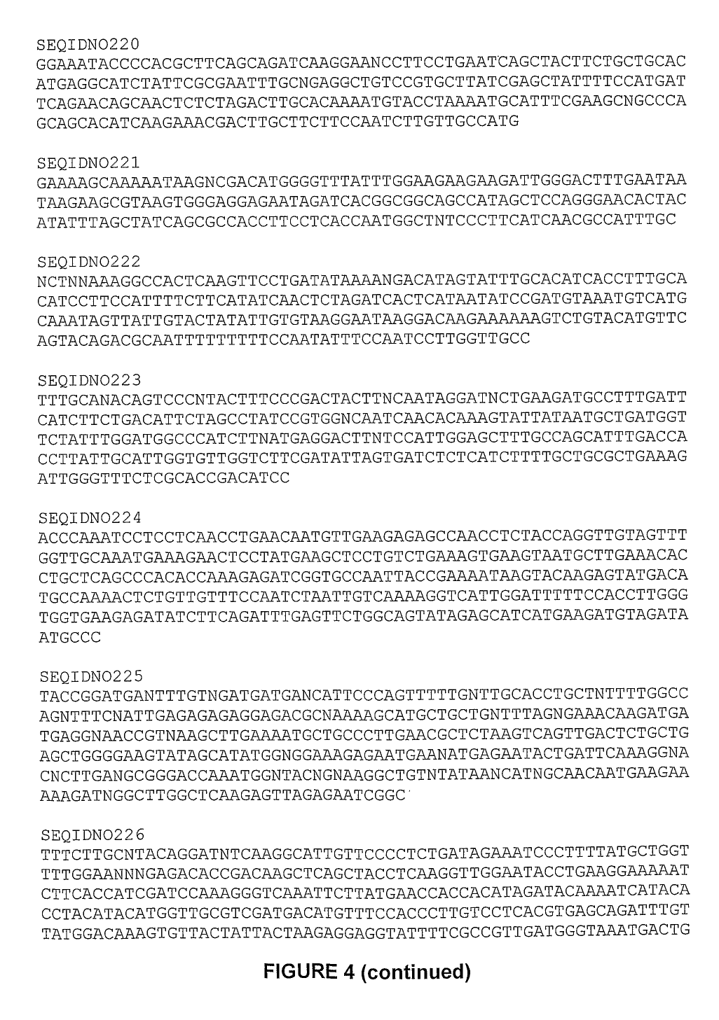

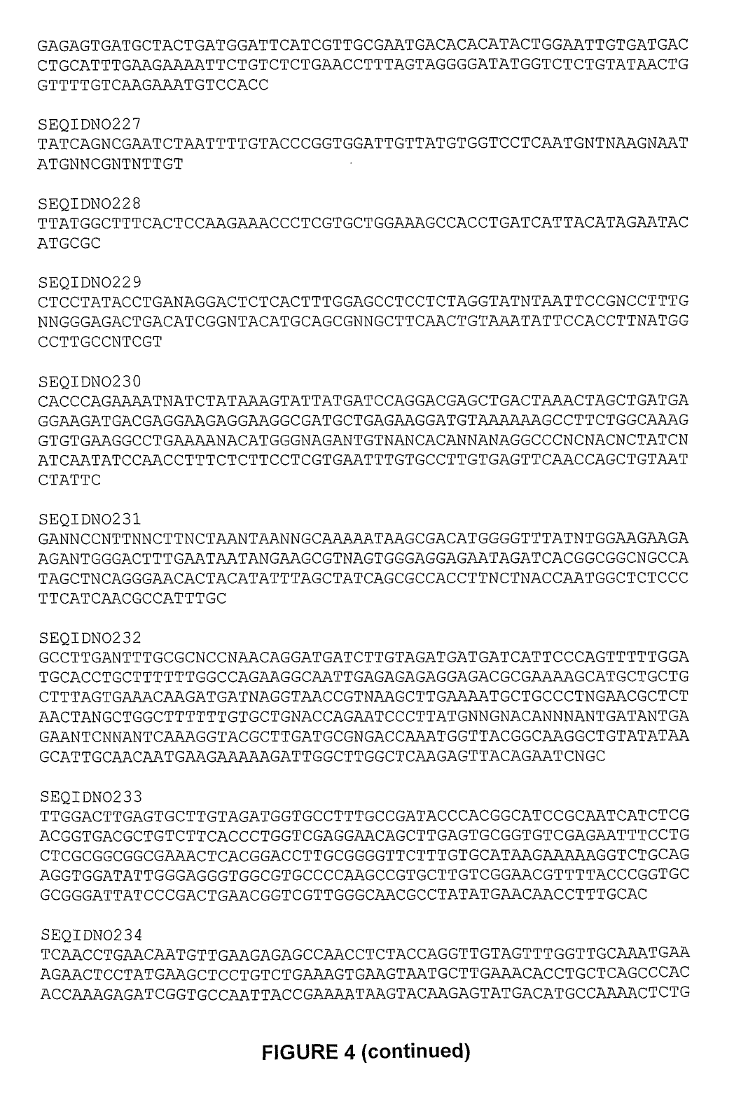

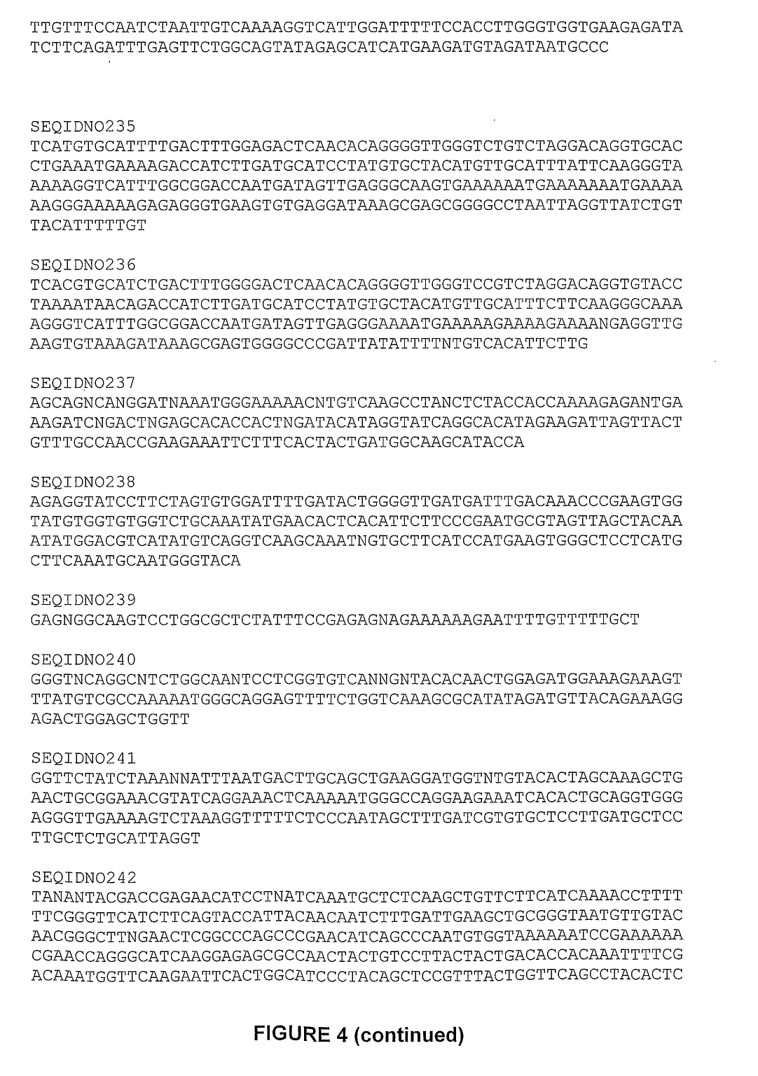

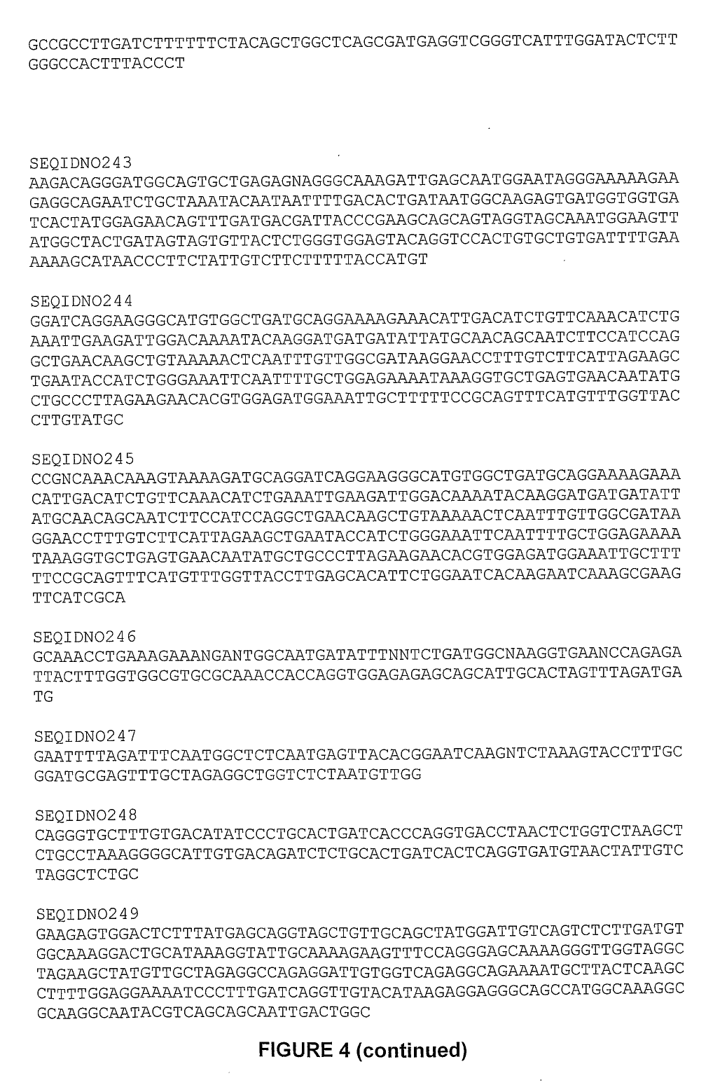

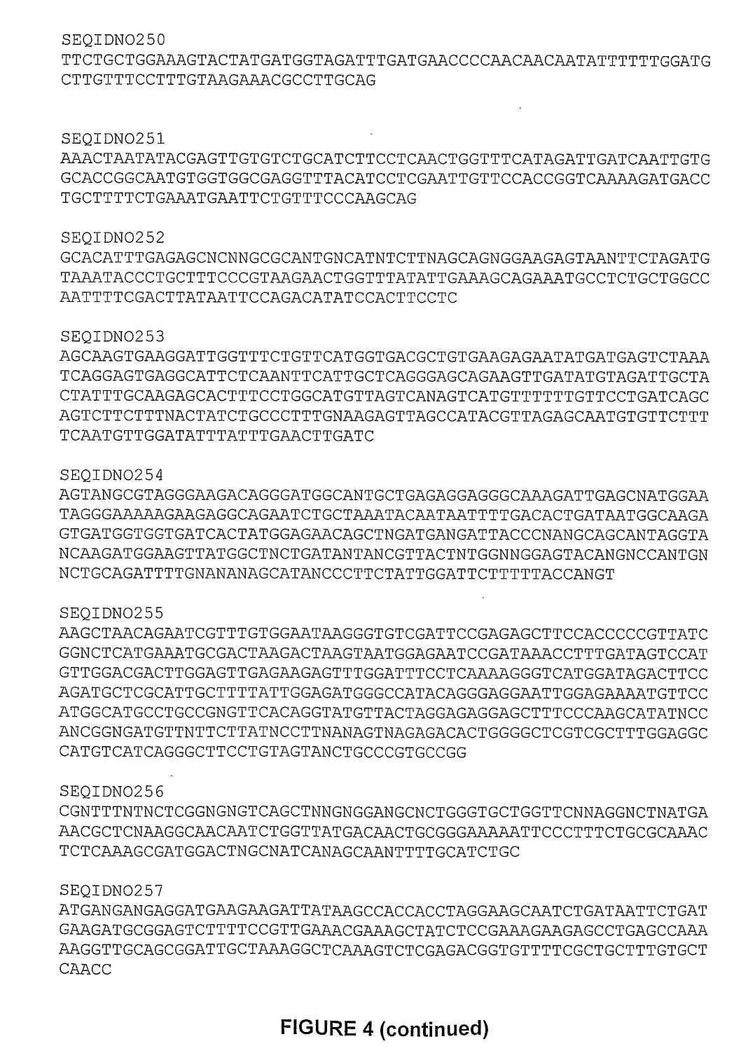

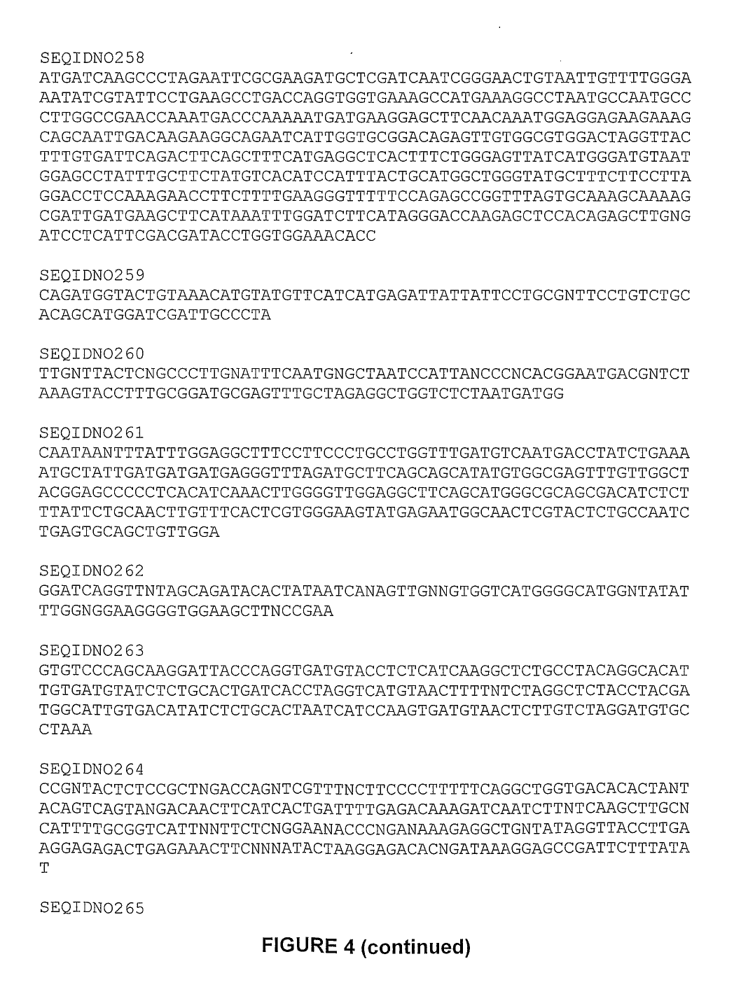

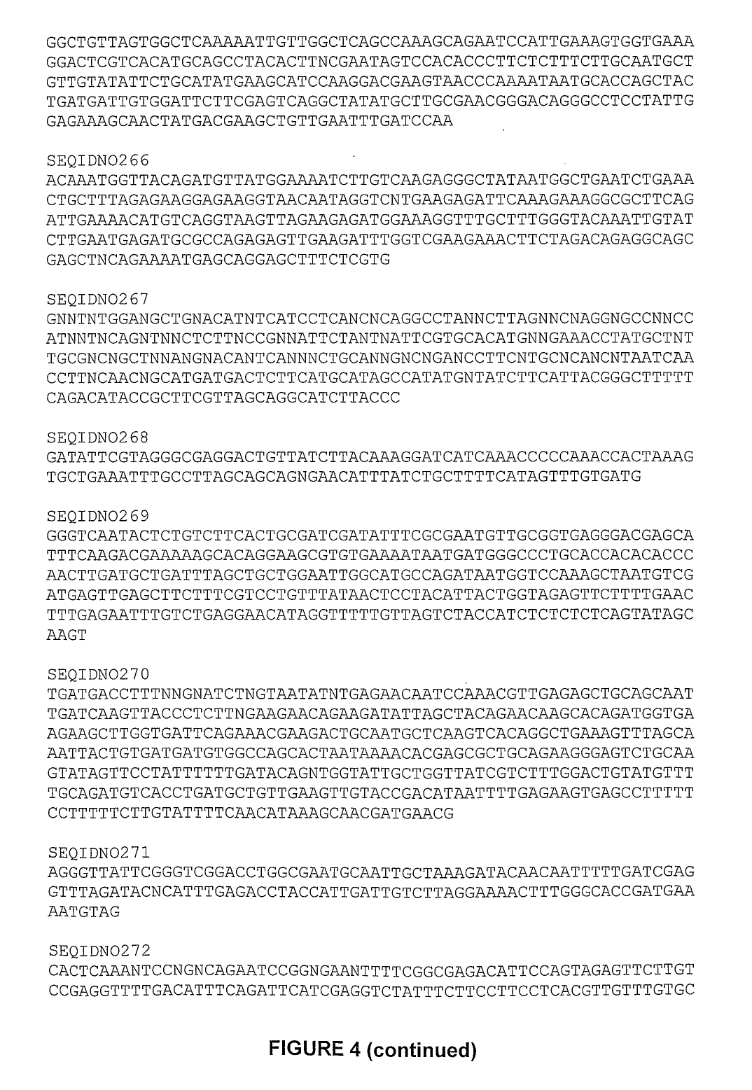

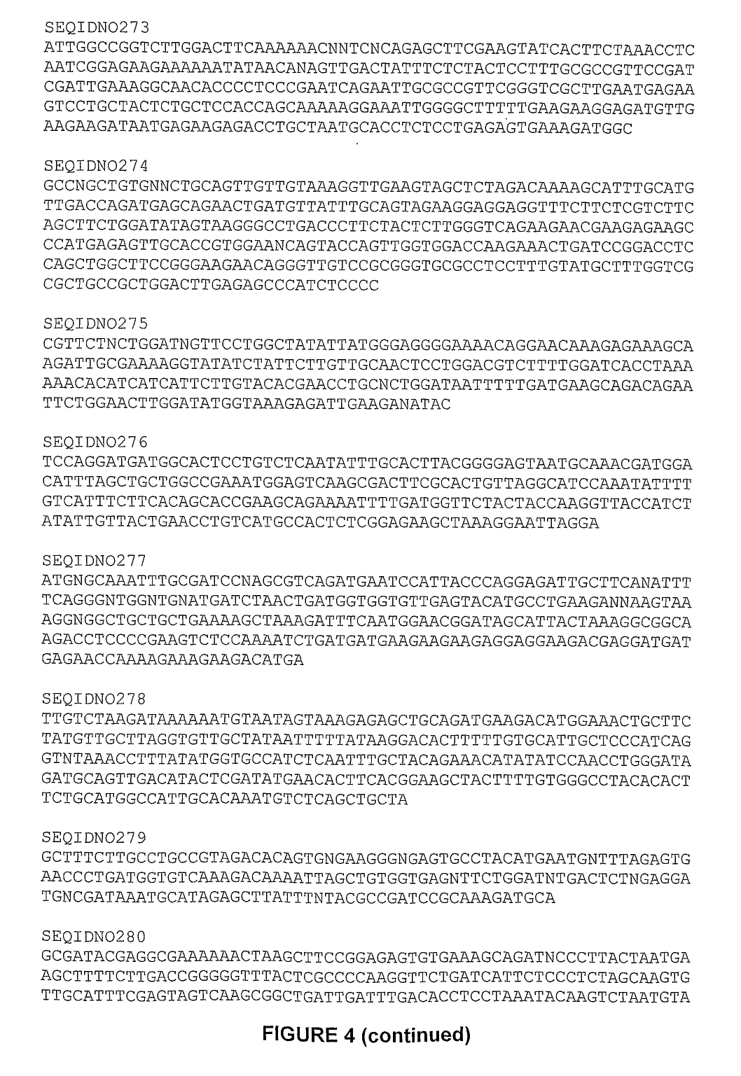

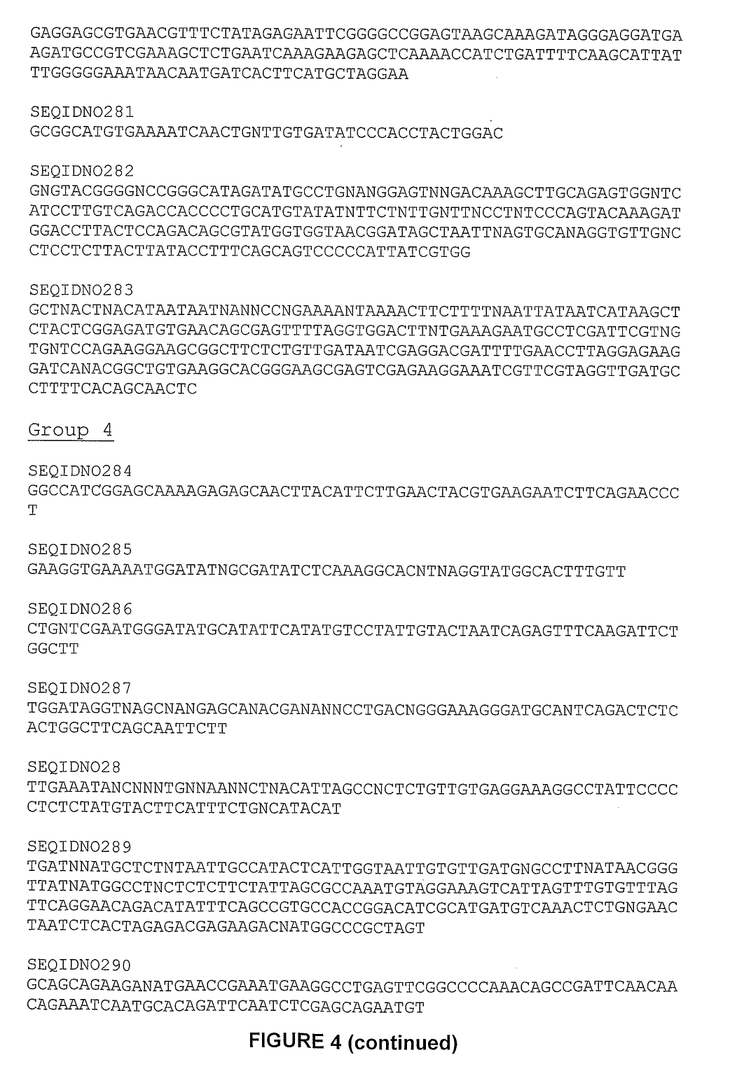

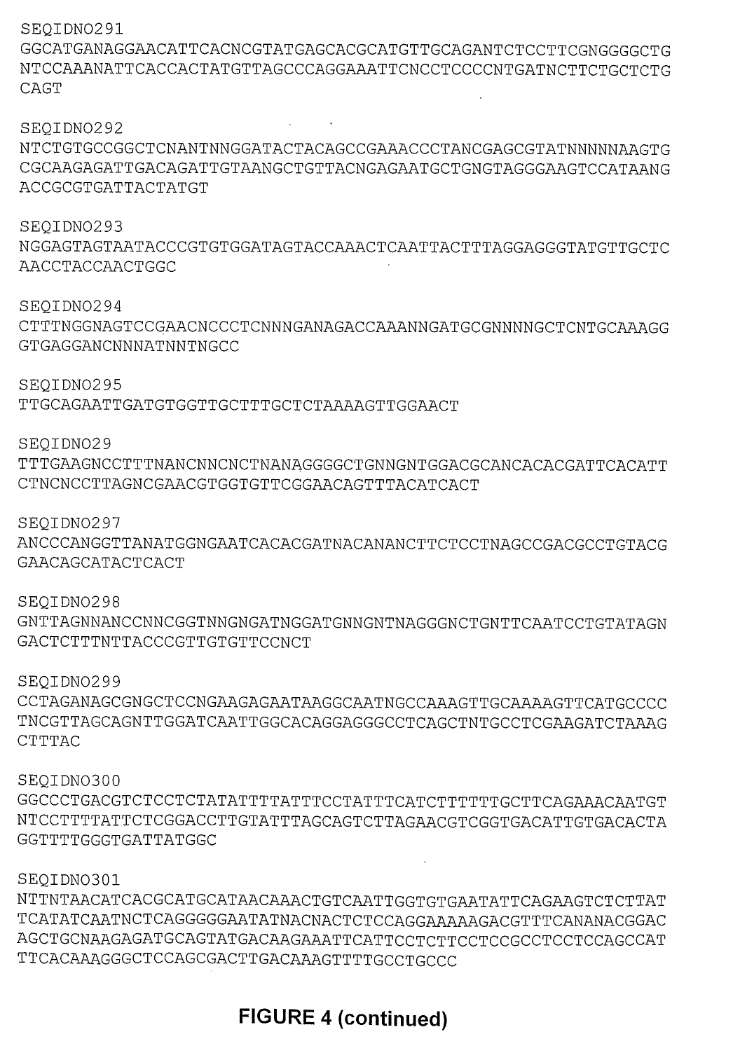

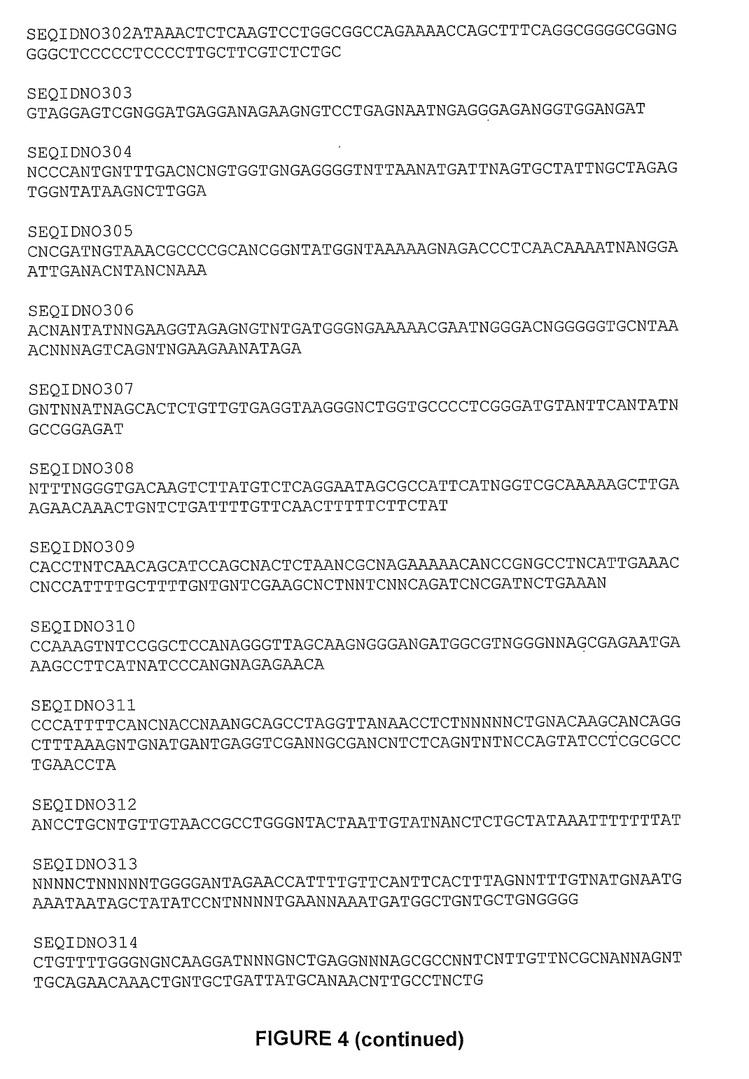

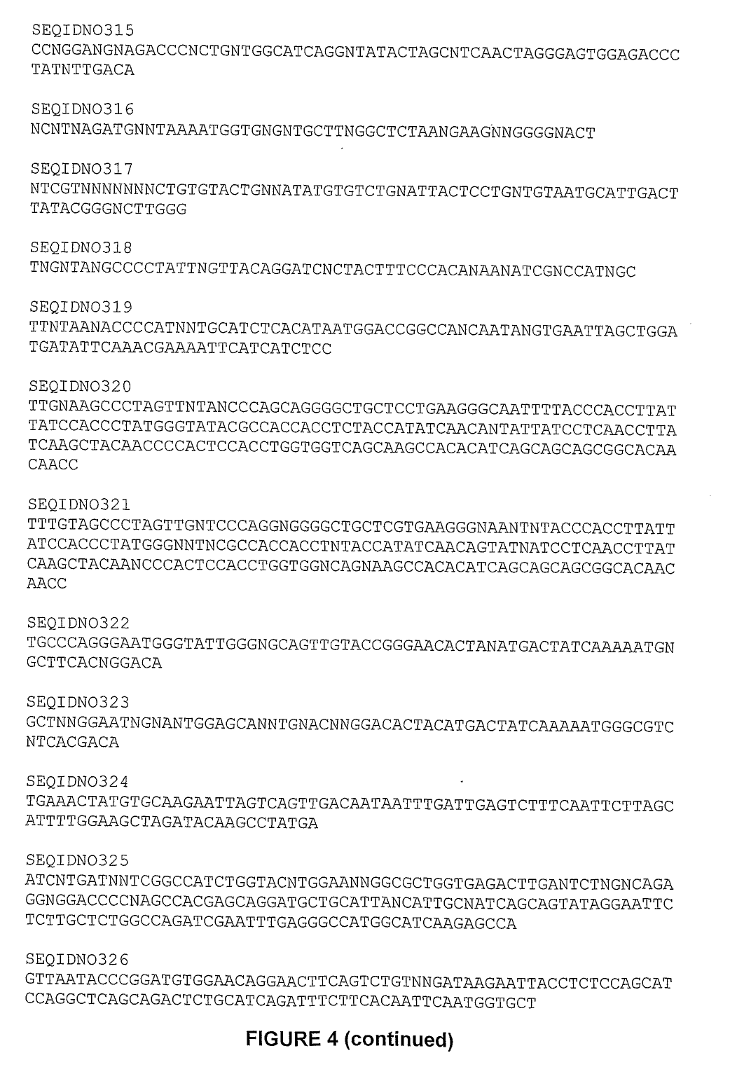

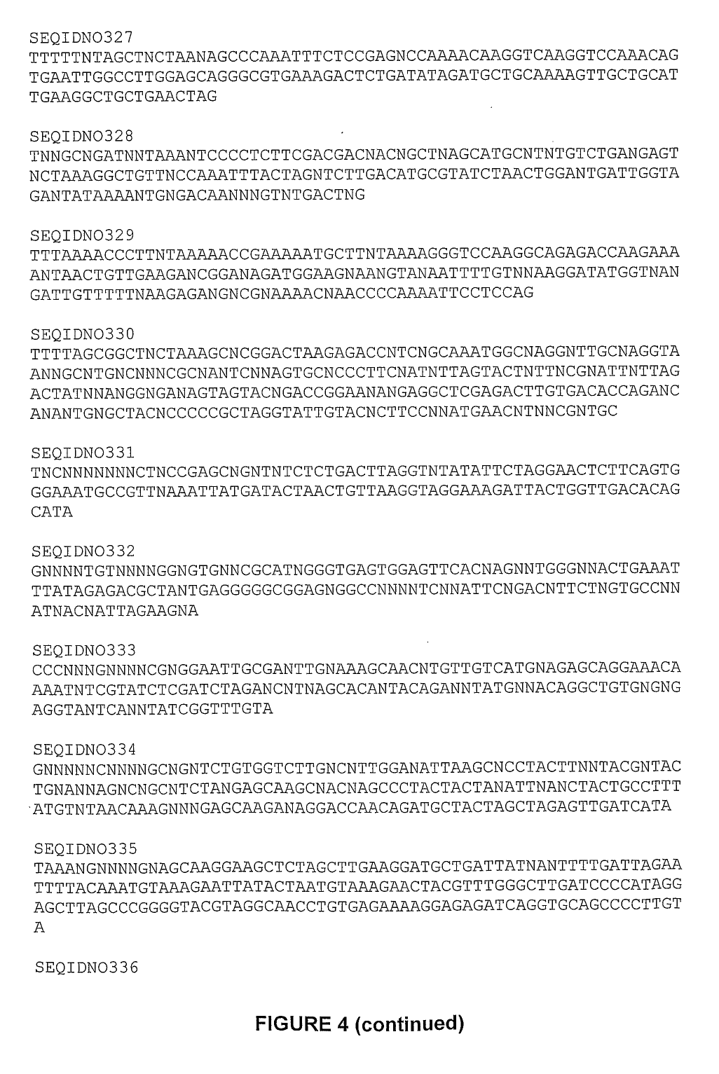

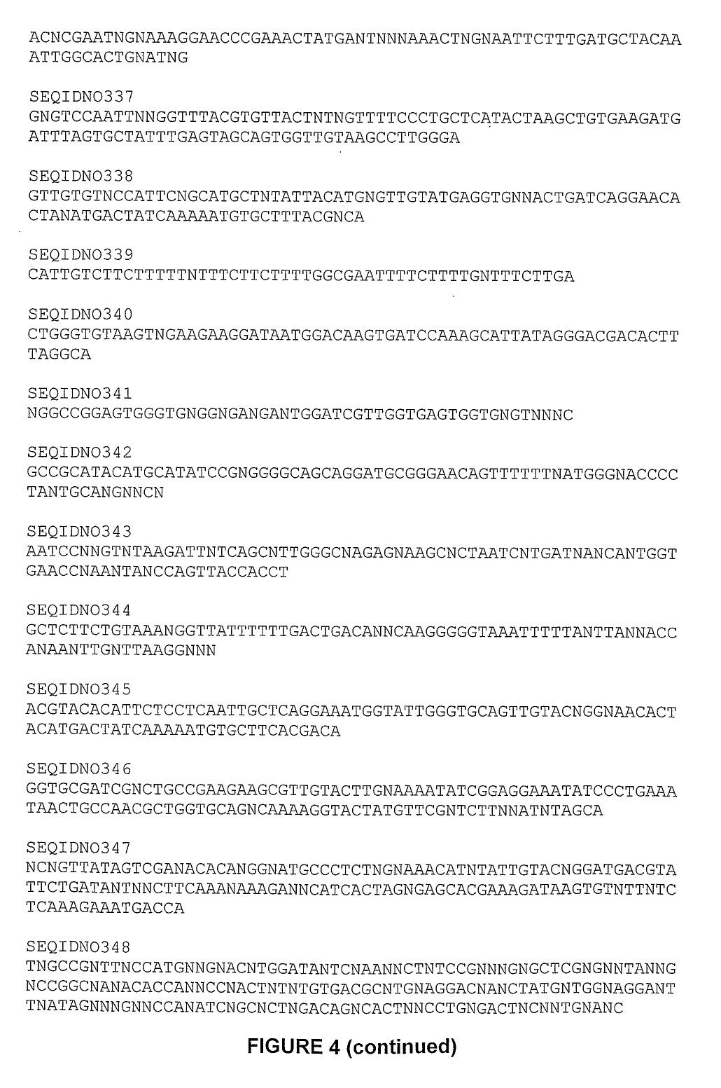

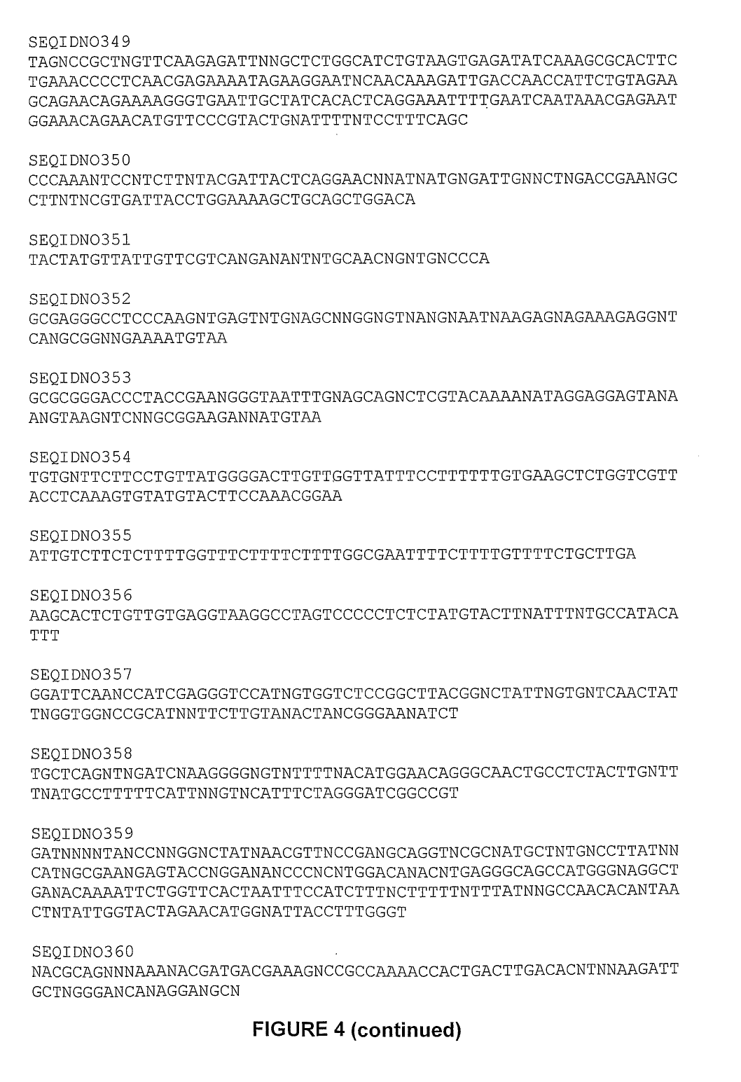

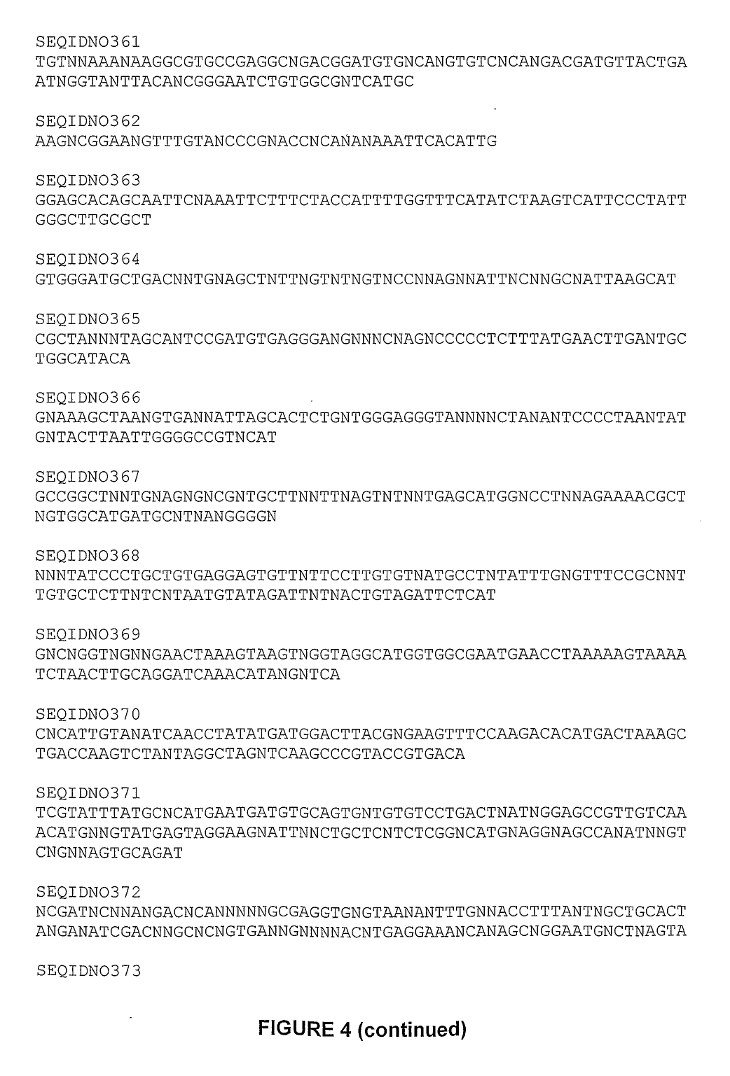

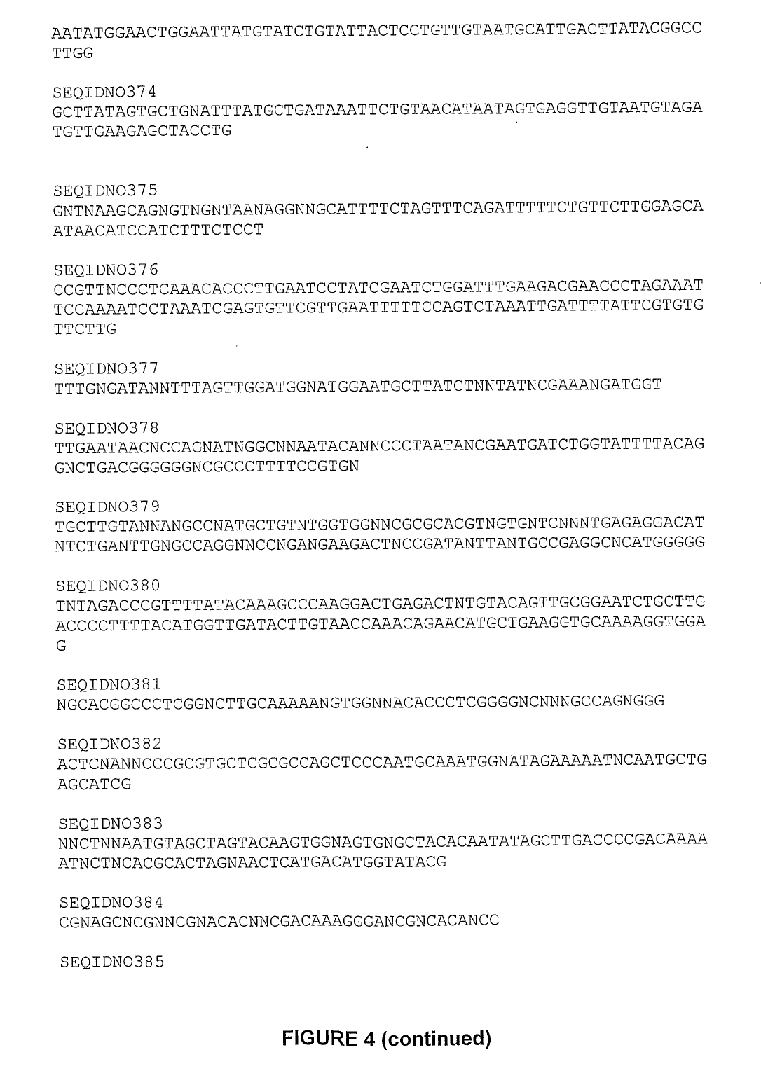

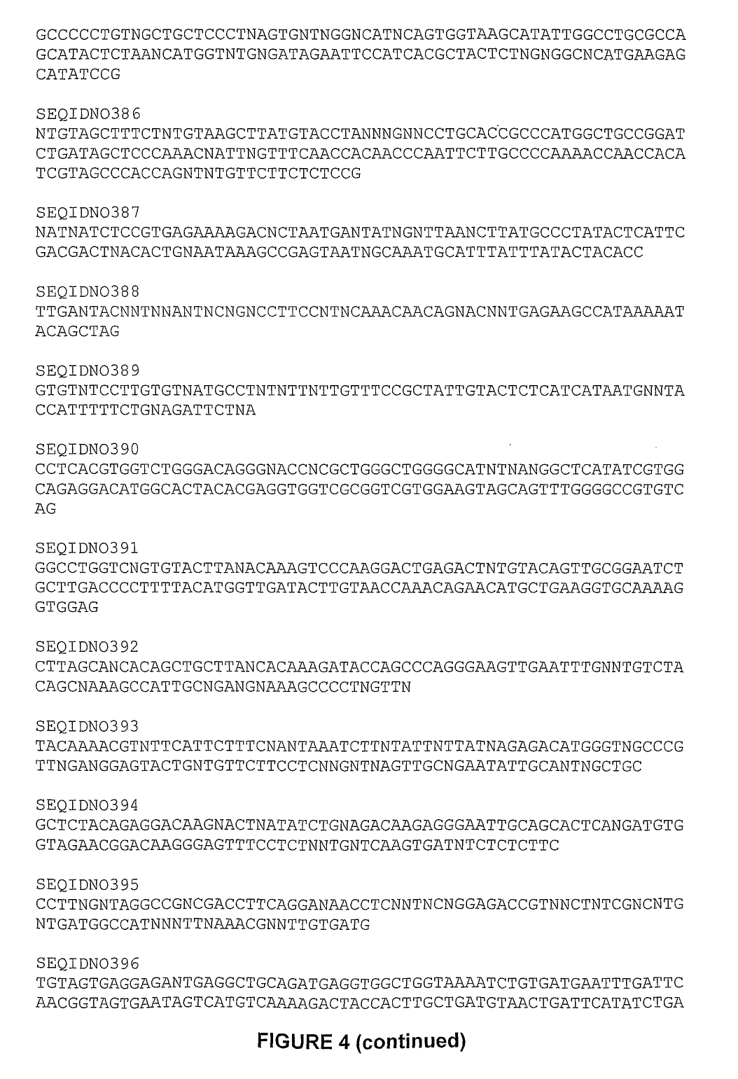

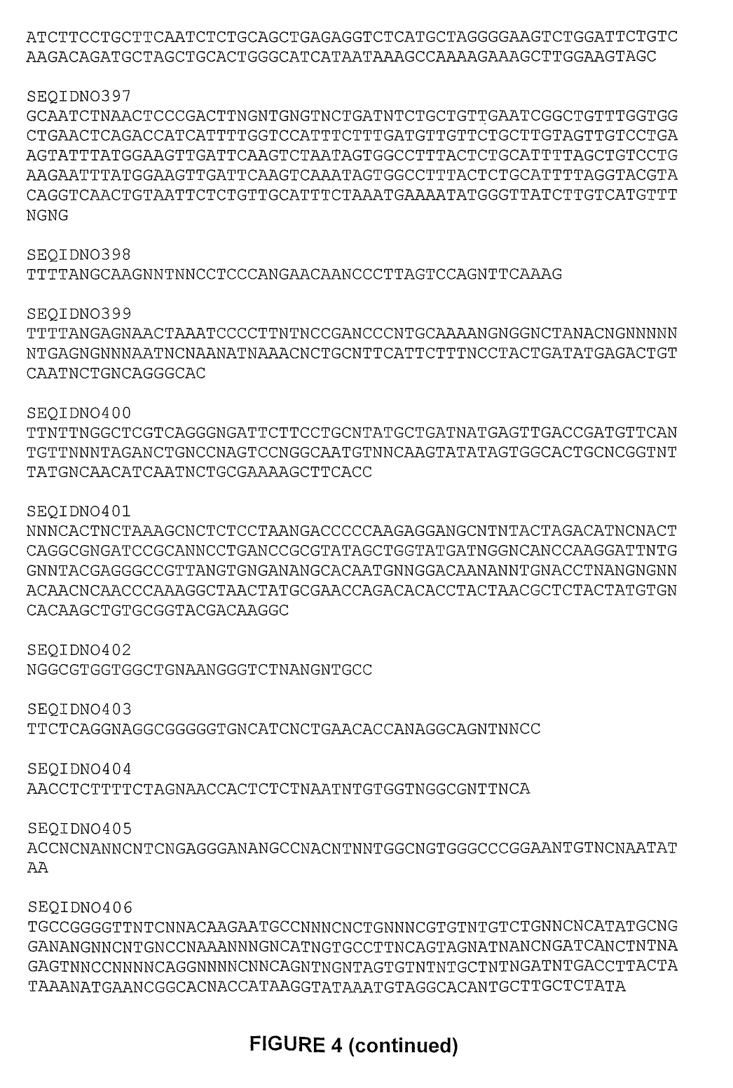

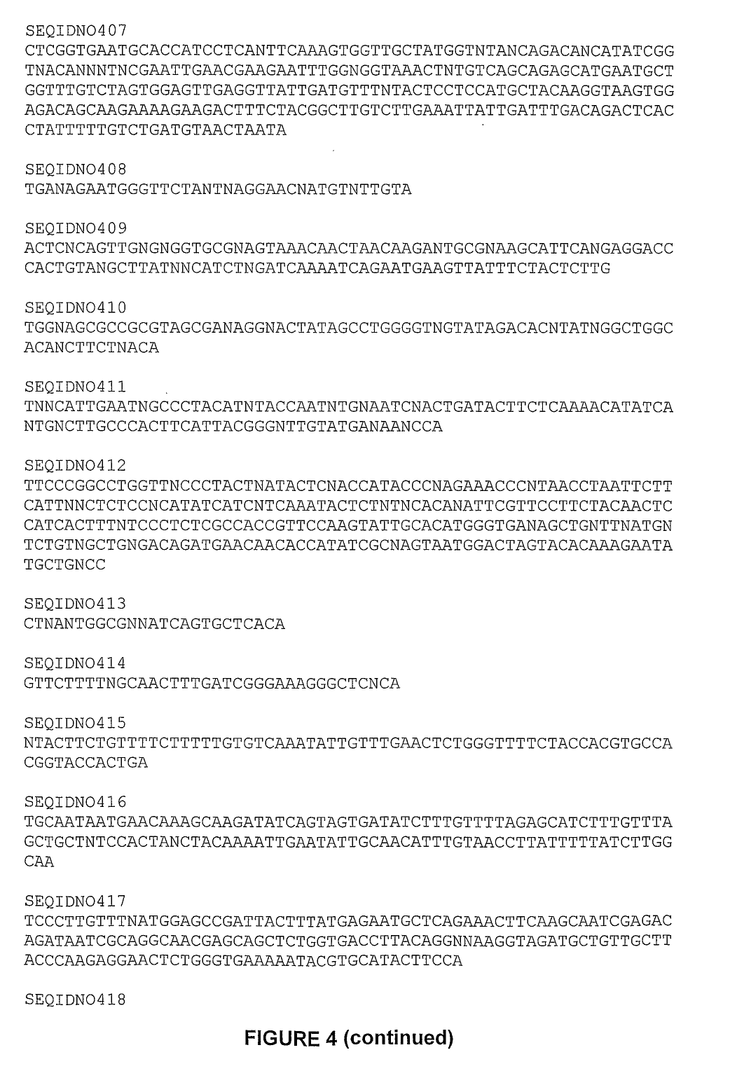

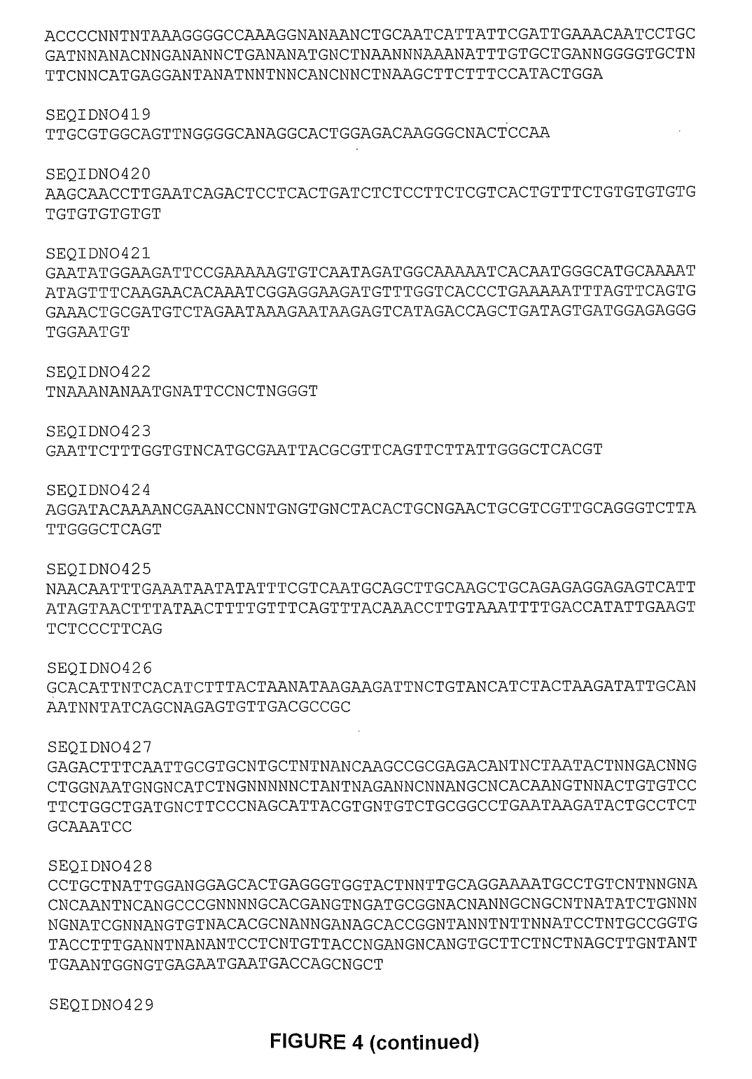

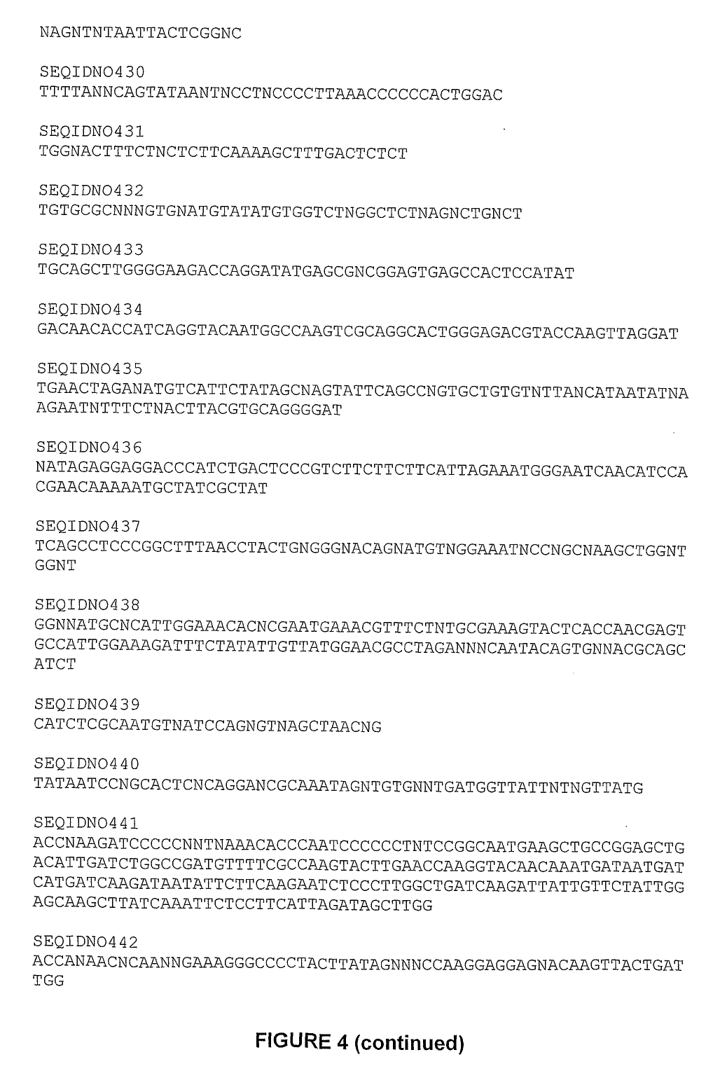

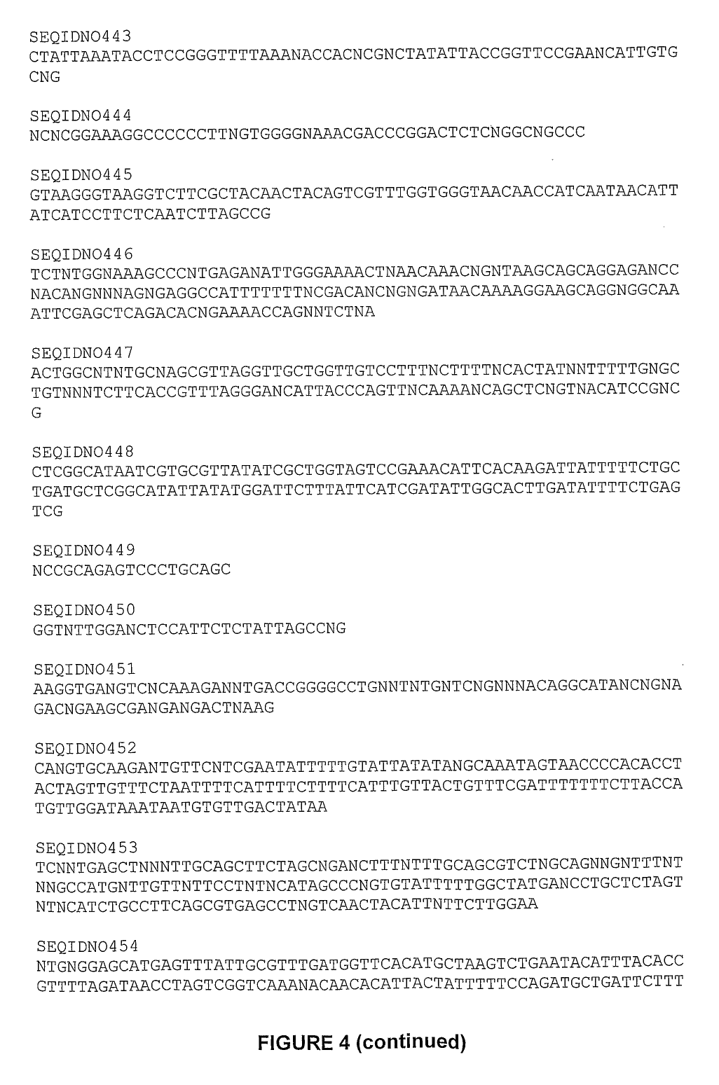

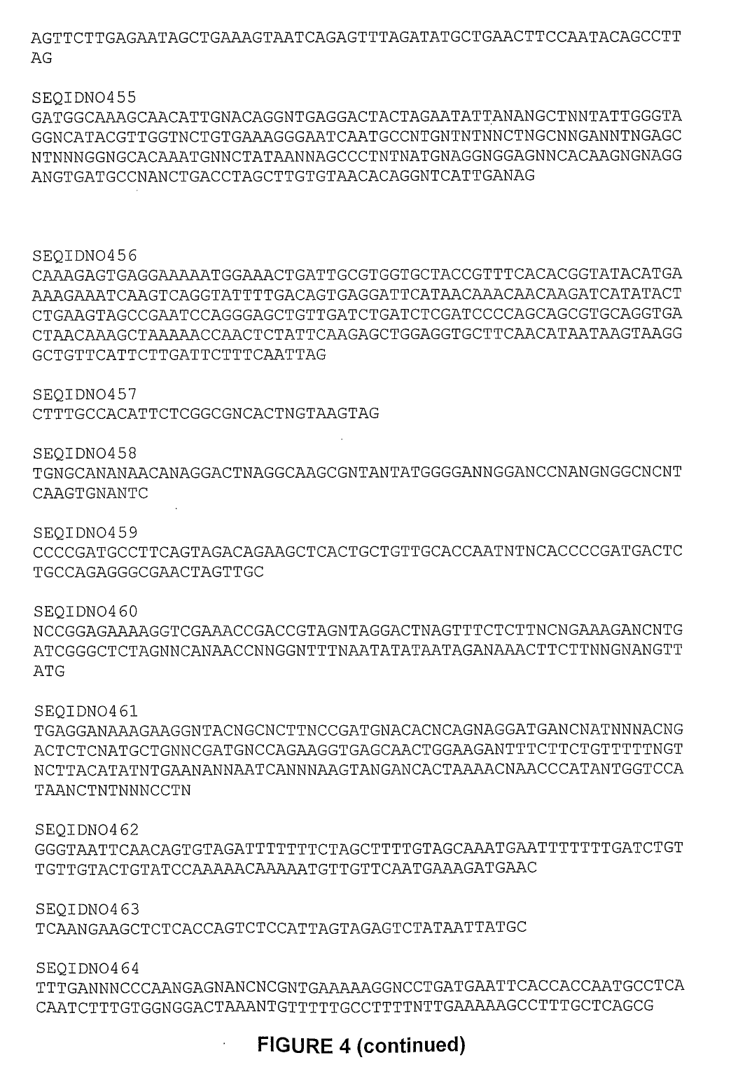

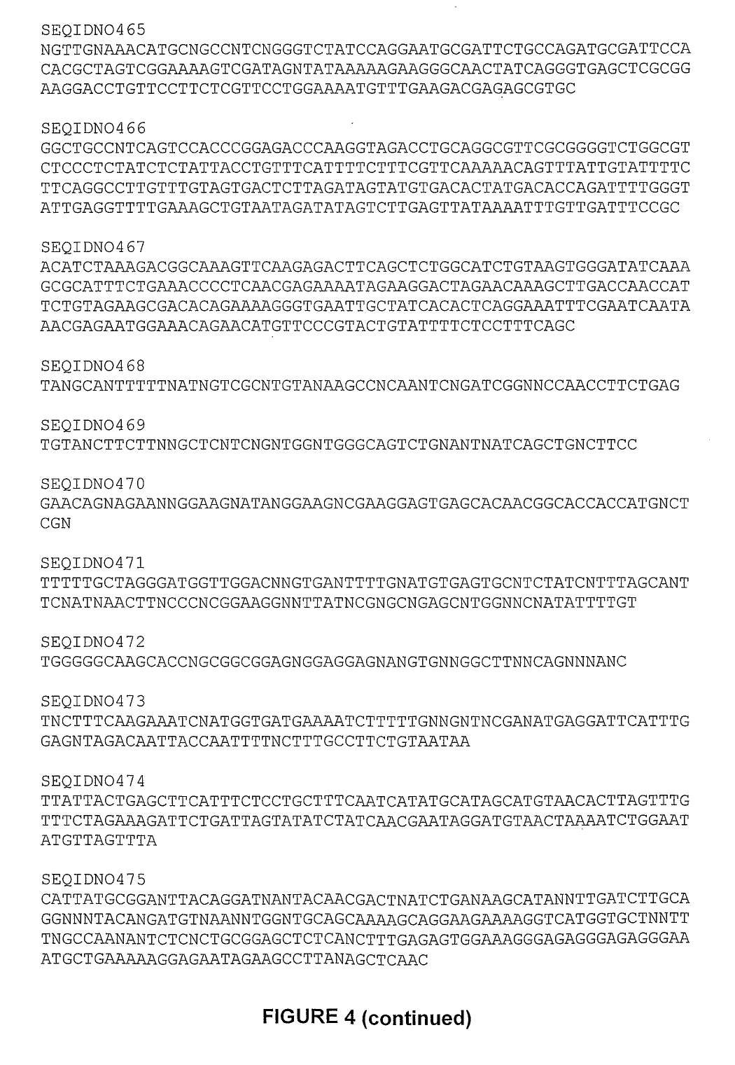

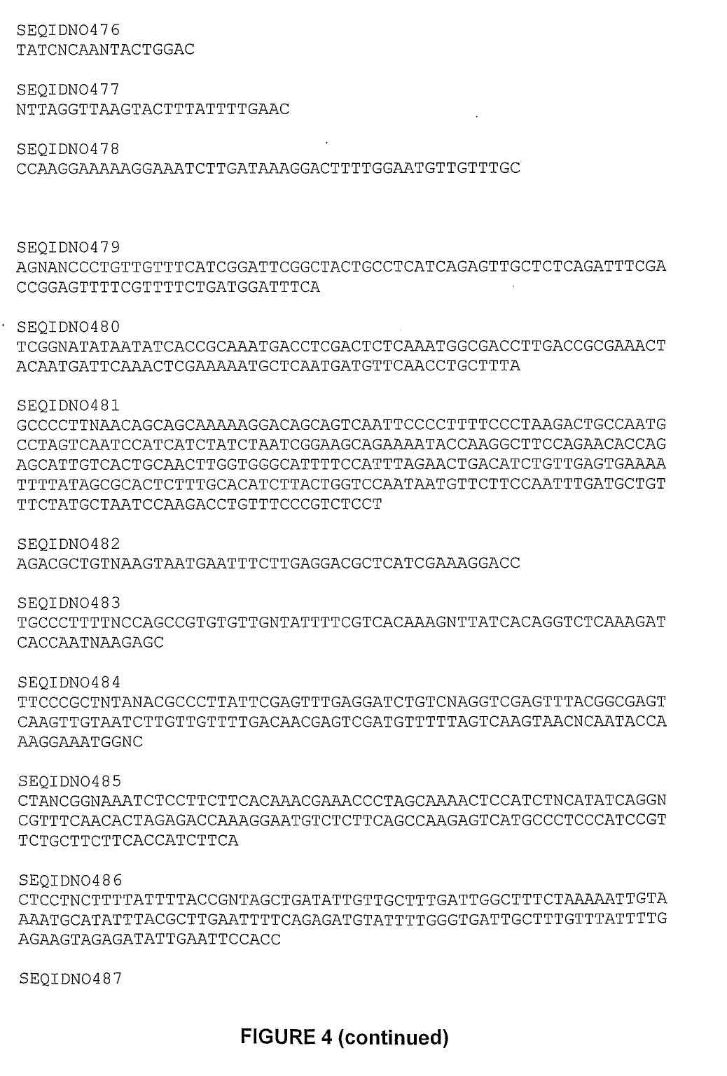

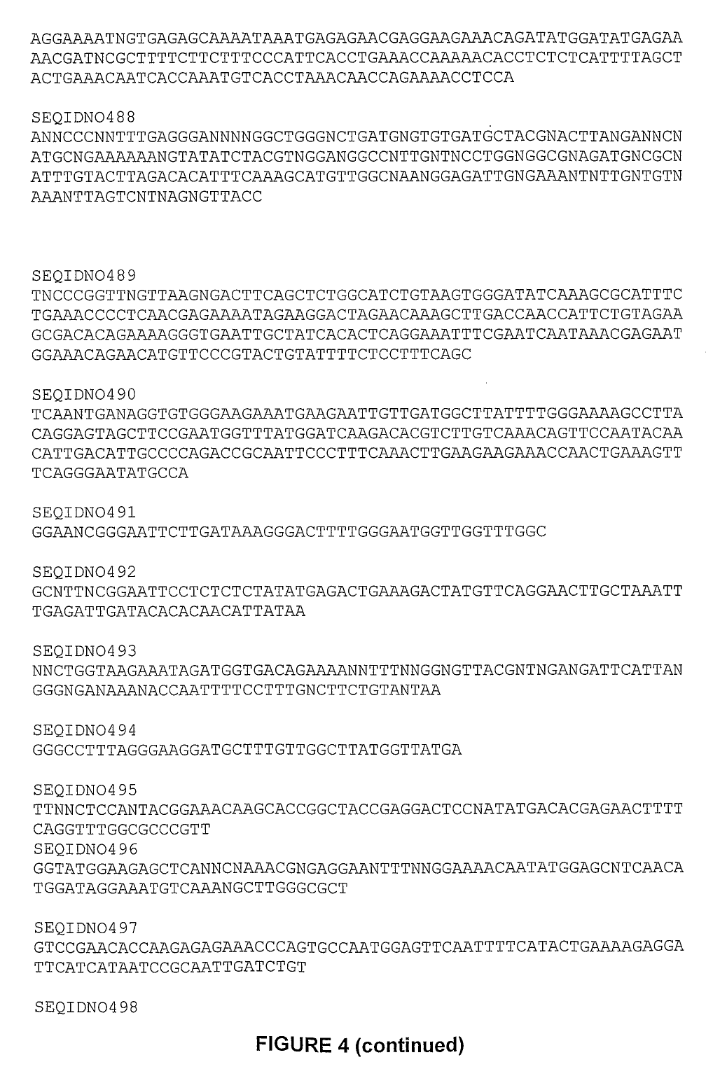

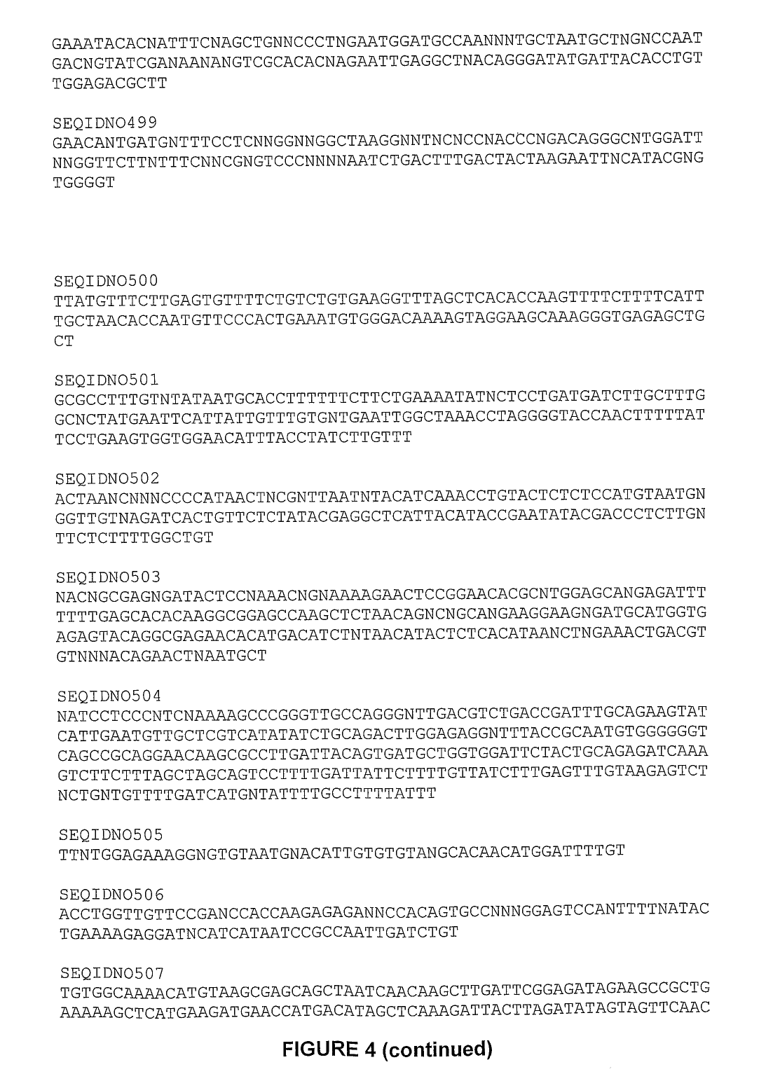

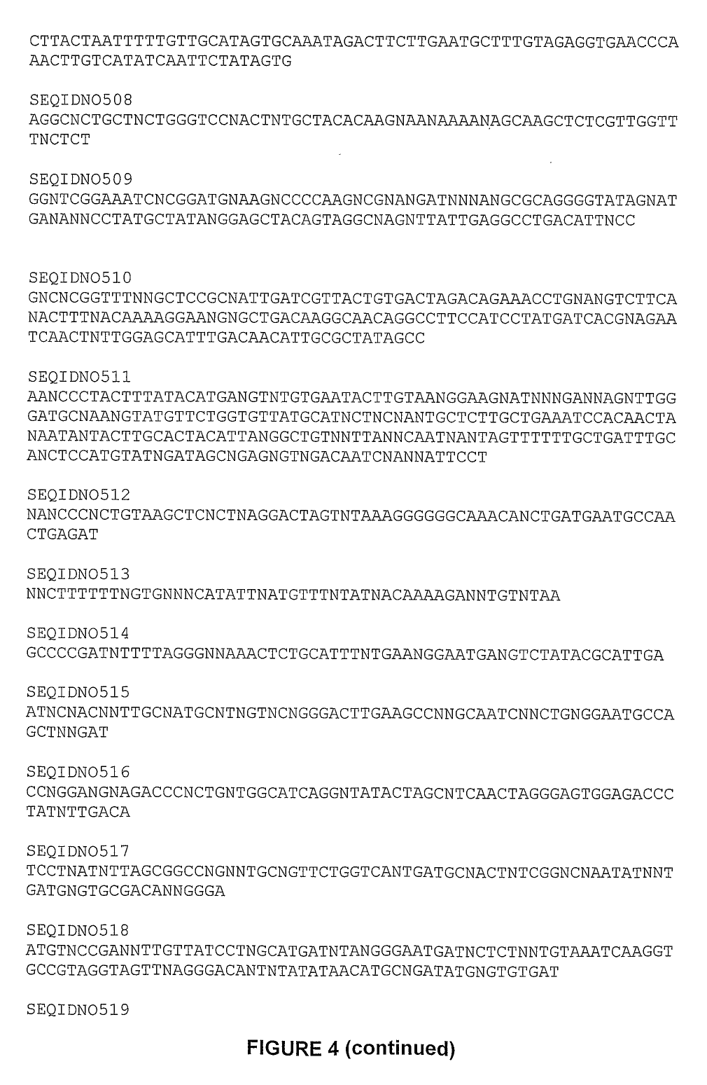

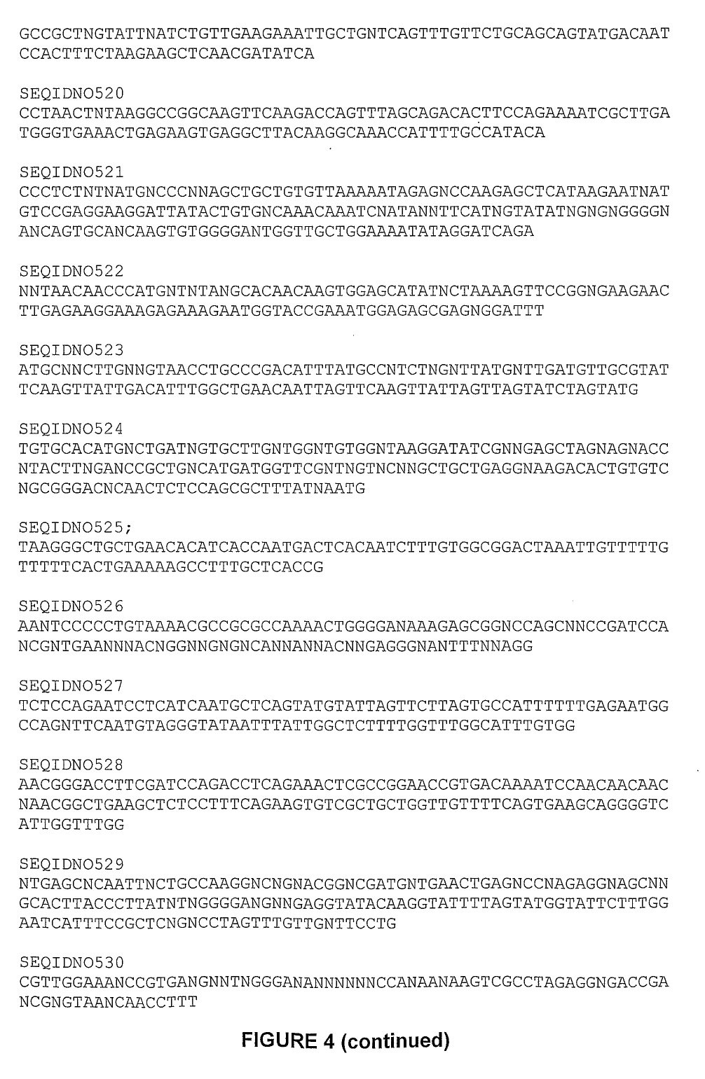

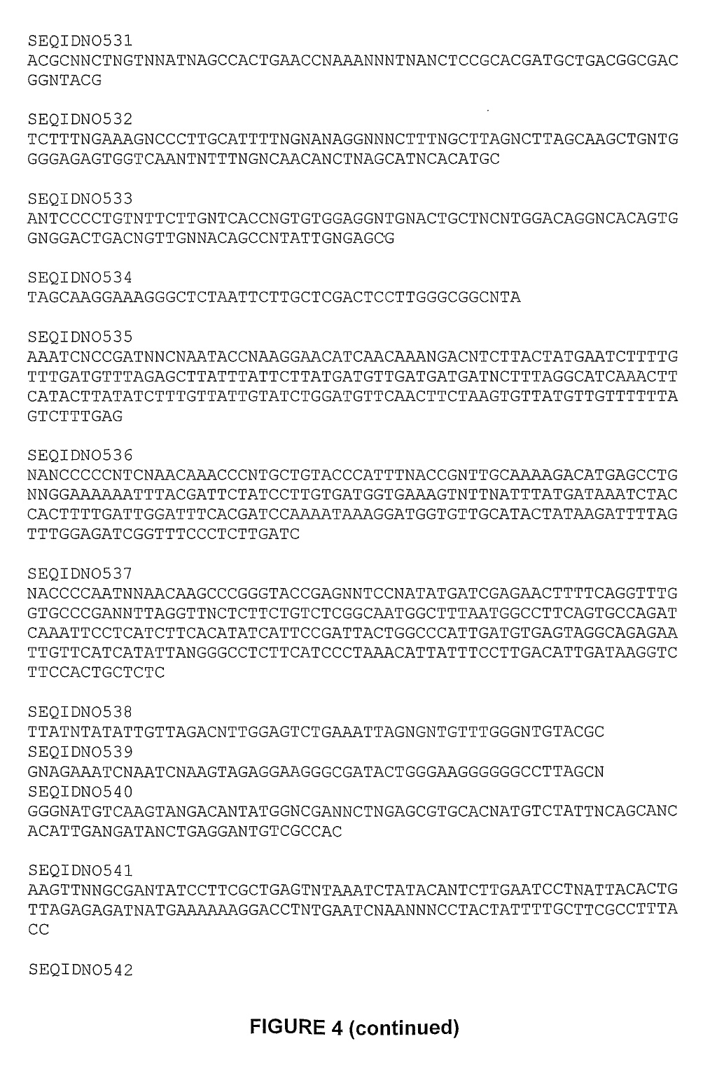

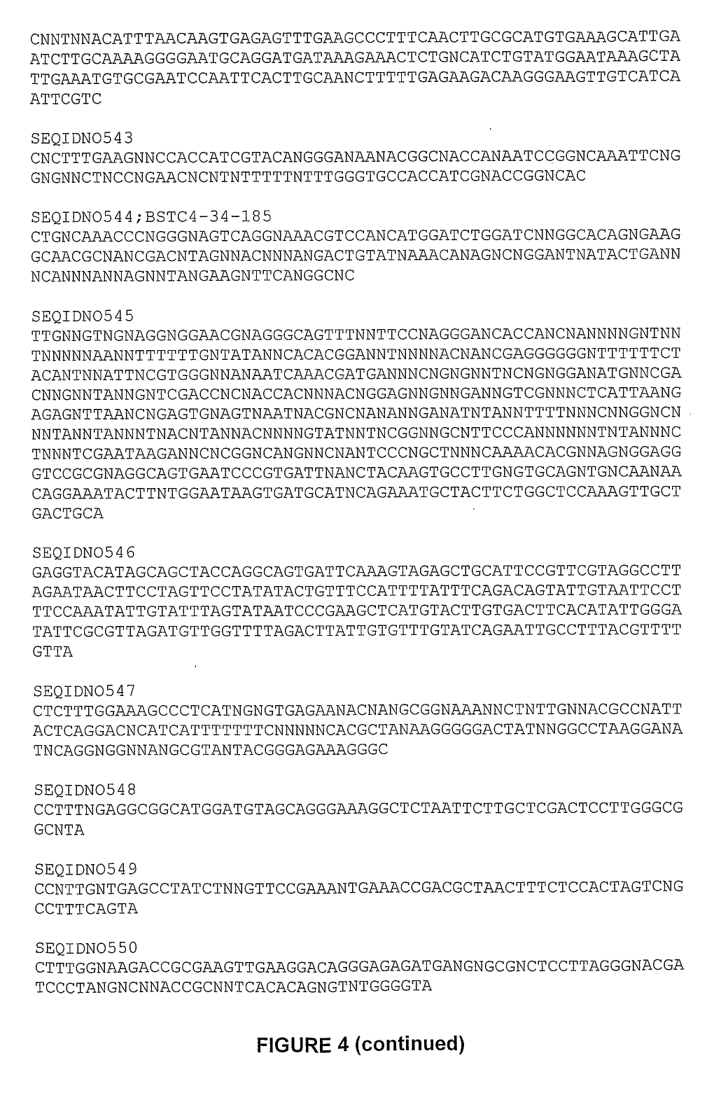

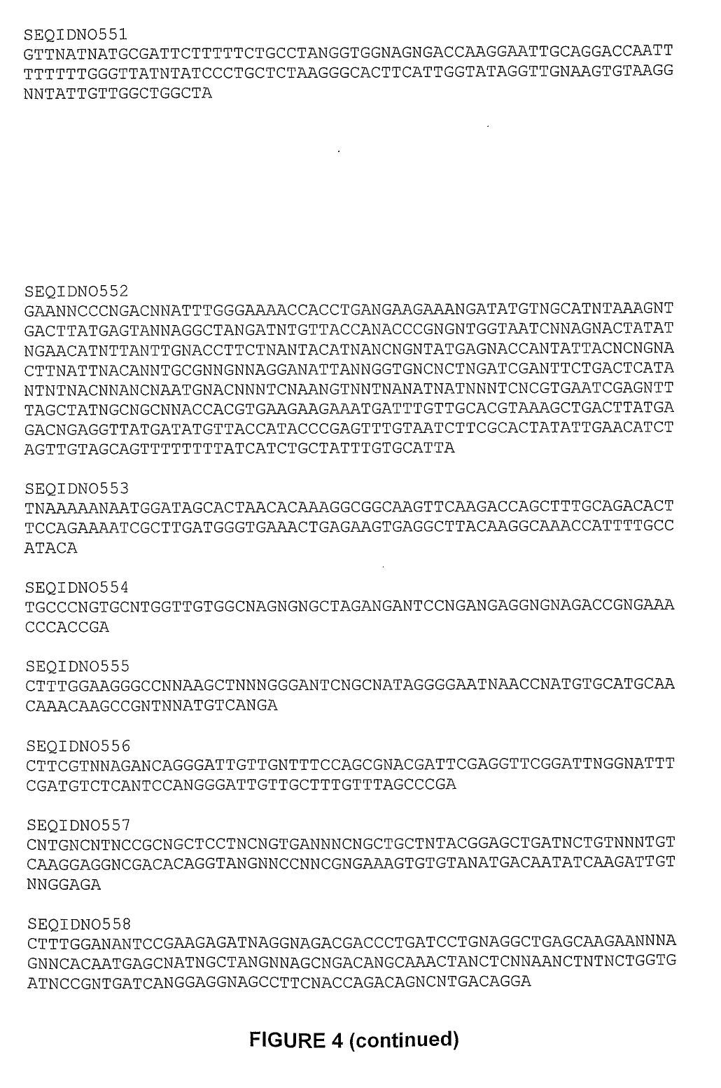

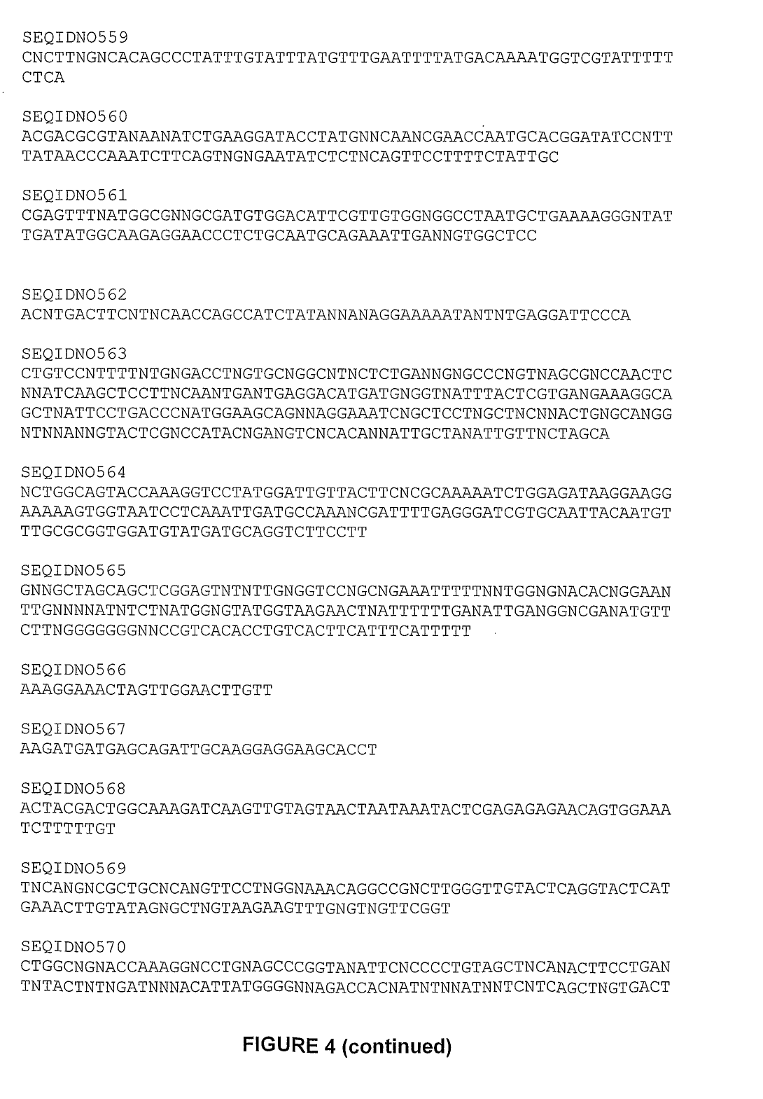

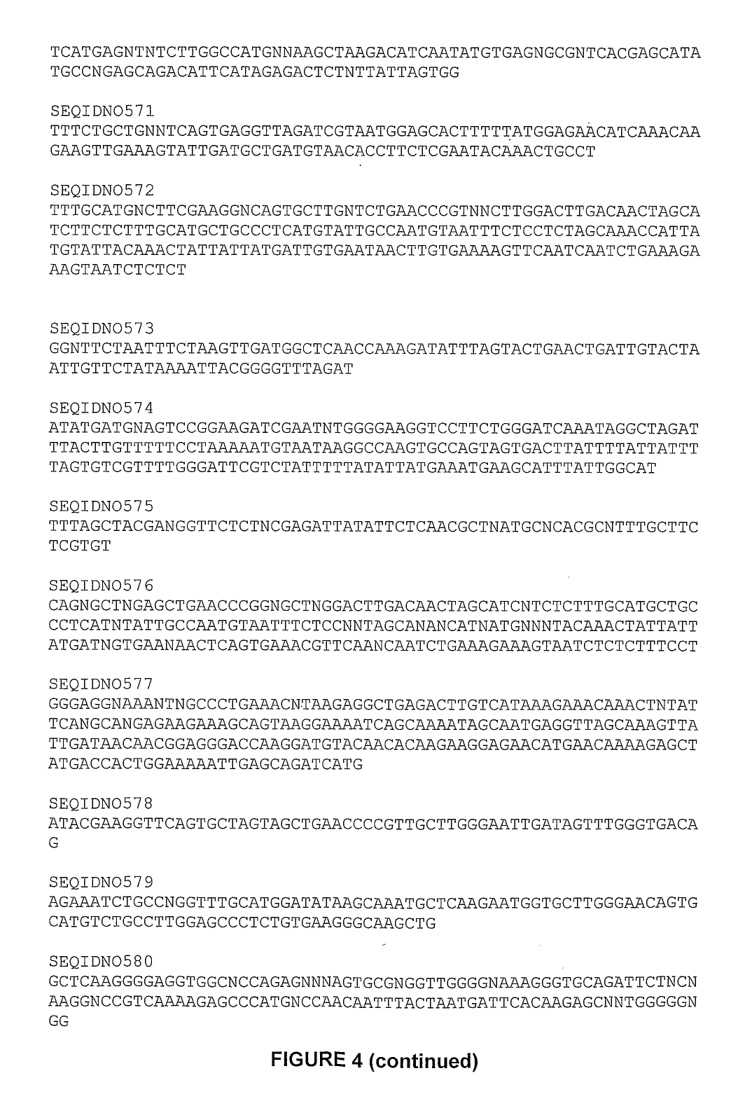

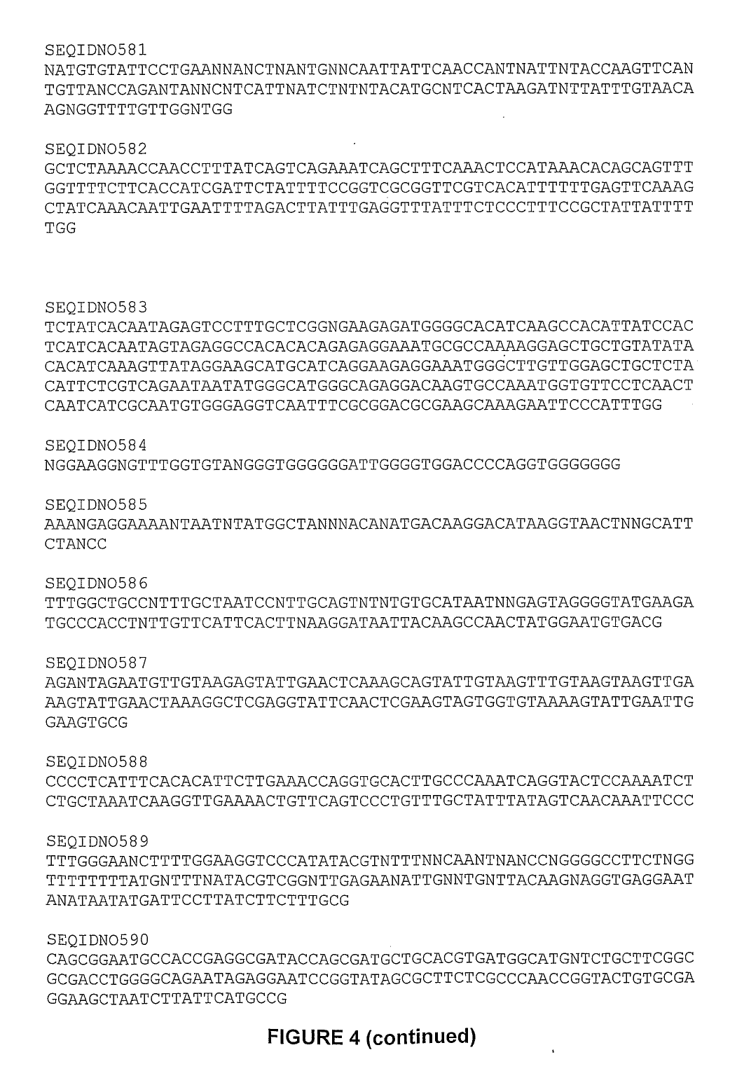

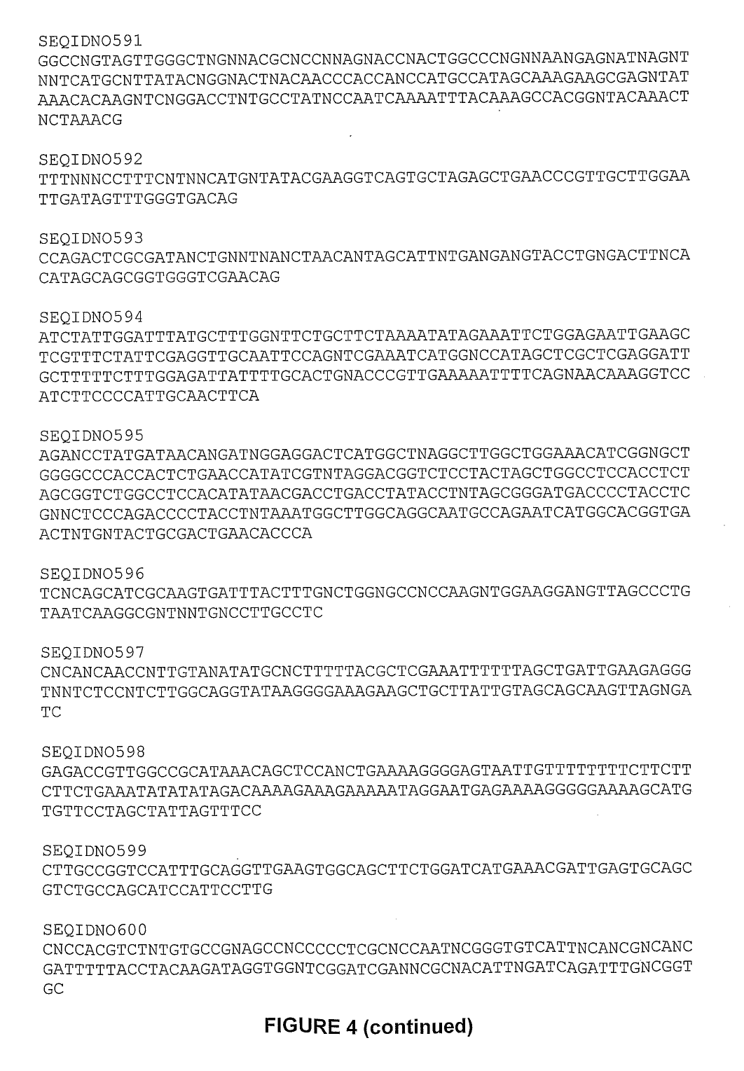

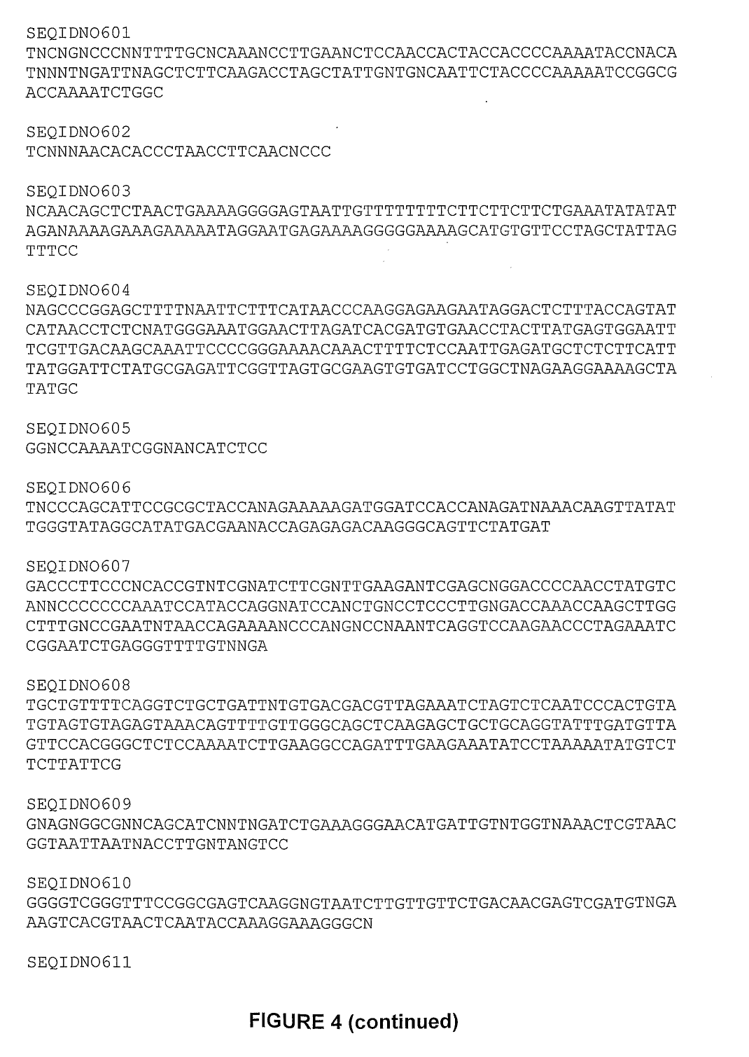

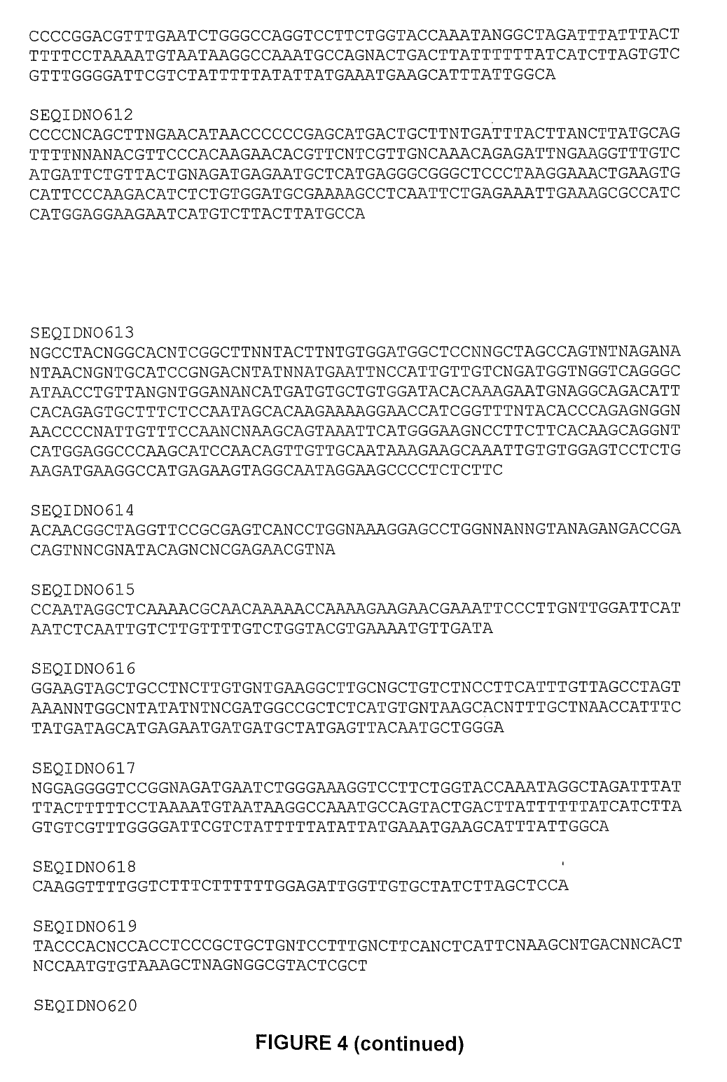

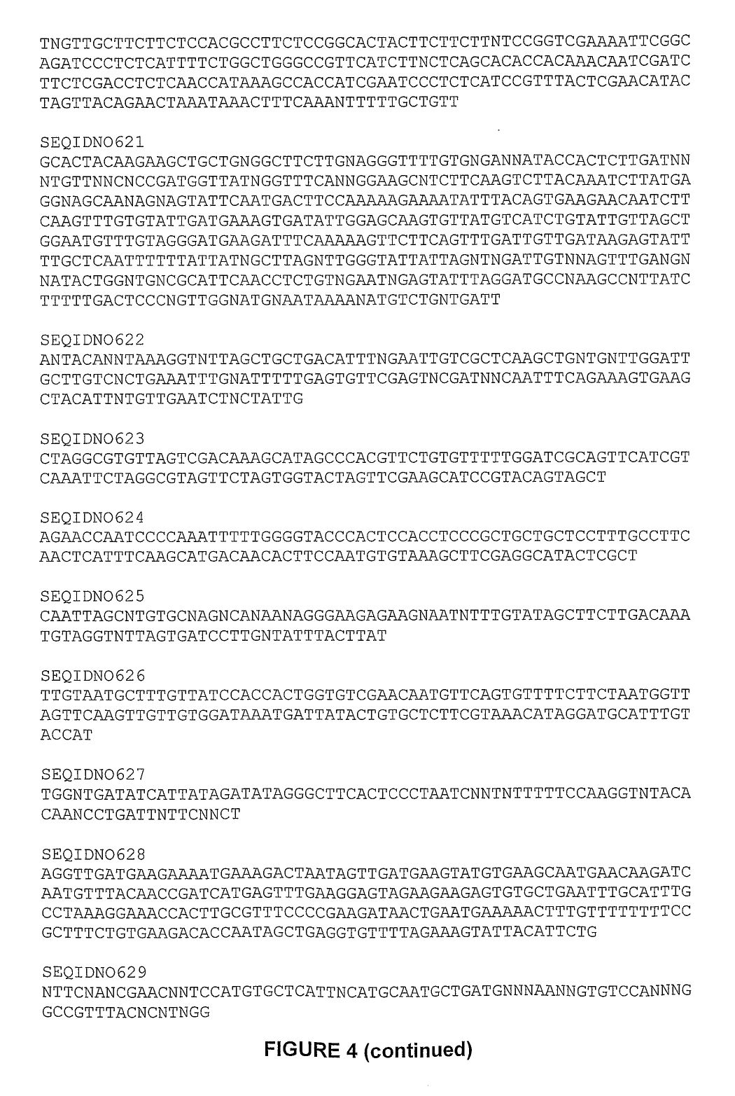

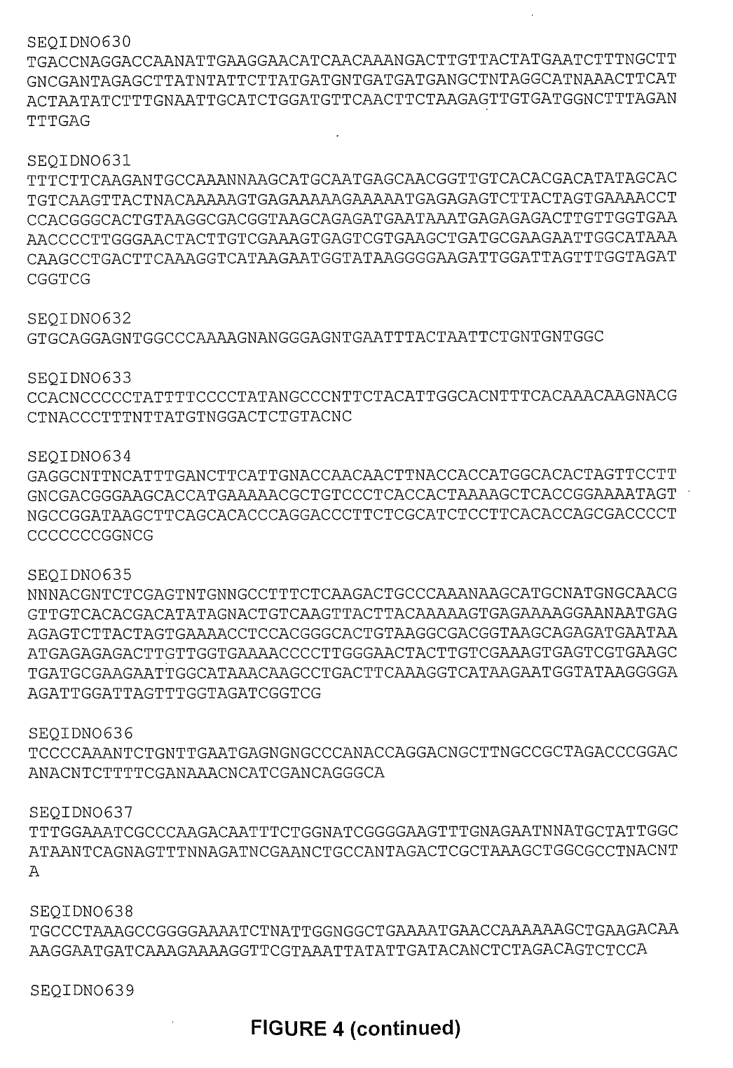

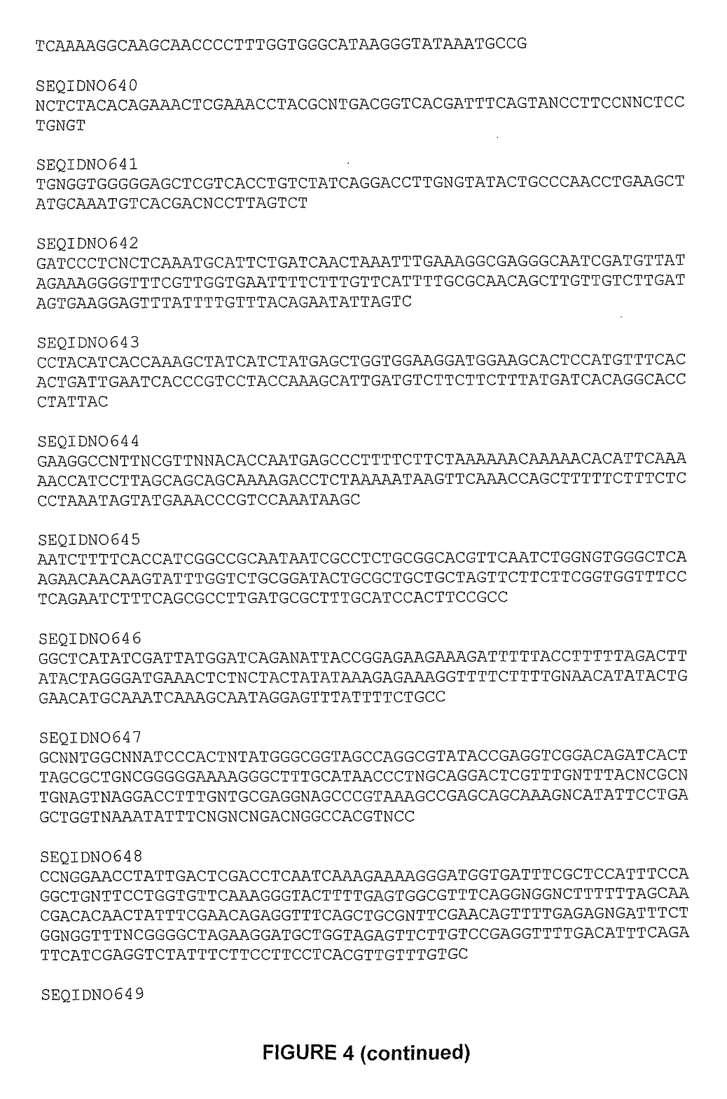

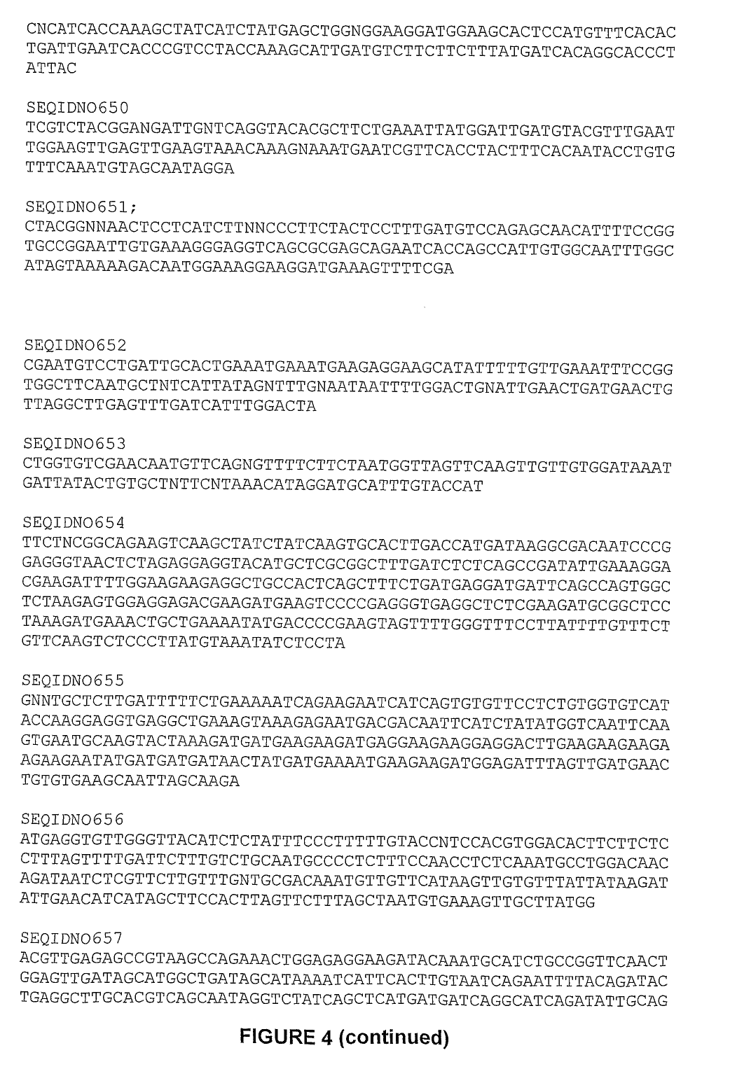

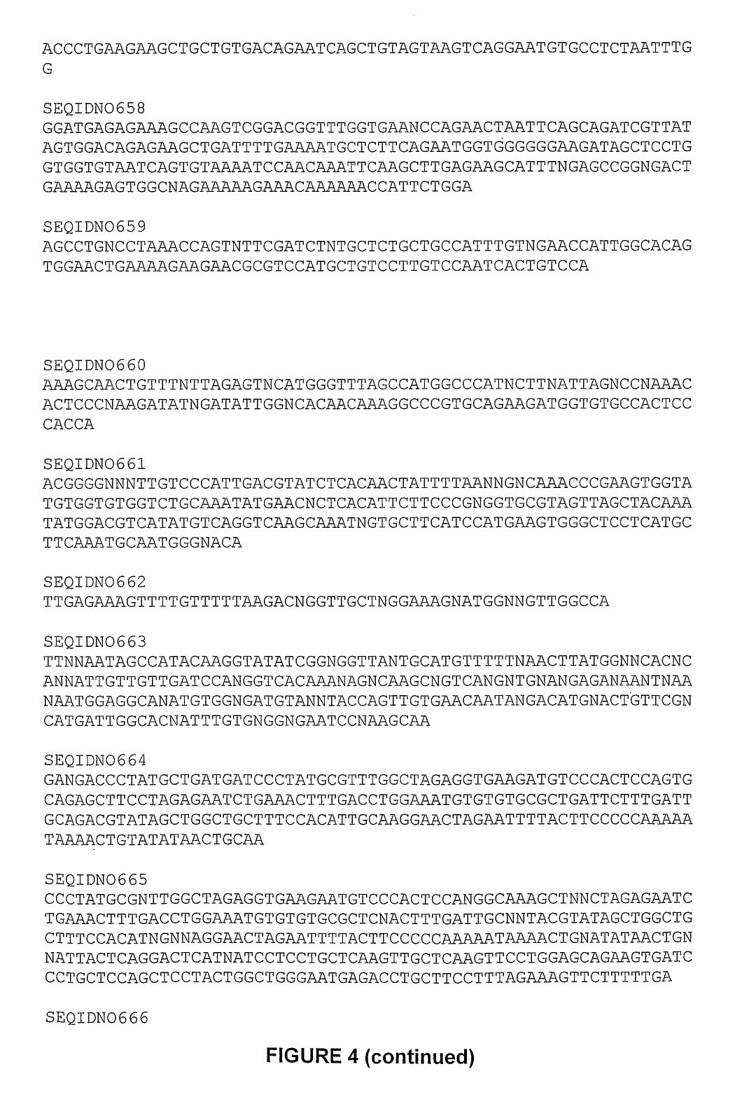

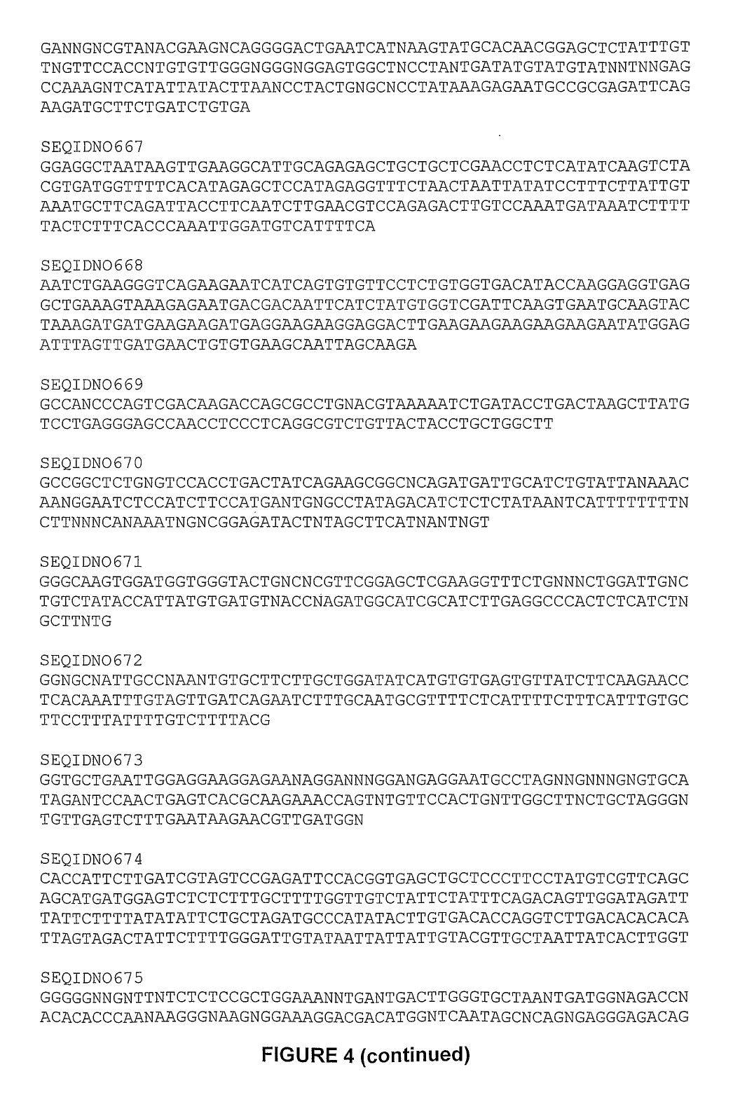

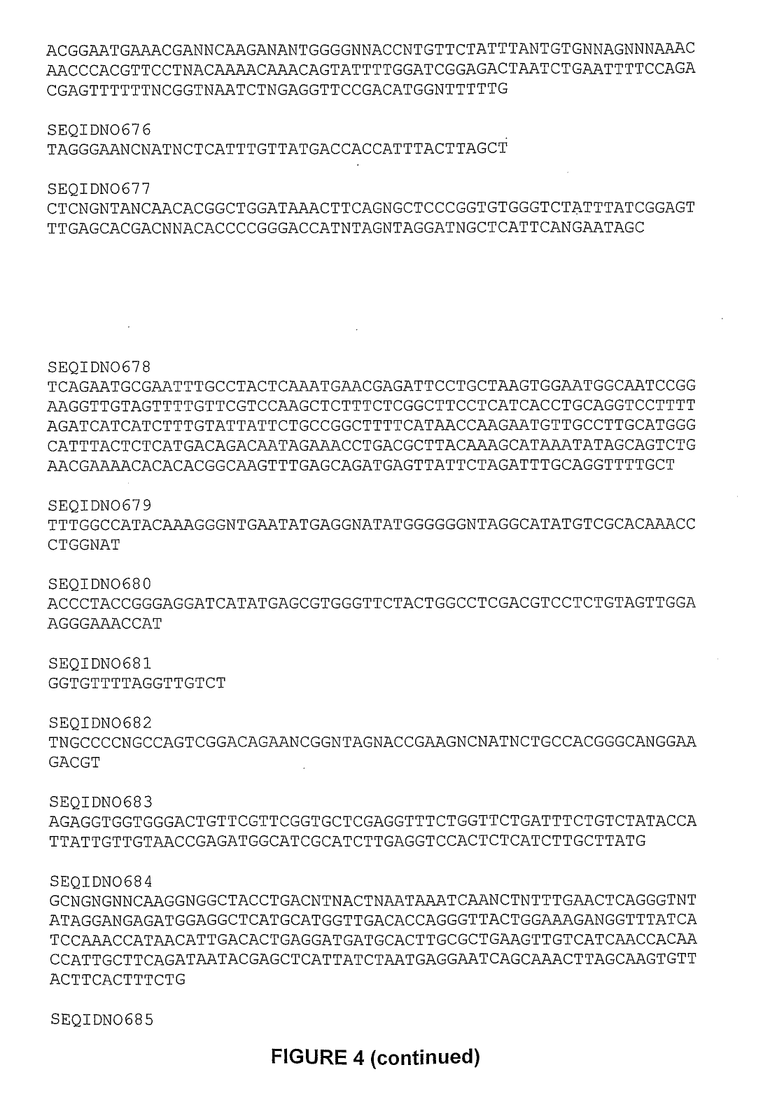

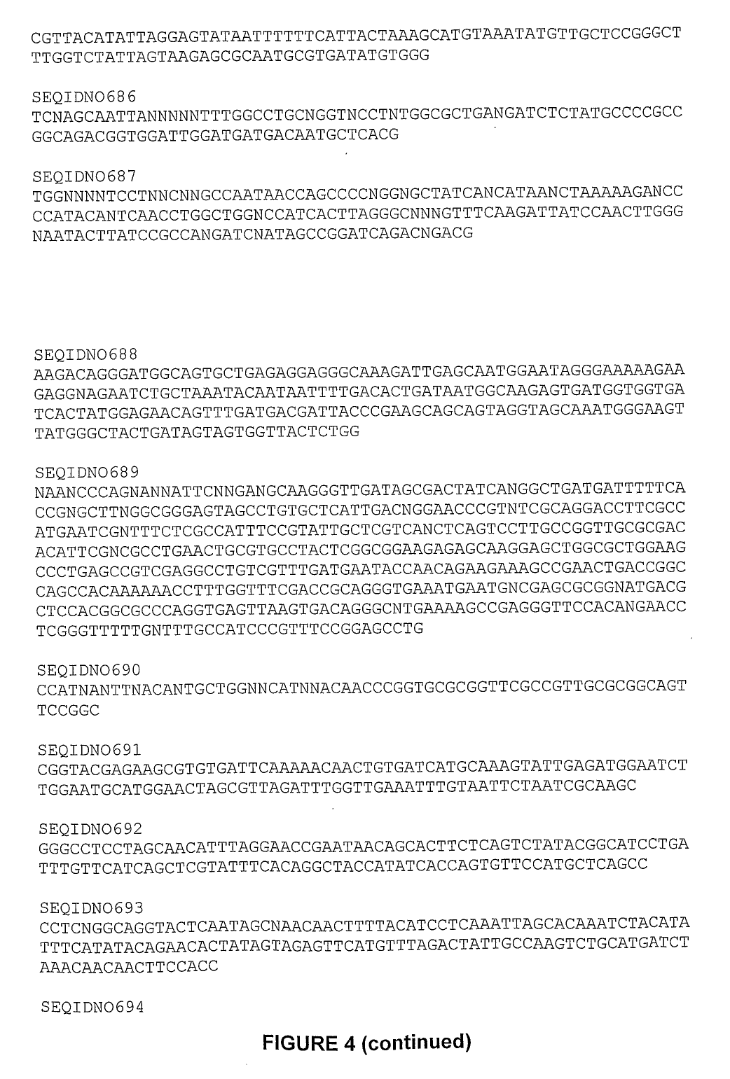

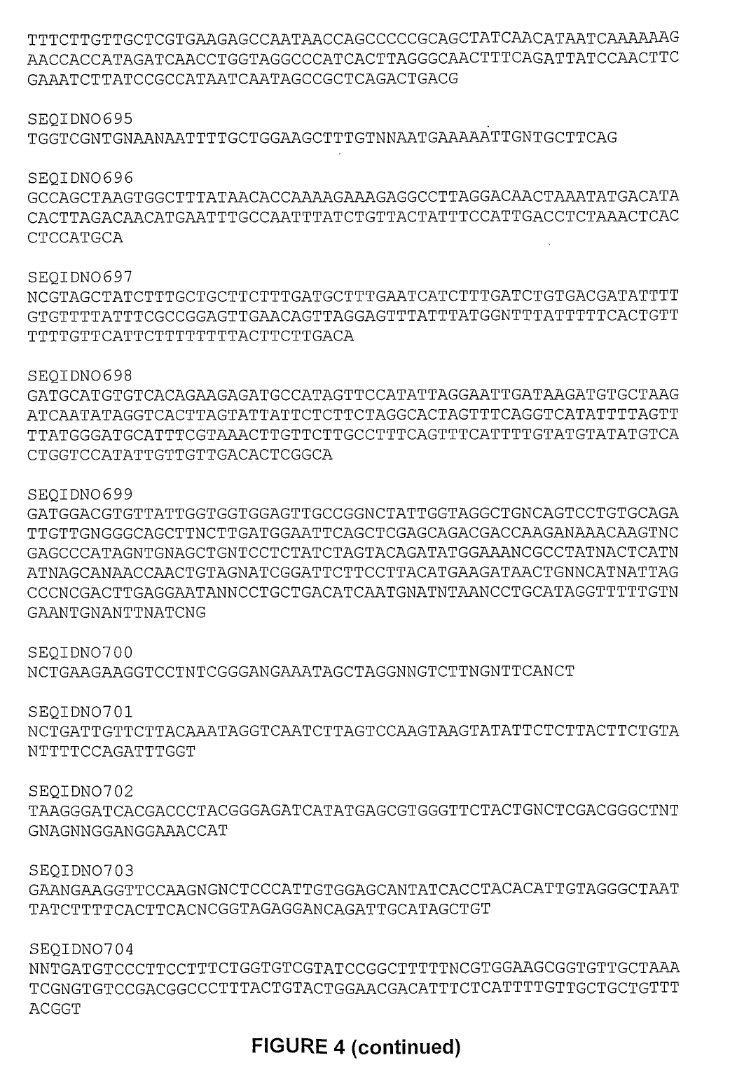

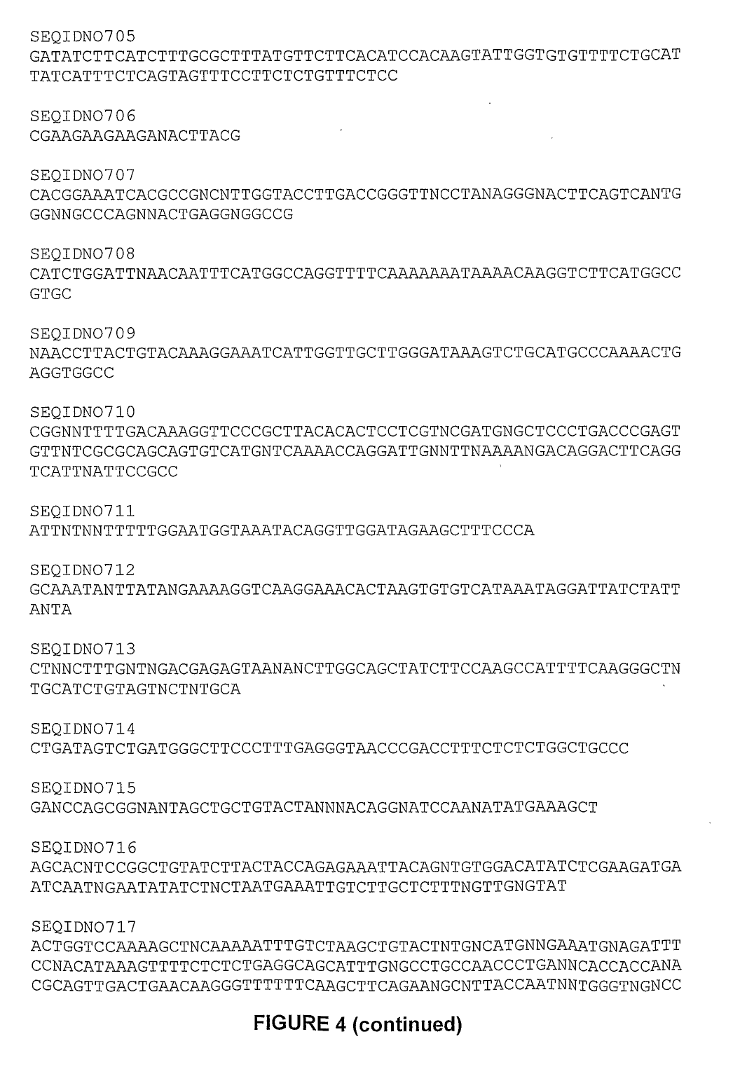

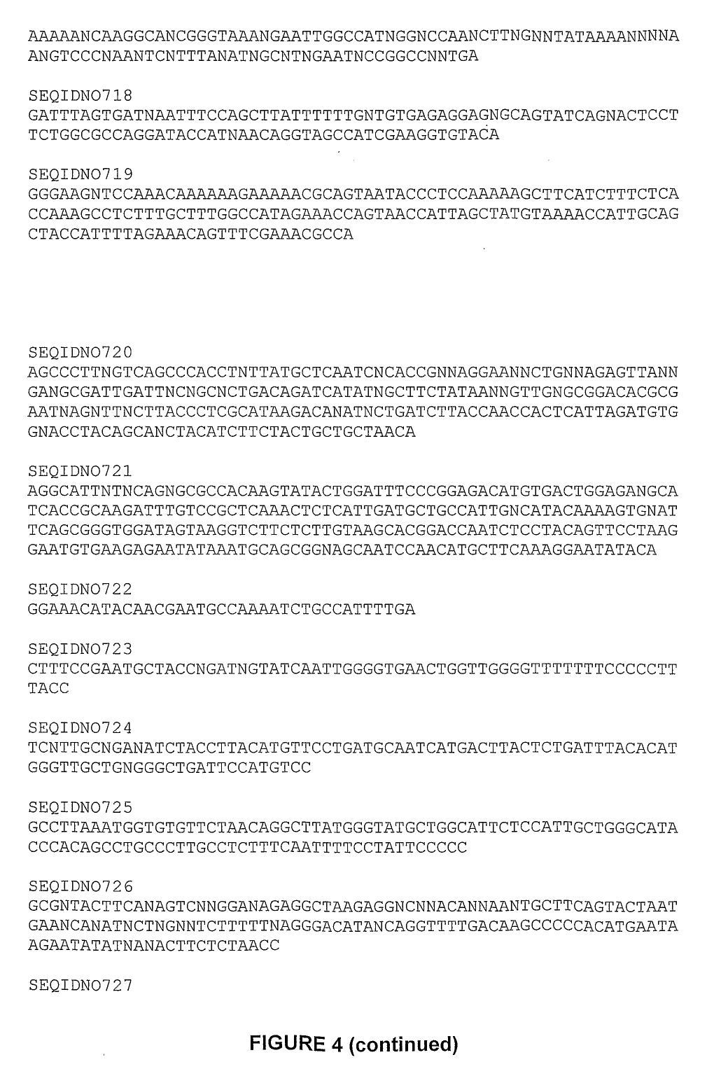

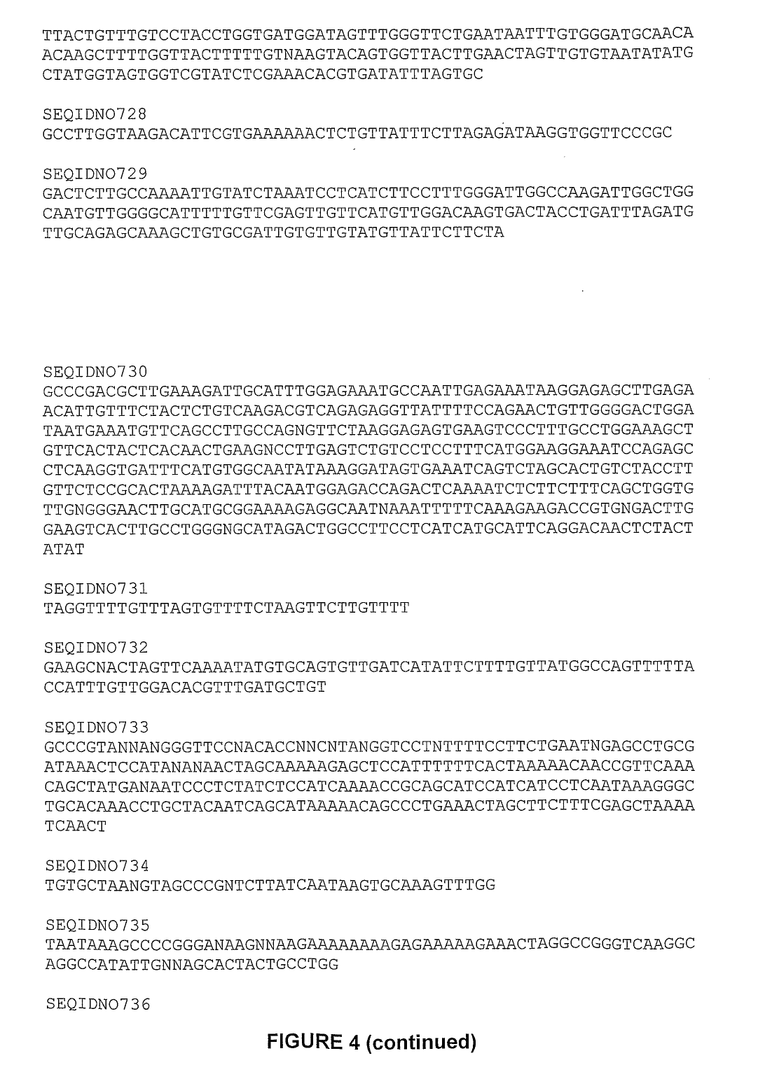

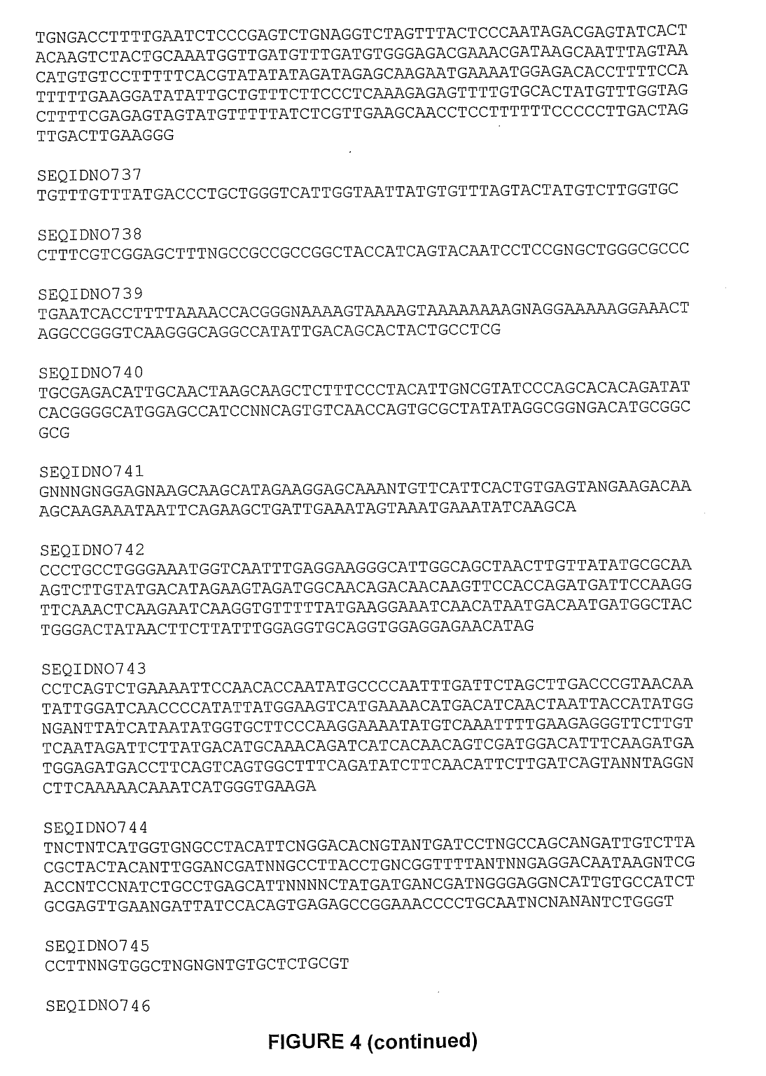

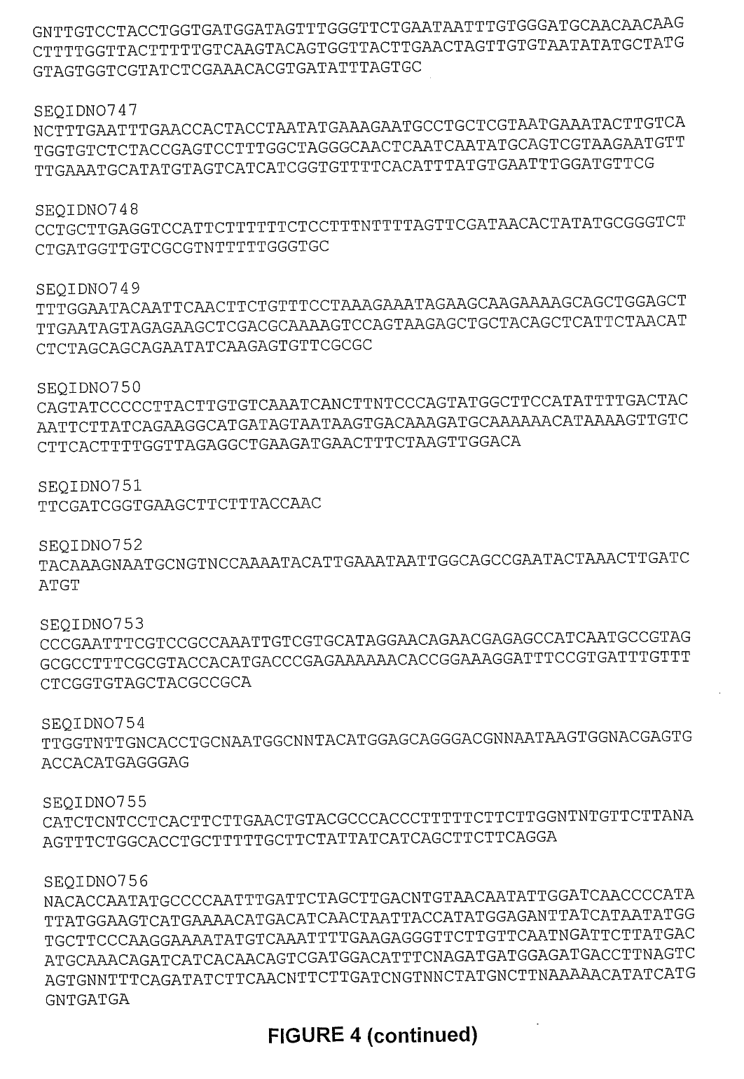

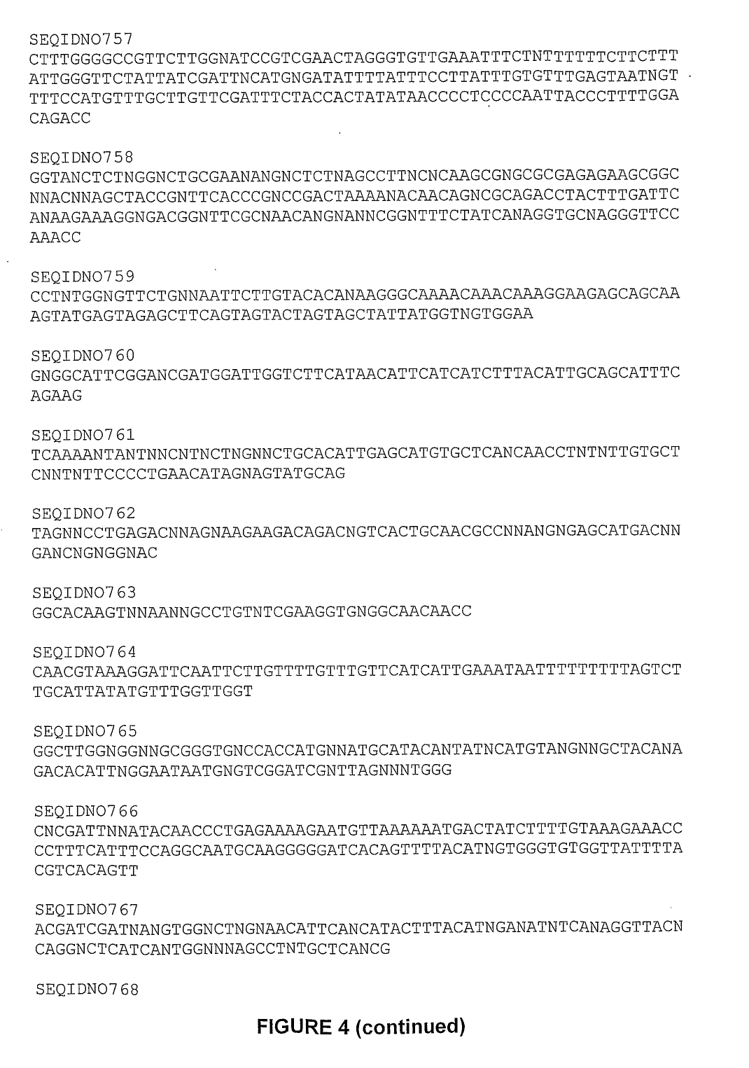

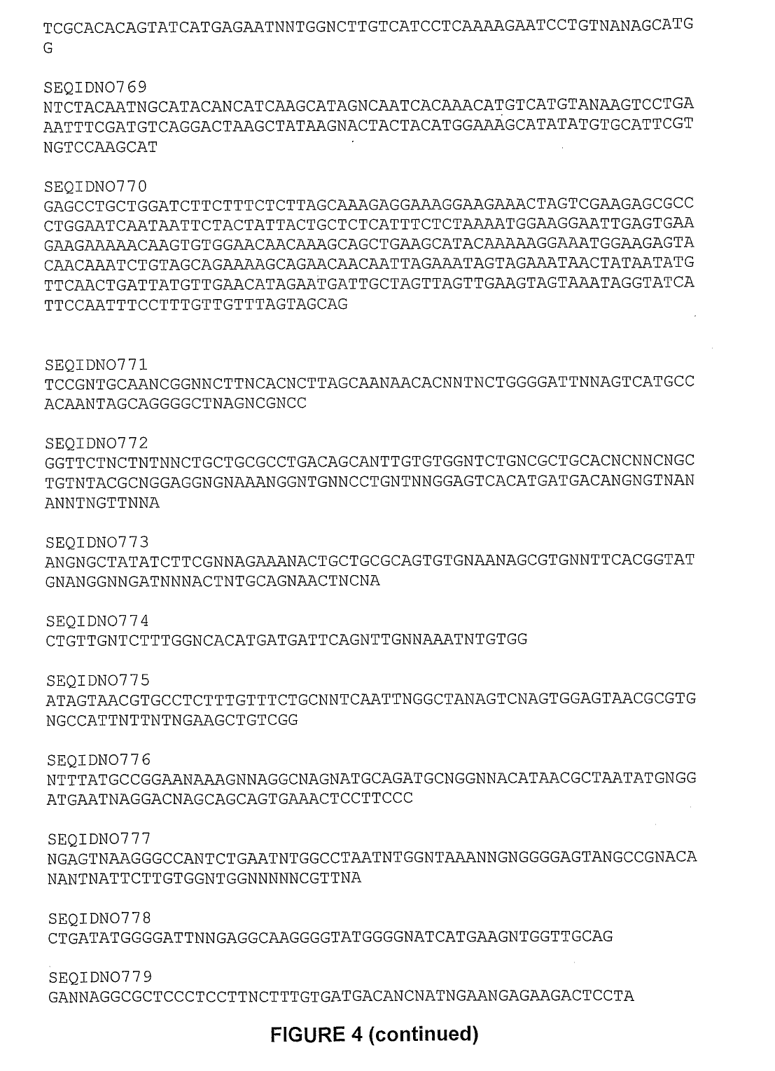

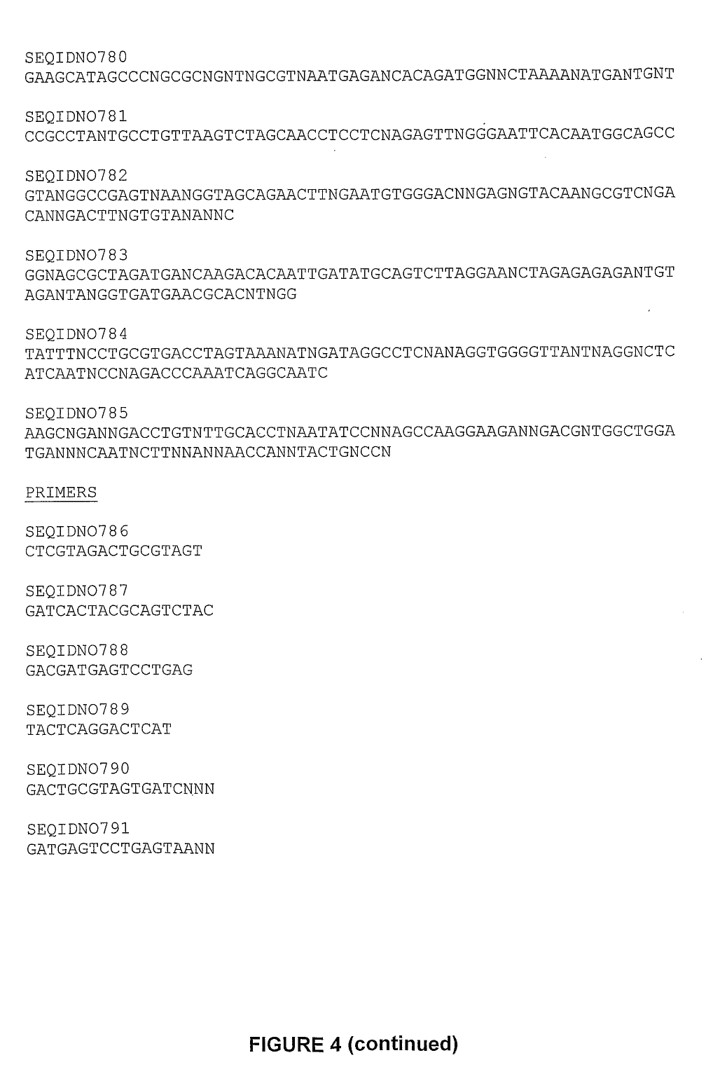

FIG. 4 shows the sequences identified by the methods of the present invention and represented by SEQ ID NO 1 to SEQ ID NO 785

### EXAMPLES

### Example 1

A cDNA-AFLP based expression profiling of sequence obtained from samples of a synchronized tobacco BY2 cell line system, was used to identify genes that are upregulated during the cell cycle, an essential biological process needed for the viability and growth of the tobacco cell line system.

A genome-wide expression analysis of cell cycle-modulated genes in the tobacco Bright Yellow-2 (BY2) cell line was performed. This unique cell line can be synchronized to high levels with different types of inhibitors of cell cycle progression (Nagata et al., Int. Rev. Cytol., 132 1-30, 1992; Planchais et al., FEBS Lett., 476 78-83, 2000). Because of the lack of extensive molecular resources such as genomic sequences, cDNA clones or expressed sequence tags (ESTs) for tobacco, a microarray-based approach cannot be used for a transcriptome analysis. Therefore, the cDNA-AFLP technology was used to identify and characterize cell cycle-modulated genes in BY2. cDNA-AFLP is a sensitive and reproducible fragment-based technology that has a number of advantages over other methods for genome-wide expression analysis (Breyne and Zabeau, Curr. Opin. Plant Biol., 4 136-142, 2001): it does not require prior sequence information, it allows identification of novel genes, and it provides quantitative expression profiles. After a detailed analysis, it was found that around 10% of the transcripts analyzed is periodically expressed. This comprehensive collection of plant cell cycle-modulated genes provides a basis for selecting and validating novel and unexpected agrochemical target genes

Synchronization of BY2 cells and sampling of material. Tobacco BY2-(Nicotiana tabacum L. cv. Bright Yellow-2) cultured cell suspension were synchronized by blocking cells in early S-phase with aphidicolin as follows. Cultured cell suspension of Nicotiana tabacum L. cv. Bright Yellow 2 were maintained as described (Nagata et al., Int. Rev. Cytol., 132 1-30, 1992). For synchronization, a 7-day-old stationary culture was diluted 10-fold in fresh medium supplemented with aphidicolin (Sigma-Aldrich, St. Louis, Mo.; 5 mg/l), a DNA-polymerase a inhibiting drug. After 24 h, cells were released from the block by several washings with fresh medium and resumed their cell cycle progression. After the drug had been washed, samples were taken every hour, starting from the release from the aphidicolin block (time 0) until 11 h later. The mitotic index was determined by counting the number of cells undergoing mitosis under fluorescence microscopy after the DNA had been stained with 5 mg/l 4′,6-diamidino-2-phenylindole (Sigma-Aldrich). DNA content was measured by flow cytometry. This was done as follows A subsample was used to check cell cycle progression and synchrony levels. After the DNA had been stained with 5 mg/l 4′,6-diamidino-2-phenylindole (Sigma-Aldrich), the mitotic index was determined under fluorescence microscopy by counting the number of cells undergoing mitosis. A mitotic peak of approximately 40% was obtained 8 h after washing. For flow cytometry, cells were first incubated in a buffered enzyme solution (2% cellulase and 0.1% pectolyase in 0.66 M sorbitol) for 20 min at 37° C. After the suspension had been washed and resuspended in Galbraith buffer (Galbraith et al., Science, 220 1049-1051, 1983), it was filtered through a 30-μm nylon mesh to purify the DAPI-stained nuclei. The fluorescence intensity was measured using a BRYTE HS flow cytometer (Bio-Rad, Hercules, Calif.). Exit from S-phase was observed 4 h after aphidicolin release and the level of synchrony was shown to be sufficiently high throughout the time course.

RNA extraction and cDNA synthesis. Total RNA was prepared by using LiCl precipitation (Sambrook et al., 1989) and poly(A⁺) RNA was extracted from 500 μg of total RNA using Oligotex columns (Qiagen, Hilden, Germany) according to the manufacturer's instructions. Starting from 1 μg of poly(A⁺) RNA, first-strand cDNA was synthesized by reverse transcription with a biotinylated oligo-dT₂₅ primer (Genset, Paris, France) and Superscript II (Life Technologies, Gaithersburg, Md.). Second-strand synthesis was done by strand displacement with Escherichia coli ligase (Life Technologies), DNA polymerase I (USB, Cleveland, Ohio.) and RNAse-H (USB).

cDNA-AFLP analysis. Five hundred ng of double-stranded cDNA was used for AFLP analysis as described (Vos et al., Nucl. Acids Res., 23 4407-4414, 1995; Bachem et al., Plant J., 9 745-753, 1996) with modifications. The restriction enzymes used were BstYI and MseI (Biolabs) and the digestion was done in two separate steps. After the first restriction digest with one of the enzymes, the 3′ end fragments were collected on Dyna beads (Dynal, Oslo, Norway) by means of their biotinylated tail, while the other fragments were washed away. After digestion with the second enzyme, the released restriction fragments were collected and used as templates in the subsequent AFLP steps. The adapters used were: for BstYI, 5′-CTCGTAGACTGCGTAGT-3′ and 5′-GATCACTACGCAGTCTAC-3′, and for MseI, 5′-GACGATGAGTCCTGAG-3′ and 5′-TACTCAGGACTCAT-3′; the primers for BstYI and MseI were 5′-GACTGCGTAGTGATC(T/C)N₁₋₂-3′ and 5′-GATGAGTCCTGAGTAAN₁₋₂-3′, respectively. For preamplifications, a MseI primer without selective nucleotides was combined with a BstYI primer containing either a T or a C as 3′ most nucleotide. PCR conditions were as described Vos et at., Nucl. Acids Res., 23 4407-4414, 1995). The obtained amplification mixtures were diluted 600-fold and 5 μl was used for selective amplifications using a P³³-labeled BstYI primer and the Amplitaq-Gold polymerase (Roche Diagnostics, Brussels, Belgium). Amplification products were separated on 5% polyacrylamide gels using the Sequigel system (Biorad). Dried gels were exposed to Kodak Biomax films as well as scanned in a phospholmager (Amersham Pharmacia Biotech, Little Chalfont, UK).

Quantitative measurements of the expression profiles and data analysis. Gel images were analyzed quantitatively with the AFLP-QuantarPro image analysis software (Keygene Nev., Wageningen, The Netherlands). This software was designed for accurate lane definition, fragment detection, and quantification of band intensities. All visible AFLP fragments were scored and individual band intensities were measured per lane. The obtained data were used to determine the quantitative expression profile of each transcript. The raw data were corrected for differences in total lane intensities, after which each individual gene expression profile was variance-normalized. This was done as follows.

The obtained raw data were first corrected for differences in total lane intensities which may occur due to loading errors or differences in the efficiency of PCR amplification with a given primer combination for one or more time points. The correction factors were calculated based on constant bands throughout the time course. For each primer combination, a minimum of 10 invariable bands was selected and the intensity values were summed per lane. Each of the summed values was divided by the maximal summed value to give the correction factors. Finally, all raw values generated by QuantarPro were divided by these correction factors.

Subsequently, each individual gene expression profile was variance-normalized by standard statistical approaches as used for microarray-derived data (Tavazoie et al., Nature Genet., 22 281-285, 1999). For each transcript, the mean expression value across the time course was subtracted from each individual data point after which the obtained value was divided by the standard deviation. A coefficient of variation (CV) was calculated by dividing the standard deviation by the mean. This CV was used to establish a cut-off value and all expression profiles with a CV less than 0.25 were considered as constitutive throughout the time course.

The Cluster and TreeView software (Eisen et al., PNAS, 95 14863-14868, 1998) was used for hierarchical, average linkage clustering. Quality-based clustering was done with a newly developed software program (De Smet et al., Bioinformatics 2002 May; 18(5): 735-46). This program is related to K-means clustering, except that the number of clusters does not need to be defined in advance and that the expression profiles that do not fit in any cluster are rejected. The minimal number of tags in a cluster and the required probability of genes belonging to a cluster were set to 10 and 0.95, respectively. With these parameters, 86% of all the tags were grouped in 21 distinct clusters.

Characterization of AFLP fragments. Bands corresponding to differentially expressed transcripts were isolated from the gel and eluted DNA was reamplified under the same conditions as for selective amplification. Sequence information was obtained either by direct sequencing of the reamplified polymerase chain reaction product with the selective BstYI primer or after cloning the fragments in pGEM-T easy (Promega, Madison, Wis.) or sequencing of individual clones. The obtained sequences were compared against nucleotide and protein sequences present in the publicly available databases by BLAST sequence alignments (Altschul et al., Nucl. Acids Res., 25 3389-3402, 1997). When available, tag sequences were replaced with longer EST or isolated cDNA sequences to increase the chance of finding significant homology. Based on the homology, transcript tags were classified in functional groups as shown in Table 1.

### Experimental Results

Identification and Characterization of Cell Cycle-Modulated Genes

Tobacco BY2 cells were synchronized by blocking cells in early S-phase with aphidicolin, an inhibitor of DNA polymerase a. After the inhibitor had been released, 12 time points with an 1-h interval were sampled, covering the cell cycle from S-phase until M-to-G1 transition. Flow cytometry and determination of the mitotic index showed that the majority of cells exit S-phase 4 h after release from blocking and that the peak of mitosis is reached at 8 h. From each time point, extracted mRNA was subjected to cDNA-AFLP-based transcript profiling.

Quantitative temporal accumulation patterns of approximately 10,000 transcript tags were determined and analyzed. In total, around 1,340 transcript tags were modulated significantly during the cell cycle. Hierarchical clustering of the expression profiles resulted in four large groups with the peak of expression in S-, early G2-, late G2-, or M-phase. Within each of these groups, several smaller clusters of genes with similar expression patterns could be distinguished. By quality-based clustering 21 different clusters were identified (see: http://www.plantgeneticsigenomics/CCMgenes). In agreement with the hierarchical clustering, the four largest clusters (clusters 1 to 4 in FIG. 1) correspond to the S-, early G2-, late G2-, and M-phases and together contain 65% of all the tags. An additional cluster (cluster 5 in FIG. 1C), not clearly separated in the hierarchical clustering, includes the genes with peak expression in G1-phase and contains another 5% of the tags. The remaining clusters are much smaller and most often (e.g., clusters 6, 9, 10, and 18) include genes with a narrow temporal expression pattern. In addition to these clusters, three small groups of genes displaying elevated expression during two cell cycle phases were distinguished also by quality-based clustering (FIG. 1D).

After the transcript tags had been sequenced, homology searches revealed that 36.5% of the tags were significantly homologous to genes of known functions, 13.1% of the tags matched a cDNA or genomic sequence without allocated function, whereas for 50.4% of the tags no homology with a known sequence was found. Genes of known function belong to diverse functional classes (Table 1) revealing that several biological processes are at least partially under temporal transcriptional control during the cell cycle in plants. In general, the observed transcript accumulation profiles and cell cycle specificity correlate well with the functional properties of the corresponding genes. It is interesting that the number of transcription factors with G2-phase specificity is high, which may be related with the induction of genes involved in M-phase-specific processes. The overrepresentation of RNA-processing genes in the M-phase might indicate that post-transcriptional regulation is involved in gene activity during mitosis. Because de novo transcription is severely reduced during mitosis (Gottesfeld et al., Trends Bioch. Sci., 22 197-202, 1997). RNA-processing could provide an alternative regulatory mechanism. Intriguingly, transcript tags with homology to a gene of unknown function are overrepresented in the M-phase as well (Table 1). The principal differences in cell cycle events between plants and other organisms occur during mitosis; therefore, the inventors believe that several of these transcripts correspond to still uncharacterised plant-specific genes triggering these events. Remarkably, several of the tags homologous to a publicly available sequence have no Arabidopsis homologue, indicating that, in addition to conserved genes, different plant species possess also unique sets of cell cycle-modulated genes. Although many of these tags may be too short to significantly match with an Arabidopsis sequence, analysis of longer cDNA clones corresponding to a subset of tags has revealed that approximately 25% of the sequences remain novel.

In Tables 1 to 4 a selection of 785 sequence tags are shown. This selection was based on the criterion if the tags were full length or that showed homology with genes known to be involved in the cell cycle (group 2 SEQ ID NOs 22 to 118), or on the criterion that they show homology with genes of unknown function (group 3 SEQ ID NOs 119 to 283) or on the criterion that the sequences showed no homology with the sequences in that existing databases (group 4 SEQ ID NOs 284-785). A first group (SEQ ID Nos 1 to 21) represent a smaller selection of tags which are used in the target validation method described in the present invention, more particularly, that were used in example 2.

The Core Cell Cycle Machinery

Several tags coincide with genes belonging to the core cell cycle machinery and exhibiting distinct expression profiles. Transcript tags from five B1- or B2-type cyclins as well as from a D2-type cyclin show mitotic accumulation and exhibit a narrow temporal expression profile, confirming previous studies (Mironov et al., Plant Cell, 11 509-521, 1999; Sorrell et al., Plant Physiol., 119 343-351, 1999). Based on the transcription patterns, the six A-type cyclins fall into three groups that sequentially appear during the cell cycle, adding new data to earlier observations (Reichheld et al., PNAS, 93 13819-13824, 1996). Two groups have quite a broad window of transcript accumulation; one group, homologous to A3-type cyclins, is expressed during S-phase and disappears during G2-phase and the other group, corresponding to A2-type cyclins comes up at mid S-phase and goes down during M-phase, except for one transcript that is specific for S-phase. The third group, containing an A1-type cyclin, has the same expression pattern as the B- and D2-type cyclins. Several tags derived from genes encoding the plant-specific B-type cyclin-dependent kinases (CDKs) were also identified. CDKB1 and CDKB2 peak at the G2-to-M transition, slightly before the mitotic cyclins as describe (Porceddu et al., J. Biol. Chem., 276 36354-36360, 2001). In contrast to what has been observed in partially synchronized alfalfa cell cultures (Magyar et at., Plant Cell, 9 223-235, 1997), the transcript levels of the tags homologous to a C-type CDK accumulate differentially during the cell cycle. The transcripts are present during late M-phase and early S-phase, suggesting that CDKC is active during the G1-phase.

In addition to these well-characterized cell cycle-regulatory genes, also several tags were identified herein derived from genes encoding transcription factors and protein kinases or phosphatases with a known or putative role in cell cycle control. One tag with a sharp peak of transcript accumulation 1 h before the B- and D-type cyclins corresponds to a 3R-MYB transcription factor. Recently, a 3R-MYB has been shown to activate B-type cyclins and other genes with a so-called M-phase-specific activator domain (Ito et al., Plant Cell, 13 1891-1905, 2001). Another tag peaking in M-phase is homologous to the CCR4 associated protein CAF. CAF forms a complex with CCR4 and DBF2, resulting in a transcriptional activator involved in the regulation of diverse processes including cell wall integrity, methionine biosynthesis and M-to-G1 transition (Liu et al., EMBO J., 16 5289-5298, 1997). A majority of the tags with similarity to protein kinases and phosphatases show M-phase-specific accumulation (Table 1). Although the true identity and putative cell cycle related function remains unclear for the majority, one is highly homologous to a dual-specificity phosphatase. This type of phosphatases plays a crucial role in cell cycle control in yeast and animals (Coleman and Dunphy, Curr. Opin. Cell Biol., 6 877-882, 1994). Another M-phase-specific tag is homologous to prohibitin. In the mammalian cell cycle, prohibitin represses E2F-mediated transcription via interaction with retinoblastoma (Rb), thereby blocking cellular proliferation (Wang et al., Oncogene, 18 3501-3510, 1999).

Protein degradation by the ubiquitin-proteasome pathway also plays an important role in the control of cell cycle progression at both G1-to-S transition and exit from mitosis. Although there is little evidence for cell cycle-modulated expression of the genes encoding the various components of the ubiquitin-proteasome complexes, some proteins accumulate in a cell cycle-dependent way (del Pozo and Estelle, Plant Mol. Biol., 44 123-128, 2000). Furthermore, several tags were isolated herein from genes encoding ubiquitin-conjugating enzyme (E3), ubiquitin-protein ligase (E2), and proteasome components with an M-phase-specific expression pattern. Another transcript tag that accumulates during late M-phase is similar to cathepsin B-like proteins, which are proteolytically active and degrade diverse nuclear proteins, including Rb (Fu et al., FEBS Lett., 421 89-93, 1998).

Whereas all the core cell cycle regulatory genes have been identified that control the G2-to-M transition for which the expression is known to be cell cycle modulated, genes such as Rb and E2F, controlling G1-to-S transition were not found. These genes were probably missed because the G1-to-S transition was not included in the present analysis, what is supported by the finding that the early targets of E2F, such as polymerase a and ribonucleotide reductase, are already present at high levels at the beginning of the time course.

Genes Involved in DNA Replication and Modification

In agreement with the studies performed in yeast and human fibroblasts, transcripts encoding proteins involved in DNA replication and modification accumulated during S-phase and exhibited broad temporal expression profiles. Different replication factors, DNA polymerase α, and the histones H3 and H4 are already present at the onset of the time course, indicating that they are induced before the time point of the aphidicolin arrest. Interestingly, most of the histones H1, H2A, and H2B appear somewhat later than H3 and H4, what might reflect that they are deposited into the nucleosomes after H3 and H4 (Luger et al., Nature, 389 251-260, 1997; Tyler et al., Nature, 402 555-560, 1999). The profile of the homologue of the anti-silencing function 1 (ASF1) protein is similar to that of the histones H3 and H4, in agreement with the fact that the three proteins are part of the replication-coupling assembly factor complex that mediates chromatin assembly (Tyler et al., Nature, 402 555-560, 1999). Genes encoding high-mobility group proteins reach the highest accumulation during late G2, consistent with the subsequent steps involved in the folding and structuring of the chromatin. Tags derived from genes encoding proteins involved in DNA modification, such as S-adenosyl-L-methionine (SAM) synthase and cytosine-5-methyl- transferase are found in the histone cluster. Tags from methionine synthase genes, which provide the precursor for SAM synthase, accumulate during M-phase, in contrast to yeast, where these genes are expressed during late S-phase (Spellman et al., Mol. Cell Biol., 9 3273-3297, 1998).

Genes involved in chromatin remodelling and transcriptional activation or repression have been identified as well. One gene is a histone deacetylase with highest transcript accumulation during the G2-phase and another belongs to the SNF2 family of chromodomain proteins with an M-phase-specific expression pattern. Interestingly, one tag corresponds to a mammalian inhibitor of growth 1 (p33-ING1) protein. The human ING1 protein has DNA-binding activity and might be involved in chromatin-mediated transcriptional regulation (Cheung and Li, Exp. Cell Res., 268 1-6, 2001). This protein accumulates during S-phase (Garkavtsev and Riabowel, Mol. Cell Biol., 17 2014-2019, 1997), what is in agreement with the expression profile we observed. The yeast homologues of ING1 are components of the histone acetyltransferase complex and show similarity to the Rb-binding protein 2 (Loewith et al., Mol. Cell Biol., 20 3807-3816, 2000). Another tag, homologous to the Arabidopsis MSI3 protein, follows a similar expression profile. MSI-like proteins are involved in the regulation of histone acetylation and deacetylation and in chromatin formation (Ach et al., Plant Cell, 9 1595-1606, 1997).

The expression profiles of the different ribonucleotide reductase (RNR) genes are more complex. One gene is already expressed at high levels at the beginning of the time course and its expression is restricted to the S-phase as described (Chabouté et al., Plant Mol. Biol., 38 797-806, 1998), whereas, in contrast, another one is highly expressed in S-phase and reappears at lower levels during M-phase and a third one is M-phase-specific. This latter expression profile has also been described for a RNR gene from Xenopus where the encoded protein appears to be involved in microtubulin nucleation (Takada et al., Mol. Cell Biol., 11 4173-4187, 2000).

Numerous other transcript tags with S-phase specificity were found in addition to the ones involved in DNA replication and modification. Most interestingly, one of these tags is homologous to a mammalian gene encoding a TRAF-interacting protein (TRIP), which is a component of the tumor necrosis factor (TNF) signaling complex, and promotes cell death when complexed with TRAF (Lee et al., J. Exp. Medicine, 185 1275-1285, 1997). Another S-phase-specific tag shows homology to the RING finger domain of inhibitor of apoptosis proteins, which are also involved in the TNF signaling pathway.

Modulated Expression of Genes Required for Mitosis and Cytokinesis

Several paralogous genes that encode either α- or β-tubulin were highly induced and accumulated prior to the mitotic index peak or during early M-phase. The inventors found that in BY2, tubulin genes are highly cell cycle modulated. This transcriptional regulation is in agreement with previous demonstrations of de novo transcription of α- and β-tubulin genes during different cellular processes (Stotz et al., Plant Mol. Biol., 41 601-614, 1999). In the present analysis, no γ-tubulin genes were found, confirming published data that the amount of γ-tubulin is constant in dividing BY2 cells (Stoppin-Mellet et al., Plant Biol., 2 290-296, 2000). Most of the kinesins identified herein, fall in the same cluster as the tubulins peaking prior to mitosis. Interestingly, two tags have a distinct transcription pattern and appear in another gene cluster. Their window of transcript accumulation is very narrow and coincides with the peak of mitosis. Most interestingly, these tags correspond to the plant-specific phragmoplast-associated type of kinesin, PAKRPI (Lee and Liu, Curr. Biol., 10 797-800, 2000). A chromokinesin not yet described in plants was identified as well. This type of motor proteins use DNA as cargo and play a role in chromosome segregation and metaphase alignment (Wang et al., J. Cell Biol., 128 761-768, 1995).

Among the M-phase-specific kinases, two were unambiguously recognized herein as playing a role in cytokinesis. One is Aurora, a protein kinase with a key role in the control of chromosome segregation, centrosome separation, and cytokinesis in yeast and animals (Bischoff and Plowman, Trends Cell Biol., 9 454-459, 1999) but not described in plants yet. The other is NRK1, a mitogen-activated protein kinase which is phosphorylated by NPK1, a kinase involved in regulating the outward redistribution of phragmoplast microtubules (Nishihama et al., Genes Dev., 15 352-363, 2001).

Hormonal Regulation and Cell Cycle-Modulated Gene Expression

A number of genes belonging to the class of auxin-induced genes were also differentially expressed. Cell cycle-modulated expression of auxin-induced genes has never been observed before although auxins together with cytokinins are the two major groups of plant hormones that affect cell division (Stals and Inzé, Trends Plant Sci., 6 359-364, 2001). The genes as identified herein fall into two groups based on their transcript accumulation profiles (data not shown). The first group displays an early S-phase-specific expression pattern and consists of the parA, parB and parC genes. Induction of the par genes is most often observed in response to stress conditions (Abel & Theologis, Plant Phys. 111, 9-17, 1996). The fact that the transcripts rapidly disappear after release from the cell cycle-blocking agent might indicate a stress response rather than a cell cycle dependent auxin response.

More interesting is the second group of genes with transcripts accumulating during early M-phase. This group includes the auxin response factor 1 (ARF1), an auxin transporter as well as different members of the early auxin response AUX/IAA gene family. ARF1 is a transcription factor that binds to a particular auxin response element (Ulmasov et al., Science, 276 1865-1868, 1997). Additional studies suggest that the activity of ARF1 is controlled by its dimerization with members of the AUX1/IAA family (Walker and Estelle, Curr. Opin. Plant boil., 1 434-439,1998). The similarity in temporal expression profiles the inventors observed supports these findings and suggests that these proteins mediate an auxin response necessary for cell cycle progression

By using tobacco BY2 as model system together with cDNA-AFLP-based transcript profiling, it is described herein for the first time how a comprehensive inventory of plant cell cycle-modulated genes can be made. Although the obtained data confirm earlier results and observations, in addition, numerous novel findings were made. The obtained data are a very useful basis for selecting and validating agrochemical target genes.

### Example 2

In this example it is described how plant genes are evaluated for assessment of their essential character in the biological process, thus how they are validated as good candidate targets for agrochemicals.

The Tobacco Rattle Virus (TVR) is used to induce silencing of target genes. In case of an essential gene the simlencing will result in a lethal effect on the plant and therefore, the suystem allows to validate good candidates as targets for herbicides.

The TRV based system is used in this example in combination with series of candidate genes, more particularly with the candidate targets as represented herein as group 1 sequences consisting of the SEQ ID NOs 1 to 21. The identification technique of the present invention (see example 1) allowed to identify new genes that are potential new herbicide targets, because of their putative function in various key processes crucial for cell life, their expression at a certain developmental stage crucial for cell life, their role in metabolism and/or maintenance of cell living state.

This example illustrates the validation of these candidate genes as novel targets for agrochemicals, via the technique of the virus-induced gene silencing (VIGS).

Gene Silencing Mechanism

The virus-induced gene silencing (VIGS) is a manifestation of an RNA-mediated defence mechanism that is related to post-transcriptional gene silencing (PTGS) in transgenic plants (Ratcliff et al., Plant J., 25 237-245, 2001). The method uses a vector with an infectious cDNA of tobacco rattle virus (TRV) modified (see below) to facilitate insertion of target sequences and modified for efficient infection of plants (e.g. tobacco). The vector mediates VIGS of endogenous genes in the absence of specific virus-induced symptoms.

The RNA-mediated defence is triggered by the virus vectors, and targets both the viral genome and the host gene corresponding to the insert. As a result, the symptoms in the infected plant are similar to loss-of-function mutants or reduced-expression mutants in the host gene. The presence of a negative growth phenotype suggests that the targeted gene is a potential herbicide target.

The process of constructing a virus vector and monitoring symptoms on infected plants is completed within a few weeks, such that virus-induced gene silencing (VIGS) provides a simple, rapid means of assigning function to genes that have been sequenced but are otherwise uncharacterized. The determination of new herbicide target genes is performed in a few weeks including gene cloning, transformation steps and tobacco plant analyses.

The TRV construct is shown to target host RNAs in the growing points of plants (Ratcliff et al., Plant J., 25 237-245, 2001) such as meristems and actively dividing cells.

It has been shown that this vector overcomes many of the problem features of PVX, TMV and TGMV. For example, the TRV vector induces very mild symptoms, infects large areas of adjacent cells and silences gene expression in growing points such as meristems and actively dividing cells. Infection of tobacco plants on the leaves with TRV based constructs will affect growth and development of upper parts of the infected leaves and allow screening for growth parameters.

Construction of TRV Vectors Used in the Validation Process of the Present Invention

TRV is a positive-strand RNA virus with a bipartite genome. Proteins encoded by RNA 1 are sufficient for replication and movement within the host plant, while proteins encoded by RNA 2 allow virion formation and nematode-mediated transmission between plants (reviewed by MacFarlane, J. Gen. Virol., 80 2799-2807,1999).

The downregulation system is composed of separate cDNA clones of TRV RNA 1 and RNA 2 under the control of cauliflower mosaic virus (CaMV) 35S promoters on the transferred T-DNA of plant binary transformation vectors.

The TRV RNA 1 construct (pBINTRA6) contains a full-length infectious cDNA clone in which the RNA polymerase ORF is interrupted by intron 3 of the Arabidopsis Col-0 nitrate reductase NIA1 gene (Wilkinson and Crawford, Mol. Gen. Genet., 239 289-297, 1993), necessary to prevent expression of a TRV-encoded protein that is toxic to E. coli. This vector has been given the internal reference number p3209.

The TRV RNA 2 construct (pTV00), contains a multiple cloning site (MCS), leaving only the 5′ and 3′ untranslated regions and the viral coat protein (Ratcliff et al., Plant Cell, 11 1207-1215, 1999). This vector has the internal reference number p3930 and contains a Gateway™ cassette and the gene of interest to be tested. The genes as presented in SEQ ID NO 1 to 21 are each cloned in this vector.

cDNAs were amplified using Gateway compatible primers and the cDNAs were entered into Entry Clones by BP recombination reactions. Subsequently the entry clones comprising the gene according to any one of SEQ ID NO 1 to 21 were checked via Bang restiction digest. The genes of interest were then entered into destination vectors by LR recombination reactions and the destination vectors were checked via ECORV restriction digestions. These expression clones were electroporated into the Argobacterium strain GV3101 agro and the plasmid pBintra6 was electroporated into pMP90 agro.

Inoculation

To inoculate plants, Agrobacterium cultures carrying pBINTRA6 (strain C58C1 RifR containing pMP90 plasmid) and pTV00 (strain GV3101 containing pMP90 plasmid) were grown and mixed and infiltrated to the leaves of Nicotiana benthamiana as previously described (English et al., Plant J., 12 597-603, 1997). Briefly, virus infection was achieved by Agrobacterium-mediated transient gene expression. Agrobacterium containing the TRV cloning vectors were grown overnight in L brith (+Tc+Km), Agrobacterium containing the helper plasmid was grown overnight in 10 ml YEB+Rif+Km. The culture was centrifuged and resuspended in 10 ml of 10 mM MgCl₂, 1 mM MES-pH5.6 and 100 μM acetosyringone and kept at room temperature for 2 h. Separate cultures containing pBINTRA6 and TRV cloning vectors were mixed in a ratio of 1:10. The culture was then infiltrated to the underside of two leaves of three-weeks old plants using a 2 ml syringe without a needle. In two independent experiments 6 plants per agroabcterium clone were infected. In this way the cloned genes (SEQ ID NO 1-21) were transferred into the cells of the infiltrated region, and could be transcribed into the viral cDNAs in the leave cells. These transcripts then serve as an inoculum to initiate systemic infection of the plant. Consequently the VIGS system is activated, resulting in the downregulation of the host cell gene, corresponding to the cloned gene of interest. All experiments involving virus-infected material was carried out in controlled growth chambers. N. benthamiana plants were germinated ad grown individually on universal potting ground in pots at 25° C. during the day (16 h) and 20° C. during the night (8 h).

The plants were phenotypically evaluated on a daily basis. Particular attention was given to visible leaf damage and growth inhibition. The effects of the suppression of gene activity using the VIGS system is measured by the phenotypic aspect of the plants, including leaf defects such as growth retardation, yellow or necrotic spots, early senescence, etc. The effects of the downregulation of genes identified by the methods of the invention are also measured on the flower structure and the flowering capacities of the transformed plants.

The severity of the phenotype is linked to the level of suppression of the geneactivity and indicates the degree in which the gene is essential for the plant Therefore the phenotype is an indication of the degree in which the gene is a valid target for a herbicide.

Phenotypes of the Infected Plants

1. Co-suppression of the gene leads to loss of gene transcription and protein expression in the virus infected leaf and induces leaf growth modification, including leaf wrinkling, curling, wilting, leading to cell death and/or plant death.

2. Co-suppression of the geneleads to loss of gene transcription and protein expression in the virus infected leaf and induces leaf yellowing or senescence, or cell death and necrosis, leading to plant death.

3. Co-suppression of the gene leads to loss of gene transcription and protein expression in the virus infected leaf and induces any of the following phenotypic symptoms: chlorotic regions around infection, crisp or crunchy leaf texture around infection, numerous surface lumps on either leaf surface, abnormal trichomes, abnormal leaf size, reduced growth, reduced final size, altered vascular leaf system, altered water movement in leaf, leading to cell death and/or plant death.

4. Co-suppression of the gene leads to loss of gene transcription and protein expression in the virus infected leaf and induces any of the following anatomical symptoms: clumps of modified cells on the surface of the leaf (either abaxial or adaxial), individual cells detached from the epidermis, swollen or modified trichome cells, modification of leaf tissue structure, cell size, cell number, tissue composition, parenchyme, epidermis, etc, leading to cell death and/or plant death.

5. co-suppression of gene X leads to loss of gene transcription and protein expression in the virus infected leaf and induces any of the following biochemical symptoms, enzyme activity and products, degradation of leaf components and effects in neighboring leaves, stem, vascular system, degradation of cell wall structure, communication between cells, modification of cell-cell signaling leading to cell death and/or plant death.

The genes identified by the present invention can be utilized to examine herbicide tolerance mechanisms in a variety of plants cells, including gymnosperms, monocots and dicots. It is particularly useful in crop plant cells such as rice, corn, wheat, barley, rye, sugar beet, etc

### Example 3

Significant phenotypic alterations could be observed in plants infiltrated with Agrobacterium containing pBINTRA6+Bstt44-4-340 (SEQ ID NO 18, acetolactate synthetase) and pBINTRA6+Bstt2-42-520 (or T4-32-7) (SEQ ID NO 21, prohibitin) and pBINTRA6+Bstt230 (SEQ ID NO 11, B-type CDK).

At 10 days post-infiltration the first symptoms were visible. The symptoms were persistent until the end of the experiment and could be observed in at least 5 out of the 6 infiltrated plants.

The phenotypes of the plants transformed with acetolactate synthase are further described.

In two separate replicated experiments, specific phenotypes on each plant infected with the acetolactate synthetase downregulation construct were observed (FIG. 2). Winkling and wrapping of the leaves as well as some chlorotic spots were observed. Thus acetolactate downregulation provoked a general growth arrest accompanied with chlorotic and necrotic areas. These observations were in line with previous reports, wherein acetolactate synthetase is described as a useful herbicide target.

The phenotypes of the plants transformed with prohibitin are further described. In two separate replicated experiments, specific phenotypes on each plant infected with the prohibitin downregulation construct were observed (FIG. 2). These plants showed strong wrinkling of the leaves about 20 days after infection, corresponding to the expected occurrence of silencing events. Thus the downregulation of prohibitin provokes a severe leaf distortion and general growth arrest.

The phenotype of the plants inoculated with a B-type CDK downregulation construct are shown in FIG. 3. A late (from 30 days after inoculation) but strong negative effect on the plant growth was observed. The plants started to grow much slower and lost their apical dominance, resulting in the increased appearance of lateral branches.

TABLE 1
Functional classification of transcript tags

| | | S | | G2 | | M | | G1 |
|---|---|---|---|---|---|---|---|---|
| Function | Tags | 27.7% | | 15.8% | | 52.9% | | 3.6% |
| Cell cycle control | 30 | 5/8 | (0.078) | 8/5 | (0.068) | 14/16 | (0.114) | 3/1 |
| Cell wall | 35 | 6/10 | (0.047) | 4/6 | (0.136) | 25/18 | (7.1e⁻³) | 0/1 |
| Cytoskeleton | 43 | 1/12 | (1.2e⁻⁵) | 4/7 | (0.090) | 38/22 | (2.1e⁻⁷) | 0/2 |
| Hormone response | 13 | 6/4 | (0.113) | 1/2 | (0.277) | 6/7 | (0.185) | 0/0 |
| Kinases/phosphatases¹ | 27 | 4/8 | (0.039) | 1/4 | (0.059) | 19/14 | (0.025) | 3/1 |
| Protein synthesis | 50 | 15/14 | (0.116) | 5/8 | (0.087) | 29/26 | (0.079) | 1/2 |
| Proteolysis | 21 | 2/6 | (0.026) | 1/3 | (0.144) | 17/11 | (0.039) | 1/1 |
| Replication and modification | 74 | 57/20 | (4.2e⁻¹⁹) | 8/12 | (1.0e⁻⁵) | 8/39 | (1.0e⁻¹⁸) | 1/3 |
| RNA processing | 20 | 1/6 | (6.8e−3) | 1/3 | (0.137) | 18/11 | (8.1e⁻⁴) | 0/0 |
| Signal transduction | 10 | 1/3 | (0.121) | 3/2 | (0.201) | 6/5 | (0.205) | 0/0 |
| Stress response | 20 | 6/6 | (0.192) | 2/3 | (0.229) | 10/10 | (0.159) | 2/1 |
| Transcription factors | 27 | 4/8 | (0.039) | 10/4 | (3.0e⁻³) | 12/14 | (0.112) | 1/1 |
| Transport and secretion² | 31 | 5/9 | (0.047) | 2/5 | (0.076) | 21/16 | (0.031) | 3/1 |
| Unknown | 175 | 37/48 | (0.015) | 19/28 | (0.014) | 112/93 | (8.3e⁻⁴) | 7/6 |

The total number of tags and the observed/expected number of tags within the different cell cycle phases for each functional group is given together with the probability values between parentheses as calculated based on the binomial distribution function, except for the G1-phase because the values were too small. A significant enrichment (P < e⁻³) of tags of a functional group within a particular cell cycle phase is indicated in bold.
¹Only kinases and phosphatases with unknown biological function.
²Except small GTP-binding proteins, which are classified under signal transduction.

TABLE 2
overview of group 1 of sequences used for validation of candidate target genes

| SEQ ID NO | CDS NO | Tag Name | Function | Fase |
|---|---|---|---|---|
| 1 | 2216 | 18R1850_C4-32-33_1E2 | catalase | ?? |
| 2 | 2217 | Bstt2-31-215 | phytoene desasturase | ?? |
| 3 | 2218 | Bstc13-1-145 | L-ascorbate peroxidase | M-G1 |
| 4 | 2219 | Bstc21-4-280 | GTP-bindingprotein | M |
| 5 | 2220 | Bstc33-2-310 | vacuolarsortingreceptor | M |
| 6 | 2221 | Bstc4-34-170 | probable cinnamyl alcohol dehydrogenase | G1/S-S; M-G1 |
| 7 | 2222 | Bstt34-3-470 | kinesin | M |
| 8 | 2223 | Bstt12-3-410 | B-typeCDK | M |
| 9 | 2224 | Bstt14-3-458 | squalene mono-oxygenase | G1/S-S |
| 10 | 2225 | Bstt12-1-230 | kinesin-likeprotein | M |
| 11 | 2226 | Bstt23-4-230 | B-typeCDK | M |
| 12 | 2227 | Bstt2-42-225 | B-typeCDK | M |
| 13 | 2228 | Bstt31-4-208 | arabinogalactan protein precursor | G2/M-M |
| 14 | 2229 | Bstt 3-41-205 | arabinogalactan protein precursor | G2/M-M |
| 15 | 2230 | Bstt33-4-285 | chorismate synthase | S-G2 |
| 16 | 2231 | Bstt2-31-215 | kinesin-likeprotein | M |
| 17 | 2232 | Bstt41-2-400 | endo-beta-1,4glucanase | M |
| 18 | 2233 | Bstt44-4-340 | acetolactate synthase | G2/S-G2-M-G1 |
| 19 | 2234 | G17-2-13 G17-2-13 | WRKY transcription factor | ?? |
| 20 | 2235 | mapk9-ntf6.seq | mapkinase phragmoplast associated NTF6 | ?? |
| 21 | 2236 | Bstt2-42-520 | prohibitin | ?? |

TABLE 3
overview of group 2 sequences of full-length sequences that are cell cycle
modulated and of which some are involved in the cell cycle process

| SEQ ID | CDS | |
|---|---|---|
| NO | NO | Gene name |
| 22 | 0613 | Protein kinase mRNA, complete, N. tabacum, 2073 bp |
| 23 | 0614 | BY2 AA041K03 probable DNA-binding protein GBP16 - rice T02069, N. tabacum, 834 bp |
| 24 | 0615 | BY2 AA042C09 probable nuclear DNA-binding protein G2p [imported] in Arabidopsis T51151, N. tabacum, |
| | | 1185bp |
| 25 | 0616 | BY2-AA044J17 transcription regulator-like in Arabidopsis AB025604, N. tabacum, 1893 bp |
| 26 | 0617 | BY2 AA044J23 ATP-dependent RNA helicase CA3 of the DEAD/DEAH box family; Dbp3p; BY2- |
| | | AA044J23P19G01 RNA helicase RH5 in Arabidopsis T51739 N. tabacum, 1593 bp |
| 27 | 0618 | BY2-AA046C15 protein phosphatase 2C-like in Arabidopsis BAB08417 AB025622, N. tabacum, 732 bp |
| 28 | 0619 | BY2-AA047G13 14-3-3-like protein C P93343, N. tabacum, 70 bp |
| 29 | 0620 | BY2-AA054L09 protein kinase tousled in Arabidopsis A49318 N. tabacum, 2037 bp |
| 30 | 0621 | BY2-AA066H11P19H05 phosphoprotein phosphatase 2A regulatory chain T03684 N. tabacum, 1764 bp |
| 31 | 0622 | BY2-AA069L10 transcription factor-like protein in Arabidopsis BAB09482 AB012246, N. tabacum, 831 bp |
| 32 | 0623 | BY2-AA073K06 SET protein, phospatase 2A inhibitor in Arabidopsis AAG52377.1 AC011765, N. tabacum |
| 33 | 0624 | BY2-AA073MP19B07 phosphoprotein phosphatase 2A regulatory chain T03684, N. tabacum, 1764bp |
| 34 | 0625 | BY2-AA075H12 Putative phospatase 2A inhibitor in Arabidopsis AC011809_9 AC011809, N. tabacum, |
| | | 783bp |
| 35 | 0626 | BY2-AA076O02P19B08 hypothetical protein kinase in Arabidopsis T47727, N. tabacum, 2514 bp |
| 36 | 0627 | BY2-AA079J13 putative casein kinase I in Arabidopsis AAG51841.1 AC010926_4, N. tabacum, 1401 bp |
| 37 | 0628 | BY2-AA080G14 porin I 36K in potato S46959, N. tabacum, 393 bp |
| 38 | 0629 | BY2-AA081P13p21E02 separation anxiety protein-like in Arabidopsis CAB96669.1 AL360314, N. tabacum, |
| | | 492bp |
| 39 | 0630 | Complementary copy of 0630, N. tabacum, 975 bp |
| 40 | 0631 | BY2-AA085N17p21H04 14-3-3-like protein in potato 16R P93784 N. tabacum 768 bp |
| 41 | 0632 | BY2-AA087C16p21G03 AP2 domain transcription factor homolog in potato T07784 N. tabacum, 891 bp |
| 42 | 0633 | BY2-AA088B13 putative RING zinc finger protein in Arabidopsis CAB80936.1 AL161491 N. tabacum |
| | | 1248bp |
| 43 | 0634 | BY2-AA095M08 protein kinase homolog in Arabidopsis T02181 N. tabacum 858 |
| 44 | 0635 | BY2-AA096M07 peptidyl-prolyl cis-trans isomerase-like protein BAB10691.1 AB015468 N. tabacum |
| | | 450bp |
| 45 | 0636 | BY2-AA096M12 zinc finger protein-like in Arabidopsis BAB09106.1 AB017069 N. tabacum 1518 bp |
| 46 | 0637 | BY2-AA096M22 cell division-like protein in Arabidopsis T45963 N. tabacum 687 bp |
| 47 | 0638_1 | BY2-AA098B08p21D11 similarity to DAG protein in Arabidopsis BAA97063.1 AP000370 N. tabacum |
| | | 1146bp |
| 48 | 0638_2 | Icl_AA091G16p21F05 N. tabacum 891 bp |
| 49 | 0639 | BY2-AA109N15 GAMM1 protein-like in Arabidopsis BAB08430.1 AB017067 N. tabacum 888 bp, (MYG1) |
| | | FAMILY, proliferation associated |
| 50 | 0640 | Complementary copy of 0640 N. tabacum, 891 bp |
| 51 | 0641 | BY2-AA114N16 unknown protein in Arabidopsis BAB03019.1 AP001297; candidate tumor suppressor |
| | | p33 ING1 homolog in Homo sapiens N. tabacum 720 bp |
| 52 | 0642 | BY2-AA115P21p22D02 NAC2 Arabidopsis AAF09254.1 AF201456_1N. tabacum 699 bp |
| 53 | 0643 | BY2-AA119N11p22G04 serine/threonine-specific protein kinase-like protein BAB09338.1 AB016879 N. tabacum |
| | | 1293 bp |
| 54 | 0662 | BY2-AA041E04 >pir\|\|T06678 hypothetical protein T17F15.80 - Arabidopsis thaliana |
| 55 | 0663 | BY2-AA043A01 >gb\|AAD24540.1\|AF113545_1 (AF113545) vacuole-associated annexin VCaB42 |
| | | [Nicotiana tabacum] |
| 56 | 0664 | BY2-AA044C02 >dbj\|BAA02028.1\|(D11470) chloroplast elongation factor TuB(EF-TuB) [Nicotiana |
| | | tabacum] |
| 57 | 0665 | BY2-AA044L14 dbj\|BAA97319.1\|(AB020754) gene_id: MYN8.3~pir\|\|T02891~similar to unknown protein |
| 58 | 0666 | BY2-AA045P04p01G10 sp\|Q43681\|NLTP_VIGUN PROBABLE NONSPECIFIC LIPID-TRANSFER |
| | | PROTEIN AKCS9 |
| 59 | 0667 | BY2-AA046C08p19E02 dbj\|BAB30364.1\|(AK016659) putative [Mus musculus] |
| 60 | 0668 | BY2-AA046E06 pir\|\|T50556 stamina pistilloidia protein Stp [imported] - garden pea |
| 61 | 0669 | BY2-AA046G14 dbj\|BAB26082.1\|(AK009117) putative [Mus musculus] |
| 62 | 0670 | BY2-AA046H23 emb\|CAA98172.1\|(Z73944) RAB8A [Lotus japonicus] |
| 63 | 0671 | BY2AA048A05 gb\|AAD15504.1\|(AC006439) putativeAAA-type ATPase [Arabidopsis thaliana] |
| 64 | 0672 | BY2-AA049K03 dbj\|BAB24909.1\|(AK007240) putative [Mus musculus] |
| 65 | 0673 | BY2-AA051A10 dbj\|BAB02543.1\|(AP000417) mitotic checkpoint protein [Arabidopsis thaliana] |
| 66 | 0674 | BY2-AA051L22p19H03 gb\|AAD48948.1\|AF147262_11 (AF147262) contains similarity to Pfam family |
| | | PF00400-WD domain |
| 67 | 0675 | BY2-AA052E10 >gb\|AAF52905.1\|(AE003628) CG4968 gene product [Drosophila melanogaster] |
| 68 | 0676 | BY2-AA052F14 >gb\|AAF79819.1\|AC007396_20 (AC007396) T4O12.22 [Arabidopsis thaliana] |
| 69 | 0677 | BY2-AA052G16p19D04 >dbj\|BAB09843.1\|(AB005246) gene_id: MUP24.12~unknown protein |
| | | [Arabidopsis thaliana] |
| 70 | 0678 | BY2-AA052N17 >gb\|AAG42914.1\|AF327533_1 (AF327533) unknown protein [Arabidopsis thaliana] |
| 71 | 0679_1 | BY2-AA053C11.1 >dbj\|BAB22857.1\|(AK003561) putative [Mus musculus] |
| 72 | 0679_2 | BY2-AA053C11.2 >gb\|AAC62883.1\|(AC005397) hypothetical protein [Arabidopsis thaliana] |
| 73 | 0680 | BY2-AA062A09 >gb\|AAF01061.1\|AF189284_1 (AF189284) nucleolar G-protein NOG1 [Trypanosoma |
| | | brucei] |
| 74 | 0681 | BY2-AA062G03 >pir\|\|T02135 hypothetical protein F8K4.10 - Arabidopsis thaliana |
| 75 | 0682 | BY2-AA065E08 >pir\|\|T00795 hypothetical protein F24L7.13 - Arabidopsis thaliana |
| 76 | 0683 | BY2-AA072K18 >emb\|CAB40381.1\|(AJ010819) GrpE protein [Arabidopsis thaliana] |
| 77 | 0684 | BY2-AA075K12 >gb\|AAD31331.1\|AC007354_4 (AC007354) T16B5.4 [Arabidopsis thaliana] |
| 78 | 0685 | BY2-AA076N08 >dbj\|BAA94770.1\|(AP001859) ESTs AU082761(S5084) D42006 |
| 79 | 0686 | BY2-AA080D01 >gb\|AAF80646.1\|AC012190_2 (AC012190) Contains similarity to F28O16.19 a putative |
| | | translation initiation protein |
| 80 | 0687 | BY2-AA081P14 >gb\|AAD32777.1\|AC007661_14 (AC007661) unknown protein [Arabidopsis thaliana |
| 81 | 0688 | BY2-AA082H04p21F02 >dbj\|BAB10171.1\|(AB016880) gene_id: MTG10.12~pir\|\|T05795~strong similarity |
| | | to unknown |
| 82 | 0689 | BY2-AA082H06p21G04 >pir\|\|T09039 hypothetical protein F26K10.110 - Arabidopsis thaliana |
| 83 | 0690 | BY2-AA082M07p21B05 >dbj\|BAB01783.1\|(AB022215) gene_id: MCB17.19~unknown protein |
| | | [Arabidopsis thaliana] |
| 84 | 0691 | BY2-AA083B24p21C04 >dbj\|BAB08247.1\|(AB006698) gene_id: MCL19.6~unknown protein [Arabidopsis |
| | | thanliana) |
| 85 | 0692 | BY2-AA083C05p21D02 >gb\|AAH02924.1\|AAH02924 (BC002924) Unknown (protein for |
| | | IMAGE: 3956179) [Homo sapiens] |
| 86 | 0693 | BY2-AA085D08p21C05 >pir\|\|T47624 hypothetical protein T5N23.10 - Arabidopsis thaliana |
| 87 | 0694 | BY2-AA085F09p21H01 >gb\|AAF79503.1\|AC002328_11 (AC002328) F20N2.15 [Arabidopsis thaliana] |
| 88 | 0695 | BY2-AA085M15p21D04 >gb\|AAF97305.1\|AC007843_8 (AC007843) Unknown protein [Arabidopsis |
| | | thaliana] |
| 89 | 0696 | BY2-AA088K23p21G05 >gb\|AAG52001.1\|AC012563_11 (AC012563) unknown protein; 64612-65506 |
| | | [Arabidopsis thaliana] |
| 90 | 0697 | BY2-AA088L24p21A07 >gb\|AAD55292.1\|AC008263_23 (AC008263) Contains PF\|00249 Myb-like DNA- |
| | | binding domain. |
| 91 | 0698 | BY2-AA089F12p21H05 >gb\|AAD55274.1\|AC008263_5 (AC008263) Strong similarity to gb\|D21805 |
| | | calcium-dependent protein kinase |
| 92 | 0699 | BY2-AA089M17 >pir\|\|T02186 hypothetical protein F14M4.16 - Arabidopsis thaliana |
| 93 | 0700 | BY2-AA090J23p21G08 >pir\|\|T48545 hypothetical protein F14F18.30 - Arabidopsis thaliana |
| 94 | 0701 | BY2-AA092F12p21H06 >emb\|CAB46854.1\|(AJ388555) hypothetical protein [Canis familiaris] |
| 95 | 0702 | BY2-AA092L20p21E07 >gb\|AAD10646.1\|(AC005223) 45643 [Arabidopsis thaliana] |
| 96 | 0703 | BY2-AA093J23p21C11 >gb\|AAG51461.1\|AC069160_7 (AC069160) unknown protein [Arabidopsis |
| | | thaliana] |
| 97 | 0704 | BY2-AA093L18p21D09 >emb\|CAC15504.1\|(AJ297917) B2-type cyclin dependent kinase [Lycopersicon |
| 98 | 0705 | BY2-AA093M19 >gb\|AAG12535.1\|AC015446_16 (AC015446) Unknown protein [Arabidopsis thaliana] |
| 99 | 0706 | BY2-AA094B12p21F10 >dbj\|BAB02118.1\|(AP000381) contains similarity to unknown |
| 100 | 0707_1 | BY2-AA096G05p21A11 dbj\|BAB02118.1\|(AP000381) contains similarity to unknown |
| 101 | 0707_2 | Icl_AA094B12p21F10 |
| 102 | 0708 | BY2-AA097G22p21D10 >gb\|AAG60065.1\|AF337913_1 (AF337913) unknown protein [Arabidopsis |
| | | thaliana |
| 103 | 0709 | BY2-AA099F04 gb\|AAG52457.1\|AC010852_14 (AC010852) hypothetical protein; 12785-11538 |
| | | [Arabidopsis thaliana] |
| 104 | 0710 | BY2-AA099N08p21H09 gb\|AAK14411.1\|AC087851_3 (AC087851) unknown protein [Oryza sativa] |
| 105 | 0711 | Icl_AA100B09 ref\|NP_009820.1\|Ybr261cp [Saccharomyces cerevisiae] |
| 106 | 0712 | BY2-AA109N02 ref\|NP_002848.1\|peroxisomal farnesylated protein; Housekeeping gene 33 kD [Homo |
| | | sapiens |
| 107 | 0713 | BY2-AA114E09p22F02 pir\|\|T51434 hypothetical protein F2G14_10 - Arabidopsis thaliana |
| 108 | 0714 | BY2-AA115B14p22C02 dbj\|BAB08888.1\|(AB012243) gene_id: MIJ24.6~ref\|NP_013897.1~similar to |
| | | unknown protein |
| 109 | 0715 | BY2-AA115F08p22C04 >gb\|BY2-AAH03900.1\|AAH03900 (BC003900) Similar to hypothetical protein |
| | | 384D8_6 [Mus musculus] |
| 110 | 0716 | BY2-AA115L12p22G01 >gb\|AAF43925.1\|AC012188_2 (AC012188) Contains similarity to PIT1 from |
| | | Arabidopsis thaliana |
| 111 | 0717 | BY2-AA116L23p22E01 >dbj\|BAB01460.1\|(AP000731) gene_id: MCB17.21~unknown protein |
| | | [Arabidopsis thaliana] |
| 112 | 0718 | BY2-AA117B12p21G12 >sp\|O23708\|PSA2_ARATH PROTEASOME SUBUNIT ALPHA TYPE 2 (20S |
| | | PROTEASOME ALPHA SUBUNIT B) |
| 113 | 0719 | BY2-AA117E08p22A03 >pir\|\|F81195 conserved hypothetical protein NMB0465 [imported] Neisseria |
| 114 | 0720 | BY2-AA117O08p22E03 >dbj\|BAB01753.1\|(AP000603) gb\|BY2-AAD10646.1~gene_id: MRP15.12 |
| 115 | 0721 | BY2-AA118D23p22E02 >emb\|CAB89490.1\|(AJ277062) CRK1 protein [Beta vulgaris], cdc2 like kinase |
| 116 | 0722 | BY2-AA119D12p22H04 >dbj\|BAB01163.1\|(AP000410) gene_id: K10D20.9~unknown protein |
| | | [Arabidopsis thaliana] |
| 117 | 0723 | BY2-AA120G12 >gb\|BY2-AAB63649.1\|(AC001645) hypothetical protein [Arabidopsis thaliana] |
| 118 | 0724 | BY2-AA120G19p22D05 >gb\|BY2-AAF69547.1\|AC008007_22 (AC008007) F12M16.18 [Arabidopsis |
| | | thaliana] |

TABLE 4
overview of group 3 sequences that show homology
with proteins of unknown function

| SEQ | | | |
|---|---|---|---|
| ID NO | Tag name and | Function | Fase |
| 119 | Bstc1-11-320 | | M-G1 |
| 120 | Bstc1-12-255 | | G2/M-M-G1 |
| 121 | Bstc1-12-275 | | G2/M-M-G1 |
| 122 | Bstc1-13-143 | unknownprotein | G2/M-M-G1 |
| 123 | Bstc1-13-160 | unknownprotein | G2/M-M-G1 |
| 124 | Bstc11-3-190 | | M-G1 |
| 125 | Bstc11-3-215 | putativeprotein | G2/M-M-G1 |
| 126 | Bstc11-3-230 | | G1/S; M-G1 |
| 127 | Bstc11-3-300 | unknown | M-G1 |
| 128 | Bstc13-4-168 | hypotheticalprotein | S-G2 |
| 129 | Bstc13-4-290 | hypotheticalprotein | M-G1 |
| 130 | Bstc14-205 | | G2/S-G2 |
| 131 | Bstc1-43-107 | | G2/S-G2 |
| 132 | Bstc14-3-165 | unknown | M-G1 |
| 133 | Bstc1-43-250 | unknown | G2/M-M-G1 |
| 134 | Bstc1-43-310 | hypotheticalprotein | G2/M-M |
| 135 | Bstc21-2-270 | hypotheticalprotein | G2/M-M-G1 |
| 136 | Bstc2-21-182 | unknown | M-G1 |
| 137 | Bstc22-1-275 | unknownprotein | G2-M-G1 |
| 138 | Bstc2-22-100 | unknown | G2-G2/M |
| 139 | Bstc2-22-155 | | G2-M |
| 140 | Bstc2-22-240 | hypotheticalprotein | M |
| 141 | Bstc22-2-270 | | G1/S; M-G1 |
| 142 | Bstc2-23-135 | | G2/S-G2-M |
| 143 | Bstc2-23-220 | unknown | G2-M-G1 |
| 144 | Bstc22-4-215 | hypotheticalprotein | G2/M-M |
| 145 | Bstc2-31-280 | | G2/M-M-G1 |
| 146 | Bstc23-2-240 | unknown | M |
| 147 | Bstc23-2-330 | putativeprotein | M |
| 148 | Bstc23-2-370 | | G1/S-S; G2/M-M-G1 |
| 149 | Bstc2-32-400 | | G1/S-S; G2/M-M-G1 |
| 150 | Bstc23-3-270 | | G1/S-S; M-G1 |
| 151 | Bstc2-33-280 | unknownprotein | G1/S-S; M-G1 |
| 152 | Bstc2-34-120 | unknown | G2/M-M-G1 |
| 153 | Bstc23-4-300 | unknown | M |
| 154 | Bstc2-41-165 | | G1/S-S |
| 155 | Bstc2-42-100 | unknown | G1/S-S |
| 156 | Bstc2-43-210 | | M-G1 |
| 157 | Bstc31-185 | unknown | G2/M-M-G1 |
| 158 | Bstc3-12-145 | unknown | S-G2 |
| 159 | Bstc3-12-290 | unknown | G2/M-M-G1 |
| 160 | Bstc31-3-400 | unknown | G2/M-M-G1 |
| 161 | Bstc32-1-122 | unknown | M-G1 |
| 162 | Bstc3-21-125 | | G1/S-S; G2/M-M-G1 |
| 163 | Bstc32-2-150 | putativeprotein | G1/S-S; G2/M-M-G1 |
| 164 | Bstc32-4-193 | | |
| 165 | Bstc32-4-370 | | G1/S-S-G2/S; M-G1 |
| 166 | Bstc3-31-350 | putativeprotein | G1/S-S-G2/S |
| 167 | Bstc33-2-145 | hypotheticalprotein | G1/S-S; G2/M-M-G1 |
| 168 | Bstc3-33-350 | | G1/S-S |
| 169 | Bstc33-360 | putativeprotein | G2/M-M-G1 |
| 170 | Bstc33-4-270 | unknown | G2/M-M |
| 171 | Bstc3-41-270 | unknown | M-G1 |
| 172 | Bstc3-41-300 | | G2/M-M-G1 |
| 173 | Bstc3-41-360 | | G2/M-M-G1 |
| 174 | Bstc3-42-175 | | M-G1 |
| 175 | Bstc3-43-135 | | G1 |
| 176 | Bstc3-43-180 | | M-G1 |
| 177 | Bstc3-43-193 | unknown | G1/S-S; G2/M-M-G1 |
| 178 | Bstc3-43-287 | | G1/S-S |
| 179 | Bstc3-44-145 | | M-G1 |
| 180 | Bstc3-44-375 | putativeprotein | M-G1 |
| 181 | Bstc4-11-120 | hypotheticalprotein | G2/M-M-G1 |
| 182 | Bstc4-11-320 | unknown | M-G1 |
| 183 | Bstc42-3-115 | unknown | M-G1 |
| 184 | Bstc42-3-125 | putativeprotein | G2/M-M-G1 |
| 185 | Bstc4-23-210 | | M-G1 |
| 186 | Bstc42-4-225 | unknown | G1/S-S-G2 |
| 187 | Bstc4-32-115 | unknownprotein | G1/S-S; G2/M-M-G1 |
| 188 | Bstc4-32-185 | unknown | G1/S-S |
| 189 | Bstc4-32-190 | unknown | G2/M-M |
| 190 | Bstc4-32-270 | unknown | G2/S-G2-M |
| 191 | Bstc4-32-410 | | G1/S-S-G2-G2/M |
| 192 | Bstc4-34-250 | | G2/M-M-G1 |
| 193 | Bstc4-41-230 | putativeprotein | G2/M-M-G1 |
| 194 | Bstc4-43-113 | unknown | W1-G1 |
| 195 | Bstc44-3-125 | | G2/M-M |
| 196 | Bstt1-12-340 | unknown | G2/M-M |
| 197 | Bstt12-2-225 | | G1/S-S-G2 |
| 198 | Bstt1-22-330 | unknown | G2/M-M-G1 |
| 199 | Bstt12-2-420 | unknownprotein | G2/M-M-G1 |
| 200 | Bstt12-2-540 | hypotheticalprotein | G2/M-M-G1 |
| 201 | Bstt1-23-155 | | M-G1 |
| 202 | Bstt12-3-215 | hypotheticalprotein | G2/M-M-G1 |
| 203 | Bstt12-3-280 | unknown | G1/S-S-G2 |
| 204 | Bstt12-3-310 | hypotheticalprotein | G1/S-S |
| 205 | Bstt12-3-350 | | G1/S-S-G2-G2/M |
| 206 | Bstt1-24-205 | | G2/M-M-G1 |
| 207 | Bstt1-24-220 | | G1/S-S-G2 |
| 208 | Bstt1-31-170 | hypotheticalprotein | G2/M-M-G1 |
| 209 | Bstt1-31-215 | unknown | G2/M-M-G1 |
| 210 | Bstt13-210 | unknown | G2/M-M-G1 |
| 211 | Bstt14-4-310 | unknownprotein | G2/M-M-G1 |
| 212 | Bstt2-11-165 | unknown | G2/M-M-G1 |
| 213 | Bstt2-12-190 | | G1/S-S-G2 |
| 214 | Bstt21-4-150 | hypotheticalprotein | G1/S-S-G2/S |
| 215 | Bstt21-4-250 | | G1/S-S; G2/M-G1 |
| 216 | Bstt21-4-470 | | G2/M-M-G1 |
| 217 | Bstt22-1-170 | unknown | S-G2 |
| 218 | Bstt2-21-190 | unknown | G2/M-M |
| 219 | Bstt22-2-190 | unknown | G2/M-M-G1 |
| 220 | Bstt22-2-290 | hypotheticalprotein | G2/M-M-G1 |
| 221 | Bstt22-3-225 | | M |
| 222 | Bstt22-3-275 | unknown | G2/M-M |
| 223 | Bstt22-3-315 | TomatoEST | G2/M-M-G1 |
| 224 | Bstt22-3-370 | unknown | G2/M-M-G1 |
| 225 | Bstt22-3-390 | putativeprotein | G2/M-M-G1 |
| 226 | Bstt22-3-480 | | G2/M-M-G1 |
| 227 | Bstt23-1-140 | | S-G2-G2/M |
| 228 | Bstt23-120 | unknownprotein | G2/M-M-G1 |
| 229 | Bstt23-1-200 | | S-G2-M |
| 230 | Bstt2-31-300 | unknown | S |
| 231 | Bstt2-32-220 | | M |
| 232 | Bstt2-32-400 | hypotheticalprotein | G2/M-M-G1 |
| 233 | Bstt23-3-350 | unknown | G2-M |
| 234 | Bstt23-370 | unknown | G2/M-M-G1 |
| 235 | Bstt24-1-320 | | S-G2 |
| 236 | Bstt24-2-310 | | G2/M-M-G1 |
| 237 | Bstt2-43-210 | unknown | G2-M |
| 238 | Bstt2-43-240 | | S-G2/S |
| 239 | Bstt31-1-100 | hypotheticalprotein | G1/S-S-G2 |
| 240 | Bstt3-11-205 | | G1/S-S-G2 |
| 241 | Bstt31-1-250 | hypotheticalprotein | G2/M-M-G1 |
| 242 | Bstt31-1-430 | hypotheticalprotein | G2/M-M-G1 |
| 243 | Bstt3-12-360 | unknownprotein | G2/M-M |
| 244 | Bstt31-3-380 | | G1/S-S |
| 245 | Bstt31-4-420 | hypotheticalprotein | G2/M-M-G1 |
| 246 | Bstt32-180 | putativeprotein | G2-M-G1 |
| 247 | Bstt3-22-160 | PotatoEST/ | G1/S-S-G2 |
| | | hypotheticalprotein | |
| 248 | Bstt32-3-175 | unknown | G2/M-M |
| 249 | Bstt32-3-325 | unknown protein | G2/M-M-G1 |
| 250 | Bstt3-24-135 | unknown | G2/M-M-G1 |
| 251 | Bstt3-24-200 | | G2/M-M-G1 |
| 252 | Bstt3-31-215 | unknownprotein | G2/M-M-G1 |
| 253 | Bstt3-31-330 | unknown | G1/S-S-G2 |
| 254 | Bstt33-1-350 | unknown | G2/M-M-G1 |
| 255 | Bstt33-1-510 | putativeprotein | G2/M-M-G1 |
| 256 | Bstt33-3-220 | unknown | G2/M-M-G1 |
| 257 | Bstt33-3-245 | unknownprotein | G2/M-M-G1 |
| 258 | Bstt3-33-550 | hypotheticalprotein | G1/S-S; M-G1 |
| 259 | Bstt33-4-140 | putativeprotein | S-G2 |
| 260 | Bstt34-2-165 | unknown | G1/S-S-G2 |
| 261 | Bstt3-42-325 | hypotheticalprotein | G2/M-M-G1 |
| 262 | Bstt3-44-150 | unknown | G2/M-M-G1 |
| 263 | Bstt3-44-250 | unknown | G2/M-M-G1 |
| 264 | Bstt34-4-310 | unknown | G2/M-M-G1 |
| 265 | Bstt3-44-345 | hypotheticalprotein | G2/M-M-G1 |
| 266 | Bstt41-2-340 | | G2/M-M-G1 |
| 267 | Bstt41-3-310 | unknown | G2/M-M |
| 268 | Bstt4-21-185 | | M-G1 |
| 269 | Bstt42-1-370 | | S-G2-G2/M |
| 270 | Bstt4-23-480 | unknown | G2/M-M-G1 |
| 271 | Bstt4-24-170 | | G2/M-M-G1 |
| 272 | Bstt43-265 | unknown | G1/S-S-G2/M |
| 273 | Bstt43-3-350 | unknown | G2/M-M-G1 |
| 274 | Bstt4-33-390 | hypotheticalprotein | G1/S-S; G2/M-M-G1 |
| 275 | Bstt4-34-280 | | G2/M-M-G1 |
| 276 | Bstt43-4-300 | unknownprotein | G2/M-M-G1 |
| 277 | Bstt43-4-330 | unknownprotein | G2/M-M-G1 |
| 278 | Bstt43-4-340 | | G2/M-M-G1 |
| 279 | Bstt44-4-250 | hypotheticalprotein | G2/M-M |
| 280 | Bstt4-44-400 | hypotheticalprotein | G2/M-M-G1 |
| 281 | MBc03-90 | unknown | S-G2 |
| 282 | MBc42-180 | unknown | G2-M-G1 |
| 283 | MBc43-210 | unknown | G1/S-S-G2 |

TABLE 5
overview group 4 sequences showing no homology to known genes

| SEQ | | | |
|---|---|---|---|
| ID NO | Tag name | Function | Fase |
| 284 | Bstc1 1-100 | unknown | G2/S-G2-M |
| 285 | Bstc1 -11-110 | unknown | S |
| 286 | Bstc1 -11-115 | unknown | G1/S-S; G2/M-M-G1 |
| 287 | Bstc1 -11-120 | | G1/S-S-G2 |
| 288 | Bstc1 1-1-125 | unknown | G2/M-M-G1 |
| 289 | Bstc1 1-1-290 | NaD | G1/S; G2/M-M-G1 |
| 290 | Bstc1 -12-155 | | G2/S-G2-M |
| 291 | Bstc1 -12-175 | unknown | S |
| 292 | Bstc1 -12-185 | unknown | G2/M-M-G1 |
| 293 | Bstc1 1-3-116 | unknown | S-G2 |
| 294 | Bstc1 1-3-118 | unknown | G2/M-M-G1 |
| 295 | Bstc1 -13-120 | | S |
| 296 | Bstc1 -13-130 | | G1/S-S; G2/M-M-G1 |
| 297 | Bstc1 -13-132 | unknown | M-G1 |
| 298 | Bstc1 -13-142 | unknown | G1/S-S |
| 299 | Bstc 11-3-187 | unknown | S-G2/S |
| 300 | Bstc1 1-3-200 | unknown | G1/S-S-G2/S |
| 301 | Bstc1 1-3-290 | unknown | G2/S-G2-M-G1 |
| 302 | Bstc1 -14-100 | unknown | G2/M-M |
| 303 | Bstc1 -14-108 | unknown | G2/M-M-G1 |
| 304 | Bstc1 1-4-130 | unknown | G1/S-S-G2 |
| 305 | Bstc1 1-4-135 | unknown | G2/M-M-G1 |
| 306 | Bstc1 1-4-140 | unknown | S-G2-M |
| 307 | Bstc1 -14-155 | | G2/M-M |
| 308 | Bstc1 -14-165 | | G2-G2/M |
| 309 | Bstc1 -14-167 | | G2-G2/M |
| 310 | Bstc1 1-4-175 | | G2/M-M-G1 |
| 311 | Bstc1 1-4-200 | unknown | G1/S-S |
| 312 | Bstc1 2-1-110 | unknown | S-G2 |
| 313 | Bstc1 -21-150 | unknown | G2/M-M-G1 |
| 314 | Bstc1 2-1-160 | unknown | G2-M-G1 |
| 315 | Bstc1 2-1-240 | unknown | M-G1 |
| 316 | Bstc1 2-1-95 | unknown | G1/S-S-G2 |
| 317 | Bstc1 -22-110 | | G2-M-G1 |
| 318 | Bstc1 2-3-103 | unknown | G2/M-M-G1 |
| 319 | Bstc1 2-3-125 | unknown | G1/S-S; G1 |
| 320 | Bstc1 2-3-235 | | M-G1 |
| 321 | Bstc1 2-3-237 | unknown | G1/S-S |
| 322 | Bstc1 2-4-130 | unknown | G2/M-M-G1 |
| 323 | Bstc1 2-4-133 | unknown | S-G2 |
| 324 | Bstc1 2-4-145 | unknown | M-G1 |
| 325 | Bstc1 2-4-235 | unknown | G2/M-M-G1 |
| 326 | Bstc1 3-1-150 | | M-G1 |
| 327 | Bstc1 3-2-170 | unknown | G2/M-M-G1 |
| 328 | Bstc1 3-2-180 | unknown | G1/S-S |
| 329 | Bstc1 3-2-190 | unknown | G1/S-S |
| 330 | Bstc1 3-2-280 | unknown | G1/S-S; G2/M-M-G1 |
| 331 | Bstc1 -41-170 | unknown | G1/S-S |
| 332 | Bstc1 -41-175 | unknown | G1/S-S |
| 333 | Bstc1 -41-180 | unknown | G1/S-S; G2/M-M-G1 |
| 334 | Bstc1 -41-210 | unknown | G1/S-S |
| 335 | Bstc1 -41-230 | | G1/S; G2/M-M-G1 |
| 336 | Bstc1 4-2-140 | unknown | M-G1 |
| 337 | Bstc1 -42-150 | unknown | G2/S-G2 |
| 338 | Bstc1 -42-80 | unknown | G1/S-S-G2 |
| 339 | Bstc1 -42-90 | unknown | G2-M |
| 340 | Bstc1 -43-105 | | G2/M-M |
| 341 | Bstc1 4-3-105 | | G1/S-S; G2/M-M |
| 342 | Bstc1 -43-110 | | G1/S-S; G2-M |
| 343 | Bstc1 4-3-130 | unknown | G2/M-M-G1 |
| 344 | Bstc1 -43-140 | unknown | S-G2 |
| 345 | Bstc1 -43-150 | | G2/M-M-G1 |
| 346 | Bstc1 -43-175 | | S-G2 |
| 347 | Bstc1 -43-185 | unknown | G1/S-S-G2/S |
| 348 | Bstc1 4-3-235 | unknown | G1/S-S |
| 349 | Bstc1 4-3-260 | unknown | G2/M-M-G1 |
| 350 | Bstc1 -43-65 | unknown | G1/S-S-G2 |
| 351 | Bstc1 -43-75 | unknown | S-G2 |
| 352 | Bstc1 -44-138 | unknown | G1/S-S-G2/S |
| 353 | Bstc1 -44-140 | unknown | G2/S-G2-M |
| 354 | Bstc1 -44-157 | unknown | G2/S-G2 |
| 355 | Bstc1 4-95 | unknown | G2/M-M |
| 356 | Bstc2 1-1-100 | unknown | G2/M-M-G1 |
| 357 | Bstc2 1-1-140 | unknown | G1/S-S-G2 |
| 358 | Bstc2 1-1-145 | unknown | M-G1 |
| 359 | Bstc2 1-1-65 | unknown | G2-M-G1 |
| 360 | Bstc2 1-2-120 | | G2/M-M |
| 361 | Bstc2 1-2-215 | | G2/M-M |
| 362 | Bstc2 1-2-75 | | S-G2-M |
| 363 | Bstc2 -13-110 | | G1/S-S; G2/M-M |
| 364 | Bstc2 -14-100 | unknown | G2/M-M-G1 |
| 365 | Bstc2 1-4-120 | unknown | M-G1 |
| 366 | Bstc2 -14-125 | unknown | G2/M-M-G1 |
| 367 | Bstc2 1-4-130 | unknown | G2/M-M-G1 |
| 368 | Bstc2 -14-135 | unknown | S-G2/S |
| 369 | Bstc2 1-4-135 | | S-G2 |
| 370 | Bstc2 1-4-155 | unknown | G2/M-M-G1 |
| 371 | Bstc2 -14-160 | | M-G1 |
| 372 | Bstc2 1-4-180 | unknown | G2/S-G2 |
| 373 | Bstc2 2-100 | unknown | G2-M |
| 374 | Bstc2 -21-120 | unknown | G1/S-S |
| 375 | Bstc2 2-1-125 | unknown | S-G2 |
| 376 | Bstc2 -21-170 | unknown | M-G1 |
| 377 | B stc22-1-98 | unknown | S-G2-G2/M |
| 378 | Bstc2 2-2-110 | unknown | G2/M-M-G1 |
| 379 | Bstc2 -22-160 | unknown | G1/S-S; G2-G2/M |
| 380 | Bstc2 2-2-165 | unknown | G1/S-S |
| 381 | Bstc2 -22-90 | | S; G2-M |
| 382 | Bstc2 -23-110 | unknown | G2/M-M |
| 383 | Bstc2 -23-140 | | M-G1 |
| 384 | Bstc2 2-3-150 | | S-G2 |
| 385 | B stc2-23-175 | | M-G1 |
| 386 | Bstc2 -23-195 | unknown | M-G1 |
| 387 | Bstc2 2-3-90 | | M-G1 |
| 388 | Bstc2 -24-100 | unknown | G2/M-M-G1 |
| 389 | Bstc2 2-4-140 | | G1/S-S-G2-M |
| 390 | Bstc2 -24-165 | | G2/M-M |
| 391 | Bstc2 -24-170 | unknown | G1/S-S |
| 392 | Bstc2 -31-140 | unknown | G2/M-M-G1 |
| 393 | Bstc2 -31-160 | | M-G1 |
| 394 | Bstc2 -31-170 | unknown | M-G1 |
| 395 | Bstc2 3-2-135 | unknown | G2/M-M-G1 |
| 396 | Bstc2 -32-285 | | G2/M-M |
| 397 | Bstc2 3-2-360 | unknown | G1/S; G2/M-M-G1 |
| 398 | Bstc2 3-2-80 | unknown | G2/M-M |
| 399 | Bstc2 3-3-175 | unknown | G1/S-S-G2 |
| 400 | Bstc2 -33-200 | unknown | G2/M-M-G1 |
| 401 | Bs tc23-3-305 | unknown | M-G1 |
| 402 | Bstc2 -33-85 | | S-G2 |
| 403 | Bstc2 -33-95 | unknown | G2/M-M-G1 |
| 404 | Bstc2 3-4-110 | unknown | G2-M |
| 405 | Bstc2 3-4-120 | unknown | G1/S-S-G2 |
| 406 | Bstc2 3-4-310 | | S-G2 |
| 407 | Bstc2 3-4-335 | | G2-M-G1 |
| 408 | Bstc2 -41-110 | unknown | S-G2 |
| 409 | Bstc2 4-2-165 | | M-G1 |
| 410 | Bstc2 -43-105 | unknown | S-G2-G2/M |
| 411 | Bstc2 -43-130 | unknown | G2/M-M |
| 412 | Bstc2 4-3-285 | | G1 |
| 413 | Bstc2 -43-77 | unknown | G2/M-M-G1 |
| 414 | Bstc2 -43-90 | unknown | G2/M-M-G1 |
| 415 | Bstc2 4-4-125 | unknown | G1/S-S |
| 416 | Bstc2 -44-175 | unknown | G2/M-M-G1 |
| 417 | Bstc2 4-4-220 | | G2/M-M-G1 |
| 418 | Bstc2 4-4-230 | | G2-G2/M |
| 419 | Bstc2 -44-95 | unknown | M-G1 |
| 420 | Bstc3 1-110 | unknown | G1/S-S |
| 421 | Bstc3 1-1-250 | | G2/M-M |
| 422 | Bstc3 1-1-77 | | M-G1 |
| 423 | Bstc3 1-1-90 | unknown | M-G1 |
| 424 | Bstc3 -12-115 | unknown | M-G1 |
| 425 | Bstc3 1-2-190 | unknown | G1/S-S-G2 |
| 426 | Bstc3 1-3-127 | unknown | G1/S-S-G2/M |
| 427 | Bstc3 1-3-235 | unknown | S-G2 |
| 428 | Bstc3 -13-330 | | G1 |
| 429 | Bstc3 1-3-60 | unknown | G2-M |
| 430 | Bstc3 1-3-80 | unknown | S-G2-M-G1 |
| 431 | Bstc3 -13-90 | unknown | G2/M-M-G1 |
| 432 | Bstc3 -13-95 | unknown | M-G1 |
| 433 | Bstc3 -14-105 | unknown | M-G1 |
| 434 | Bstc3 -14-110 | unknown | M-G1 |
| 435 | Bstc3 -14-125 | unknown | G2/M-M-G1 |
| 436 | Bstc3 -14-130 | unknown | G1/S; M-G1 |
| 437 | Bstc3 2-1-108 | unknown | G1/S-S-G2 |
| 438 | Bstc3 2-1-170 | unknown | S-G2/S |
| 439 | Bstc3 -21-70 | unknown | M-G1 |
| 440 | Bstc3 2-2-100 | unknown | G1/S-S-G2 |
| 441 | Bstc3 2-2-270 | unknown | G1/S; G2/M-M-G1 |
| 442 | Bstc3 2-2-390 | unknown | G2/M-M-G1 |
| 443 | Bstc3 2-2-93 | unknown | G2/M-M |
| 444 | Bstc3 2-3-100 | unknown | S-G2 |
| 445 | Bstc3 -23-125 | unknown | G2/M-M-G1 |
| 446 | Bstc3 2-3-155 | | S-G2-M |
| 447 | Bstc3 -23-175 | unknown | G2/M-M-G1 |
| 448 | Bstc3 -23-177 | | G2/S-G2-M-G1 |
| 449 | Bstc3 2-3-63 | unknown | S-G2 |
| 450 | Bstc3 -23-65 | | S; G2-M-G1 |
| 451 | Bstc3 -24-155 | unknown | G2/M-M-G1 |
| 452 | Bstc3 2-4-230 | unknown | G2/M-M |
| 453 | Bstc3 2-4-250 | unknown | G2/M-M-G1 |
| 454 | Bstc3 -24-255 | unknown | G2/M-M-G1 |
| 455 | Bstc3 -24-305 | | G2-M-G1 |
| 456 | Bstc3 -24-340 | unknown | G1/S-S; M-G1 |
| 457 | Bstc3 -24-90 | | M-G1 |
| 458 | Bstc3 -31-130 | unknown | G1/S-S-G2 |
| 459 | Bstc3 3-120 | unknown | G1/S-S |
| 460 | Bstc3 -31-200 | | S-G2 |
| 461 | Bstc3 -31-260 | unknown | G1/S-S |
| 462 | Bstc3 3-150 | unknown | G2/M-M-G1 |
| 463 | Bstc3 -32-105 | unknown | G2-G2/M |
| 464 | Bstc3 -32-120 | | G1/S-S; G2/M-M-G1 |
| 465 | Bstc3 -32-240 | unknown | S-G2 |
| 466 | Bstc3 -32-320 | | G1/S-S-G2; M-G1 |
| 467 | Bstc3 3-280 | unknown | G2-M-G1 |
| 468 | Bstc3 3-2-90 | unknown | S-G2 |
| 469 | Bstc3 3-3-105 | unknown | G2/M-M-G1 |
| 470 | Bstc3 3-3-115 | | G1/S-S; M-G1 |
| 471 | Bstc3 3-3-165 | | G1/S-S-G2/S |
| 472 | Bstc3 -34-110 | | G2/M-M |
| 473 | Bstc3 3-4-165 | | G2/M-M |
| 474 | Bstc3 3-4-200 | | S |
| 475 | Bstc3 -34-290 | unknown | G2/M-M-G1 |
| 476 | Bstc3 -34-85 | unknown | G2-M-G1 |
| 477 | Bstc3 -34-90 | unknown | G1/S-S |
| 478 | Bstc3 3-90 | unknown | S |
| 479 | Bstc3 4-115 | | G2-M-G1 |
| 480 | Bstc3 -41-180 | | G2/M-M-G1 |
| 481 | Bstc3 4-13-300 | unknown | G/S-S; M-G1 |
| 482 | Bstc3 4-3-100 | | M-G1 |
| 483 | Bstc3 4-3-135 | | S-G2-G2/M |
| 484 | Bstc3 4-3-190 | | S-G2-M-G1 |
| 485 | Bstc3 -43-210 | unknown | G1/S-S; M-G1 |
| 486 | Bstc3 4-3-210 | unknown | G2/S-G2-G2-G2/M |
| 487 | Bstc3 -43-240 | | G1/S-S; G2/M-M-G1 |
| 488 | Bstc3 4-3-248 | unknown | S |
| 489 | Bstc3 4-3-263 | unknown | G2/M-M-G1 |
| 490 | Bstc3 -43-280 | unknown | G2/M-M-G1 |
| 491 | Bstc3 4-3-95 | unknown | S |
| 492 | Bstc3 -44-155 | unknown | G1/S-S; M-G1 |
| 493 | Bstc3 -44-173 | | G2/M-M-G1 |
| 494 | Bstc3 4-80 | unknown | S-G2/S |
| 495 | Bstc4 -11-117 | | G2/M-M-G1 |
| 496 | Bstc4 1-1-125 | unknown | M-G1 |
| 497 | Bstc4 1-1-130 | unknown | G2-M-G1 |
| 498 | Bstc4 -11-180 | | G2/M-M-G1 |
| 499 | Bstc4 1-1-195 | unknown | G1/S-S-G2 |
| 500 | Bstc4 1-1-197 | unknown | G2/M-M-G1 |
| 501 | Bstc4 -11-210 | unknown | G1/S-S-G2/S |
| 502 | Bstc4 1-1-210 | unknown | G1/S-S-G1/S |
| 503 | Bstc4 1-1-245 | unknown | M-G1 |
| 504 | Bstc4 -11-350 | unknown | G2/M-M |
| 505 | Bstc4 1-1-90 | unknown | G2/M-M-G1 |
| 506 | Bstc4 -12-150 | unknown | G2-M-G1 |
| 507 | Bstc4 1-2-280 | | S-G2-M |
| 508 | Bstc4 -13-112 | unknown | S-G2 |
| 509 | Bstc4 1-3-170 | unknown | G1/S-S |
| 510 | Bstc4 1-3-205 | unknown | G2/M-M-G1 |
| 511 | Bstc4 -13-280 | unknown | G1/S-S-G2/S |
| 512 | Bstc4 -13-70 | unknown | G2/M-M-G1 |
| 513 | Bstc4 1-4-105 | | M-G1 |
| 514 | Bstc4 1-4-112 | unknown | G2/M-M |
| 515 | Bstc4 -14-120 | unknown | G1/S-S; M-G1 |
| 516 | Bstc4 1-4-127 | unknown | S-G2-M |
| 517 | Bstc4 1-4-145 | unknown | G2/M-M-G1 |
| 518 | Bstc4 -14-160 | unknown | G2/M-M-G1 |
| 519 | Bstc4 1-4-165 | unknown | G2-M-G1 |
| 520 | Bstc4 1-4-185 | | G1/S-S-G2 |
| 521 | Bstc4 1-4-270 | | G1/S-S; G2/M-M-G1 |
| 522 | Bstc4 2-1-150 | unknown | G2/M-M-G1 |
| 523 | Bstc4 -21-155 | | G1/S-S-G2 |
| 524 | Bstc4 -21-200 | unknown | S; G2/M-M-G1 |
| 525 | Bstc4 2-135 | unknown | G2/M-M-G1 |
| 526 | Bstc4 -22-150 | unknown | G1/S-S; G1 |
| 527 | Bstc 42-2-170 | | S-G2-M |
| 528 | Bstc4 2-2-185 | | M-G1 |
| 529 | Bstc4 2-2-220 | unknown | M-G1 |
| 530 | Bstc4 2-3-100 | unknown | M-G1 |
| 531 | Bstc4 -23-115 | unknown | M-G1 |
| 532 | Bstc4 2-3-133 | | S-G2/S |
| 533 | Bstc4 -23-135 | unknown | G2/M-M-G1 |
| 534 | Bstc4 2-4-110 | unknown | G1/S-S; G2/M-M-G1 |
| 535 | Bstc4 -24-240 | | G1/S-S-G2 |
| 536 | Bstc4 -31-260 | | G2/M-M-G1 |
| 537 | Bstc4 -31-310 | unknown | S; G2/M-M-G1 |
| 538 | Bstc4 3-3-100 | | S-G2-M |
| 539 | Bstc4 3-3-103 | unknown | G2/M-M-G1 |
| 540 | Bstc4 3-3-135 | | M-G1 |
| 541 | Bstc4 3-3-175 | | G2/M-M-G1 |
| 542 | Bstc4 3-3-250 | unknown | M-G1 |
| 543 | Bstc4 -34-135 | unknown | G2/M-M-G1 |
| 544 | Bstc4 -34-185 | | G1/S-S |
| 545 | Bstc4 3-4-200 | unknown | G2/M-M-G1 |
| 546 | Bstc4 3-4-320 | | G1/S-S |
| 547 | Bstc4 -41-100 | unknown | G2-M |
| 548 | Bstc4 -41-105 | unknown | G1/S-S; G2/M-M-G1 |
| 549 | Bstc4 -41-107 | unknown | G2/M-M-G1 |
| 550 | Bstc4 -41-125 | unknown | M-G1 |
| 551 | Bstc 4-41-180 | | G2/M-M-G1 |
| 552 | Bstc4 -41-220 | unknown | M-G1 |
| 553 | Bstc4 4-150 | unknown | G2-M-G1 |
| 554 | Bstc4 -42-110 | unknown | G2/M-M-G1 |
| 555 | Bstc4 -42-115 | unknown | G2/M-M |
| 556 | Bstc4 -42-130 | unknown | S-G2 |
| 557 | Bstc4 -42-165 | unknown | G1/S-S; M-G1 |
| 558 | Bstc4 -42-217 | unknown | G2/M-M-G1 |
| 559 | Bstc4 -43-103 | unknown | G1/S-S-G2-G2/M |
| 560 | Bstc4 4-3-167 | unknown | G2/M-M-G1 |
| 561 | Bstc4 4-3-170 | | M-G1 |
| 562 | Bstc4 4-4-120 | unknown | M-G1 |
| 563 | Bstc4 4-4-290 | unknown | G2/M-M-G1 |
| 564 | Bstt1 -11-190 | | G1/S-S |
| 565 | Bstt1 -11-200 | unknown | G1/S-S-G2-G2/M |
| 566 | Bstt1 -11-55 | unknown | G1/S-S |
| 567 | Bstt1 -11-65 | unknown | G1/S-S-G2 |
| 568 | Bstt1 -12-105 | unknown | G2/M-M |
| 569 | Bstt1 -12-115 | | G1/S-S |
| 570 | Bstt1 -12-230 | | S-G2 |
| 571 | Bstt1 -13-150 | unknown | G2/M-M |
| 572 | Bstt1 -13-230 | unknown | G2/S-G2-M |
| 573 | Bstt1 -14-125 | unknown | G1/S-S |
| 574 | Bstt1 -14-220 | unknown | G2/M-M |
| 575 | Bstt1 -21-100 | unknown | G2/M-M |
| 576 | Bstt12 -1-240 | unknown | S-G2-M |
| 577 | Bstt1 -21-250 | unknown | S; G2/M-M-G1 |
| 578 | Bstt12 -2-100 | unknown | G2/S-G2-M-G1 |
| 579 | Bstt12 -2-140 | unknown | G2/M-M-G1 |
| 580 | Bstt1 -22-160 | | G2/M-G1 |
| 581 | Bstt12 -2-215 | unknown | G2/M-M |
| 582 | Bstt1 -22-225 | | M-G1-G1/S |
| 583 | Bstt12 -2-360 | unknown | G2/M-M-G1 |
| 584 | Bstt1 -22-70 | unknown | G1/S-S |
| 585 | Bstt12 -3-115 | unknown | G1/S-S-G2 |
| 586 | Bstt1 -23-150 | unknown | G2-M-G1 |
| 587 | Bstt1 -23-170 | unknown | G2-M |
| 588 | Bstt12 -3-170 | unknown | G1/S-S |
| 589 | Bstt1 -23-180 | unknown | G2/S-G2-M |
| 590 | Bstt1 -23-185 | | G2-M-G1 |
| 591 | Bstt1 -23-235 | unknown | G2-M |
| 592 | Bstt1 -24-105 | unknown | G2/S-G2-M-G1 |
| 593 | Bstt1 -24-120 | unknown | G2/M-M-G1 |
| 594 | Bstt12 -4-260 | | G2/S-G2-G2/M |
| 595 | Bstt12 -4-320 | | G2/M-M |
| 596 | Bstt1 -31-120 | | G2/M-M-G1 |
| 597 | Bstt1 -31-180 | unknown | G2/M-M-G1 |
| 598 | Bstt13 -170 | unknown | G1/S-S-G2 |
| 599 | Bstt13 -2-150 | | G1/S-S-G2 |
| 600 | Bstt1 -32-170 | unknown | G1/S-S-G2 |
| 601 | Bstt1 -32-185 | | G1/S-S |
| 602 | Bstt13 -3-100 | unknown | G1/S-S-G2-M |
| 603 | Bstt1 -33-170 | unknown | G1/S-S-G2 |
| 604 | Bstt13 -3-320 | unknown | G2/M-M-G1 |
| 605 | Bstt1 -33-66 | | G2/M-M |
| 606 | Bstt1 -41-120 | unknown | G2/M-M |
| 607 | Bstt1 -42-264 | unknown | G2-M-G1 |
| 608 | Bstt14 -2-280 | unknown | G2/M-M-G1 |
| 609 | Bstt14 -3-120 | | S-G2 |
| 610 | Bstt14 -3-140 | unknown | G1-S-S-G2 |
| 611 | Bstt1 -43-220 | unknown | G2/S-G2-G2/M |
| 612 | Bstt1 -43-330 | unknown | G2/M-M-G1 |
| 613 | Bstt14 -3-460 | unknown | G2/M-M |
| 614 | Bstt14 -4-130 | unknown | S-G2 |
| 615 | Bstt14 -4-150 | unknown | G2 |
| 616 | Bstt14 -4-195 | | S-G2-M |
| 617 | Bstt14 -4-220 | | G2/S-G2-G2/M |
| 618 | Bstt14 -85 | nohits | G2/M-M |
| 619 | Bstt21 -1-170 | unknown | G2/M-M |
| 620 | Bstt2 -11-290 | | G2/S-G2-G2/M |
| 621 | Bstt2 -11-540 | | G1/S-S |
| 622 | Bstt21 -2-190 | | G2/M-M-G1 |
| 623 | Bstt2 -13-165 | | S-G2-M |
| 624 | Bstt2 -13-170 | unknown | G2/M-M |
| 625 | Bstt2 -14-130 | unknown | G2/M-M |
| 626 | Bstt2 -14-175 | unknown | S-G2 |
| 627 | Bstt22 -1-140 | unknown | S-G2 |
| 628 | Bstt2 -21-300 | unknown | G2/M-M |
| 629 | Bstt22 -2-110 | unknown | G1/S-G2 |
| 630 | Bstt22 -2-255 | | G1/S-S-G2-G2/M |
| 631 | Bstt22 -2-370 | | G1/S-G2 |
| 632 | Bstt22 -3-100 | unknown | G2/M-M-G1 |
| 633 | Bstt22 -3-145 | unknown | G2/M-M-G1 |
| 634 | Bstt2 -23-220 | unknown | G2-M-G1 |
| 635 | Bstt2 -23-370 | | G1/S-G2 |
| 636 | Bstt22 -4-145 | unknown | G2/M-M |
| 637 | Bstt22 -4-170 | | S-G2 |
| 638 | Bstt22 -4-175 | | G2-M |
| 639 | Bstt22 -80 | unknown | G2/M-M |
| 640 | Bstt23 -1-128 | unknown | S-G2 |
| 641 | Bstt23 -1-155 | unknown | S-G2-G2/M |
| 642 | Bstt2 -31-200 | unknown | G2/S-G2 |
| 643 | Bstt23 -170 | unknown | G2/M-M-G1 |
| 644 | Bstt2 -32-175 | unknown | G2/S-G2-G2/M |
| 645 | Bs tt23-220 | | G1/S-S-G2 |
| 646 | Bstt23 -3-200 | | G1/S-S-G2/S |
| 647 | Bstt23 -3-265 | | S-G2-G2/M |
| 648 | Bstt23 -3-330 | | G1/S-S |
| 649 | Bstt2 -34-170 | unknown | G2/M-M-G1 |
| 650 | Bstt23 -4-180 | | S-G2-M |
| 651 | Bstt23 -4-210 | | G2/M-M-G1 |
| 652 | Bstt2 -41-170 | unknown | G1/S-S-G2 |
| 653 | Bstt24 -1-170 | unknown | S-G2 |
| 654 | Bstt2 -41-390 | | S-G2 |
| 655 | Bstt2 -42-300 | | G2/M-M-G1 |
| 656 | Bstt24 -2-318 | | S-G2 |
| 657 | Bstt24 -2-320 | unknown | G2/M-M-G1 |
| 658 | Bstt24 -290 | unknown | G2/M-M |
| 659 | Bstt2 -43-150 | | S-G2 |
| 660 | Bstt2 -43-160 | | S-G2/S |
| 661 | Bstt2 -43-50 | | S |
| 662 | Bstt2 -43-65 | unknown | S-G2 |
| 663 | Bstt2 -44-230 | | G2/S-G2-M |
| 664 | Bstt2 -44-240 | unknown | G1/S-S-G2 |
| 665 | Bstt24 -4-240 | unknown | G1/S-S-G2/S |
| 666 | Bstt24 -4-260 | unknown | G1/S-S |
| 667 | Bstt24 -4-283 | unknown | G1/S-S-G2 |
| 668 | Bstt24 -4-285 | unknown | G2/M-M-G1 |
| 669 | Bstt31 -1-145 | | S-G2-M |
| 670 | Bstt31 -1-210 | | G2/M-M-G1 |
| 671 | Bstt31 -2-165 | unknown | G2/S-G2 |
| 672 | Bstt31 -2-185 | | G2/M-M-G1 |
| 673 | Bstt3 -12-200 | unknown | G2/M-M-G1 |
| 674 | Bstt3 -12-315 | | S-G2-M |
| 675 | Bstt31 -2-330 | | G2/M-M-G1 |
| 676 | Bstt3 -13-110 | unknown | S-G2-G2/M |
| 677 | Bstt31 -3-180 | | S-G2-G2/M |
| 678 | Bstt3 -13-360 | | G2/M-M |
| 679 | Bstt3 -14-130 | unknown | G2/M-M |
| 680 | Bstt3 -14-135 | unknown | G2/M-M |
| 681 | Bstt31 -50 | unknown | G1/S-S-G2-G2/M |
| 682 | Bstt32 -1-105 | | S-G2 |
| 683 | Bstt3 -21-165 | | G2/S-G2 |
| 684 | Bstt3 -21-305 | unknown | G2/M-M |
| 685 | Bstt32 -140 | unknown | S-G2/S |
| 686 | Bstt3 -22-100 | | G2/M-M-G1 |
| 687 | Bstt32 -2-210 | | S-G2-M |
| 688 | Bstt3 -22-280 | unknown | G1/S-S; M-G1 |
| 689 | Bstt32 -2-510 | unknown | S-G2-G2/M |
| 690 | Bstt32 -3-115 | | G2/S-G2 |
| 691 | Bstt32 -3-155 | unknown | S-G2 |
| 692 | Bstt32 -3-160 | | M |
| 693 | Bstt32 -3-180 | unknown | G1/S-S-G2 |
| 694 | Bstt3 -23-205 | unknown | S-G2-M |
| 695 | Bstt3 -23-65 | unknown | G2/M-M-G1 |
| 696 | Bstt32 -4-170 | unknown | S; M |
| 697 | Bstt32 -4-195 | | G1/S-S; G2/M-M-G1 |
| 698 | Bstt32 -4-260 | unknown | G1/S-S |
| 699 | Bstt3 -24-390 | | M-G1 |
| 700 | Bstt33 -1-105 | | G1/S-S-G2 |
| 701 | Bstt33 -1-128 | | S-G2 |
| 702 | Bstt33 -1-132 | unknown | G2/M-M |
| 703 | Bstt33 -1-160 | unknown | G2/M-M-G1 |
| 704 | Bstt33 -1-185 | | M-G1 |
| 705 | Bstt33 -140 | unknown | G2/M-M-G1 |
| 706 | Bstt33 -2-75 | unknown | G1/S-S-G2 |
| 707 | Bstt33 -2-85 | | G1/S-S; G2/M-G1 |
| 708 | Bstt33 -3-110 | | G1/S-S; G2/M-M-G1 |
| 709 | Bstt33 -3-125 | unknown | G2/M-M-G1 |
| 710 | Bstt3 -33-170 | unknown | S-G2/S |
| 711 | Bstt33 -4-110 | | S-G2 |
| 712 | Bstt33 -4-120 | unknown | G1/S-S-G2 |
| 713 | Bstt33 -4-130 | unknown | G2/M-M |
| 714 | Bstt33 -95 | unknown | G2/M-M |
| 715 | Bstt34 -1-110 | | S-G2-G2/M |
| 716 | Bstt34 -1-170 | | G1/S-S-G2-G2/M |
| 717 | Bstt3 -42-350 | unknown | G2/M-M-G1 |
| 718 | Bstt3 -43-145 | unknown | G2/M-M-G1 |
| 719 | Bstt3 -43-190 | unknown | G1/S-S; M-G1 |
| 720 | Bstt3 -43-265 | | G2/S-G2-M-G1 |
| 721 | Bstt3 -43-280 | unknown | G2/M-M-G1 |
| 722 | Bstt34 -70 | unknown | S |
| 723 | Bstt41 -3-100b | unknown | G2/M-M |
| 724 | Bstt41 -3-130 | unknown | G2/M-M-G1 |
| 725 | Bstt41 -3-140 | unknown | G2/M-M-G1 |
| 726 | Bstt41 -3-180 | | G2-M |
| 727 | Bstt41 -3-230 | unknown | S-G2 |
| 728 | Bstt41 -3-90 | unknown | G2/M-M-G1 |
| 729 | Bstt41 -4-210 | unknown | S-G2-M-G1 |
| 730 | Bstt4 -14-500 | | G2/M-M-G1 |
| 731 | Bstt41 -70 | unknown | G1/S-S |
| 732 | Bstt42 -1-130 | unknown | G2/M-M-G1 |
| 733 | Bstt42 -1-290 | unknown | G2/M-M |
| 734 | Bstt4 -21-60 | unknown | S-G2 |
| 735 | Bstt4 -22-100 | | M-G1 |
| 736 | Bstt4 -22-360 | | S-G2 |
| 737 | Bstt42 -3-105 | unknown | G1/S-S-G2/S |
| 738 | Bstt42 -3-110 | unknown | G2/M-M-G1 |
| 739 | Bstt4 -23-130 | | S-G2/M |
| 740 | Bstt4 -23-160 | | G2/S-G2-M |
| 741 | Bstt42 -4-150 | unknown | G1/S-S-G2 |
| 742 | Bstt4 -24-270 | unknown | G2/M-M-G1 |
| 743 | Bstt42 -4-390 | unknown | M-G1 |
| 744 | Bstt43 -1-290 | unknown | G2/M-M-G1 |
| 745 | Bstt43 -1-85 | | G1/S-S-G2/S |
| 746 | Bstt4 -32-230 | unknown | G1/S-S-G2/S |
| 747 | Bstt43 -2-238 | | G2/M |
| 748 | Bstt43 -3-145 | unknown | G1/S-S-G2 |
| 749 | Bstt43 -3-210 | | G2/M-M-G1 |
| 750 | Bstt43 -4-230 | unknown | G2/M-M-G1 |
| 751 | Bstt4 -34-75 | unknown | G2/S-G2-M |
| 752 | Bstt44 -1-125 | unknown | S-G2-G2/M |
| 753 | Bstt44 -185 | unknown | M-G1 |
| 754 | Bstt44 -2-135 | | G2/M-M-G1 |
| 755 | Bstt4 -42-150 | unknown | M |
| 756 | Bstt4 -42-390 | unknown | M-G1 |
| 757 | Bstt44 -3-240 | unknown | G2/M-M-G1 |
| 758 | Bstt44 -3-250 | unknown | S-G2-G2/M |
| 759 | Bstt4 -44-148 | | G2/M-M-G1 |
| 760 | M Bc02-100 | unknown | G2/M-M |
| 761 | M Bc02-120 | unknown | G2/M-M |
| 762 | M Bc03-110 | unknown | G2/M-M |
| 763 | M Bc03-85 | | G2/M-M |
| 764 | M Bc11-135 | unknown | G2-M |
| 765 | M Bc12-150 | | S-G2-M |
| 766 | M Bc31-185 | unknown | G2/M-M |
| 767 | M Bc32-107 | unknown | G2/M-M-G1 |
| 768 | M Bc32-110 | unknown | G2/M-M-G1 |
| 769 | M Bc41-110 | unknown | G1/S-S; G2/M-M |
| 770 | M Bc42-280 | unknown | G2-M |
| 771 | M Bc43-95 | unknown | G2-M |
| 772 | M Bc44-130 | | S-G2 |
| 773 | M Bc44-95 | unknown | G2/M-M |
| 774 | M Bt12-80 | unknown | G2/M-M |
| 775 | M Bt12-95 | | M |
| 776 | M Bt13-105 | unknown | M-G1 |
| 777 | M Bt14-100 | unknown | G2/M-M-G1 |
| 778 | M Bt14-85 | unknown | S-G2-M |
| 779 | M Bt14-90 | unknown | G2-M |
| 780 | M Bt31-95 | | S-G2-M |
| 781 | M Bt33-115 | | G2/M-M-G1 |
| 782 | M Bt33-133 | | G2-M |
| 783 | M Bt42-135 | unknown | G2-M |
| 784 | M Bt43-95 | unknown | G2-G2/M |
| 785 | M Bt44-145 | unknown | G1/S-S-G2-M |

1-16. (canceled)

17. An isolated cDNA consisting essentially of SEQ ID NO 61.

18. An isolated nucleic acid sequence consisting essentially of SEQ ID NO 61.

19. A method for the production of an agrochemical resistant plant, comprising the use of SEQ ID NO 61 by transforming a plant with SEQ ID NO 61 and regenerating a transformed plant thereafter.

20. A plant tolerant to an agrochemical, in which the expression level of the nucleic acid corresponding to SEQ ID NO 61 is modulated.
